SEQUENCE LISTING (252 sequences)

SEQ ID NO: 1 - protein, 13 aa, Artificial Sequence
  Description of Artificial Sequence Synthetic peptide
SGSSSNIGNNAVN

SEQ ID NO: 2 - protein, 7 aa, Artificial Sequence
  Description of Artificial Sequence Synthetic peptide
YDDLLPS

SEQ ID NO: 3 - protein, 11 aa, Artificial Sequence
  Description of Artificial Sequence Synthetic peptide
AAWDDSLNGPV

SEQ ID NO: 4 - protein, 7 aa, Artificial Sequence
  Description of Artificial Sequence Synthetic peptide
GFTFSSY

SEQ ID NO: 5 - protein, 6 aa, Artificial Sequence
  Description of Artificial Sequence Synthetic peptide
RYDGSN

SEQ ID NO: 6 - protein, 12 aa, Artificial Sequence
  Description of Artificial Sequence Synthetic peptide
DRGLGDGTYFDY

SEQ ID NO: 7 - protein, 110 aa, Artificial Sequence
  Description of Artificial Sequence Synthetic polypeptide
QSALTQPASVSGSPGQSITISCSGSSSNIGNNAVNWYQQLPGKAPKLLIYYDDLLPSGVSDRFSGSKSGTSAFLAISGLQSEDEADYYCAAWDDSLNGPVFGGGTKLTVL

SEQ ID NO: 8 - protein, 121 aa, Artificial Sequence
  Description of Artificial Sequence Synthetic polypeptide
QVQLVESGGGLVKPGGSLRLSCAASGFTFSSYGMHWVRQAPGKGLEWVAFIRYDGSNKYYADSVKGRFTISRDNSKNTLYLQMNSLRAEDTAVYYCAKDRGLGDGTYFDYWGQGTTVTVSS

SEQ ID NO: 9 - protein, 15 aa, Artificial Sequence
  Description of Artificial Sequence Synthetic peptide
GGGGSGGGGSGGGGS

SEQ ID NO: 10 - protein, 11 aa, Artificial Sequence
  Description of Artificial Sequence Synthetic peptide
KASQNVATHVG

SEQ ID NO: 11 - protein, 7 aa, Artificial Sequence
  Description of Artificial Sequence Synthetic peptide
SASYRYS

SEQ ID NO: 12 - protein, 9 aa, Artificial Sequence
  Description of Artificial Sequence Synthetic peptide
QQYNRYPYT

SEQ ID NO: 13 - protein, 7 aa, Artificial Sequence
  Description of Artificial Sequence Synthetic peptide
GYSFTGY

SEQ ID NO: 14 - protein, 6 aa, Artificial Sequence
  Description of Artificial Sequence Synthetic peptide
NPYNSD

SEQ ID NO: 15 - protein, 9 aa, Artificial Sequence
  Description of Artificial Sequence Synthetic peptide
VALRVALDY

SEQ ID NO: 16 - protein, 107 aa, Artificial Sequence
  Description of Artificial Sequence Synthetic polypeptide
DIQMTQSPSSLSASVGDRVTITCKASQNVATHVGWYQQKPGKAPKRLIYSASYRYSGVPSRFSGSGSGTEFTLTISNLQPEDFATYYCQQYNRYPYTFGQGTKLEIK

SEQ ID NO: 17 - protein, 118 aa, Artificial Sequence
  Description of Artificial Sequence Synthetic polypeptide
QVQLVQSGAEVKKPGASVKVSCKASGYSFTGYTMNWVRQAPGQGLEWMGLINPYNSDTNYAQKLQGRVTMTTDTSTSTAYMELRSLRSDDTAVYYCARVALRVALDYWGQGTLVTVSS

SEQ ID NO: 18 - protein, 229 aa, Artificial Sequence
  Description of Artificial Sequence Synthetic polypeptide
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 19 - protein, 229 aa, Artificial Sequence
  Description of Artificial Sequence Synthetic polypeptide
ESKYGPPCPPCPAPEAAGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 20 - protein, 20 aa, Artificial Sequence
  Description of Artificial Sequence Synthetic peptide
MYRMQLLSCIALSLALVTNS

SEQ ID NO: 21 - protein, 18 aa, Artificial Sequence
  Description of Artificial Sequence Synthetic peptide
MGWSSIILFLVATATGVH

SEQ ID NO: 22 - protein, 9 aa, Artificial Sequence
  Description of Artificial Sequence Synthetic peptide
YPYDVPDYA

SEQ ID NO: 23 - protein, 15 aa, Artificial Sequence
  Description of Artificial Sequence Synthetic peptide
RASESVTILGSHLIH

SEQ ID NO: 24 - protein, 11 aa, Artificial Sequence
  Description of Artificial Sequence Synthetic peptide
SASQDISNYLN

SEQ ID NO: 25 - protein, 15 aa, Artificial Sequence
  Description of Artificial Sequence Synthetic peptide
RASESVTILGSHLIY

SEQ ID NO: 26 - protein, 7 aa, Artificial Sequence
  Description of Artificial Sequence Synthetic peptide
LASNVQT

SEQ ID NO: 27 - protein, 7 aa, Artificial Sequence
  Description of Artificial Sequence Synthetic peptide
YTSNLHS

SEQ ID NO: 28 - protein, 7 aa, Artificial Sequence
  Description of Artificial Sequence Synthetic peptide
LASNVQT

SEQ ID NO: 29 - protein, 9 aa, Artificial Sequence
  Description of Artificial Sequence Synthetic peptide
LQSRTIPRT

SEQ ID NO: 30 - protein, 9 aa, Artificial Sequence
  Description of Artificial Sequence Synthetic peptide
QQYRKLPWT

SEQ ID NO: 31 - protein, 9 aa, Artificial Sequence
  Description of Artificial Sequence Synthetic peptide
LQSRTIPRT

SEQ ID NO: 32 - protein, 7 aa, Artificial Sequence
  Description of Artificial Sequence Synthetic peptide
GYTFTDY

SEQ ID NO: 33 - protein, 7 aa, Artificial Sequence
  Description of Artificial Sequence Synthetic peptide
GGTFSNY

SEQ ID NO: 34 - protein, 7 aa, Artificial Sequence
  Description of Artificial Sequence Synthetic peptide
GYTFRHY

SEQ ID NO: 35 - protein, 7 aa, Artificial Sequence
  Description of Artificial Sequence Synthetic peptide
INTETRE

SEQ ID NO: 36 - protein, 6 aa, Artificial Sequence
  Description of Artificial Sequence Synthetic peptide
YRGHSD

SEQ ID NO: 37 - protein, 6 aa, Artificial Sequence
  Description of Artificial Sequence Synthetic peptide
NTESGV

SEQ ID NO: 38 - protein, 8 aa, Artificial Sequence
  Description of Artificial Sequence Synthetic peptide
DYSYAMDY

SEQ ID NO: 39 - protein, 12 aa, Artificial Sequence
  Description of Artificial Sequence Synthetic peptide
GAIYNGYDVLDN

SEQ ID NO: 40 - protein, 8 aa, Artificial Sequence
  Description of Artificial Sequence Synthetic peptide
DYLYSLDF

SEQ ID NO: 41 - protein, 111 aa, Artificial Sequence
  Description of Artificial Sequence Synthetic polypeptide
DIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIK

SEQ ID NO: 42 - protein, 108 aa, Artificial Sequence
  Description of Artificial Sequence Synthetic polypeptide
DIQMTQSPSSLSASVGDRVTITCSASQDISNYLNWYQQKPGKAPKLLIYYTSNLHSGVPSRFSGSGSGTDFTLTISSLQPEDFATYYCQQYRKLPWTFGQGTKLEIKR

SEQ ID NO: 43 - protein, 111 aa, Artificial Sequence
  Description of Artificial Sequence Synthetic polypeptide
DIVLTQSPPSLAMSLGKRATISCRASESVTILGSHLIYWYQQKPGQPPTLLIQLASNVQTGVPARFSGSGSRTDFTLTIDPVEEDDVAVYYCLQSRTIPRTFGGGTKLEIK

SEQ ID NO: 44 - protein, 117 aa, Artificial Sequence
  Description of Artificial Sequence Synthetic polypeptide
QIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 45 - protein, 121 aa, Artificial Sequence
  Description of Artificial Sequence Synthetic polypeptide
QVQLVQSGAEVKKPGSSVKVSCKASGGTFSNYWMHWVRQAPGQGLEWMGATYRGHSDTYYNQKFKGRVTITADKSTSTAYMELSSLRSEDTAVYYCARGAIYNGYDVLDNWGQGTLVTVSS

SEQ ID NO: 46 - protein, 117 aa, Artificial Sequence
  Description of Artificial Sequence Synthetic polypeptide
QIQLVQSGPELKKPGETVKISCKASGYTFRHYSMNWVKQAPGKGLKWMGRINTESGVPIYADDFKGRFAFSVETSASTAYLVINNLKDEDTASYFCSNDYLYSLDFWGQGTALTVSS

SEQ ID NO: 47 - protein, 21 aa, Artificial Sequence
  Description of Artificial Sequence Synthetic peptide
LEPKSCDKTHTCPPCPDPKGT

SEQ ID NO: 48 - protein, 68 aa, Artificial Sequence
  Description of Artificial Sequence Synthetic polypeptide
FWVLVVVGGVLACYSLLVTVAFIIFWVRSKRSRLLHSDYMNMTPRRPGPTRKHYQPYAPPRDFAAYRS

SEQ ID NO: 49 - protein, 112 aa, Artificial Sequence
  Description of Artificial Sequence Synthetic polypeptide
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 50 - protein, 23 aa, Artificial Sequence
  Description of Artificial Sequence Synthetic peptide
HVGSGEGRGSLLTCGDVEENPGP

SEQ ID NO: 51 - protein, 1251 aa, Artificial Sequence
  Description of Artificial Sequence Synthetic polypeptide
ATMGWSSIILFLVATATGVHQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSGGGGSGGGGSGGGGSDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKLEPKSCDKTHTCPPCPDPKGTFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRHVGSGEGRGSLLTCGDVEENPGPMYRMQLLSCIALSLALVTNSDIQMTQSPSSLSASVGDRVTITCKASQNVATHVGWYQQKPGKAPKRLIYSASYRYSGVPSRFSGSGSGTEFTLTISNLQPEDFATYYCQQYNRYPYTFGQGTKLEIKGGGGSGGGGSGGGGSQVQLVQSGAEVKKPGASVKVSCKASGYSFTGYTMNWVRQAPGQGLEWMGLINPYNSDTNYAQKLQGRVTMTTDTSTSTAYMELRSLRSDDTAVYYCARVALRVALDYWGQGTLVTVSSPSGQAGAAASESLFVSNHAYQSALTQPASVSGSPGQSITISCSGSSSNIGNNAVNWYQQLPGKAPKLLIYYDDLLPSGVSDRFSGSKSGTSAFLAISGLQSEDEADYYCAAWDDSLNGPVFGGGTKLTVLGGGGSGGGGSGGGGSQVQLVESGGGLVKPGGSLRLSCAASGFTFSSYGMHWVRQAPGKGLEWVAFIRYDGSNKYYADSVKGRFTISRDNSKNTLYLQMNSLRAEDTAVYYCAKDRGLGDGTYFDYWGQGTTVTVSSESKYGPPCPPCPAPEAAGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKYPYDVPDYA

SEQ ID NO: 52 - protein, 1251 aa, Artificial Sequence
  Description of Artificial Sequence Synthetic polypeptide
ATMGWSSIILFLVATATGVHQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSGGGGSGGGGSGGGGSDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKLEPKSCDKTHTCPPCPDPKGTFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRHVGSGEGRGSLLTCGDVEENPGPMYRMQLLSCIALSLALVTNSQVQLVESGGGLVKPGGSLRLSCAASGFTFSSYGMHWVRQAPGKGLEWVAFIRYDGSNKYYADSVKGRFTISRDNSKNTLYLQMNSLRAEDTAVYYCAKDRGLGDGTYFDYWGQGTTVTVSSGGGGSGGGGSGGGGSQSALTQPASVSGSPGQSITISCSGSSSNIGNNAVNWYQQLPGKAPKLLIYYDDLLPSGVSDRFSGSKSGTSAFLAISGLQSEDEADYYCAAWDDSLNGPVFGGGTKLTVLPSGQAGAAASESLFVSNHAYQVQLVQSGAEVKKPGASVKVSCKASGYSFTGYTMNWVRQAPGQGLEWMGLINPYNSDTNYAQKLQGRVTMTTDTSTSTAYMELRSLRSDDTAVYYCARVALRVALDYWGQGTLVTVSSGGGGSGGGGSGGGGSDIQMTQSPSSLSASVGDRVTITCKASQNVATHVGWYQQKPGKAPKRLIYSASYRYSGVPSRFSGSGSGTEFTLTISNLQPEDFATYYCQQYNRYPYTFGQGTKLEIKESKYGPPCPPCPAPEAAGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKYPYDVPDYA

SEQ ID NO: 53 - protein, 1252 aa, Artificial Sequence
  Description of Artificial Sequence Synthetic polypeptide
ATMGWSSIILFLVATATGVHDIQMTQSPSSLSASVGDRVTITCSASQDISNYLNWYQQKPGKAPKLLIYYTSNLHSGVPSRFSGSGSGTDFTLTISSLQPEDFATYYCQQYRKLPWTFGQGTKLEIKRGGGGSGGGGSGGGGSQVQLVQSGAEVKKPGSSVKVSCKASGGTFSNYWMHWVRQAPGQGLEWMGATYRGHSDTYYNQKFKGRVTITADKSTSTAYMELSSLRSEDTAVYYCARGAIYNGYDVLDNWGQGTLVTVSSLEPKSCDKTHTCPPCPDPKGTFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRHVGSGEGRGSLLTCGDVEENPGPMYRMQLLSCIALSLALVTNSDIQMTQSPSSLSASVGDRVTITCKASQNVATHVGWYQQKPGKAPKRLIYSASYRYSGVPSRFSGSGSGTEFTLTISNLQPEDFATYYCQQYNRYPYTFGQGTKLEIKGGGGSGGGGSGGGGSQVQLVQSGAEVKKPGASVKVSCKASGYSFTGYTMNWVRQAPGQGLEWMGLINPYNSDTNYAQKLQGRVTMTTDTSTSTAYMELRSLRSDDTAVYYCARVALRVALDYWGQGTLVTVSSPSGQAGAAASESLFVSNHAYQSALTQPASVSGSPGQSITISCSGSSSNIGNNAVNWYQQLPGKAPKLLIYYDDLLPSGVSDRFSGSKSGTSAFLAISGLQSEDEADYYCAAWDDSLNGPVFGGGTKLTVLGGGGSGGGGSGGGGSQVQLVESGGGLVKPGGSLRLSCAASGFTFSSYGMHWVRQAPGKGLEWVAFIRYDGSNKYYADSVKGRFTISRDNSKNTLYLQMNSLRAEDTAVYYCAKDRGLGDGTYFDYWGQGTTVTVSSESKYGPPCPPCPAPEAAGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKYPYDVPDYA

SEQ ID NO: 54 - protein, 1252 aa, Artificial Sequence
  Description of Artificial Sequence Synthetic polypeptide
ATMGWSSIILFLVATATGVHDIQMTQSPSSLSASVGDRVTITCSASQDISNYLNWYQQKPGKAPKLLIYYTSNLHSGVPSRFSGSGSGTDFTLTISSLQPEDFATYYCQQYRKLPWTFGQGTKLEIKRGGGGSGGGGSGGGGSQVQLVQSGAEVKKPGSSVKVSCKASGGTFSNYWMHWVRQAPGQGLEWMGATYRGHSDTYYNQKFKGRVTITADKSTSTAYMELSSLRSEDTAVYYCARGAIYNGYDVLDNWGQGTLVTVSSLEPKSCDKTHTCPPCPDPKGTFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRHVGSGEGRGSLLTCGDVEENPGPMYRMQLLSCIALSLALVTNSQVQLVESGGGLVKPGGSLRLSCAASGFTFSSYGMHWVRQAPGKGLEWVAFIRYDGSNKYYADSVKGRFTISRDNSKNTLYLQMNSLRAEDTAVYYCAKDRGLGDGTYFDYWGQGTTVTVSSGGGGSGGGGSGGGGSQSALTQPASVSGSPGQSITISCSGSSSNIGNNAVNWYQQLPGKAPKLLIYYDDLLPSGVSDRFSGSKSGTSAFLAISGLQSEDEADYYCAAWDDSLNGPVFGGGTKLTVLPSGQAGAAASESLFVSNHAYQVQLVQSGAEVKKPGASVKVSCKASGYSFTGYTMNWVRQAPGQGLEWMGLINPYNSDTNYAQKLQGRVTMTTDTSTSTAYMELRSLRSDDTAVYYCARVALRVALDYWGQGTLVTVSSGGGGSGGGGSGGGGSDIQMTQSPSSLSASVGDRVTITCKASQNVATHVGWYQQKPGKAPKRLIYSASYRYSGVPSRFSGSGSGTEFTLTISNLQPEDFATYYCQQYNRYPYTFGQGTKLEIKESKYGPPCPPCPAPEAAGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKYPYDVPDYA

SEQ ID NO: 55 - DNA, 54 bp, Artificial Sequence
  Description of Artificial Sequence Synthetic oligonucleotide
atggggtggtcaagcattattctgtttctggtcgctaccgctacaggcgtccat

SEQ ID NO: 56 - DNA, 60 bp, Artificial Sequence
  Description of Artificial Sequence Synthetic oligonucleotide
atgtacaggatgcaactcctgtcttgcattgcactaagtcttgcacttgtcacaaacagt

SEQ ID NO: 57 - DNA, 45 bp, Artificial Sequence
  Description of Artificial Sequence Synthetic oligonucleotide
ggtgggggcggctctggtggcggtggcagcggcggaggtggcagt

SEQ ID NO: 58 - DNA, 333 bp, Artificial Sequence
  Description of Artificial Sequence Synthetic polynucleotide
gatattgttcttactcaatcacccccaagccttgcgatgtctcttggtaaacgagcgacaattagttgtagagcttctgaaagcgtaactattcttgggtcacatcttattcattggtatcaacaaaagccgggacaaccgcctacactcttgattcaactcgcgagcaatgttcaaacgggtgtccctgcacgcttttctgggagcggttcacgaacagattttactctcacgattgatccagtcgaagaagatgatgtcgctgtatattattgtctccaaagtaggacaataccaagaacttttggtggtggtacaaaattggaaattaaa

SEQ ID NO: 59 - DNA, 324 bp, Artificial Sequence
  Description of Artificial Sequence Synthetic polynucleotide
gacatccagatgacccagagccctagctcactgagcgccagcgtgggcgacagggtgaccattacctgctccgccagccaggacatcagcaactacctgaactggtaccagcagaagcccggcaaggcccccaagctgctgatctactacacctccaacctgcactccggcgtgcccagcaggttcagcggaagcggcagcggcaccgatttcaccctgaccatctccagcctgcagcccgaggacttcgccacctactactgccagcagtacaggaagctcccctggactttcggccagggcaccaaactggagatcaagcgt

SEQ ID NO: 60 - DNA, 333 bp, Artificial Sequence
  Description of Artificial Sequence Synthetic polynucleotide
gacatcgtgctgacccagagcccccccagcctggccatgtctctgggcaagagagccaccatcagctgccgggccagcgagagcgtgaccatcctgggcagccacctgatctactggtatcagcagaagcctggccagccccccaccctgctgatccagctggctagcaatgtgcagaccggcgtgcccgccagattcagcggcagcggcagcagaaccgacttcaccctgaccatcgaccccgtggaagaggacgacgtggccgtgtactactgcctgcagagccggaccatcccccggacctttggcggaggaacaaagctggaaatcaag

SEQ ID NO: 61 - DNA, 333 bp, Artificial Sequence
  Description of Artificial Sequence Synthetic polynucleotide
gacatcgtgctgacccagagcccccccagcctggccatgtctctgggcaagagagccaccatcagctgccgggccagcgagagcgtgaccatcctgggcagccacctgatccactggtatcagcagaagcccggccagccccccaccctgctgatccagctcgccagcaatgtgcagaccggcgtgcccgccagattcagcggcagcggcagcagaaccgacttcaccctgaccatcgaccccgtggaagaggacgacgtggccgtgtactactgcctgcagagccggaccatcccccggacctttggcggaggcaccaaactggaaatcaag

SEQ ID NO: 62 - DNA, 351 bp, Artificial Sequence
  Description of Artificial Sequence Synthetic polynucleotide
cagatccagctggtgcagtccggccccgagctgaagaagcccggcgagaccgtgaagatctcctgcaaggcctccggctacaccttcaccgactactccatcaactgggtgaagcgggcccccggcaagggcctgaagtggatgggctggatcaacaccgagacccgggagcccgcctacgcctacgacttccggggccggttcgccttctccctggagacctccgcctccaccgcctacctgcagatcaacaacctgaagtacgaggacaccgccacctacttctgcgccctggactactcctacgccatggactactggggccagggcacctccgtgaccgtgtcctcc

SEQ ID NO: 63 - DNA, 363 bp, Artificial Sequence
  Description of Artificial Sequence Synthetic polynucleotide
caggtgcagctggtccagagcggcgccgaagtgaagaagcccggcagctccgtgaaagtgagctgcaaggccagcggcggcaccttcagcaactactggatgcactgggtgaggcaggcccccggacagggcctggagtggatgggcgccacctacaggggccacagcgacacctactacaaccagaagttcaagggccgggtgaccatcaccgccgacaagagcaccagcaccgcctacatggaactgagcagcctcaggagcgaggacaccgctgtgtattactgcgccaggggcgccatctacaacggctacgacgtgctggacaactggggccagggcacactagtgaccgtgtccagc

SEQ ID NO: 64 - DNA, 405 bp, Artificial Sequence
  Description of Artificial Sequence Synthetic polynucleotide
atgggatggagctctatcatcctcttcttggtagcaacagctacaggtgtccaccagattcagctggtgcagagcggccctgagctgaagaaacccggcgagacagtgaagatcagctgcaaggcctccggctacaccttccggcactacagcatgaactgggtgaaacaggcccctggcaagggcctgaagtggatgggccggatcaacaccgagagcggcgtgcccatctacgccgacgacttcaagggcagattcgccttcagcgtggaaaccagcgccagcaccgcctacctggtgatcaacaacctgaaggacgaggataccgccagctacttctgcagcaacgactacctgtacagcctggacttctggggccagggcaccgccctgaccgtgtccagc

SEQ ID NO: 65 - DNA, 351 bp, Artificial Sequence
  Description of Artificial Sequence Synthetic polynucleotide
cagattcagctggtgcagagcggccctgagctgaagaaacccggcgagacagtgaagatcagctgcaaggcctccggctacaccttcaccgactacagcatcaactgggtgaaaagagcccctggcaagggcctgaagtggatgggctggatcaacaccgagacaagagagcccgcctacgcctacgacttccggggcagattcgccttcagcctggaaaccagcgccagcaccgcctacctgcagatcaacaacctgaagtacgaggacaccgccacctacttttgcgccctggactacagctacgccatggactactggggccagggcaccagcgtgaccgtgtccagc

SEQ ID NO: 66 - DNA, 63 bp, Artificial Sequence
  Description of Artificial Sequence Synthetic oligonucleotide
ctcgagcccaaatcttgtgacaaaactcacacatgcccaccgtgcccggatcccaaaggtacc

SEQ ID NO: 67 - DNA, 204 bp, Artificial Sequence
  Description of Artificial Sequence Synthetic polynucleotide
ttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcc

SEQ ID NO: 68 - DNA, 336 bp, Artificial Sequence
  Description of Artificial Sequence Synthetic polynucleotide
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 69 - DNA, 69 bp, Artificial Sequence
  Description of Artificial Sequence Synthetic oligonucleotide
cacgtgggttctggagaaggacgcggttccttgttgacgtgtggcgatgtagaggaaaatccgggtcca

SEQ ID NO: 70 - DNA, 60 bp, Artificial Sequence
  Description of Artificial Sequence Synthetic oligonucleotide
ccgagcggccaggcgggcgcggcggcatcggagtccctgtttgtgtcaaatcacgcctac

SEQ ID NO: 71 - DNA, 321 bp, Artificial Sequence
  Description of Artificial Sequence Synthetic polynucleotide
gacatccagatgacccagagccccagcagcctgagcgccagcgtgggcgaccgggtgaccatcacctgcaaggccagccagaacgtggccacccacgtgggctggtaccagcagaagcccggcaaggcccccaagcggctgatctacagcgccagctaccggtacagcggcgtgcccagccggttcagcggcagcggcagcggcaccgagttcaccctgaccatcagcaacctgcagcccgaggacttcgccacctactactgccagcagtacaaccggtacccctacaccttcggccagggcaccaagctggagatcaag

SEQ ID NO: 72 - DNA, 354 bp, Artificial Sequence
  Description of Artificial Sequence Synthetic polynucleotide
caggtgcagctggtgcagagcggcgccgaggtgaagaagcccggcgccagcgtgaaggtgagctgcaaggccagcggctacagcttcaccggctacaccatgaactgggtgcggcaggcccccggccagggcctggagtggatgggcctgatcaacccctacaacagcgacaccaactacgcccagaagctgcagggccgggtgaccatgaccaccgacaccagcaccagcaccgcctacatggagctgcggagcctgcggagcgacgacaccgccgtgtactactgcgcccgggtggccctgcgggtggccctggactactggggccagggcaccctggtgaccgtgagcagc

SEQ ID NO: 73 - DNA, 330 bp, Artificial Sequence
  Description of Artificial Sequence Synthetic polynucleotide
cagtcagcgcttacgcagccggcgtcggtgtcgggttccccgggtcagtcgatcacgatcagctgtagtgggagcagctccaacatcggtaacaacgcagtgaactggtatcagcaactgccgggaaaagcgccgaaactgctgatttactatgatgatttgctgccaagtggagttagtgaccgcttttccggcagtaaatcgggtacctcggcttttctggctatttcgggtctccagagcgaggatgaagctgattattattgcgccgcatgggatgatagcttaaatggcccagtttttggcggcggtactaaactgaccgtgctg

SEQ ID NO: 74 - DNA, 363 bp, Artificial Sequence
  Description of Artificial Sequence Synthetic polynucleotide
caagtgcagctggttgaatccggtggcggtctggtcaagccgggcggctctttgcgtctgagctgtgccgcgtcgggttttaccttcagctcttatggtatgcattgggtgcgtcaggcgcctggcaaaggtctggagtgggttgcgttcatccgctacgatgggtctaacaaatattatgccgactcagtaaaaggacgcttcactattagccgcgacaatagcaaaaataccctgtacctgcaaatgaatagcctgcgcgccgaagataccgccgtttactattgcgctaaagatcgtggcctgggtgatggtacgtacttcgattactggggtcagggcaccaccgttaccgttagttca

SEQ ID NO: 75 - DNA, 687 bp, Artificial Sequence
  Description of Artificial Sequence Synthetic polynucleotide
gaaagcaaatatggcccgccgtgcccgccgtgcccggcgccggaagcggcgggcggcccgagcgtgtttctgtttccgccgaaaccgaaagataccctgatgattagccgcaccccggaagtgacctgcgtggtggtggatgtgagccaggaagatccggaagtgcagtttaactggtatgtggatggcgtggaagtgcataacgcgaaaaccaaaccgcgcgaagaacagtttcagagcacctatcgcgtggtgagcgtgctgaccgtgctgcatcaggattggctgaacggcaaagaatataaatgcaaagtgagcaacaaaggcctgccgagcagcattgaaaaaaccattagcaaagcgaaaggccagccgcgcgaaccgcaggtgtataccctgccgccgagccaggaagaaatgaccaaaaaccaggtgagcctgacctgcctggtgaaaggcttttatccgagcgatattgcggtggaatgggaaagcaacggccagccggaaaacaactataaaaccaccccgccggtgctggatagcgatggcagcttttttctgtatagccgcctgaccgtggataaaagccgctggcaggaaggcaacgtgtttagctgcagcgtgatgcatgaagcgctgcataaccattatacccagaaaagcctgagcctgagcctgggcaaa

SEQ ID NO: 76 - DNA, 27 bp, Artificial Sequence
  Description of Artificial Sequence Synthetic oligonucleotide
tacccatacgatgttccagattacgct

SEQ ID NO: 77 - DNA, 3756 bp, Artificial Sequence
  Description of Artificial Sequence Synthetic polynucleotide
gccaccatggggtggtcaagcattattctgtttctggtcgctaccgctacaggcgtccatcagatccagctggtgcagtccggccccgagctgaagaagcccggcgagaccgtgaagatctcctgcaaggcctccggctacaccttcaccgactactccatcaactgggtgaagcgggcccccggcaagggcctgaagtggatgggctggatcaacaccgagacccgggagcccgcctacgcctacgacttccggggccggttcgccttctccctggagacctccgcctccaccgcctacctgcagatcaacaacctgaagtacgaggacaccgccacctacttctgcgccctggactactcctacgccatggactactggggccagggcacctccgtgaccgtgtcctccggtgggggcggctctggtggcggtggcagcggcggaggtggcagtgatattgttcttactcaatcacccccaagccttgcgatgtctcttggtaaacgagcgacaattagttgtagagcttctgaaagcgtaactattcttgggtcacatcttattcattggtatcaacaaaagccgggacaaccgcctacactcttgattcaactcgcgagcaatgttcaaacgggtgtccctgcacgcttttctgggagcggttcacgaacagattttactctcacgattgatccagtcgaagaagatgatgtcgctgtatattattgtctccaaagtaggacaataccaagaacttttggtggtggtacaaaattggaaattaaactcgagcccaaatcttgtgacaaaactcacacatgcccaccgtgcccggatcccaaaggtaccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgccacgtgggttctggagaaggacgcggttccttgttgacgtgtggcgatgtagaggaaaatccgggtccaatgtacaggatgcaactcctgtcttgcattgcactaagtcttgcacttgtcacaaacagtgacatccagatgacccagagccccagcagcctgagcgccagcgtgggcgaccgggtgaccatcacctgcaaggccagccagaacgtggccacccacgtgggctggtaccagcagaagcccggcaaggcccccaagcggctgatctacagcgccagctaccggtacagcggcgtgcccagccggttcagcggcagcggcagcggcaccgagttcaccctgaccatcagcaacctgcagcccgaggacttcgccacctactactgccagcagtacaaccggtacccctacaccttcggccagggcaccaagctggagatcaagggtgggggcggctctggtggcggtggcagcggcggaggtggcagtcaggtgcagctggtgcagagcggcgccgaggtgaagaagcccggcgccagcgtgaaggtgagctgcaaggccagcggctacagcttcaccggctacaccatgaactgggtgcggcaggcccccggccagggcctggagtggatgggcctgatcaacccctacaacagcgacaccaactacgcccagaagctgcagggccgggtgaccatgaccaccgacaccagcaccagcaccgcctacatggagctgcggagcctgcggagcgacgacaccgccgtgtactactgcgcccgggtggccctgcgggtggccctggactactggggccagggcaccctggtgaccgtgagcagcccgagcggccaggcgggcgcggcggcatcggagtccctgtttgtgtcaaatcacgcctaccagtcagcgcttacgcagccggcgtcggtgtcgggttccccgggtcagtcgatcacgatcagctgtagtgggagcagctccaacatcggtaacaacgcagtgaactggtatcagcaactgccgggaaaagcgccgaaactgctgatttactatgatgatttgctgccaagtggagttagtgaccgcttttccggcagtaaatcgggtacctcggcttttctggctatttcgggtctccagagcgaggatgaagctgattattattgcgccgcatgggatgatagcttaaatggcccagtttttggcggcggtactaaactgaccgtgctgggtgggggcggctctggtggcggtggcagcggcggaggtggcagtcaagtgcagctggttgaatccggtggcggtctggtcaagccgggcggctctttgcgtctgagctgtgccgcgtcgggttttaccttcagctcttatggtatgcattgggtgcgtcaggcgcctggcaaaggtctggagtgggttgcgttcatccgctacgatgggtctaacaaatattatgccgactcagtaaaaggacgcttcactattagccgcgacaatagcaaaaataccctgtacctgcaaatgaatagcctgcgcgccgaagataccgccgtttactattgcgctaaagatcgtggcctgggtgatggtacgtacttcgattactggggtcagggcaccaccgttaccgttagttcagaaagcaaatatggcccgccgtgcccgccgtgcccggcgccggaagcggcgggcggcccgagcgtgtttctgtttccgccgaaaccgaaagataccctgatgattagccgcaccccggaagtgacctgcgtggtggtggatgtgagccaggaagatccggaagtgcagtttaactggtatgtggatggcgtggaagtgcataacgcgaaaaccaaaccgcgcgaagaacagtttcagagcacctatcgcgtggtgagcgtgctgaccgtgctgcatcaggattggctgaacggcaaagaatataaatgcaaagtgagcaacaaaggcctgccgagcagcattgaaaaaaccattagcaaagcgaaaggccagccgcgcgaaccgcaggtgtataccctgccgccgagccaggaagaaatgaccaaaaaccaggtgagcctgacctgcctggtgaaaggcttttatccgagcgatattgcggtggaatgggaaagcaacggccagccggaaaacaactataaaaccaccccgccggtgctggatagcgatggcagcttttttctgtatagccgcctgaccgtggataaaagccgctggcaggaaggcaacgtgtttagctgcagcgtgatgcatgaagcgctgcataaccattatacccagaaaagcctgagcctgagcctgggcaaatacccatacgatgttccagattacgcttaa

SEQ ID NO: 78 - DNA, 3756 bp, Artificial Sequence
  Description of Artificial Sequence Synthetic polynucleotide
gccaccatggggtggtcaagcattattctgtttctggtcgctaccgctacaggcgtccatcagatccagctggtgcagtccggccccgagctgaagaagcccggcgagaccgtgaagatctcctgcaaggcctccggctacaccttcaccgactactccatcaactgggtgaagcgggcccccggcaagggcctgaagtggatgggctggatcaacaccgagacccgggagcccgcctacgcctacgacttccggggccggttcgccttctccctggagacctccgcctccaccgcctacctgcagatcaacaacctgaagtacgaggacaccgccacctacttctgcgccctggactactcctacgccatggactactggggccagggcacctccgtgaccgtgtcctccggtgggggcggctctggtggcggtggcagcggcggaggtggcagtgatattgttcttactcaatcacccccaagccttgcgatgtctcttggtaaacgagcgacaattagttgtagagcttctgaaagcgtaactattcttgggtcacatcttattcattggtatcaacaaaagccgggacaaccgcctacactcttgattcaactcgcgagcaatgttcaaacgggtgtccctgcacgcttttctgggagcggttcacgaacagattttactctcacgattgatccagtcgaagaagatgatgtcgctgtatattattgtctccaaagtaggacaataccaagaacttttggtggtggtacaaaattggaaattaaactcgagcccaaatcttgtgacaaaactcacacatgcccaccgtgcccggatcccaaaggtaccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgccacgtgggttctggagaaggacgcggttccttgttgacgtgtggcgatgtagaggaaaatccgggtccaatgtacaggatgcaactcctgtcttgcattgcactaagtcttgcacttgtcacaaacagtcaagtgcagctggttgaatccggtggcggtctggtcaagccgggcggctctttgcgtctgagctgtgccgcgtcgggttttaccttcagctcttatggtatgcattgggtgcgtcaggcgcctggcaaaggtctggagtgggttgcgttcatccgctacgatgggtctaacaaatattatgccgactcagtaaaaggacgcttcactattagccgcgacaatagcaaaaataccctgtacctgcaaatgaatagcctgcgcgccgaagataccgccgtttactattgcgctaaagatcgtggcctgggtgatggtacgtacttcgattactggggtcagggcaccaccgttaccgttagttcaggtgggggcggctctggtggcggtggcagcggcggaggtggcagtcagtcagcgcttacgcagccggcgtcggtgtcgggttccccgggtcagtcgatcacgatcagctgtagtgggagcagctccaacatcggtaacaacgcagtgaactggtatcagcaactgccgggaaaagcgccgaaactgctgatttactatgatgatttgctgccaagtggagttagtgaccgcttttccggcagtaaatcgggtacctcggcttttctggctatttcgggtctccagagcgaggatgaagctgattattattgcgccgcatgggatgatagcttaaatggcccagtttttggcggcggtactaaactgaccgtgctgccgagcggccaggcgggcgcggcggcatcggagtccctgtttgtgtcaaatcacgcctaccaggtgcagctggtgcagagcggcgccgaggtgaagaagcccggcgccagcgtgaaggtgagctgcaaggccagcggctacagcttcaccggctacaccatgaactgggtgcggcaggcccccggccagggcctggagtggatgggcctgatcaacccctacaacagcgacaccaactacgcccagaagctgcagggccgggtgaccatgaccaccgacaccagcaccagcaccgcctacatggagctgcggagcctgcggagcgacgacaccgccgtgtactactgcgcccgggtggccctgcgggtggccctggactactggggccagggcaccctggtgaccgtgagcagcggtgggggcggctctggtggcggtggcagcggcggaggtggcagtgacatccagatgacccagagccccagcagcctgagcgccagcgtgggcgaccgggtgaccatcacctgcaaggccagccagaacgtggccacccacgtgggctggtaccagcagaagcccggcaaggcccccaagcggctgatctacagcgccagctaccggtacagcggcgtgcccagccggttcagcggcagcggcagcggcaccgagttcaccctgaccatcagcaacctgcagcccgaggacttcgccacctactactgccagcagtacaaccggtacccctacaccttcggccagggcaccaagctggagatcaaggaaagcaaatatggcccgccgtgcccgccgtgcccggcgccggaagcggcgggcggcccgagcgtgtttctgtttccgccgaaaccgaaagataccctgatgattagccgcaccccggaagtgacctgcgtggtggtggatgtgagccaggaagatccggaagtgcagtttaactggtatgtggatggcgtggaagtgcataacgcgaaaaccaaaccgcgcgaagaacagtttcagagcacctatcgcgtggtgagcgtgctgaccgtgctgcatcaggattggctgaacggcaaagaatataaatgcaaagtgagcaacaaaggcctgccgagcagcattgaaaaaaccattagcaaagcgaaaggccagccgcgcgaaccgcaggtgtataccctgccgccgagccaggaagaaatgaccaaaaaccaggtgagcctgacctgcctggtgaaaggcttttatccgagcgatattgcggtggaatgggaaagcaacggccagccggaaaacaactataaaaccaccccgccggtgctggatagcgatggcagcttttttctgtatagccgcctgaccgtggataaaagccgctggcaggaaggcaacgtgtttagctgcagcgtgatgcatgaagcgctgcataaccattatacccagaaaagcctgagcctgagcctgggcaaatacccatacgatgttccagattacgcttaa

SEQ ID NO: 79 - DNA, 3759 bp, Artificial Sequence
  Description of Artificial Sequence Synthetic polynucleotide
gccaccatggggtggtcaagcattattctgtttctggtcgctaccgctacaggcgtccatgacatccagatgacccagagccctagctcactgagcgccagcgtgggcgacagggtgaccattacctgctccgccagccaggacatcagcaactacctgaactggtaccagcagaagcccggcaaggcccccaagctgctgatctactacacctccaacctgcactccggcgtgcccagcaggttcagcggaagcggcagcggcaccgatttcaccctgaccatctccagcctgcagcccgaggacttcgccacctactactgccagcagtacaggaagctcccctggactttcggccagggcaccaaactggagatcaagcgtggtgggggcggctctggtggcggtggcagcggcggaggtggcagtcaggtgcagctggtccagagcggcgccgaagtgaagaagcccggcagctccgtgaaagtgagctgcaaggccagcggcggcaccttcagcaactactggatgcactgggtgaggcaggcccccggacagggcctggagtggatgggcgccacctacaggggccacagcgacacctactacaaccagaagttcaagggccgggtgaccatcaccgccgacaagagcaccagcaccgcctacatggaactgagcagcctcaggagcgaggacaccgctgtgtattactgcgccaggggcgccatctacaacggctacgacgtgctggacaactggggccagggcacactagtgaccgtgtccagcctcgagcccaaatcttgtgacaaaactcacacatgcccaccgtgcccggatcccaaaggtaccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgccacgtgggttctggagaaggacgcggttccttgttgacgtgtggcgatgtagaggaaaatccgggtccaatgtacaggatgcaactcctgtcttgcattgcactaagtcttgcacttgtcacaaacagtgacatccagatgacccagagccccagcagcctgagcgccagcgtgggcgaccgggtgaccatcacctgcaaggccagccagaacgtggccacccacgtgggctggtaccagcagaagcccggcaaggcccccaagcggctgatctacagcgccagctaccggtacagcggcgtgcccagccggttcagcggcagcggcagcggcaccgagttcaccctgaccatcagcaacctgcagcccgaggacttcgccacctactactgccagcagtacaaccggtacccctacaccttcggccagggcaccaagctggagatcaagggtgggggcggctctggtggcggtggcagcggcggaggtggcagtcaggtgcagctggtgcagagcggcgccgaggtgaagaagcccggcgccagcgtgaaggtgagctgcaaggccagcggctacagcttcaccggctacaccatgaactgggtgcggcaggcccccggccagggcctggagtggatgggcctgatcaacccctacaacagcgacaccaactacgcccagaagctgcagggccgggtgaccatgaccaccgacaccagcaccagcaccgcctacatggagctgcggagcctgcggagcgacgacaccgccgtgtactactgcgcccgggtggccctgcgggtggccctggactactggggccagggcaccctggtgaccgtgagcagcccgagcggccaggcgggcgcggcggcatcggagtccctgtttgtgtcaaatcacgcctaccagtcagcgcttacgcagccggcgtcggtgtcgggttccccgggtcagtcgatcacgatcagctgtagtgggagcagctccaacatcggtaacaacgcagtgaactggtatcagcaactgccgggaaaagcgccgaaactgctgatttactatgatgatttgctgccaagtggagttagtgaccgcttttccggcagtaaatcgggtacctcggcttttctggctatttcgggtctccagagcgaggatgaagctgattattattgcgccgcatgggatgatagcttaaatggcccagtttttggcggcggtactaaactgaccgtgctgggtgggggcggctctggtggcggtggcagcggcggaggtggcagtcaagtgcagctggttgaatccggtggcggtctggtcaagccgggcggctctttgcgtctgagctgtgccgcgtcgggttttaccttcagctcttatggtatgcattgggtgcgtcaggcgcctggcaaaggtctggagtgggttgcgttcatccgctacgatgggtctaacaaatattatgccgactcagtaaaaggacgcttcactattagccgcgacaatagcaaaaataccctgtacctgcaaatgaatagcctgcgcgccgaagataccgccgtttactattgcgctaaagatcgtggcctgggtgatggtacgtacttcgattactggggtcagggcaccaccgttaccgttagttcagaaagcaaatatggcccgccgtgcccgccgtgcccggcgccggaagcggcgggcggcccgagcgtgtttctgtttccgccgaaaccgaaagataccctgatgattagccgcaccccggaagtgacctgcgtggtggtggatgtgagccaggaagatccggaagtgcagtttaactggtatgtggatggcgtggaagtgcataacgcgaaaaccaaaccgcgcgaagaacagtttcagagcacctatcgcgtggtgagcgtgctgaccgtgctgcatcaggattggctgaacggcaaagaatataaatgcaaagtgagcaacaaaggcctgccgagcagcattgaaaaaaccattagcaaagcgaaaggccagccgcgcgaaccgcaggtgtataccctgccgccgagccaggaagaaatgaccaaaaaccaggtgagcctgacctgcctggtgaaaggcttttatccgagcgatattgcggtggaatgggaaagcaacggccagccggaaaacaactataaaaccaccccgccggtgctggatagcgatggcagcttttttctgtatagccgcctgaccgtggataaaagccgctggcaggaaggcaacgtgtttagctgcagcgtgatgcatgaagcgctgcataaccattatacccagaaaagcctgagcctgagcctgggcaaatacccatacgatgttccagattacgcttaa

SEQ ID NO: 80 - DNA, 3759 bp, Artificial Sequence
  Description of Artificial Sequence Synthetic polynucleotide
gccaccatggggtggtcaagcattattctgtttctggtcgctaccgctacaggcgtccatgacatccagatgacccagagccctagctcactgagcgccagcgtgggcgacagggtgaccattacctgctccgccagccaggacatcagcaactacctgaactggtaccagcagaagcccggcaaggcccccaagctgctgatctactacacctccaacctgcactccggcgtgcccagcaggttcagcggaagcggcagcggcaccgatttcaccctgaccatctccagcctgcagcccgaggacttcgccacctactactgccagcagtacaggaagctcccctggactttcggccagggcaccaaactggagatcaagcgtggtgggggcggctctggtggcggtggcagcggcggaggtggcagtcaggtgcagctggtccagagcggcgccgaagtgaagaagcccggcagctccgtgaaagtgagctgcaaggccagcggcggcaccttcagcaactactggatgcactgggtgaggcaggcccccggacagggcctggagtggatgggcgccacctacaggggccacagcgacacctactacaaccagaagttcaagggccgggtgaccatcaccgccgacaagagcaccagcaccgcctacatggaactgagcagcctcaggagcgaggacaccgctgtgtattactgcgccaggggcgccatctacaacggctacgacgtgctggacaactggggccagggcacactagtgaccgtgtccagcctcgagcccaaatcttgtgacaaaactcacacatgcccaccgtgcccggatcccaaaggtaccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgccacgtgggttctggagaaggacgcggttccttgttgacgtgtggcgatgtagaggaaaatccgggtccaatgtacaggatgcaactcctgtcttgcattgcactaagtcttgcacttgtcacaaacagtcaagtgcagctggttgaatccggtggcggtctggtcaagccgggcggctctttgcgtctgagctgtgccgcgtcgggttttaccttcagctcttatggtatgcattgggtgcgtcaggcgcctggcaaaggtctggagtgggttgcgttcatccgctacgatgggtctaacaaatattatgccgactcagtaaaaggacgcttcactattagccgcgacaatagcaaaaataccctgtacctgcaaatgaatagcctgcgcgccgaagataccgccgtttactattgcgctaaagatcgtggcctgggtgatggtacgtacttcgattactggggtcagggcaccaccgttaccgttagttcaggtgggggcggctctggtggcggtggcagcggcggaggtggcagtcagtcagcgcttacgcagccggcgtcggtgtcgggttccccgggtcagtcgatcacgatcagctgtagtgggagcagctccaacatcggtaacaacgcagtgaactggtatcagcaactgccgggaaaagcgccgaaactgctgatttactatgatgatttgctgccaagtggagttagtgaccgcttttccggcagtaaatcgggtacctcggcttttctggctatttcgggtctccagagcgaggatgaagctgattattattgcgccgcatgggatgatagcttaaatggcccagtttttggcggcggtactaaactgaccgtgctgccgagcggccaggcgggcgcggcggcatcggagtccctgtttgtgtcaaatcacgcctaccaggtgcagctggtgcagagcggcgccgaggtgaagaagcccggcgccagcgtgaaggtgagctgcaaggccagcggctacagcttcaccggctacaccatgaactgggtgcggcaggcccccggccagggcctggagtggatgggcctgatcaacccctacaacagcgacaccaactacgcccagaagctgcagggccgggtgaccatgaccaccgacaccagcaccagcaccgcctacatggagctgcggagcctgcggagcgacgacaccgccgtgtactactgcgcccgggtggccctgcgggtggccctggactactggggccagggcaccctggtgaccgtgagcagcggtgggggcggctctggtggcggtggcagcggcggaggtggcagtgacatccagatgacccagagccccagcagcctgagcgccagcgtgggcgaccgggtgaccatcacctgcaaggccagccagaacgtggccacccacgtgggctggtaccagcagaagcccggcaaggcccccaagcggctgatctacagcgccagctaccggtacagcggcgtgcccagccggttcagcggcagcggcagcggcaccgagttcaccctgaccatcagcaacctgcagcccgaggacttcgccacctactactgccagcagtacaaccggtacccctacaccttcggccagggcaccaagctggagatcaaggaaagcaaatatggcccgccgtgcccgccgtgcccggcgccggaagcggcgggcggcccgagcgtgtttctgtttccgccgaaaccgaaagataccctgatgattagccgcaccccggaagtgacctgcgtggtggtggatgtgagccaggaagatccggaagtgcagtttaactggtatgtggatggcgtggaagtgcataacgcgaaaaccaaaccgcgcgaagaacagtttcagagcacctatcgcgtggtgagcgtgctgaccgtgctgcatcaggattggctgaacggcaaagaatataaatgcaaagtgagcaacaaaggcctgccgagcagcattgaaaaaaccattagcaaagcgaaaggccagccgcgcgaaccgcaggtgtataccctgccgccgagccaggaagaaatgaccaaaaaccaggtgagcctgacctgcctggtgaaaggcttttatccgagcgatattgcggtggaatgggaaagcaacggccagccggaaaacaactataaaaccaccccgccggtgctggatagcgatggcagcttttttctgtatagccgcctgaccgtggataaaagccgctggcaggaaggcaacgtgtttagctgcagcgtgatgcatgaagcgctgcataaccattatacccagaaaagcctgagcctgagcctgggcaaatacccatacgatgttccagattacgcttaa

SEQ ID NO: 81 - protein, 20 aa, Artificial Sequence
  Description of Artificial Sequence Synthetic peptide
PSGQAGAAASESLFVSNHAY

SEQ ID NO: 82 - DNA, 48 bp, Artificial Sequence
  Description of Artificial Sequence Synthetic oligonucleotide
ctcgagcccaaatcttgtgacaaaactcacacatgcccaccgtgcccg

SEQ ID NO: 83 - protein, 16 aa, Artificial Sequence
  Description of Artificial Sequence Synthetic peptide
LEPKSCDKTHTCPPCP

SEQ ID NO: 84 - protein, 51 aa, Homo sapiens
PAKPTTTPAPRPPTPAPTIASQPLSLRPEACRPAAGGAVHTRGLDFACDIY

SEQ ID NO: 85 - protein, 49 aa, Mus musculus
KVNSTTTKPVLRTPSPVHPTGTSQPQRPEDCRPRGSVKGTGLDFACDIY

SEQ ID NO: 86 - protein, 51 aa, Felis catus
PVKPTTTPAPRPPTQAPITTSQRVSLRPGTCQPSAGSTVEASGLDLSCDIY

SEQ ID NO: 87 - protein, 21 aa, Homo sapiens
IYIWAPLAGTCGVLLLSLVIT

SEQ ID NO: 88 - protein, 21 aa, Mus musculus
IWAPLAGICVALLLSLIITLI

SEQ ID NO: 89 - protein, 21 aa, Rattus sp.
IWAPLAGICAVLLLSLVITLI

SEQ ID NO: 90 - DNA, 81 bp, Unknown
  Description of Unknown CD28 transmembrane sequence
ttttgggtgctggtggtggttggtggagtcctggcttgctatagcttgctagtaacagtggcctttattattttctgggtg

SEQ ID NO: 91 - protein, 27 aa, Unknown
  Description of Unknown CD28 transmembrane sequence
FWVLVVVGGVLACYSLLVTVAFIIFWV

SEQ ID NO: 92 - protein, 42 aa, Artificial Sequence
  Description of Artificial Sequence Synthetic polypeptide
KRGRKKLLYIFKQPFMRPVQTTQEEDGCSCRFPEEEEGGCEL

SEQ ID NO: 93 - DNA, 126 bp, Artificial Sequence
  Description of Artificial Sequence Synthetic polynucleotide
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 94 - protein, 220 aa, Artificial Sequence
  Description of Artificial Sequence Synthetic polypeptide
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 95 - protein, 41 aa, Artificial Sequence
  Description of Artificial Sequence Synthetic polypeptide
RSKRSRLLHSDYMNMTPRRPGPTRKHYQPYAPPRDFAAYRS

SEQ ID NO: 96 - DNA, 123 bp, Artificial Sequence
  Description of Artificial Sequence Synthetic polynucleotide
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 97 - DNA, 105 bp, Artificial Sequence
  Description of Artificial Sequence Synthetic polynucleotide
acaaaaaagaagtattcatccagtgtgcacgaccctaacggtgaatacatgttcatgagagcagtgaacacagccaaaaaatccagactcacagatgtgacccta

SEQ ID NO: 98 - DNA, 108 bp, Artificial Sequence
  Description of Artificial Sequence Synthetic polynucleotide
agggaccagaggctgccccccgatgcccacaagccccctgggggaggcagtttccggacccccatccaagaggagcaggccgacgcccactccaccctggccaagatc

SEQ ID NO: 99 - protein, 20 aa, Artificial Sequence
  Description of Artificial Sequence Synthetic peptide
RPRRSPAQDGKVYINMPGRG

SEQ ID NO: 100 - protein, 21 aa, Artificial Sequence
  Description of Artificial Sequence Synthetic peptide
ESPYQELQGQRSDVYSDLNTQ

SEQ ID NO: 101 - protein, 112 aa, Artificial Sequence; identical to SEQ ID NO: 49
  Description of Artificial Sequence Synthetic polypeptide

SEQ ID NO: 102 - DNA, 339 bp, Artificial Sequence
  Description of Artificial Sequence Synthetic polynucleotide
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 103 - protein, 114 aa, Homo sapiens
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 104 - protein, 162 aa, Homo sapiens
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 105 - protein, 114 aa, Homo sapiens; identical to SEQ ID NO: 103

SEQ ID NO: 106 - protein, 133 aa, Homo sapiens
GIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 107 - protein, 65 aa, Homo sapiens
ITCPPPMSVEHADIWVKSYSLYSRERYICNSGFKRKAGTSSLTECVLNKATNVAHWTTPSLKCIR

SEQ ID NO: 108 - protein, 26 aa, Artificial Sequence
  Description of Artificial Sequence Synthetic peptide
SGGGSGGGGSGGGGSGGGGSGGGSLQ

SEQ ID NO: 109 - protein, 61 aa, Artificial Sequence
  Description of Artificial Sequence Synthetic polypeptide
  MOD_RES (3)..(3): P or A
  MOD_RES (5)..(5): M or V
  MOD_RES (13)..(13): W, R or Q
  MOD_RES (20)..(20): Y or H
  MOD_RES (26)..(26): I or V
  MOD_RES (51)..(51): V or A
  MOD_RES (53)..(53): V or A
CPXPXSVEHADIXVKSYSLXSRERYXCNSGFKRKAGTSSLTECVLNKATNXAXWTTPSLKC

SEQ ID NO: 110 - protein, 146 aa, Homo sapiens
MRSSPGNMERIVICLMVIFLGTLVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQK

SEQ ID NO: 111 - protein, 123 aa, Homo sapiens
VHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQK

SEQ ID NO: 112 - protein, 122 aa, Homo sapiens
HKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQK

SEQ ID NO: 113 - protein, 162 aa, Homo sapiens
MRSSPGNMERIVICLMVIFLGTLVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 114 - protein, 139 aa, Homo sapiens
VHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 115 - protein, 138 aa, Homo sapiens
HKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 116 - protein, 153 aa, Homo sapiens
MRSSPGNMERIVICLMVIFLGTLVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKVSTLSFI

SEQ ID NO: 117 - protein, 130 aa, Homo sapiens
VHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKVSTLSFI

SEQ ID NO: 118 - protein, 129 aa, Homo sapiens
HKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKVSTLSFI

SEQ ID NO: 119 - protein, 49 aa, Artificial Sequence
  Description of Artificial Sequence Synthetic polypeptide
AVGQDTQEVIVVPHSLPFKVVVISAILALVVLTIISLIILIMLWQKKPR

SEQ ID NO: 120 - protein, 335 aa, Artificial Sequence
  Description of Artificial Sequence Synthetic polypeptide
RKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 121 - protein, 136 aa, Artificial Sequence
  Description of Artificial Sequence Synthetic polypeptide
CPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVV

SEQ ID NO: 122 - protein, 17 aa, Artificial Sequence
  Description of Artificial Sequence Synthetic peptide
MWLQSLLLLGTVACSIS

SEQ ID NO: 123 - protein, 29 aa, Artificial Sequence
  Description of Artificial Sequence Synthetic peptide
MRISKPHLRSISIQCYLCLLLNSHFLTEA

SEQ ID NO: 124 - protein, 24 aa, Artificial Sequence
  Description of Artificial Sequence Synthetic peptide
MRSSPGNMERIVICLMVIFLGTLV

SEQ ID NO: 125 - protein, 22 aa, Artificial Sequence
  Description of Artificial Sequence Synthetic peptide
MLLLVTSLLLCELPHPAFLLIP

SEQ ID NO: 126 - protein, 19 aa, Artificial Sequence
  Description of Artificial Sequence Synthetic peptide
MTRLTVLALLAGLLASSRA

SEQ ID NO: 127 - DNA, 57 bp, Artificial Sequence
  Description of Artificial Sequence Synthetic oligonucleotide
atgactaggttgacagtcctcgccttgcttgctggattgcttgccagttctcgagcc

SEQ ID NO: 128 - protein, 8 aa, Artificial Sequence
  Description of Artificial Sequence Synthetic peptide
  MOD_RES (2)..(2): V or I
  MOD_RES (4)..(4): Any amino acid generally thought to be self- cleaving
DXEXNPGP

SEQ ID NO: 129 - protein, 5 aa, Artificial Sequence
  Description of Artificial Sequence Synthetic peptide
GGGGS

SEQ ID NO: 130 - protein, 75 aa, Artificial Sequence
  Description of Artificial Sequence Synthetic polypeptide
  SITE (1)..(75): This sequence may encompass 1-15 “Gly Gly Gly Gly Ser” repeating units
GGGGSGGGGSGGGGSGGGGSGGGGSGGGGSGGGGSGGGGSGGGGSGGGGSGGGGSGGGGSGGGGSGGGGSGGGGS

SEQ ID NO: 131 - protein, 17 aa, Artificial Sequence
  Description of Artificial Sequence Synthetic peptide
MWLQSLLLLGTVACSIS

SEQ ID NO: 132 - protein, 157 aa, Artificial Sequence
  Description of Artificial Sequence Synthetic polypeptide
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVV

SEQ ID NO: 133 - protein, 22 aa, Artificial Sequence
  Description of Artificial Sequence Synthetic peptide
GSGATNFSLLKQAGDVEENPGP

SEQ ID NO: 134 - protein, 18 aa, Artificial Sequence
  Description of Artificial Sequence Synthetic peptide
EGRGSLLTCGDVEENPGP

SEQ ID NO: 135 - protein, 19 aa, Artificial Sequence
  Description of Artificial Sequence Synthetic peptide
AEGRGSLLTCGDVEENPGP

SEQ ID NO: 136 - protein, 4 aa, Artificial Sequence
  Description of Artificial Sequence Synthetic peptide
  MOD_RES (2)..(3): Any amino acid
YXXQ

SEQ ID NO: 137 - protein, 4 aa, Artificial Sequence
  Description of Artificial Sequence Synthetic peptide
YRHQ

SEQ ID NO: 138 - protein, 1597 aa, Artificial Sequence
  Description of Artificial Sequence Synthetic polypeptide
MGWSSIILFLVATATGVHQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSGGGGSGGGGSGGGGSDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKLEPKSCDKTHTCPPCPDPKGTFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRHVGSGEGRGSLLTCGDVEENPGPMYRMQLLSCIALSLALVTNSDIQMTQSPSSLSASVGDRVTITCKASQNVATHVGWYQQKPGKAPKRLIYSASYRYSGVPSRFSGSGSGTEFTLTISNLQPEDFATYYCQQYNRYPYTFGQGTKLEIKGGGGSGGGGSGGGGSQVQLVQSGAEVKKPGASVKVSCKASGYSFTGYTMNWVRQAPGQGLEWMGLINPYNSDTNYAQKLQGRVTMTTDTSTSTAYMELRSLRSDDTAVYYCARVALRVALDYWGQGTLVTVSSPSGQAGAAASESLFVSNHAYQSALTQPASVSGSPGQSITISCSGSSSNIGNNAVNWYQQLPGKAPKLLIYYDDLLPSGVSDRFSGSKSGTSAFLAISGLQSEDEADYYCAAWDDSLNGPVFGGGTKLTVLGGGGSGGGGSGGGGSQVQLVESGGGLVKPGGSLRLSCAASGFTFSSYGMHWVRQAPGKGLEWVAFIRYDGSNKYYADSVKGRFTISRDNSKNTLYLQMNSLRAEDTAVYYCAKDRGLGDGTYFDYWGQGTTVTVSSESKYGPPCPPCPAPEAAGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGLNAEGRGSLLTCGDVEENPGPVDMTRLTVLALLAGLLASSRAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSQLGSGATNFSLLKQAGDVEENPGPMGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVV

SEQ ID NO: 139 - protein, 1597 aa, Artificial Sequence
  Description of Artificial Sequence Synthetic polypeptide
MGWSSIILFLVATATGVHQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSGGGGSGGGGSGGGGSDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKLEPKSCDKTHTCPPCPDPKGTFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRHVGSGEGRGSLLTCGDVEENPGPMYRMQLLSCIALSLALVTNSQVQLVESGGGLVKPGGSLRLSCAASGFTFSSYGMHWVRQAPGKGLEWVAFIRYDGSNKYYADSVKGRFTISRDNSKNTLYLQMNSLRAEDTAVYYCAKDRGLGDGTYFDYWGQGTTVTVSSGGGGSGGGGSGGGGSQSALTQPASVSGSPGQSITISCSGSSSNIGNNAVNWYQQLPGKAPKLLIYYDDLLPSGVSDRFSGSKSGTSAFLAISGLQSEDEADYYCAAWDDSLNGPVFGGGTKLTVLPSGQAGAAASESLFVSNHAYQVQLVQSGAEVKKPGASVKVSCKASGYSFTGYTMNWVRQAPGQGLEWMGLINPYNSDTNYAQKLQGRVTMTTDTSTSTAYMELRSLRSDDTAVYYCARVALRVALDYWGQGTLVTVSSGGGGSGGGGSGGGGSDIQMTQSPSSLSASVGDRVTITCKASQNVATHVGWYQQKPGKAPKRLIYSASYRYSGVPSRFSGSGSGTEFTLTISNLQPEDFATYYCQQYNRYPYTFGQGTKLEIKESKYGPPCPPCPAPEAAGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGLNAEGRGSLLTCGDVEENPGPVDMTRLTVLALLAGLLASSRAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSQLGSGATNFSLLKQAGDVEENPGPMGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVV

SEQ ID NO: 140 - DNA, 63 bp, Artificial Sequence
  Description of Artificial Sequence Synthetic oligonucleotide
ggttctggagaaggacgcggttccttgttgacgtgtggcgatgtagaggaaaatccgggtcca

SEQ ID NO: 141 - DNA, 321 bp, Artificial Sequence
  Description of Artificial Sequence Synthetic polynucleotide
gatatacaaatgactcaaagtccttctagtttgtccgcatccgtcggagatcgtgtaactattacgtgtaaagcatctcaaaatgttgcgacacatgtcgggtggtatcaacaaaagcctgggaaagctcctaaaagactcatttattcagcttcatatcgctattcaggggttccaagtcgcttttcaggatccggatctggaacggaatttacacttaccatatccaatcttcaaccggaagattttgcgacttattattgtcaacagtacaatagatacccttatacatttggacagggtactaagcttgaaataaaa

SEQ ID NO: 142 - DNA, 45 bp, Artificial Sequence
  Description of Artificial Sequence Synthetic oligonucleotide
ggcggcggcggttccggcgggggggggtctggtggtggcggatct

SEQ ID NO: 143 - DNA, 354 bp, Artificial Sequence
  Description of Artificial Sequence Synthetic polynucleotide
caagtccaactcgttcaatcaggggctgaagtcaagaaaccaggagcatctgtcaaagtcagttgtaaagctagtggatattcctttactggatatacaatgaattgggtcaggcaagctccagggcaaggtctcgaatggatggggcttattaatccgtataatagtgatacaaattatgcgcaaaagctccaaggtagagttactatgacaacggatacatccacttctacagcttatatggaacttcgctcccttcgctcagatgatacagctgtctattattgtgctcgtgtcgcgttgagggttgctcttgattattggggtcaaggtacgttggtcacagttagttca

SEQ ID NO: 144 - DNA, 60 bp, Artificial Sequence
  Description of Artificial Sequence Synthetic oligonucleotide
ccatctggacaagccggagctgcagcctctgaaagccttttcgtaagcaaccatgcgtat

SEQ ID NO: 145 - DNA, 330 bp, Artificial Sequence
  Description of Artificial Sequence Synthetic polynucleotide
caaagtgcactcactcaaccagcatcagttagcggaagtcctggccaatccattaccatatcctgctcaggttctagtagcaatattggaaataatgccgttaattggtaccaacagttgcctggcaaggcccctaagctcttgatctattacgacgacctcttgccttccggggtcagcgataggttctctggttcaaagagtggaacgagcgcattcctcgccatatccgggctgcaatcagaagacgaggcagactactactgtgctgcttgggacgactccctgaacggtcccgtcttcggtggtggaaccaagctcacggtgctc

SEQ ID NO: 146 - DNA, 45 bp, Artificial Sequence
  Description of Artificial Sequence Synthetic oligonucleotide
ggcggtggtgggtcaggaggaggcggatccggcggcggcgggagc

SEQ ID NO: 147 - DNA, 687 bp, Artificial Sequence
  Description of Artificial Sequence Synthetic polynucleotide
gagtccaagtacggaccaccctgtccaccatgtcccgcccctgaggcagcaggcggtccaagtgtcttccttttcccacccaagcccaaggacacactcatgatatcacggacacctgaggtcacttgtgtagtcgttgacgtcagtcaagaggaccccgaggtccaattcaactggtacgtggacggggtagaggtccacaatgctaagactaagccaagagaggagcaattccaatccacgtacagagtagtatcagttctcactgtcttgcaccaagactggctcaatgggaaggagtacaagtgtaaggtaagtaataaggggctccccagtagtatagagaagacgatctctaaggccaaggggcaacccagggagccacaagtctacacactcccaccctcacaagaggagatgacaaagaatcaagttagtttgacgtgtttggttaagggattctacccatctgacatagcagtagagtgggagtccaatggacaacctgagaataattacaagactactcccccagtcttggactccgacggttctttcttcctctacagtaggctcacagtagacaagtctagatggcaagaggggaatgtcttcagttgttctgttatgcacgaggcactccacaatcactacacacaaaagtccctctctctttcactcgggaag

SEQ ID NO: 148 - DNA, 54 bp, Artificial Sequence
  Description of Artificial Sequence Synthetic oligonucleotide
gagggcagaggaagtcttctaacatgcggtgacgtggaggagaatcccggccct

SEQ ID NO: 149 - DNA, 57 bp, Artificial Sequence
  Description of Artificial Sequence Synthetic oligonucleotide
gctgaaggaaggggctccctgctgacctgtggcgatgtcgaagaaaacccaggtcct

SEQ ID NO: 150 - DNA, 87 bp, Artificial Sequence
  Description of Artificial Sequence Synthetic oligonucleotide
atgagaatttcgaaaccacatttgagaagtatttccatccagtgctacttgtgtttacttctaaacagtcattttctaactgaagct

SEQ ID NO: 151 - DNA, 57 bp, Artificial Sequence
  Description of Artificial Sequence Synthetic oligonucleotide
atgacccgcctcacagtcctcgcgctcctcgccggactgctcgcctcttcacgggca

SEQ ID NO: 152 - DNA, 399 bp, Artificial Sequence
  Description of Artificial Sequence Synthetic polynucleotide
ggcattcatgtcttcattttgggctgtttcagtgcagggcttcctaaaacagaagccaactgggtgaatgtaatcagcgacctcaagaagatcgaggacttgatccagtccatgcacatagatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttct

SEQ ID NO: 153 - DNA, 399 bp, Artificial Sequence
  Description of Artificial Sequence Synthetic polynucleotide
ggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttct

SEQ ID NO: 154 - DNA, 486 bp, Homo sapiens
atgagaatttcgaaaccacatttgagaagtatttccatccagtgctacttgtgtttacttctaaacagtcattttctaactgaagctggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttct

SEQ ID NO: 155 - DNA, 390 bp, Homo sapiens
gtccacaaatcaagctcccaaggtcaagatcgccacatgattagaatgcgtcaacttatagatattgttgatcagctgaaaaattatgtgaatgacttggtccctgaatttctgccagctccagaagatgtagagacaaactgtgagtggtcagctttttcctgctttcagaaggcccaactaaagtcagcaaatacaggaaacaatgaaaggataatcaatgtatcaattaaaaagctgaagaggaaaccaccttccacaaatgcagggagaagacagaaacacagactaacatgcccttcatgtgattcttatgagaaaaaaccacccaaagaattcctagaaagattcaaatcacttctccaaaaggtatctaccttaagtttcatt

SEQ ID NO: 156 - DNA, 387 bp, Homo sapiens
cacaaatcaagctcccaaggtcaagatcgccacatgattagaatgcgtcaacttatagatattgttgatcagctgaaaaattatgtgaatgacttggtccctgaatttctgccagctccagaagatgtagagacaaactgtgagtggtcagctttttcctgctttcagaaggcccaactaaagtcagcaaatacaggaaacaatgaaaggataatcaatgtatcaattaaaaagctgaagaggaaaccaccttccacaaatgcagggagaagacagaaacacagactaacatgcccttcatgtgattcttatgagaaaaaaccacccaaagaattcctagaaagattcaaatcacttctccaaaaggtatctaccttaagtttcatt

SEQ ID NO: 157 - DNA, 459 bp, Homo sapiens
atgagatccagtcctggcaacatggagaggattgtcatctgtctgatggtcatcttcttggggacactggtccacaaatcaagctcccaaggtcaagatcgccacatgattagaatgcgtcaacttatagatattgttgatcagctgaaaaattatgtgaatgacttggtccctgaatttctgccagctccagaagatgtagagacaaactgtgagtggtcagctttttcctgctttcagaaggcccaactaaagtcagcaaatacaggaaacaatgaaaggataatcaatgtatcaattaaaaagctgaagaggaaaccaccttccacaaatgcagggagaagacagaaacacagactaacatgcccttcatgtgattcttatgagaaaaaaccacccaaagaattcctagaaagattcaaatcacttctccaaaaggtatctaccttaagtttcatt

SEQ ID NO: 158 - DNA, 399 bp, Homo sapiens
ggaatacatgtattcattcttggatgtttcagtgccggtctcccgaaaaccgaagccaactgggtgaatgtgatcagtgaccttaagaagatagaggaccttatccagagcatgcacattgatgctacactctataccgaaagtgatgttcacccatcatgcaaagtaacagccatgaagtgctttcttctggagcttcaagttattagccttgagtccggcgatgccagtattcatgatactgtagaaaatctgattatccttgcgaacaacagtttgtccagcaatgggaatgttactgaatctgggtgtaaagaatgtgaggaacttgaggaaaagaatatcaaggagttcttgcagtcatttgtgcatattgtccaaatgttcataaataccagt

SEQ ID NO: 159 - DNA, 66 bp, Artificial Sequence
  Description of Artificial Sequence Synthetic oligonucleotide
ggcagcggcgccacaaacttctctctgctaaagcaagcaggtgatgttgaagaaaaccccgggcct

SEQ ID NO: 160 - DNA, 66 bp, Artificial Sequence
  Description of Artificial Sequence Synthetic oligonucleotide
gggagtggagctaccaatttctcactcctgaaacaggcgggcgacgtcgaggagaatcctggcccc

SEQ ID NO: 161 - DNA, 66 bp, Artificial Sequence
  Description of Artificial Sequence Synthetic oligonucleotide
atgcttctcctggtgacaagccttctgctctgtgagttaccacacccagcattcctcctgatccca

SEQ ID NO: 162 - DNA, 1005 bp, Artificial Sequence
  Description of Artificial Sequence Synthetic polynucleotide
cgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatg

SEQ ID NO: 163 - DNA, 471 bp, Artificial Sequence
  Description of Artificial Sequence Synthetic polynucleotide
atggggacatcccttctttgttggatggctctctgtttgctcggagccgaccacgctgacgcctgtccttactctaatccctctttgtgttcaggtggtggtgggtcagagttgcctacacaaggtacattctcaaatgttagtacaaatgtcagtcccgccaagccaacgacgacggcatgtccctactctaatccaagtttgtgttctggtggcggtggatctccagcccccagaccacccactcctgctcccactatcgcctctcaacccctctcactcagacctgaggcatgtagaccagccgctggtggggccgtacacacaagaggattggacttcgcctgtgacatctacatatgggcccctctcgctgggacgtgtggagtcttgctcctctccttggtaatcacactctactgtaatcaccggaatcgcaggagagtctgtaagtgtccaagacccgtagtc

SEQ ID NO: 164 - DNA, 147 bp, Artificial Sequence
  Description of Artificial Sequence Synthetic polynucleotide
gctgtgggccaggacacgcaggaggtcatcgtggtgccacactccttgccctttaaggtggtggtgatctcagccatcctggccctggtggtgctcaccatcatctcccttatcatcctcatcatgctttggcagaagaagccacgt

SEQ ID NO: 165 - DNA, 78 bp, Artificial Sequence
  Description of Artificial Sequence Synthetic oligonucleotide
agcggcggtggcagcggcggtggcggtagcggcggtggcggtagcggcggtggcggtagcggcggtggcagcctgcag

SEQ ID NO: 166 - DNA, 45 bp, Artificial Sequence
  Description of Artificial Sequence Synthetic oligonucleotide
ggcggtggcggtagcggcggtggcggtagcggcggtggcggtagc

SEQ ID NO: 167 - DNA, 195 bp, Homo sapiens
atcacctgccccccccccatgagcgtggagcacgccgacatctgggtgaagagctacagcctgtacagcagagagagatacatctgcaacagcggcttcaagagaaaggccggcaccagcagcctgaccgagtgcgtgctgaacaaggccaccaacgtggcccactggaccacccccagcctgaagtgcatcaga

SEQ ID NO: 168 - DNA, 4791 bp, Artificial Sequence
  Description of Artificial Sequence Synthetic polynucleotide
atggggtggtcaagcattattctgtttctggtcgctaccgctacaggcgtccatcagatccagctggtgcagtccggccccgagctgaagaagcccggcgagaccgtgaagatctcctgcaaggcctccggctacaccttcaccgactactccatcaactgggtgaagcgggcccccggcaagggcctgaagtggatgggctggatcaacaccgagacccgggagcccgcctacgcctacgacttccggggccggttcgccttctccctggagacctccgcctccaccgcctacctgcagatcaacaacctgaagtacgaggacaccgccacctacttctgcgccctggactactcctacgccatggactactggggccagggcacctccgtgaccgtgtcctccggtgggggcggctctggtggcggtggcagcggcggaggtggcagtgatattgttcttactcaatcacccccaagccttgcgatgtctcttggtaaacgagcgacaattagttgtagagcttctgaaagcgtaactattcttgggtcacatcttattcattggtatcaacaaaagccgggacaaccgcctacactcttgattcaactcgcgagcaatgttcaaacgggtgtccctgcacgcttttctgggagcggttcacgaacagattttactctcacgattgatccagtcgaagaagatgatgtcgctgtatattattgtctccaaagtaggacaataccaagaacttttggtggtggtacaaaattggaaattaaactcgagcccaaatcttgtgacaaaactcacacatgcccaccgtgcccggatcccaaaggtaccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgccacgtgggttctggagaaggacgcggttccttgttgacgtgtggcgatgtagaggaaaatccgggtccaatgtacaggatgcaactcctgtcttgcattgcactaagtcttgcacttgtcacaaacagtgacatccagatgacccagagccccagcagcctgagcgccagcgtgggcgaccgggtgaccatcacctgcaaggccagccagaacgtggccacccacgtgggctggtaccagcagaagcccggcaaggcccccaagcggctgatctacagcgccagctaccggtacagcggcgtgcccagccggttcagcggcagcggcagcggcaccgagttcaccctgaccatcagcaacctgcagcccgaggacttcgccacctactactgccagcagtacaaccggtacccctacaccttcggccagggcaccaagctggagatcaagggtgggggcggctctggtggcggtggcagcggcggaggtggcagtcaggtgcagctggtgcagagcggcgccgaggtgaagaagcccggcgccagcgtgaaggtgagctgcaaggccagcggctacagcttcaccggctacaccatgaactgggtgcggcaggcccccggccagggcctggagtggatgggcctgatcaacccctacaacagcgacaccaactacgcccagaagctgcagggccgggtgaccatgaccaccgacaccagcaccagcaccgcctacatggagctgcggagcctgcggagcgacgacaccgccgtgtactactgcgcccgggtggccctgcgggtggccctggactactggggccagggcaccctggtgaccgtgagcagcccgagcggccaggcgggcgcggcggcatcggagtccctgtttgtgtcaaatcacgcctaccagtcagcgcttacgcagccggcgtcggtgtcgggttccccgggtcagtcgatcacgatcagctgtagtgggagcagctccaacatcggtaacaacgcagtgaactggtatcagcaactgccgggaaaagcgccgaaactgctgatttactatgatgatttgctgccaagtggagttagtgaccgcttttccggcagtaaatcgggtacctcggcttttctggctatttcgggtctccagagcgaggatgaagctgattattattgcgccgcatgggatgatagcttaaatggcccagtttttggcggcggtactaaactgaccgtgctgggtgggggcggctctggtggcggtggcagcggcggaggtggcagtcaagtgcagctggttgaatccggtggcggtctggtcaagccgggcggctctttgcgtctgagctgtgccgcgtcgggttttaccttcagctcttatggtatgcattgggtgcgtcaggcgcctggcaaaggtctggagtgggttgcgttcatccgctacgatgggtctaacaaatattatgccgactcagtaaaaggacgcttcactattagccgcgacaatagcaaaaataccctgtacctgcaaatgaatagcctgcgcgccgaagataccgccgtttactattgcgctaaagatcgtggcctgggtgatggtacgtacttcgactattggggacaagggaccacagttacagtcagctccgagtccaagtacggtcccccttgtcccccatgtcctgctcctgaggctgccggtggtccctcagtcttccttttcccacccaagcccaaggacacgctcatgatctctaggactcccgaggtcacttgtgtcgtcgttgacgtcagtcaagaggacccagaggtacaattcaactggtacgtcgatggagttgaggtccacaatgctaagacgaagcctcgcgaggagcaattccaatctacatacagagttgtctcagttttgactgttttgcaccaagactggctcaatggtaaggaatacaagtgtaaggtttccaataagggtttgccctctagtatcgagaagactatctccaaggccaagggacaacctagggagccacaagtctacactctcccaccaagtcaagaggagatgactaagaatcaagtatccctcacttgtttggtcaaggggttctacccaagtgacatagcagttgagtgggagtccaatgggcaaccagagaataattacaagacgactccgcccgttttggactccgacgggtctttcttcctctacagtaggcttacggttgacaagtcccgatggcaagaggggaatgtattctcttgttctgttatgcacgaggcccttcacaatcactacacacaaaagtctctctcactttcactcgggaagggccttaatgcagaagggcgagggtctctcttgacttgtggagatgttgaagaaaaccctggtcctgtcgacatgacccgcctcacagtcctcgcgctcctcgccggactgctcgcctcttcacgggcaggaatacatgtattcattcttggatgtttcagtgccggtctcccgaaaaccgaagccaactgggtgaatgtgatcagtgaccttaagaagatagaggaccttatccagagcatgcacattgatgctacactctataccgaaagtgatgttcacccatcatgcaaagtaacagccatgaagtgctttcttctggagcttcaagttattagccttgagtccggcgatgccagtattcatgatactgtagaaaatctgattatccttgcgaacaacagtttgtccagcaatgggaatgttactgaatctgggtgtaaagaatgtgaggaacttgaggaaaagaatatcaaggagttcttgcagtcatttgtgcatattgtccaaatgttcataaataccagtcaattggggagtggagctaccaatttctcactcctgaaacaggcgggcgacgtcgaggagaatcctggccccatggggacatcccttctttgttggatggctctctgtttgctcggagccgaccacgctgacgcctgtccttactctaatccctctttgtgttcaggtggtggtgggtcagagttgcctacacaaggtacattctcaaatgttagtacaaatgtcagtcccgccaagccaacgacgacggcatgtccctactctaatccaagtttgtgttctggtggcggtggatctccagcccccagaccacccactcctgctcccactatcgcctctcaacccctctcactcagacctgaggcatgtagaccagccgctggtggggccgtacacacaagaggattggacttcgcctgtgacatctacatatgggcccctctcgctgggacgtgtggagtcttgctcctctccttggtaatcacactctactgtaatcaccggaatcgcaggagagtctgtaagtgtccaagacccgtagtc

SEQ ID NO: 169 - DNA, 4791 bp, Artificial Sequence
  Description of Artificial Sequence Synthetic polynucleotide
atggggtggtcaagcattattctgtttctggtcgctaccgctacaggcgtccatcagatccagctggtgcagtccggccccgagctgaagaagcccggcgagaccgtgaagatctcctgcaaggcctccggctacaccttcaccgactactccatcaactgggtgaagcgggcccccggcaagggcctgaagtggatgggctggatcaacaccgagacccgggagcccgcctacgcctacgacttccggggccggttcgccttctccctggagacctccgcctccaccgcctacctgcagatcaacaacctgaagtacgaggacaccgccacctacttctgcgccctggactactcctacgccatggactactggggccagggcacctccgtgaccgtgtcctccggtgggggcggctctggtggcggtggcagcggcggaggtggcagtgatattgttcttactcaatcacccccaagccttgcgatgtctcttggtaaacgagcgacaattagttgtagagcttctgaaagcgtaactattcttgggtcacatcttattcattggtatcaacaaaagccgggacaaccgcctacactcttgattcaactcgcgagcaatgttcaaacgggtgtccctgcacgcttttctgggagcggttcacgaacagattttactctcacgattgatccagtcgaagaagatgatgtcgctgtatattattgtctccaaagtaggacaataccaagaacttttggtggtggtacaaaattggaaattaaactcgagcccaaatcttgtgacaaaactcacacatgcccaccgtgcccggatcccaaaggtaccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgccacgtgggttctggagaaggacgcggttccttgttgacgtgtggcgatgtagaggaaaatccgggtccaatgtacaggatgcaactcctgtcttgcattgcactaagtcttgcacttgtcacaaacagtcaagtgcagctggttgaatccggtggcggtctggtcaagccgggcggctctttgcgtctgagctgtgccgcgtcgggttttaccttcagctcttatggtatgcattgggtgcgtcaggcgcctggcaaaggtctggagtgggttgcgttcatccgctacgatgggtctaacaaatattatgccgactcagtaaaaggacgcttcactattagccgcgacaatagcaaaaataccctgtacctgcaaatgaatagcctgcgcgccgaagataccgccgtttactattgcgctaaagatcgtggcctgggtgatggtacgtacttcgattactggggtcagggcaccaccgttaccgttagttcaggcggtggtgggtcaggaggaggcggatccggcggcggcgggagccaaagtgcactcactcaaccagcatcagttagcggaagtcctggccaatccattaccatatcctgctcaggttctagtagcaatattggaaataatgccgttaattggtaccaacagttgcctggcaaggcccctaagctcttgatctattacgacgacctcttgccttccggggtcagcgataggttctctggttcaaagagtggaacgagcgcattcctcgccatatccgggctgcaatcagaagacgaggcagactactactgtgctgcttgggacgactccctgaacggtcccgtcttcggtggtggaaccaagctcacggtgctcccatctggacaagccggagctgcagcctctgaaagccttttcgtaagcaaccatgcgtatcaagtccaactcgttcaatcaggggctgaagtcaagaaaccaggagcatctgtcaaagtcagttgtaaagctagtggatattcctttactggatatacaatgaattgggtcaggcaagctccagggcaaggtctcgaatggatggggcttattaatccgtataatagtgatacaaattatgcgcaaaagctccaaggtagagttactatgacaacggatacatccacttctacagcttatatggaacttcgctcccttcgctcagatgatacagctgtctattattgtgctcgtgtcgcgttgagggttgctcttgattattggggtcaaggtacgttggtcacagttagttcaggcggcggcggttccggcgggggggggtctggtggtggcggatctgatatacaaatgactcaaagtccttctagtttgtccgcatccgtcggagatcgtgtaactattacgtgtaaagcatctcaaaatgttgcgacacatgtcgggtggtatcaacaaaagcctgggaaagctcctaaaagactcatttattcagcttcatatcgctattcaggggttccaagtcgcttttcaggatccggatctggaacggaatttacacttaccatatccaatcttcaaccggaagattttgcgacttattattgtcaacagtacaatagatacccttatacatttggacagggtactaagcttgaaataaaagagtccaagtacggaccaccctgtccaccatgtcccgcccctgaggcagcaggcggtccaagtgtcttccttttcccacccaagcccaaggacacactcatgatatcacggacacctgaggtcacttgtgtagtcgttgacgtcagtcaagaggaccccgaggtccaattcaactggtacgtggacggggtagaggtccacaatgctaagactaagccaagagaggagcaattccaatccacgtacagagtagtatcagttctcactgtcttgcaccaagactggctcaatgggaaggagtacaagtgtaaggtaagtaataaggggctccccagtagtatagagaagacgatctctaaggccaaggggcaacccagggagccacaagtctacacactcccaccctcacaagaggagatgacaaagaatcaagttagtttgacgtgtttggttaagggattctacccatctgacatagcagtagagtgggagtccaatggacaacctgagaataattacaagactactcccccagtcttggactccgacggttctttcttcctctacagtaggctcacagtagacaagtctagatggcaagaggggaatgtcttcagttgttctgttatgcacgaggcactccacaatcactacacacaaaagtccctctctctttcactcgggaaggggcttaatgctgaaggaaggggctccctgctgacctgtggcgatgtcgaagaaaacccaggtcctgtcgacatgacccgcctcacagtcctcgcgctcctcgccggactgctcgcctcttcacgggcaggaatacatgtattcattcttggatgtttcagtgccggtctcccgaaaaccgaagccaactgggtgaatgtgatcagtgaccttaagaagatagaggaccttatccagagcatgcacattgatgctacactctataccgaaagtgatgttcacccatcatgcaaagtaacagccatgaagtgctttcttctggagcttcaagttattagccttgagtccggcgatgccagtattcatgatactgtagaaaatctgattatccttgcgaacaacagtttgtccagcaatgggaatgttactgaatctgggtgtaaagaatgtgaggaacttgaggaaaagaatatcaaggagttcttgcagtcatttgtgcatattgtccaaatgttcataaataccagtcaattggggagtggagctaccaatttctcactcctgaaacaggcgggcgacgtcgaggagaatcctggccccatggggacatcccttctttgttggatggctctctgtttgctcggagccgaccacgctgacgcctgtccttactctaatccctctttgtgttcaggtggtggtgggtcagagttgcctacacaaggtacattctcaaatgttagtacaaatgtcagtcccgccaagccaacgacgacggcatgtccctactctaatccaagtttgtgttctggtggcggtggatctccagcccccagaccacccactcctgctcccactatcgcctctcaacccctctcactcagacctgaggcatgtagaccagccgctggtggggccgtacacacaagaggattggacttcgcctgtgacatctacatatgggcccctctcgctgggacgtgtggagtcttgctcctctccttggtaatcacactctactgtaatcaccggaatcgcaggagagtctgtaagtgtccaagacccgtagtc

SEQ ID NO: 170 - protein, 323 aa, Artificial Sequence
  Description of Artificial Sequence Synthetic polypeptide
MPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKR

SEQ ID NO: 171 - DNA, 45 bp, Artificial Sequence
  Description of Artificial Sequence Synthetic oligonucleotide
cgggccagcgagagcgtgaccatcctgggcagccacctgatccac

SEQ ID NO: 172 - DNA, 21 bp, Artificial Sequence
  Description of Artificial Sequence Synthetic oligonucleotide
ctcgccagcaatgtgcagacc

SEQ ID NO: 173 - DNA, 27 bp, Artificial Sequence
  Description of Artificial Sequence Synthetic oligonucleotide
ctgcagagccggaccatcccccggacc

SEQ ID NO: 174 - DNA, 21 bp, Artificial Sequence
  Description of Artificial Sequence Synthetic oligonucleotide
ggctacaccttcaccgactac

SEQ ID NO: 175 - DNA, 21 bp, Artificial Sequence
  Description of Artificial Sequence Synthetic oligonucleotide
atcaacaccgagacaagagag

SEQ ID NO: 176 - DNA, 24 bp, Artificial Sequence
  Description of Artificial Sequence Synthetic oligonucleotide
gactacagctacgccatggactac

SEQ ID NO: 177 - DNA, 33 bp, Artificial Sequence
  Description of Artificial Sequence Synthetic oligonucleotide
tccgccagccaggacatcagcaactacctgaac

SEQ ID NO: 178 - DNA, 21 bp, Artificial Sequence
  Description of Artificial Sequence Synthetic oligonucleotide
tacacctccaacctgcactcc

SEQ ID NO: 179 - DNA, 27 bp, Artificial Sequence
  Description of Artificial Sequence Synthetic oligonucleotide
cagcagtacaggaagctcccctggact

SEQ ID NO: 180 - DNA, 21 bp, Artificial Sequence
  Description of Artificial Sequence Synthetic oligonucleotide
ggcggcaccttcagcaactac

SEQ ID NO: 181 - DNA, 18 bp, Artificial Sequence
  Description of Artificial Sequence Synthetic oligonucleotide
tacaggggccacagcgac

SEQ ID NO: 182 - DNA, 36 bp, Artificial Sequence
  Description of Artificial Sequence Synthetic oligonucleotide
ggcgccatctacaacggctacgacgtgctggacaac

SEQ ID NO: 183 - DNA, 33 bp, Artificial Sequence
  Description of Artificial Sequence Synthetic oligonucleotide
aaggccagccagaacgtggccacccacgtgggc

SEQ ID NO: 184 - DNA, 21 bp, Artificial Sequence
  Description of Artificial Sequence Synthetic oligonucleotide
agcgccagctaccggtacagc

SEQ ID NO: 185 - DNA, 27 bp, Artificial Sequence
  Description of Artificial Sequence Synthetic oligonucleotide
cagcagtacaaccggtacccctacacc

SEQ ID NO: 186 - DNA, 21 bp, Artificial Sequence
  Description of Artificial Sequence Synthetic oligonucleotide
ggctacagcttcaccggctac

SEQ ID NO: 187 - DNA, 18 bp, Artificial Sequence
  Description of Artificial Sequence Synthetic oligonucleotide
aacccctacaacagcgac

SEQ ID NO: 188 - DNA, 27 bp, Artificial Sequence
  Description of Artificial Sequence Synthetic oligonucleotide
gtggccctgcgggtggccctggactac

SEQ ID NO: 189 - DNA, 30 bp, Artificial Sequence
  Description of Artificial Sequence Synthetic oligonucleotide
agtgggagcagctccaacatcggtaacaac

SEQ ID NO: 190 - DNA, 21 bp, Artificial Sequence
  Description of Artificial Sequence Synthetic oligonucleotide
tatgatgatttgctgccaagt

SEQ ID NO: 191 - DNA, 33 bp, Artificial Sequence
  Description of Artificial Sequence Synthetic oligonucleotide
gccgcatgggatgatagcttaaatggcccagtt

SEQ ID NO: 192 - DNA, 21 bp, Artificial Sequence
  Description of Artificial Sequence Synthetic oligonucleotide
ggttttaccttcagctcttat

SEQ ID NO: 193 - DNA, 18 bp, Artificial Sequence
  Description of Artificial Sequence Synthetic oligonucleotide
cgctacgatgggtctaac

SEQ ID NO: 194 - DNA, 36 bp, Artificial Sequence
  Description of Artificial Sequence Synthetic oligonucleotide
gatcgtggcctgggtgatggtacgtacttcgattac

SEQ ID NO: 195 - DNA, 969 bp, Artificial Sequence
  Description of Artificial Sequence Synthetic polynucleotide
atgccacctcctcgcctcctcttcttcctcctcttcctcacccccatggaagtcaggcccgaggaacctctagtggtgaaggtggaagagggagataacgctgtgctgcagtgcctcaaggggacctcagatggccccactcagcagctgacctggtctcgggagtccccgcttaaacccttcttaaaactcagcctggggctgccaggcctgggaatccacatgaggcccctggccatctggcttttcatcttcaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagccaggacctcaccatggcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatggagacgggtctgttgttgccccgggccacagctcaagacgctggaaagtattattgtcaccgtggcaacctgaccatgtcattccacctggagatcactgctcggccagtactatggcactggctgctgaggactggtggctggaaggtctcagctgtgactttggcttatctgatcttctgcctgtgttcccttgtgggcattcttcatcttcaaagagccctggtcctgaggaggaaaaga

SEQ ID NO: 196 - protein, 464 aa, Artificial Sequence
  Description of Artificial Sequence Synthetic polypeptide
ATMGWSSIILFLVATATGVHQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSGGGGSGGGGSGGGGSDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKLEPKSCDKTHTCPPCPDPKGTFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 197 - DNA, 1392 bp, Artificial Sequence
  Description of Artificial Sequence Synthetic polynucleotide
gccaccatggggtggtcaagcattattctgtttctggtcgctaccgctacaggcgtccatcagatccagctggtgcagtccggccccgagctgaagaagcccggcgagaccgtgaagatctcctgcaaggcctccggctacaccttcaccgactactccatcaactgggtgaagcgggcccccggcaagggcctgaagtggatgggctggatcaacaccgagacccgggagcccgcctacgcctacgacttccggggccggttcgccttctccctggagacctccgcctccaccgcctacctgcagatcaacaacctgaagtacgaggacaccgccacctacttctgcgccctggactactcctacgccatggactactggggccagggcacctccgtgaccgtgtcctccggtgggggcggctctggtggcggtggcagcggcggaggtggcagtgatattgttcttactcaatcacccccaagccttgcgatgtctcttggtaaacgagcgacaattagttgtagagcttctgaaagcgtaactattcttgggtcacatcttattcattggtatcaacaaaagccgggacaaccgcctacactcttgattcaactcgcgagcaatgttcaaacgggtgtccctgcacgcttttctgggagcggttcacgaacagattttactctcacgattgatccagtcgaagaagatgatgtcgctgtatattattgtctccaaagtaggacaataccaagaacttttggtggtggtacaaaattggaaattaaactcgagcccaaatcttgtgacaaaactcacacatgcccaccgtgcccggatcccaaaggtaccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 198 - protein, 814 aa, Artificial Sequence
  Description of Artificial Sequence Synthetic polypeptide
ATMGWSSIILFLVATATGVHDIQMTQSPSSLSASVGDRVTITCSASQDISNYLNWYQQKPGKAPKLLIYYTSNLHSGVPSRFSGSGSGTDFTLTISSLQPEDFATYYCQQYRKLPWTFGQGTKLEIKRGGGGSGGGGSGGGGSQVQLVQSGAEVKKPGSSVKVSCKASGGTFSNYWMHWVRQAPGQGLEWMGATYRGHSDTYYNQKFKGRVTITADKSTSTAYMELSSLRSEDTAVYYCARGAIYNGYDVLDNWGQGTLVTVSSLEPKSCDKTHTCPPCPDPKGTFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRHVGSGEGRGSLLTCGDVEENPGPLINMPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKR

SEQ ID NO: 199 - DNA, 2442 bp, Artificial Sequence
  Description of Artificial Sequence Synthetic polynucleotide
gccaccatggggtggtcaagcattattctgtttctggtcgctaccgctacaggcgtccatgacatccagatgacccagagccctagctcactgagcgccagcgtgggcgacagggtgaccattacctgctccgccagccaggacatcagcaactacctgaactggtaccagcagaagcccggcaaggcccccaagctgctgatctactacacctccaacctgcactccggcgtgcccagcaggttcagcggaagcggcagcggcaccgatttcaccctgaccatctccagcctgcagcccgaggacttcgccacctactactgccagcagtacaggaagctcccctggactttcggccagggcaccaaactggagatcaagcgtggtgggggcggctctggtggcggtggcagcggcggaggtggcagtcaggtgcagctggtccagagcggcgccgaagtgaagaagcccggcagctccgtgaaagtgagctgcaaggccagcggcggcaccttcagcaactactggatgcactgggtgaggcaggcccccggacagggcctggagtggatgggcgccacctacaggggccacagcgacacctactacaaccagaagttcaagggccgggtgaccatcaccgccgacaagagcaccagcaccgcctacatggaactgagcagcctcaggagcgaggacaccgctgtgtattactgcgccaggggcgccatctacaacggctacgacgtgctggacaactggggccagggcacactagtgaccgtgtccagcctcgagcccaaatcttgtgacaaaactcacacatgcccaccgtgcccggatcccaaaggtaccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgccacgtgggttctggagaaggacgcggttccttgttgacgtgtggcgatgtagaggaaaatccgggtccattaattaacatgccacctcctcgcctcctcttcttcctcctcttcctcacccccatggaagtcaggcccgaggaacctctagtggtgaaggtggaagagggagataacgctgtgctgcagtgcctcaaggggacctcagatggccccactcagcagctgacctggtctcgggagtccccgcttaaacccttcttaaaactcagcctggggctgccaggcctgggaatccacatgaggcccctggccatctggcttttcatcttcaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagccaggacctcaccatggcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatggagacgggtctgttgttgccccgggccacagctcaagacgctggaaagtattattgtcaccgtggcaacctgaccatgtcattccacctggagatcactgctcggccagtactatggcactggctgctgaggactggtggctggaaggtctcagctgtgactttggcttatctgatcttctgcctgtgttcccttgtgggcattcttcatcttcaaagagccctggtcctgaggaggaaaaga

SEQ ID NO: 200 - DNA, 51 bp, Artificial Sequence
  Description of Artificial Sequence Synthetic oligonucleotide
atgtggctgcagagcctgctgctcttgggcactgtggcctgcagcatctct

SEQ ID NO: 201 - protein, 890 aa, Artificial Sequence
  Description of Artificial Sequence Synthetic polypeptide
MPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKRGLNAEGRGSLLTCGDVEENPGPVDMYRMQLLSCIALSLALVTNSGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSYPYDVPDYAQLGSGATNFSLLKQAGDVEENPGPMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 202 - DNA, 2670 bp, Artificial Sequence
  Description of Artificial Sequence Synthetic polynucleotide
atgccacctcctcgcctcctcttcttcctcctcttcctcacccccatggaagtcaggcccgaggaacctctagtggtgaaggtggaagagggagataacgctgtgctgcagtgcctcaaggggacctcagatggccccactcagcagctgacctggtctcgggagtccccgcttaaacccttcttaaaactcagcctggggctgccaggcctgggaatccacatgaggcccctggccatctggcttttcatcttcaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagccaggacctcaccatggcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatggagacgggtctgttgttgccccgggccacagctcaagacgctggaaagtattattgtcaccgtggcaacctgaccatgtcattccacctggagatcactgctcggccagtactatggcactggctgctgaggactggtggctggaaggtctcagctgtgactttggcttatctgatcttctgcctgtgttcccttgtgggcattcttcatcttcaaagagccctggtcctgaggaggaaaagaggtttaaacgccgagggcagaggaagtcttctaacatgcggtgacgtggaggagaatcccggccctgtcgacatgtacaggatgcaactcctgtcttgcattgcactaagtcttgcacttgtcacaaacagtggcattcatgtcttcattttgggctgtttcagtgcagggcttcctaaaacagaagccaactgggtgaatgtaatcagcgacctcaagaagatcgaggacttgatccagtccatgcacatagatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttacccatacgatgttccagattacgctcaattgggcagcggcgccacaaacttctctctgctaaagcaagcaggtgatgttgaagaaaaccccgggcctatgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatg

SEQ ID NO: 203 - protein, 1029 aa, Artificial Sequence
  Description of Artificial Sequence Synthetic polypeptide
MGWSSIILFLVATATGVHQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSGGGGSGGGGSGGGGSDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKLEPKSCDKTHTCPPCPDPKGTFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGLNAEGRGSLLTCGDVEENPGPVDMYRMQLLSCIALSLALVTNSGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSYPYDVPDYAQLGSGATNFSLLKQAGDVEENPGPMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 204 - DNA, 3087 bp, Artificial Sequence
  Description of Artificial Sequence Synthetic polynucleotide
atggggtggtcaagcattattctgtttctggtcgctaccgctacaggcgtccatcagatccagctggtgcagtccggccccgagctgaagaagcccggcgagaccgtgaagatctcctgcaaggcctccggctacaccttcaccgactactccatcaactgggtgaagcgggcccccggcaagggcctgaagtggatgggctggatcaacaccgagacccgggagcccgcctacgcctacgacttccggggccggttcgccttctccctggagacctccgcctccaccgcctacctgcagatcaacaacctgaagtacgaggacaccgccacctacttctgcgccctggactactcctacgccatggactactggggccagggcacctccgtgaccgtgtcctccggtgggggcggctctggtggcggtggcagcggcggaggtggcagtgatattgttcttactcaatcacccccaagccttgcgatgtctcttggtaaacgagcgacaattagttgtagagcttctgaaagcgtaactattcttgggtcacatcttattcattggtatcaacaaaagccgggacaaccgcctacactcttgattcaactcgcgagcaatgttcaaacgggtgtccctgcacgcttttctgggagcggttcacgaacagattttactctcacgattgatccagtcgaagaagatgatgtcgctgtatattattgtctccaaagtaggacaataccaagaacttttggtggtggtacaaaattggaaattaaactcgagcccaaatcttgtgacaaaactcacacatgcccaccgtgcccggatcccaaaggtaccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggtttaaacgccgagggcagaggaagtcttctaacatgcggtgacgtggaggagaatcccggccctgtcgacatgtacaggatgcaactcctgtcttgcattgcactaagtcttgcacttgtcacaaacagtggcattcatgtcttcattttgggctgtttcagtgcagggcttcctaaaacagaagccaactgggtgaatgtaatcagcgacctcaagaagatcgaggacttgatccagtccatgcacatagatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttacccatacgatgttccagattacgctcaattgggcagcggcgccacaaacttctctctgctaaagcaagcaggtgatgttgaagaaaaccccgggcctatgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatg

SEQ ID NO: 205 - protein, 1816 aa, Artificial Sequence
  Description of Artificial Sequence Synthetic polypeptide
MGWSSIILFLVATATGVHQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSGGGGSGGGGSGGGGSDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKLEPKSCDKTHTCPPCPDPKGTFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRHVGSGEGRGSLLTCGDVEENPGPMYRMQLLSCIALSLALVTNSDIQMTQSPSSLSASVGDRVTITCKASQNVATHVGWYQQKPGKAPKRLIYSASYRYSGVPSRFSGSGSGTEFTLTISNLQPEDFATYYCQQYNRYPYTFGQGTKLEIKGGGGSGGGGSGGGGSQVQLVQSGAEVKKPGASVKVSCKASGYSFTGYTMNWVRQAPGQGLEWMGLINPYNSDTNYAQKLQGRVTMTTDTSTSTAYMELRSLRSDDTAVYYCARVALRVALDYWGQGTLVTVSSPSGQAGAAASESLFVSNHAYQSALTQPASVSGSPGQSITISCSGSSSNIGNNAVNWYQQLPGKAPKLLIYYDDLLPSGVSDRFSGSKSGTSAFLAISGLQSEDEADYYCAAWDDSLNGPVFGGGTKLTVLGGGGSGGGGSGGGGSQVQLVESGGGLVKPGGSLRLSCAASGFTFSSYGMHWVRQAPGKGLEWVAFIRYDGSNKYYADSVKGRFTISRDNSKNTLYLQMNSLRAEDTAVYYCAKDRGLGDGTYFDYWGQGTTVTVSSESKYGPPCPPCPAPEAAGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKYPYDVPDYAGLNAEGRGSLLTCGDVEENPGPVDMYRMQLLSCIALSLALVTNSGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSYPYDVPDYAQLGSGATNFSLLKQAGDVEENPGPMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 206 - DNA, 5448 bp, Artificial Sequence
  Description of Artificial Sequence Synthetic polynucleotide
atggggtggtcaagcattattctgtttctggtcgctaccgctacaggcgtccatcagatccagctggtgcagtccggccccgagctgaagaagcccggcgagaccgtgaagatctcctgcaaggcctccggctacaccttcaccgactactccatcaactgggtgaagcgggcccccggcaagggcctgaagtggatgggctggatcaacaccgagacccgggagcccgcctacgcctacgacttccggggccggttcgccttctccctggagacctccgcctccaccgcctacctgcagatcaacaacctgaagtacgaggacaccgccacctacttctgcgccctggactactcctacgccatggactactggggccagggcacctccgtgaccgtgtcctccggtgggggcggctctggtggcggtggcagcggcggaggtggcagtgatattgttcttactcaatcacccccaagccttgcgatgtctcttggtaaacgagcgacaattagttgtagagcttctgaaagcgtaactattcttgggtcacatcttattcattggtatcaacaaaagccgggacaaccgcctacactcttgattcaactcgcgagcaatgttcaaacgggtgtccctgcacgcttttctgggagcggttcacgaacagattttactctcacgattgatccagtcgaagaagatgatgtcgctgtatattattgtctccaaagtaggacaataccaagaacttttggtggtggtacaaaattggaaattaaactcgagcccaaatcttgtgacaaaactcacacatgcccaccgtgcccggatcccaaaggtaccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgccacgtgggttctggagaaggacgcggttccttgttgacgtgtggcgatgtagaggaaaatccgggtccaatgtacaggatgcaactcctgtcttgcattgcactaagtcttgcacttgtcacaaacagtgacatccagatgacccagagccccagcagcctgagcgccagcgtgggcgaccgggtgaccatcacctgcaaggccagccagaacgtggccacccacgtgggctggtaccagcagaagcccggcaaggcccccaagcggctgatctacagcgccagctaccggtacagcggcgtgcccagccggttcagcggcagcggcagcggcaccgagttcaccctgaccatcagcaacctgcagcccgaggacttcgccacctactactgccagcagtacaaccggtacccctacaccttcggccagggcaccaagctggagatcaagggtgggggcggctctggtggcggtggcagcggcggaggtggcagtcaggtgcagctggtgcagagcggcgccgaggtgaagaagcccggcgccagcgtgaaggtgagctgcaaggccagcggctacagcttcaccggctacaccatgaactgggtgcggcaggcccccggccagggcctggagtggatgggcctgatcaacccctacaacagcgacaccaactacgcccagaagctgcagggccgggtgaccatgaccaccgacaccagcaccagcaccgcctacatggagctgcggagcctgcggagcgacgacaccgccgtgtactactgcgcccgggtggccctgcgggtggccctggactactggggccagggcaccctggtgaccgtgagcagcccgagcggccaggcgggcgcggcggcatcggagtccctgtttgtgtcaaatcacgcctaccagtcagcgcttacgcagccggcgtcggtgtcgggttccccgggtcagtcgatcacgatcagctgtagtgggagcagctccaacatcggtaacaacgcagtgaactggtatcagcaactgccgggaaaagcgccgaaactgctgatttactatgatgatttgctgccaagtggagttagtgaccgcttttccggcagtaaatcgggtacctcggcttttctggctatttcgggtctccagagcgaggatgaagctgattattattgcgccgcatgggatgatagcttaaatggcccagtttttggcggcggtactaaactgaccgtgctgggtgggggcggctctggtggcggtggcagcggcggaggtggcagtcaagtgcagctggttgaatccggtggcggtctggtcaagccgggcggctctttgcgtctgagctgtgccgcgtcgggttttaccttcagctcttatggtatgcattgggtgcgtcaggcgcctggcaaaggtctggagtgggttgcgttcatccgctacgatgggtctaacaaatattatgccgactcagtaaaaggacgcttcactattagccgcgacaatagcaaaaataccctgtacctgcaaatgaatagcctgcgcgccgaagataccgccgtttactattgcgctaaagatcgtggcctgggtgatggtacgtacttcgattactggggtcagggcaccaccgttaccgttagttcagaaagcaaatatggcccgccgtgcccgccgtgcccggcgccggaagcggcgggcggcccgagcgtgtttctgtttccgccgaaaccgaaagataccctgatgattagccgcaccccggaagtgacctgcgtggtggtggatgtgagccaggaagatccggaagtgcagtttaactggtatgtggatggcgtggaagtgcataacgcgaaaaccaaaccgcgcgaagaacagtttcagagcacctatcgcgtggtgagcgtgctgaccgtgctgcatcaggattggctgaacggcaaagaatataaatgcaaagtgagcaacaaaggcctgccgagcagcattgaaaaaaccattagcaaagcgaaaggccagccgcgcgaaccgcaggtgtataccctgccgccgagccaggaagaaatgaccaaaaaccaggtgagcctgacctgcctggtgaaaggcttttatccgagcgatattgcggtggaatgggaaagcaacggccagccggaaaacaactataaaaccaccccgccggtgctggatagcgatggcagcttttttctgtatagccgcctgaccgtggataaaagccgctggcaggaaggcaacgtgtttagctgcagcgtgatgcatgaagcgctgcataaccattatacccagaaaagcctgagcctgagcctgggcaaatacccatacgatgttccagattacgctggtttaaacgccgagggcagaggaagtcttctaacatgcggtgacgtggaggagaatcccggccctgtcgacatgtacaggatgcaactcctgtcttgcattgcactaagtcttgcacttgtcacaaacagtggcattcatgtcttcattttgggctgtttcagtgcagggcttcctaaaacagaagccaactgggtgaatgtaatcagcgacctcaagaagatcgaggacttgatccagtccatgcacatagatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttacccatacgatgttccagattacgctcaattgggcagcggcgccacaaacttctctctgctaaagcaagcaggtgatgttgaagaaaaccccgggcctatgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatg

SEQ ID NO: 207 - protein, 1816 aa, Artificial Sequence
  Description of Artificial Sequence Synthetic polypeptide
MGWSSIILFLVATATGVHQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSGGGGSGGGGSGGGGSDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKLEPKSCDKTHTCPPCPDPKGTFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRHVGSGEGRGSLLTCGDVEENPGPMYRMQLLSCIALSLALVTNSQVQLVESGGGLVKPGGSLRLSCAASGFTFSSYGMHWVRQAPGKGLEWVAFIRYDGSNKYYADSVKGRFTISRDNSKNTLYLQMNSLRAEDTAVYYCAKDRGLGDGTYFDYWGQGTTVTVSSGGGGSGGGGSGGGGSQSALTQPASVSGSPGQSITISCSGSSSNIGNNAVNWYQQLPGKAPKLLIYYDDLLPSGVSDRFSGSKSGTSAFLAISGLQSEDEADYYCAAWDDSLNGPVFGGGTKLTVLPSGQAGAAASESLFVSNHAYQVQLVQSGAEVKKPGASVKVSCKASGYSFTGYTMNWVRQAPGQGLEWMGLINPYNSDTNYAQKLQGRVTMTTDTSTSTAYMELRSLRSDDTAVYYCARVALRVALDYWGQGTLVTVSSGGGGSGGGGSGGGGSDIQMTQSPSSLSASVGDRVTITCKASQNVATHVGWYQQKPGKAPKRLIYSASYRYSGVPSRFSGSGSGTEFTLTISNLQPEDFATYYCQQYNRYPYTFGQGTKLEIKESKYGPPCPPCPAPEAAGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKYPYDVPDYAGLNAEGRGSLLTCGDVEENPGPVDMYRMQLLSCIALSLALVTNSGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSYPYDVPDYAQLGSGATNFSLLKQAGDVEENPGPMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 208 - DNA, 5448 bp, Artificial Sequence
  Description of Artificial Sequence Synthetic polynucleotide
atggggtggtcaagcattattctgtttctggtcgctaccgctacaggcgtccatcagatccagctggtgcagtccggccccgagctgaagaagcccggcgagaccgtgaagatctcctgcaaggcctccggctacaccttcaccgactactccatcaactgggtgaagcgggcccccggcaagggcctgaagtggatgggctggatcaacaccgagacccgggagcccgcctacgcctacgacttccggggccggttcgccttctccctggagacctccgcctccaccgcctacctgcagatcaacaacctgaagtacgaggacaccgccacctacttctgcgccctggactactcctacgccatggactactggggccagggcacctccgtgaccgtgtcctccggtgggggcggctctggtggcggtggcagcggcggaggtggcagtgatattgttcttactcaatcacccccaagccttgcgatgtctcttggtaaacgagcgacaattagttgtagagcttctgaaagcgtaactattcttgggtcacatcttattcattggtatcaacaaaagccgggacaaccgcctacactcttgattcaactcgcgagcaatgttcaaacgggtgtccctgcacgcttttctgggagcggttcacgaacagattttactctcacgattgatccagtcgaagaagatgatgtcgctgtatattattgtctccaaagtaggacaataccaagaacttttggtggtggtacaaaattggaaattaaactcgagcccaaatcttgtgacaaaactcacacatgcccaccgtgcccggatcccaaaggtaccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgccacgtgggttctggagaaggacgcggttccttgttgacgtgtggcgatgtagaggaaaatccgggtccaatgtacaggatgcaactcctgtcttgcattgcactaagtcttgcacttgtcacaaacagtcaagtgcagctggttgaatccggtggcggtctggtcaagccgggcggctctttgcgtctgagctgtgccgcgtcgggttttaccttcagctcttatggtatgcattgggtgcgtcaggcgcctggcaaaggtctggagtgggttgcgttcatccgctacgatgggtctaacaaatattatgccgactcagtaaaaggacgcttcactattagccgcgacaatagcaaaaataccctgtacctgcaaatgaatagcctgcgcgccgaagataccgccgtttactattgcgctaaagatcgtggcctgggtgatggtacgtacttcgattactggggtcagggcaccaccgttaccgttagttcaggtgggggcggctctggtggcggtggcagcggcggaggtggcagtcagtcagcgcttacgcagccggcgtcggtgtcgggttccccgggtcagtcgatcacgatcagctgtagtgggagcagctccaacatcggtaacaacgcagtgaactggtatcagcaactgccgggaaaagcgccgaaactgctgatttactatgatgatttgctgccaagtggagttagtgaccgcttttccggcagtaaatcgggtacctcggcttttctggctatttcgggtctccagagcgaggatgaagctgattattattgcgccgcatgggatgatagcttaaatggcccagtttttggcggcggtactaaactgaccgtgctgccgagcggccaggcgggcgcggcggcatcggagtccctgtttgtgtcaaatcacgcctaccaggtgcagctggtgcagagcggcgccgaggtgaagaagcccggcgccagcgtgaaggtgagctgcaaggccagcggctacagcttcaccggctacaccatgaactgggtgcggcaggcccccggccagggcctggagtggatgggcctgatcaacccctacaacagcgacaccaactacgcccagaagctgcagggccgggtgaccatgaccaccgacaccagcaccagcaccgcctacatggagctgcggagcctgcggagcgacgacaccgccgtgtactactgcgcccgggtggccctgcgggtggccctggactactggggccagggcaccctggtgaccgtgagcagcggtgggggcggctctggtggcggtggcagcggcggaggtggcagtgacatccagatgacccagagccccagcagcctgagcgccagcgtgggcgaccgggtgaccatcacctgcaaggccagccagaacgtggccacccacgtgggctggtaccagcagaagcccggcaaggcccccaagcggctgatctacagcgccagctaccggtacagcggcgtgcccagccggttcagcggcagcggcagcggcaccgagttcaccctgaccatcagcaacctgcagcccgaggacttcgccacctactactgccagcagtacaaccggtacccctacaccttcggccagggcaccaagctggagatcaaggaaagcaaatatggcccgccgtgcccgccgtgcccggcgccggaagcggcgggcggcccgagcgtgtttctgtttccgccgaaaccgaaagataccctgatgattagccgcaccccggaagtgacctgcgtggtggtggatgtgagccaggaagatccggaagtgcagtttaactggtatgtggatggcgtggaagtgcataacgcgaaaaccaaaccgcgcgaagaacagtttcagagcacctatcgcgtggtgagcgtgctgaccgtgctgcatcaggattggctgaacggcaaagaatataaatgcaaagtgagcaacaaaggcctgccgagcagcattgaaaaaaccattagcaaagcgaaaggccagccgcgcgaaccgcaggtgtataccctgccgccgagccaggaagaaatgaccaaaaaccaggtgagcctgacctgcctggtgaaaggcttttatccgagcgatattgcggtggaatgggaaagcaacggccagccggaaaacaactataaaaccaccccgccggtgctggatagcgatggcagcttttttctgtatagccgcctgaccgtggataaaagccgctggcaggaaggcaacgtgtttagctgcagcgtgatgcatgaagcgctgcataaccattatacccagaaaagcctgagcctgagcctgggcaaatacccatacgatgttccagattacgctggtttaaacgccgagggcagaggaagtcttctaacatgcggtgacgtggaggagaatcccggccctgtcgacatgtacaggatgcaactcctgtcttgcattgcactaagtcttgcacttgtcacaaacagtggcattcatgtcttcattttgggctgtttcagtgcagggcttcctaaaacagaagccaactgggtgaatgtaatcagcgacctcaagaagatcgaggacttgatccagtccatgcacatagatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttacccatacgatgttccagattacgctcaattgggcagcggcgccacaaacttctctctgctaaagcaagcaggtgatgttgaagaaaaccccgggcctatgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatg

SEQ ID NO: 209 - protein, 1010 aa, Artificial Sequence
  Description of Artificial Sequence Synthetic polypeptide
MPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKRGLNAEGRGSLLTCGDVEENPGPVDMYRMQLLSCIALSLALVTNSMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSGGGGSGGGGSGGGGSGGGSLQITCPPPMSVEHADIWVKSYSLYSRERYICNSGFKRKAGTSSLTECVLNKATNVAHWTTPSLKCIRYPYDVPDYAQLGSGATNFSLLKQAGDVEENPGPMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 210 - DNA, 3030 bp, Artificial Sequence
  Description of Artificial Sequence Synthetic polynucleotide
atgccacctcctcgcctcctcttcttcctcctcttcctcacccccatggaagtcaggcccgaggaacctctagtggtgaaggtggaagagggagataacgctgtgctgcagtgcctcaaggggacctcagatggccccactcagcagctgacctggtctcgggagtccccgcttaaacccttcttaaaactcagcctggggctgccaggcctgggaatccacatgaggcccctggccatctggcttttcatcttcaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagccaggacctcaccatggcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatggagacgggtctgttgttgccccgggccacagctcaagacgctggaaagtattattgtcaccgtggcaacctgaccatgtcattccacctggagatcactgctcggccagtactatggcactggctgctgaggactggtggctggaaggtctcagctgtgactttggcttatctgatcttctgcctgtgttcccttgtgggcattcttcatcttcaaagagccctggtcctgaggaggaaaagaggtttaaacgccgagggcagaggaagtcttctaacatgcggtgacgtggaggagaatcccggccctgtcgacatgtacaggatgcaactcctgtcttgcattgcactaagtcttgcacttgtcacaaacagtatgagaatttcgaaaccacatttgagaagtatttccatccagtgctacttgtgtttacttctaaacagtcattttctaactgaagctggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttctagcggcggtggcagcggcggtggcggtagcggcggtggcggtagcggcggtggcggtagcggcggtggcagcctgcagatcacctgccccccccccatgagcgtggagcacgccgacatctgggtgaagagctacagcctgtacagcagagagagatacatctgcaacagcggcttcaagagaaaggccggcaccagcagcctgaccgagtgcgtgctgaacaaggccaccaacgtggcccactggaccacccccagcctgaagtgcatcagatacccatacgatgttccagattacgctcaattgggcagcggcgccacaaacttctctctgctaaagcaagcaggtgatgttgaagaaaaccccgggcctatgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatg

SEQ ID NO: 211 - protein, 1149 aa, Artificial Sequence
  Description of Artificial Sequence Synthetic polypeptide
MGWSSIILFLVATATGVHQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSGGGGSGGGGSGGGGSDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKLEPKSCDKTHTCPPCPDPKGTFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGLNAEGRGSLLTCGDVEENPGPVDMYRMQLLSCIALSLALVTNSMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSGGGGSGGGGSGGGGSGGGSLQITCPPPMSVEHADIWVKSYSLYSRERYICNSGFKRKAGTSSLTECVLNKATNVAHWTTPSLKCIRYPYDVPDYAQLGSGATNFSLLKQAGDVEENPGPMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 212 - DNA, 3447 bp, Artificial Sequence
  Description of Artificial Sequence Synthetic polynucleotide
atggggtggtcaagcattattctgtttctggtcgctaccgctacaggcgtccatcagatccagctggtgcagtccggccccgagctgaagaagcccggcgagaccgtgaagatctcctgcaaggcctccggctacaccttcaccgactactccatcaactgggtgaagcgggcccccggcaagggcctgaagtggatgggctggatcaacaccgagacccgggagcccgcctacgcctacgacttccggggccggttcgccttctccctggagacctccgcctccaccgcctacctgcagatcaacaacctgaagtacgaggacaccgccacctacttctgcgccctggactactcctacgccatggactactggggccagggcacctccgtgaccgtgtcctccggtgggggcggctctggtggcggtggcagcggcggaggtggcagtgatattgttcttactcaatcacccccaagccttgcgatgtctcttggtaaacgagcgacaattagttgtagagcttctgaaagcgtaactattcttgggtcacatcttattcattggtatcaacaaaagccgggacaaccgcctacactcttgattcaactcgcgagcaatgttcaaacgggtgtccctgcacgcttttctgggagcggttcacgaacagattttactctcacgattgatccagtcgaagaagatgatgtcgctgtatattattgtctccaaagtaggacaataccaagaacttttggtggtggtacaaaattggaaattaaactcgagcccaaatcttgtgacaaaactcacacatgcccaccgtgcccggatcccaaaggtaccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggtttaaacgccgagggcagaggaagtcttctaacatgcggtgacgtggaggagaatcccggccctgtcgacatgtacaggatgcaactcctgtcttgcattgcactaagtcttgcacttgtcacaaacagtatgagaatttcgaaaccacatttgagaagtatttccatccagtgctacttgtgtttacttctaaacagtcattttctaactgaagctggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttctagcggcggtggcagcggcggtggcggtagcggcggtggcggtagcggcggtggcggtagcggcggtggcagcctgcagatcacctgccccccccccatgagcgtggagcacgccgacatctgggtgaagagctacagcctgtacagcagagagagatacatctgcaacagcggcttcaagagaaaggccggcaccagcagcctgaccgagtgcgtgctgaacaaggccaccaacgtggcccactggaccacccccagcctgaagtgcatcagatacccatacgatgttccagattacgctcaattgggcagcggcgccacaaacttctctctgctaaagcaagcaggtgatgttgaagaaaaccccgggcctatgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatg

SEQ ID NO: 213 - protein, 1936 aa, Artificial Sequence
  Description of Artificial Sequence Synthetic polypeptide
MGWSSIILFLVATATGVHQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSGGGGSGGGGSGGGGSDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKLEPKSCDKTHTCPPCPDPKGTFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRHVGSGEGRGSLLTCGDVEENPGPMYRMQLLSCIALSLALVTNSDIQMTQSPSSLSASVGDRVTITCKASQNVATHVGWYQQKPGKAPKRLIYSASYRYSGVPSRFSGSGSGTEFTLTISNLQPEDFATYYCQQYNRYPYTFGQGTKLEIKGGGGSGGGGSGGGGSQVQLVQSGAEVKKPGASVKVSCKASGYSFTGYTMNWVRQAPGQGLEWMGLINPYNSDTNYAQKLQGRVTMTTDTSTSTAYMELRSLRSDDTAVYYCARVALRVALDYWGQGTLVTVSSPSGQAGAAASESLFVSNHAYQSALTQPASVSGSPGQSITISCSGSSSNIGNNAVNWYQQLPGKAPKLLIYYDDLLPSGVSDRFSGSKSGTSAFLAISGLQSEDEADYYCAAWDDSLNGPVFGGGTKLTVLGGGGSGGGGSGGGGSQVQLVESGGGLVKPGGSLRLSCAASGFTFSSYGMHWVRQAPGKGLEWVAFIRYDGSNKYYADSVKGRFTISRDNSKNTLYLQMNSLRAEDTAVYYCAKDRGLGDGTYFDYWGQGTTVTVSSESKYGPPCPPCPAPEAAGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKYPYDVPDYAGLNAEGRGSLLTCGDVEENPGPVDMYRMQLLSCIALSLALVTNSMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSGGGGSGGGGSGGGGSGGGSLQITCPPPMSVEHADIWVKSYSLYSRERYICNSGFKRKAGTSSLTECVLNKATNVAHWTTPSLKCIRYPYDVPDYAQLGSGATNFSLLKQAGDVEENPGPMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 214 - DNA, 5808 bp, Artificial Sequence
  Description of Artificial Sequence Synthetic polynucleotide
atggggtggtcaagcattattctgtttctggtcgctaccgctacaggcgtccatcagatccagctggtgcagtccggccccgagctgaagaagcccggcgagaccgtgaagatctcctgcaaggcctccggctacaccttcaccgactactccatcaactgggtgaagcgggcccccggcaagggcctgaagtggatgggctggatcaacaccgagacccgggagcccgcctacgcctacgacttccggggccggttcgccttctccctggagacctccgcctccaccgcctacctgcagatcaacaacctgaagtacgaggacaccgccacctacttctgcgccctggactactcctacgccatggactactggggccagggcacctccgtgaccgtgtcctccggtgggggcggctctggtggcggtggcagcggcggaggtggcagtgatattgttcttactcaatcacccccaagccttgcgatgtctcttggtaaacgagcgacaattagttgtagagcttctgaaagcgtaactattcttgggtcacatcttattcattggtatcaacaaaagccgggacaaccgcctacactcttgattcaactcgcgagcaatgttcaaacgggtgtccctgcacgcttttctgggagcggttcacgaacagattttactctcacgattgatccagtcgaagaagatgatgtcgctgtatattattgtctccaaagtaggacaataccaagaacttttggtggtggtacaaaattggaaattaaactcgagcccaaatcttgtgacaaaactcacacatgcccaccgtgcccggatcccaaaggtaccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgccacgtgggttctggagaaggacgcggttccttgttgacgtgtggcgatgtagaggaaaatccgggtccaatgtacaggatgcaactcctgtcttgcattgcactaagtcttgcacttgtcacaaacagtgacatccagatgacccagagccccagcagcctgagcgccagcgtgggcgaccgggtgaccatcacctgcaaggccagccagaacgtggccacccacgtgggctggtaccagcagaagcccggcaaggcccccaagcggctgatctacagcgccagctaccggtacagcggcgtgcccagccggttcagcggcagcggcagcggcaccgagttcaccctgaccatcagcaacctgcagcccgaggacttcgccacctactactgccagcagtacaaccggtacccctacaccttcggccagggcaccaagctggagatcaagggtgggggcggctctggtggcggtggcagcggcggaggtggcagtcaggtgcagctggtgcagagcggcgccgaggtgaagaagcccggcgccagcgtgaaggtgagctgcaaggccagcggctacagcttcaccggctacaccatgaactgggtgcggcaggcccccggccagggcctggagtggatgggcctgatcaacccctacaacagcgacaccaactacgcccagaagctgcagggccgggtgaccatgaccaccgacaccagcaccagcaccgcctacatggagctgcggagcctgcggagcgacgacaccgccgtgtactactgcgcccgggtggccctgcgggtggccctggactactggggccagggcaccctggtgaccgtgagcagcccgagcggccaggcgggcgcggcggcatcggagtccctgtttgtgtcaaatcacgcctaccagtcagcgcttacgcagccggcgtcggtgtcgggttccccgggtcagtcgatcacgatcagctgtagtgggagcagctccaacatcggtaacaacgcagtgaactggtatcagcaactgccgggaaaagcgccgaaactgctgatttactatgatgatttgctgccaagtggagttagtgaccgcttttccggcagtaaatcgggtacctcggcttttctggctatttcgggtctccagagcgaggatgaagctgattattattgcgccgcatgggatgatagcttaaatggcccagtttttggcggcggtactaaactgaccgtgctgggtgggggcggctctggtggcggtggcagcggcggaggtggcagtcaagtgcagctggttgaatccggtggcggtctggtcaagccgggcggctctttgcgtctgagctgtgccgcgtcgggttttaccttcagctcttatggtatgcattgggtgcgtcaggcgcctggcaaaggtctggagtgggttgcgttcatccgctacgatgggtctaacaaatattatgccgactcagtaaaaggacgcttcactattagccgcgacaatagcaaaaataccctgtacctgcaaatgaatagcctgcgcgccgaagataccgccgtttactattgcgctaaagatcgtggcctgggtgatggtacgtacttcgattactggggtcagggcaccaccgttaccgttagttcagaaagcaaatatgggccaccttgtcccccatgtccagccccagaggccgcaggcggtccctcagtgtttctgtttccgccgaaaccgaaagataccctgatgattagccgcaccccggaagtgacctgcgtggtggtggatgtgagccaggaagatccggaagtgcagtttaactggtatgtggatggcgtggaagtgcataacgcgaaaaccaaaccgcgcgaagaacagtttcagagcacctatcgcgtggtgagcgtgctgaccgtgctgcatcaggattggctgaacggcaaagaatataaatgcaaagtgagcaacaaaggcctgccgagcagcattgaaaaaaccattagcaaagcgaaaggccagccgcgcgaaccgcaggtgtataccctgccgccgagccaggaagaaatgaccaaaaaccaggtgagcctgacctgcctggtgaaaggcttttatccgagcgatattgcggtggaatgggaaagcaacggccagccggaaaacaactataaaaccaccccgccggtgctggatagcgatggcagcttttttctgtatagccgcctgaccgtggataaaagccgctggcaggaaggcaacgtgtttagctgcagcgtgatgcatgaagcgctgcataaccattatacccagaaaagcctgagcctgagcctgggcaaatacccatacgatgttccagattacgctggtttaaacgccgagggcagaggaagtcttctaacatgcggtgacgtggaggagaatcccggccctgtcgacatgtacaggatgcaactcctgtcttgcattgcactaagtcttgcacttgtcacaaacagtatgagaatttcgaaaccacatttgagaagtatttccatccagtgctacttgtgtttacttctaaacagtcattttctaactgaagctggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttctagcggcggtggcagcggcggtggcggtagcggcggtggcggtagcggcggtggcggtagcggcggtggcagcctgcagatcacctgccccccccccatgagcgtggagcacgccgacatctgggtgaagagctacagcctgtacagcagagagagatacatctgcaacagcggcttcaagagaaaggccggcaccagcagcctgaccgagtgcgtgctgaacaaggccaccaacgtggcccactggaccacccccagcctgaagtgcatcagatacccatacgatgttccagattacgctcaattgggcagcggcgccacaaacttctctctgctaaagcaagcaggtgatgttgaagaaaaccccgggcctatgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatg

SEQ ID NO: 215 - protein, 1936 aa, Artificial Sequence
  Description of Artificial Sequence Synthetic polypeptide
MGWSSIILFLVATATGVHQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSGGGGSGGGGSGGGGSDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKLEPKSCDKTHTCPPCPDPKGTFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRHVGSGEGRGSLLTCGDVEENPGPMYRMQLLSCIALSLALVTNSQVQLVESGGGLVKPGGSLRLSCAASGFTFSSYGMHWVRQAPGKGLEWVAFIRYDGSNKYYADSVKGRFTISRDNSKNTLYLQMNSLRAEDTAVYYCAKDRGLGDGTYFDYWGQGTTVTVSSGGGGSGGGGSGGGGSQSALTQPASVSGSPGQSITISCSGSSSNIGNNAVNWYQQLPGKAPKLLIYYDDLLPSGVSDRFSGSKSGTSAFLAISGLQSEDEADYYCAAWDDSLNGPVFGGGTKLTVLPSGQAGAAASESLFVSNHAYQVQLVQSGAEVKKPGASVKVSCKASGYSFTGYTMNWVRQAPGQGLEWMGLINPYNSDTNYAQKLQGRVTMTTDTSTSTAYMELRSLRSDDTAVYYCARVALRVALDYWGQGTLVTVSSGGGGSGGGGSGGGGSDIQMTQSPSSLSASVGDRVTITCKASQNVATHVGWYQQKPGKAPKRLIYSASYRYSGVPSRFSGSGSGTEFTLTISNLQPEDFATYYCQQYNRYPYTFGQGTKLEIKESKYGPPCPPCPAPEAAGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKYPYDVPDYAGLNAEGRGSLLTCGDVEENPGPVDMYRMQLLSCIALSLALVTNSMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSGGGGSGGGGSGGGGSGGGSLQITCPPPMSVEHADIWVKSYSLYSRERYICNSGFKRKAGTSSLTECVLNKATNVAHWTTPSLKCIRYPYDVPDYAQLGSGATNFSLLKQAGDVEENPGPMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 216 - DNA, 5808 bp, Artificial Sequence
  Description of Artificial Sequence Synthetic polynucleotide
atggggtggtcaagcattattctgtttctggtcgctaccgctacaggcgtccatcagatccagctggtgcagtccggccccgagctgaagaagcccggcgagaccgtgaagatctcctgcaaggcctccggctacaccttcaccgactactccatcaactgggtgaagcgggcccccggcaagggcctgaagtggatgggctggatcaacaccgagacccgggagcccgcctacgcctacgacttccggggccggttcgccttctccctggagacctccgcctccaccgcctacctgcagatcaacaacctgaagtacgaggacaccgccacctacttctgcgccctggactactcctacgccatggactactggggccagggcacctccgtgaccgtgtcctccggtgggggcggctctggtggcggtggcagcggcggaggtggcagtgatattgttcttactcaatcacccccaagccttgcgatgtctcttggtaaacgagcgacaattagttgtagagcttctgaaagcgtaactattcttgggtcacatcttattcattggtatcaacaaaagccgggacaaccgcctacactcttgattcaactcgcgagcaatgttcaaacgggtgtccctgcacgcttttctgggagcggttcacgaacagattttactctcacgattgatccagtcgaagaagatgatgtcgctgtatattattgtctccaaagtaggacaataccaagaacttttggtggtggtacaaaattggaaattaaactcgagcccaaatcttgtgacaaaactcacacatgcccaccgtgcccggatcccaaaggtaccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgccacgtgggttctggagaaggacgcggttccttgttgacgtgtggcgatgtagaggaaaatccgggtccaatgtacaggatgcaactcctgtcttgcattgcactaagtcttgcacttgtcacaaacagtcaagtgcagctggttgaatccggtggcggtctggtcaagccgggcggctctttgcgtctgagctgtgccgcgtcgggttttaccttcagctcttatggtatgcattgggtgcgtcaggcgcctggcaaaggtctggagtgggttgcgttcatccgctacgatgggtctaacaaatattatgccgactcagtaaaaggacgcttcactattagccgcgacaatagcaaaaataccctgtacctgcaaatgaatagcctgcgcgccgaagataccgccgtttactattgcgctaaagatcgtggcctgggtgatggtacgtacttcgattactggggtcagggcaccaccgttaccgttagttcaggtgggggcggctctggtggcggtggcagcggcggaggtggcagtcagtcagcgcttacgcagccggcgtcggtgtcgggttccccgggtcagtcgatcacgatcagctgtagtgggagcagctccaacatcggtaacaacgcagtgaactggtatcagcaactgccgggaaaagcgccgaaactgctgatttactatgatgatttgctgccaagtggagttagtgaccgcttttccggcagtaaatcgggtacctcggcttttctggctatttcgggtctccagagcgaggatgaagctgattattattgcgccgcatgggatgatagcttaaatggcccagtttttggcggcggtactaaactgaccgtgctgccgagcggccaggcgggcgcggcggcatcggagtccctgtttgtgtcaaatcacgcctaccaggtgcagctggtgcagagcggcgccgaggtgaagaagcccggcgccagcgtgaaggtgagctgcaaggccagcggctacagcttcaccggctacaccatgaactgggtgcggcaggcccccggccagggcctggagtggatgggcctgatcaacccctacaacagcgacaccaactacgcccagaagctgcagggccgggtgaccatgaccaccgacaccagcaccagcaccgcctacatggagctgcggagcctgcggagcgacgacaccgccgtgtactactgcgcccgggtggccctgcgggtggccctggactactggggccagggcaccctggtgaccgtgagcagcggtgggggcggctctggtggcggtggcagcggcggaggtggcagtgacatccagatgacccagagccccagcagcctgagcgccagcgtgggcgaccgggtgaccatcacctgcaaggccagccagaacgtggccacccacgtgggctggtaccagcagaagcccggcaaggcccccaagcggctgatctacagcgccagctaccggtacagcggcgtgcccagccggttcagcggcagcggcagcggcaccgagttcaccctgaccatcagcaacctgcagcccgaggacttcgccacctactactgccagcagtacaaccggtacccctacaccttcggccagggcaccaagctggagatcaaggaaagcaaatatggcccgccgtgcccgccgtgcccggcgccggaagcggcgggcggcccgagcgtgtttctgtttccgccgaaaccgaaagataccctgatgattagccgcaccccggaagtgacctgcgtggtggtggatgtgagccaggaagatccggaagtgcagtttaactggtatgtggatggcgtggaagtgcataacgcgaaaaccaaaccgcgcgaagaacagtttcagagcacctatcgcgtggtgagcgtgctgaccgtgctgcatcaggattggctgaacggcaaagaatataaatgcaaagtgagcaacaaaggcctgccgagcagcattgaaaaaaccattagcaaagcgaaaggccagccgcgcgaaccgcaggtgtataccctgccgccgagccaggaagaaatgaccaaaaaccaggtgagcctgacctgcctggtgaaaggcttttatccgagcgatattgcggtggaatgggaaagcaacggccagccggaaaacaactataaaaccaccccgccggtgctggatagcgatggcagcttttttctgtatagccgcctgaccgtggataaaagccgctggcaggaaggcaacgtgtttagctgcagcgtgatgcatgaagcgctgcataaccattatacccagaaaagcctgagcctgagcctgggcaaatacccatacgatgttccagattacgctggtttaaacgccgagggcagaggaagtcttctaacatgcggtgacgtggaggagaatcccggccctgtcgacatgtacaggatgcaactcctgtcttgcattgcactaagtcttgcacttgtcacaaacagtatgagaatttcgaaaccacatttgagaagtatttccatccagtgctacttgtgtttacttctaaacagtcattttctaactgaagctggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttctagcggcggtggcagcggcggtggcggtagcggcggtggcggtagcggcggtggcggtagcggcggtggcagcctgcagatcacctgccccccccccatgagcgtggagcacgccgacatctgggtgaagagctacagcctgtacagcagagagagatacatctgcaacagcggcttcaagagaaaggccggcaccagcagcctgaccgagtgcgtgctgaacaaggccaccaacgtggcccactggaccacccccagcctgaagtgcatcagatacccatacgatgttccagattacgctcaattgggcagcggcgccacaaacttctctctgctaaagcaagcaggtgatgttgaagaaaaccccgggcctatgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatg

SEQ ID NO: 217 - protein, 940 aa, Artificial Sequence
  Description of Artificial Sequence Synthetic polypeptide
MPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKRGLNAEGRGSLLTCGDVEENPGPVDMYRMQLLSCIALSLALVTNSGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSEQKLISEEDLAVGQDTQEVIVVPHSLPFKVVVISAILALVVLTIISLIILIMLWQKKPRQLGSGATNFSLLKQAGDVEENPGPMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 218 - DNA, 2820 bp, Artificial Sequence
  Description of Artificial Sequence Synthetic polynucleotide
atgccacctcctcgcctcctcttcttcctcctcttcctcacccccatggaagtcaggcccgaggaacctctagtggtgaaggtggaagagggagataacgctgtgctgcagtgcctcaaggggacctcagatggccccactcagcagctgacctggtctcgggagtccccgcttaaacccttcttaaaactcagcctggggctgccaggcctgggaatccacatgaggcccctggccatctggcttttcatcttcaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagccaggacctcaccatggcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatggagacgggtctgttgttgccccgggccacagctcaagacgctggaaagtattattgtcaccgtggcaacctgaccatgtcattccacctggagatcactgctcggccagtactatggcactggctgctgaggactggtggctggaaggtctcagctgtgactttggcttatctgatcttctgcctgtgttcccttgtgggcattcttcatcttcaaagagccctggtcctgaggaggaaaagaggtttaaacgccgagggcagaggaagtcttctaacatgcggtgacgtggaggagaatcccggccctgtcgacatgtacaggatgcaactcctgtcttgcattgcactaagtcttgcacttgtcacaaacagtggcattcatgtcttcattttgggctgtttcagtgcagggcttcctaaaacagaagccaactgggtgaatgtaatcagcgacctcaagaagatcgaggacttgatccagtccatgcacatagatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttctgaacaaaaactcatctcagaagaggatctggctgtgggccaggacacgcaggaggtcatcgtggtgccacactccttgccctttaaggtggtggtgatctcagccatcctggccctggtggtgctcaccatcatctcccttatcatcctcatcatgctttggcagaagaagccacgtcaattgggcagcggcgccacaaacttctctctgctaaagcaagcaggtgatgttgaagaaaaccccgggcctatgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatg

SEQ ID NO: 219 - protein, 1079 aa, Artificial Sequence
  Description of Artificial Sequence Synthetic polypeptide
MGWSSIILFLVATATGVHQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSGGGGSGGGGSGGGGSDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKLEPKSCDKTHTCPPCPDPKGTFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGLNAEGRGSLLTCGDVEENPGPVDMYRMQLLSCIALSLALVTNSGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSEQKLISEEDLAVGQDTQEVIVVPHSLPFKVVVISAILALVVLTIISLIILIMLWQKKPRQLGSGATNFSLLKQAGDVEENPGPMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 220 - DNA, 3237 bp, Artificial Sequence
  Description of Artificial Sequence Synthetic polynucleotide
atggggtggtcaagcattattctgtttctggtcgctaccgctacaggcgtccatcagatccagctggtgcagtccggccccgagctgaagaagcccggcgagaccgtgaagatctcctgcaaggcctccggctacaccttcaccgactactccatcaactgggtgaagcgggcccccggcaagggcctgaagtggatgggctggatcaacaccgagacccgggagcccgcctacgcctacgacttccggggccggttcgccttctccctggagacctccgcctccaccgcctacctgcagatcaacaacctgaagtacgaggacaccgccacctacttctgcgccctggactactcctacgccatggactactggggccagggcacctccgtgaccgtgtcctccggtgggggcggctctggtggcggtggcagcggcggaggtggcagtgatattgttcttactcaatcacccccaagccttgcgatgtctcttggtaaacgagcgacaattagttgtagagcttctgaaagcgtaactattcttgggtcacatcttattcattggtatcaacaaaagccgggacaaccgcctacactcttgattcaactcgcgagcaatgttcaaacgggtgtccctgcacgcttttctgggagcggttcacgaacagattttactctcacgattgatccagtcgaagaagatgatgtcgctgtatattattgtctccaaagtaggacaataccaagaacttttggtggtggtacaaaattggaaattaaactcgagcccaaatcttgtgacaaaactcacacatgcccaccgtgcccggatcccaaaggtaccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggtttaaacgccgagggcagaggaagtcttctaacatgcggtgacgtggaggagaatcccggccctgtcgacatgtacaggatgcaactcctgtcttgcattgcactaagtcttgcacttgtcacaaacagtggcattcatgtcttcattttgggctgtttcagtgcagggcttcctaaaacagaagccaactgggtgaatgtaatcagcgacctcaagaagatcgaggacttgatccagtccatgcacatagatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttctgaacaaaaactcatctcagaagaggatctggctgtgggccaggacacgcaggaggtcatcgtggtgccacactccttgccctttaaggtggtggtgatctcagccatcctggccctggtggtgctcaccatcatctcccttatcatcctcatcatgctttggcagaagaagccacgtcaattgggcagcggcgccacaaacttctctctgctaaagcaagcaggtgatgttgaagaaaaccccgggcctatgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatg

SEQ ID NO: 221 - protein, 1866 aa, Artificial Sequence
  Description of Artificial Sequence Synthetic polypeptide
MGWSSIILFLVATATGVHQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSGGGGSGGGGSGGGGSDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKLEPKSCDKTHTCPPCPDPKGTFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRHVGSGEGRGSLLTCGDVEENPGPMYRMQLLSCIALSLALVTNSDIQMTQSPSSLSASVGDRVTITCKASQNVATHVGWYQQKPGKAPKRLIYSASYRYSGVPSRFSGSGSGTEFTLTISNLQPEDFATYYCQQYNRYPYTFGQGTKLEIKGGGGSGGGGSGGGGSQVQLVQSGAEVKKPGASVKVSCKASGYSFTGYTMNWVRQAPGQGLEWMGLINPYNSDTNYAQKLQGRVTMTTDTSTSTAYMELRSLRSDDTAVYYCARVALRVALDYWGQGTLVTVSSPSGQAGAAASESLFVSNHAYQSALTQPASVSGSPGQSITISCSGSSSNIGNNAVNWYQQLPGKAPKLLIYYDDLLPSGVSDRFSGSKSGTSAFLAISGLQSEDEADYYCAAWDDSLNGPVFGGGTKLTVLGGGGSGGGGSGGGGSQVQLVESGGGLVKPGGSLRLSCAASGFTFSSYGMHWVRQAPGKGLEWVAFIRYDGSNKYYADSVKGRFTISRDNSKNTLYLQMNSLRAEDTAVYYCAKDRGLGDGTYFDYWGQGTTVTVSSESKYGPPCPPCPAPEAAGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKYPYDVPDYAGLNAEGRGSLLTCGDVEENPGPVDMYRMQLLSCIALSLALVTNSGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSEQKLISEEDLAVGQDTQEVIVVPHSLPFKVVVISAILALVVLTIISLIILIMLWQKKPRQLGSGATNFSLLKQAGDVEENPGPMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 222 - DNA, 5598 bp, Artificial Sequence
  Description of Artificial Sequence Synthetic polynucleotide
atggggtggtcaagcattattctgtttctggtcgctaccgctacaggcgtccatcagatccagctggtgcagtccggccccgagctgaagaagcccggcgagaccgtgaagatctcctgcaaggcctccggctacaccttcaccgactactccatcaactgggtgaagcgggcccccggcaagggcctgaagtggatgggctggatcaacaccgagacccgggagcccgcctacgcctacgacttccggggccggttcgccttctccctggagacctccgcctccaccgcctacctgcagatcaacaacctgaagtacgaggacaccgccacctacttctgcgccctggactactcctacgccatggactactggggccagggcacctccgtgaccgtgtcctccggtgggggcggctctggtggcggtggcagcggcggaggtggcagtgatattgttcttactcaatcacccccaagccttgcgatgtctcttggtaaacgagcgacaattagttgtagagcttctgaaagcgtaactattcttgggtcacatcttattcattggtatcaacaaaagccgggacaaccgcctacactcttgattcaactcgcgagcaatgttcaaacgggtgtccctgcacgcttttctgggagcggttcacgaacagattttactctcacgattgatccagtcgaagaagatgatgtcgctgtatattattgtctccaaagtaggacaataccaagaacttttggtggtggtacaaaattggaaattaaactcgagcccaaatcttgtgacaaaactcacacatgcccaccgtgcccggatcccaaaggtaccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgccacgtgggttctggagaaggacgcggttccttgttgacgtgtggcgatgtagaggaaaatccgggtccaatgtacaggatgcaactcctgtcttgcattgcactaagtcttgcacttgtcacaaacagtgacatccagatgacccagagccccagcagcctgagcgccagcgtgggcgaccgggtgaccatcacctgcaaggccagccagaacgtggccacccacgtgggctggtaccagcagaagcccggcaaggcccccaagcggctgatctacagcgccagctaccggtacagcggcgtgcccagccggttcagcggcagcggcagcggcaccgagttcaccctgaccatcagcaacctgcagcccgaggacttcgccacctactactgccagcagtacaaccggtacccctacaccttcggccagggcaccaagctggagatcaagggtgggggcggctctggtggcggtggcagcggcggaggtggcagtcaggtgcagctggtgcagagcggcgccgaggtgaagaagcccggcgccagcgtgaaggtgagctgcaaggccagcggctacagcttcaccggctacaccatgaactgggtgcggcaggcccccggccagggcctggagtggatgggcctgatcaacccctacaacagcgacaccaactacgcccagaagctgcagggccgggtgaccatgaccaccgacaccagcaccagcaccgcctacatggagctgcggagcctgcggagcgacgacaccgccgtgtactactgcgcccgggtggccctgcgggtggccctggactactggggccagggcaccctggtgaccgtgagcagcccgagcggccaggcgggcgcggcggcatcggagtccctgtttgtgtcaaatcacgcctaccagtcagcgcttacgcagccggcgtcggtgtcgggttccccgggtcagtcgatcacgatcagctgtagtgggagcagctccaacatcggtaacaacgcagtgaactggtatcagcaactgccgggaaaagcgccgaaactgctgatttactatgatgatttgctgccaagtggagttagtgaccgcttttccggcagtaaatcgggtacctcggcttttctggctatttcgggtctccagagcgaggatgaagctgattattattgcgccgcatgggatgatagcttaaatggcccagtttttggcggcggtactaaactgaccgtgctgggtgggggcggctctggtggcggtggcagcggcggaggtggcagtcaagtgcagctggttgaatccggtggcggtctggtcaagccgggcggctctttgcgtctgagctgtgccgcgtcgggttttaccttcagctcttatggtatgcattgggtgcgtcaggcgcctggcaaaggtctggagtgggttgcgttcatccgctacgatgggtctaacaaatattatgccgactcagtaaaaggacgcttcactattagccgcgacaatagcaaaaataccctgtacctgcaaatgaatagcctgcgcgccgaagataccgccgtttactattgcgctaaagatcgtggcctgggtgatggtacgtacttcgattactggggtcagggcaccaccgttaccgttagttcagaaagcaaatatgggccaccttgtcccccatgtccagccccagaggccgcaggcggtccctcagtgtttctgtttccgccgaaaccgaaagataccctgatgattagccgcaccccggaagtgacctgcgtggtggtggatgtgagccaggaagatccggaagtgcagtttaactggtatgtggatggcgtggaagtgcataacgcgaaaaccaaaccgcgcgaagaacagtttcagagcacctatcgcgtggtgagcgtgctgaccgtgctgcatcaggattggctgaacggcaaagaatataaatgcaaagtgagcaacaaaggcctgccgagcagcattgaaaaaaccattagcaaagcgaaaggccagccgcgcgaaccgcaggtgtataccctgccgccgagccaggaagaaatgaccaaaaaccaggtgagcctgacctgcctggtgaaaggcttttatccgagcgatattgcggtggaatgggaaagcaacggccagccggaaaacaactataaaaccaccccgccggtgctggatagcgatggcagcttttttctgtatagccgcctgaccgtggataaaagccgctggcaggaaggcaacgtgtttagctgcagcgtgatgcatgaagcgctgcataaccattatacccagaaaagcctgagcctgagcctgggcaaatacccatacgatgttccagattacgctggtttaaacgccgagggcagaggaagtcttctaacatgcggtgacgtggaggagaatcccggccctgtcgacatgtacaggatgcaactcctgtcttgcattgcactaagtcttgcacttgtcacaaacagtggcattcatgtcttcattttgggctgtttcagtgcagggcttcctaaaacagaagccaactgggtgaatgtaatcagcgacctcaagaagatcgaggacttgatccagtccatgcacatagatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttctgaacaaaaactcatctcagaagaggatctggctgtgggccaggacacgcaggaggtcatcgtggtgccacactccttgccctttaaggtggtggtgatctcagccatcctggccctggtggtgctcaccatcatctcccttatcatcctcatcatgctttggcagaagaagccacgtcaattgggcagcggcgccacaaacttctctctgctaaagcaagcaggtgatgttgaagaaaaccccgggcctatgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatg

SEQ ID NO: 223 - protein, 1866 aa, Artificial Sequence
  Description of Artificial Sequence Synthetic polypeptide
MGWSSIILFLVATATGVHQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSGGGGSGGGGSGGGGSDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKLEPKSCDKTHTCPPCPDPKGTFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRHVGSGEGRGSLLTCGDVEENPGPMYRMQLLSCIALSLALVTNSQVQLVESGGGLVKPGGSLRLSCAASGFTFSSYGMHWVRQAPGKGLEWVAFIRYDGSNKYYADSVKGRFTISRDNSKNTLYLQMNSLRAEDTAVYYCAKDRGLGDGTYFDYWGQGTTVTVSSGGGGSGGGGSGGGGSQSALTQPASVSGSPGQSITISCSGSSSNIGNNAVNWYQQLPGKAPKLLIYYDDLLPSGVSDRFSGSKSGTSAFLAISGLQSEDEADYYCAAWDDSLNGPVFGGGTKLTVLPSGQAGAAASESLFVSNHAYQVQLVQSGAEVKKPGASVKVSCKASGYSFTGYTMNWVRQAPGQGLEWMGLINPYNSDTNYAQKLQGRVTMTTDTSTSTAYMELRSLRSDDTAVYYCARVALRVALDYWGQGTLVTVSSGGGGSGGGGSGGGGSDIQMTQSPSSLSASVGDRVTITCKASQNVATHVGWYQQKPGKAPKRLIYSASYRYSGVPSRFSGSGSGTEFTLTISNLQPEDFATYYCQQYNRYPYTFGQGTKLEIKESKYGPPCPPCPAPEAAGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKYPYDVPDYAGLNAEGRGSLLTCGDVEENPGPVDMYRMQLLSCIALSLALVTNSGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSEQKLISEEDLAVGQDTQEVIVVPHSLPFKVVVISAILALVVLTIISLIILIMLWQKKPRQLGSGATNFSLLKQAGDVEENPGPMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 224 - DNA, 5598 bp, Artificial Sequence
  Description of Artificial Sequence Synthetic polynucleotide
atggggtggtcaagcattattctgtttctggtcgctaccgctacaggcgtccatcagatccagctggtgcagtccggccccgagctgaagaagcccggcgagaccgtgaagatctcctgcaaggcctccggctacaccttcaccgactactccatcaactgggtgaagcgggcccccggcaagggcctgaagtggatgggctggatcaacaccgagacccgggagcccgcctacgcctacgacttccggggccggttcgccttctccctggagacctccgcctccaccgcctacctgcagatcaacaacctgaagtacgaggacaccgccacctacttctgcgccctggactactcctacgccatggactactggggccagggcacctccgtgaccgtgtcctccggtgggggcggctctggtggcggtggcagcggcggaggtggcagtgatattgttcttactcaatcacccccaagccttgcgatgtctcttggtaaacgagcgacaattagttgtagagcttctgaaagcgtaactattcttgggtcacatcttattcattggtatcaacaaaagccgggacaaccgcctacactcttgattcaactcgcgagcaatgttcaaacgggtgtccctgcacgcttttctgggagcggttcacgaacagattttactctcacgattgatccagtcgaagaagatgatgtcgctgtatattattgtctccaaagtaggacaataccaagaacttttggtggtggtacaaaattggaaattaaactcgagcccaaatcttgtgacaaaactcacacatgcccaccgtgcccggatcccaaaggtaccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgccacgtgggttctggagaaggacgcggttccttgttgacgtgtggcgatgtagaggaaaatccgggtccaatgtacaggatgcaactcctgtcttgcattgcactaagtcttgcacttgtcacaaacagtcaagtgcagctggttgaatccggtggcggtctggtcaagccgggcggctctttgcgtctgagctgtgccgcgtcgggttttaccttcagctcttatggtatgcattgggtgcgtcaggcgcctggcaaaggtctggagtgggttgcgttcatccgctacgatgggtctaacaaatattatgccgactcagtaaaaggacgcttcactattagccgcgacaatagcaaaaataccctgtacctgcaaatgaatagcctgcgcgccgaagataccgccgtttactattgcgctaaagatcgtggcctgggtgatggtacgtacttcgattactggggtcagggcaccaccgttaccgttagttcaggtgggggcggctctggtggcggtggcagcggcggaggtggcagtcagtcagcgcttacgcagccggcgtcggtgtcgggttccccgggtcagtcgatcacgatcagctgtagtgggagcagctccaacatcggtaacaacgcagtgaactggtatcagcaactgccgggaaaagcgccgaaactgctgatttactatgatgatttgctgccaagtggagttagtgaccgcttttccggcagtaaatcgggtacctcggcttttctggctatttcgggtctccagagcgaggatgaagctgattattattgcgccgcatgggatgatagcttaaatggcccagtttttggcggcggtactaaactgaccgtgctgccgagcggccaggcgggcgcggcggcatcggagtccctgtttgtgtcaaatcacgcctaccaggtgcagctggtgcagagcggcgccgaggtgaagaagcccggcgccagcgtgaaggtgagctgcaaggccagcggctacagcttcaccggctacaccatgaactgggtgcggcaggcccccggccagggcctggagtggatgggcctgatcaacccctacaacagcgacaccaactacgcccagaagctgcagggccgggtgaccatgaccaccgacaccagcaccagcaccgcctacatggagctgcggagcctgcggagcgacgacaccgccgtgtactactgcgcccgggtggccctgcgggtggccctggactactggggccagggcaccctggtgaccgtgagcagcggtgggggcggctctggtggcggtggcagcggcggaggtggcagtgacatccagatgacccagagccccagcagcctgagcgccagcgtgggcgaccgggtgaccatcacctgcaaggccagccagaacgtggccacccacgtgggctggtaccagcagaagcccggcaaggcccccaagcggctgatctacagcgccagctaccggtacagcggcgtgcccagccggttcagcggcagcggcagcggcaccgagttcaccctgaccatcagcaacctgcagcccgaggacttcgccacctactactgccagcagtacaaccggtacccctacaccttcggccagggcaccaagctggagatcaaggaaagcaaatatggcccgccgtgcccgccgtgcccggcgccggaagcggcgggcggcccgagcgtgtttctgtttccgccgaaaccgaaagataccctgatgattagccgcaccccggaagtgacctgcgtggtggtggatgtgagccaggaagatccggaagtgcagtttaactggtatgtggatggcgtggaagtgcataacgcgaaaaccaaaccgcgcgaagaacagtttcagagcacctatcgcgtggtgagcgtgctgaccgtgctgcatcaggattggctgaacggcaaagaatataaatgcaaagtgagcaacaaaggcctgccgagcagcattgaaaaaaccattagcaaagcgaaaggccagccgcgcgaaccgcaggtgtataccctgccgccgagccaggaagaaatgaccaaaaaccaggtgagcctgacctgcctggtgaaaggcttttatccgagcgatattgcggtggaatgggaaagcaacggccagccggaaaacaactataaaaccaccccgccggtgctggatagcgatggcagcttttttctgtatagccgcctgaccgtggataaaagccgctggcaggaaggcaacgtgtttagctgcagcgtgatgcatgaagcgctgcataaccattatacccagaaaagcctgagcctgagcctgggcaaatacccatacgatgttccagattacgctggtttaaacgccgagggcagaggaagtcttctaacatgcggtgacgtggaggagaatcccggccctgtcgacatgtacaggatgcaactcctgtcttgcattgcactaagtcttgcacttgtcacaaacagtggcattcatgtcttcattttgggctgtttcagtgcagggcttcctaaaacagaagccaactgggtgaatgtaatcagcgacctcaagaagatcgaggacttgatccagtccatgcacatagatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttctgaacaaaaactcatctcagaagaggatctggctgtgggccaggacacgcaggaggtcatcgtggtgccacactccttgccctttaaggtggtggtgatctcagccatcctggccctggtggtgctcaccatcatctcccttatcatcctcatcatgctttggcagaagaagccacgtcaattgggcagcggcgccacaaacttctctctgctaaagcaagcaggtgatgttgaagaaaaccccgggcctatgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatg

SEQ ID NO: 225 - protein, 955 aa, Artificial Sequence
  Description of Artificial Sequence Synthetic polypeptide
MPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKRGLNAEGRGSLLTCGDVEENPGPMWLQSLLLLGTVACSISMRSSPGNMERIVICLMVIFLGTLVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKVSTLSFIEQKLISEEDLAVGQDTQEVIVVPHSLPFKVVVISAILALVVLTIISLIILIMLWQKKPRQLGSGATNFSLLKQAGDVEENPGPMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 226 - DNA, 2876 bp, Artificial Sequence
  Description of Artificial Sequence Synthetic polynucleotide
atgccacctcctcgcctcctcttcttcctcctcttcctcacccccatggaagtcaggcccgaggaacctctagtggtgaaggtggaagagggagataacgctgtgctgcagtgcctcaaggggacctcagatggccccactcagcagctgacctggtctcgggagtccccgcttaaacccttcttaaaactcagcctggggctgccaggcctgggaatccacatgaggcccctggccatctggcttttcatcttcaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagccaggacctcaccatggcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatggagacgggtctgttgttgccccgggccacagctcaagacgctggaaagtattattgtcaccgtggcaacctgaccatgtcattccacctggagatcactgctcggccagtactatggcactggctgctgaggactggtggctggaaggtctcagctgtgactttggcttatctgatcttctgcctgtgttcccttgtgggcattcttcatcttcaaagagccctggtcctgaggaggaaaagaggtttaaacgccgagggcagaggaagtcttctaacatgcggtgacgtggaggagaatcccggccctgtcgacccaccatgtggctgcagagcctgctgctcttgggcactgtggcctgcagcatctctatgagatccagtcctggcaacatggagaggattgtcatctgtctgatggtcatcttcttggggacactggtccacaaatcaagctcccaaggtcaagatcgccacatgattagaatgcgtcaacttatagatattgttgatcagctgaaaaattatgtgaatgacttggtccctgaatttctgccagctccagaagatgtagagacaaactgtgagtggtcagctttttcctgctttcagaaggcccaactaaagtcagcaaatacaggaaacaatgaaaggataatcaatgtatcaattaaaaagctgaagaggaaaccaccttccacaaatgcagggagaagacagaaacacagactaacatgcccttcatgtgattcttatgagaaaaaaccacccaaagaattcctagaaagattcaaatcacttctccaaaaggtatctaccttaagtttcattgaacaaaaactcatctcagaagaggatctggctgtgggccaggacacgcaggaggtcatcgtggtgccacactccttgccctttaaggtggtggtgatctcagccatcctggccctggtggtgctcaccatcatctcccttatcatcctcatcatgctttggcagaagaagccacgtcaattgggcagcggcgccacaaacttctctctgctaaagcaagcaggtgatgttgaagaaaaccccgggcctatgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatg

SEQ ID NO: 227 - protein, 1094 aa, Artificial Sequence
  Description of Artificial Sequence Synthetic polypeptide
MGWSSIILFLVATATGVHQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSGGGGSGGGGSGGGGSDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKLEPKSCDKTHTCPPCPDPKGTFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGLNAEGRGSLLTCGDVEENPGPMWLQSLLLLGTVACSISMRSSPGNMERIVICLMVIFLGTLVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKVSTLSFIEQKLISEEDLAVGQDTQEVIVVPHSLPFKVVVISAILALVVLTIISLIILIMLWQKKPRQLGSGATNFSLLKQAGDVEENPGPMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 228 - DNA, 3293 bp, Artificial Sequence
  Description of Artificial Sequence Synthetic polynucleotide
atggggtggtcaagcattattctgtttctggtcgctaccgctacaggcgtccatcagatccagctggtgcagtccggccccgagctgaagaagcccggcgagaccgtgaagatctcctgcaaggcctccggctacaccttcaccgactactccatcaactgggtgaagcgggcccccggcaagggcctgaagtggatgggctggatcaacaccgagacccgggagcccgcctacgcctacgacttccggggccggttcgccttctccctggagacctccgcctccaccgcctacctgcagatcaacaacctgaagtacgaggacaccgccacctacttctgcgccctggactactcctacgccatggactactggggccagggcacctccgtgaccgtgtcctccggtgggggcggctctggtggcggtggcagcggcggaggtggcagtgatattgttcttactcaatcacccccaagccttgcgatgtctcttggtaaacgagcgacaattagttgtagagcttctgaaagcgtaactattcttgggtcacatcttattcattggtatcaacaaaagccgggacaaccgcctacactcttgattcaactcgcgagcaatgttcaaacgggtgtccctgcacgcttttctgggagcggttcacgaacagattttactctcacgattgatccagtcgaagaagatgatgtcgctgtatattattgtctccaaagtaggacaataccaagaacttttggtggtggtacaaaattggaaattaaactcgagcccaaatcttgtgacaaaactcacacatgcccaccgtgcccggatcccaaaggtaccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggtttaaacgccgagggcagaggaagtcttctaacatgcggtgacgtggaggagaatcccggccctgtcgacccaccatgtggctgcagagcctgctgctcttgggcactgtggcctgcagcatctctatgagatccagtcctggcaacatggagaggattgtcatctgtctgatggtcatcttcttggggacactggtccacaaatcaagctcccaaggtcaagatcgccacatgattagaatgcgtcaacttatagatattgttgatcagctgaaaaattatgtgaatgacttggtccctgaatttctgccagctccagaagatgtagagacaaactgtgagtggtcagctttttcctgctttcagaaggcccaactaaagtcagcaaatacaggaaacaatgaaaggataatcaatgtatcaattaaaaagctgaagaggaaaccaccttccacaaatgcagggagaagacagaaacacagactaacatgcccttcatgtgattcttatgagaaaaaaccacccaaagaattcctagaaagattcaaatcacttctccaaaaggtatctaccttaagtttcattgaacaaaaactcatctcagaagaggatctggctgtgggccaggacacgcaggaggtcatcgtggtgccacactccttgccctttaaggtggtggtgatctcagccatcctggccctggtggtgctcaccatcatctcccttatcatcctcatcatgctttggcagaagaagccacgtcaattgggcagcggcgccacaaacttctctctgctaaagcaagcaggtgatgttgaagaaaaccccgggcctatgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatg

SEQ ID NO: 229 - protein, 1881 aa, Artificial Sequence
  Description of Artificial Sequence Synthetic polypeptide
MGWSSIILFLVATATGVHQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSGGGGSGGGGSGGGGSDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKLEPKSCDKTHTCPPCPDPKGTFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRHVGSGEGRGSLLTCGDVEENPGPMYRMQLLSCIALSLALVTNSDIQMTQSPSSLSASVGDRVTITCKASQNVATHVGWYQQKPGKAPKRLIYSASYRYSGVPSRFSGSGSGTEFTLTISNLQPEDFATYYCQQYNRYPYTFGQGTKLEIKGGGGSGGGGSGGGGSQVQLVQSGAEVKKPGASVKVSCKASGYSFTGYTMNWVRQAPGQGLEWMGLINPYNSDTNYAQKLQGRVTMTTDTSTSTAYMELRSLRSDDTAVYYCARVALRVALDYWGQGTLVTVSSPSGQAGAAASESLFVSNHAYQSALTQPASVSGSPGQSITISCSGSSSNIGNNAVNWYQQLPGKAPKLLIYYDDLLPSGVSDRFSGSKSGTSAFLAISGLQSEDEADYYCAAWDDSLNGPVFGGGTKLTVLGGGGSGGGGSGGGGSQVQLVESGGGLVKPGGSLRLSCAASGFTFSSYGMHWVRQAPGKGLEWVAFIRYDGSNKYYADSVKGRFTISRDNSKNTLYLQMNSLRAEDTAVYYCAKDRGLGDGTYFDYWGQGTTVTVSSESKYGPPCPPCPAPEAAGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKYPYDVPDYAGLNAEGRGSLLTCGDVEENPGPMWLQSLLLLGTVACSISMRSSPGNMERIVICLMVIFLGTLVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKVSTLSFIEQKLISEEDLAVGQDTQEVIVVPHSLPFKVVVISAILALVVLTIISLIILIMLWQKKPRQLGSGATNFSLLKQAGDVEENPGPMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 230 - DNA, 5654 bp, Artificial Sequence
  Description of Artificial Sequence Synthetic polynucleotide
atggggtggtcaagcattattctgtttctggtcgctaccgctacaggcgtccatcagatccagctggtgcagtccggccccgagctgaagaagcccggcgagaccgtgaagatctcctgcaaggcctccggctacaccttcaccgactactccatcaactgggtgaagcgggcccccggcaagggcctgaagtggatgggctggatcaacaccgagacccgggagcccgcctacgcctacgacttccggggccggttcgccttctccctggagacctccgcctccaccgcctacctgcagatcaacaacctgaagtacgaggacaccgccacctacttctgcgccctggactactcctacgccatggactactggggccagggcacctccgtgaccgtgtcctccggtgggggcggctctggtggcggtggcagcggcggaggtggcagtgatattgttcttactcaatcacccccaagccttgcgatgtctcttggtaaacgagcgacaattagttgtagagcttctgaaagcgtaactattcttgggtcacatcttattcattggtatcaacaaaagccgggacaaccgcctacactcttgattcaactcgcgagcaatgttcaaacgggtgtccctgcacgcttttctgggagcggttcacgaacagattttactctcacgattgatccagtcgaagaagatgatgtcgctgtatattattgtctccaaagtaggacaataccaagaacttttggtggtggtacaaaattggaaattaaactcgagcccaaatcttgtgacaaaactcacacatgcccaccgtgcccggatcccaaaggtaccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgccacgtgggttctggagaaggacgcggttccttgttgacgtgtggcgatgtagaggaaaatccgggtccaatgtacaggatgcaactcctgtcttgcattgcactaagtcttgcacttgtcacaaacagtgacatccagatgacccagagccccagcagcctgagcgccagcgtgggcgaccgggtgaccatcacctgcaaggccagccagaacgtggccacccacgtgggctggtaccagcagaagcccggcaaggcccccaagcggctgatctacagcgccagctaccggtacagcggcgtgcccagccggttcagcggcagcggcagcggcaccgagttcaccctgaccatcagcaacctgcagcccgaggacttcgccacctactactgccagcagtacaaccggtacccctacaccttcggccagggcaccaagctggagatcaagggtgggggcggctctggtggcggtggcagcggcggaggtggcagtcaggtgcagctggtgcagagcggcgccgaggtgaagaagcccggcgccagcgtgaaggtgagctgcaaggccagcggctacagcttcaccggctacaccatgaactgggtgcggcaggcccccggccagggcctggagtggatgggcctgatcaacccctacaacagcgacaccaactacgcccagaagctgcagggccgggtgaccatgaccaccgacaccagcaccagcaccgcctacatggagctgcggagcctgcggagcgacgacaccgccgtgtactactgcgcccgggtggccctgcgggtggccctggactactggggccagggcaccctggtgaccgtgagcagcccgagcggccaggcgggcgcggcggcatcggagtccctgtttgtgtcaaatcacgcctaccagtcagcgcttacgcagccggcgtcggtgtcgggttccccgggtcagtcgatcacgatcagctgtagtgggagcagctccaacatcggtaacaacgcagtgaactggtatcagcaactgccgggaaaagcgccgaaactgctgatttactatgatgatttgctgccaagtggagttagtgaccgcttttccggcagtaaatcgggtacctcggcttttctggctatttcgggtctccagagcgaggatgaagctgattattattgcgccgcatgggatgatagcttaaatggcccagtttttggcggcggtactaaactgaccgtgctgggtgggggcggctctggtggcggtggcagcggcggaggtggcagtcaagtgcagctggttgaatccggtggcggtctggtcaagccgggcggctctttgcgtctgagctgtgccgcgtcgggttttaccttcagctcttatggtatgcattgggtgcgtcaggcgcctggcaaaggtctggagtgggttgcgttcatccgctacgatgggtctaacaaatattatgccgactcagtaaaaggacgcttcactattagccgcgacaatagcaaaaataccctgtacctgcaaatgaatagcctgcgcgccgaagataccgccgtttactattgcgctaaagatcgtggcctgggtgatggtacgtacttcgattactggggtcagggcaccaccgttaccgttagttcagaaagcaaatatgggccaccttgtcccccatgtccagccccagaggccgcaggcggtccctcagtgtttctgtttccgccgaaaccgaaagataccctgatgattagccgcaccccggaagtgacctgcgtggtggtggatgtgagccaggaagatccggaagtgcagtttaactggtatgtggatggcgtggaagtgcataacgcgaaaaccaaaccgcgcgaagaacagtttcagagcacctatcgcgtggtgagcgtgctgaccgtgctgcatcaggattggctgaacggcaaagaatataaatgcaaagtgagcaacaaaggcctgccgagcagcattgaaaaaaccattagcaaagcgaaaggccagccgcgcgaaccgcaggtgtataccctgccgccgagccaggaagaaatgaccaaaaaccaggtgagcctgacctgcctggtgaaaggcttttatccgagcgatattgcggtggaatgggaaagcaacggccagccggaaaacaactataaaaccaccccgccggtgctggatagcgatggcagcttttttctgtatagccgcctgaccgtggataaaagccgctggcaggaaggcaacgtgtttagctgcagcgtgatgcatgaagcgctgcataaccattatacccagaaaagcctgagcctgagcctgggcaaatacccatacgatgttccagattacgctggtttaaacgccgagggcagaggaagtcttctaacatgcggtgacgtggaggagaatcccggccctgtcgacccaccatgtggctgcagagcctgctgctcttgggcactgtggcctgcagcatctctatgagatccagtcctggcaacatggagaggattgtcatctgtctgatggtcatcttcttggggacactggtccacaaatcaagctcccaaggtcaagatcgccacatgattagaatgcgtcaacttatagatattgttgatcagctgaaaaattatgtgaatgacttggtccctgaatttctgccagctccagaagatgtagagacaaactgtgagtggtcagctttttcctgctttcagaaggcccaactaaagtcagcaaatacaggaaacaatgaaaggataatcaatgtatcaattaaaaagctgaagaggaaaccaccttccacaaatgcagggagaagacagaaacacagactaacatgcccttcatgtgattcttatgagaaaaaaccacccaaagaattcctagaaagattcaaatcacttctccaaaaggtatctaccttaagtttcattgaacaaaaactcatctcagaagaggatctggctgtgggccaggacacgcaggaggtcatcgtggtgccacactccttgccctttaaggtggtggtgatctcagccatcctggccctggtggtgctcaccatcatctcccttatcatcctcatcatgctttggcagaagaagccacgtcaattgggcagcggcgccacaaacttctctctgctaaagcaagcaggtgatgttgaagaaaaccccgggcctatgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatg

SEQ ID NO: 231 - protein, 1881 aa, Artificial Sequence
  Description of Artificial Sequence Synthetic polypeptide
MGWSSIILFLVATATGVHQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSGGGGSGGGGSGGGGSDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKLEPKSCDKTHTCPPCPDPKGTFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRHVGSGEGRGSLLTCGDVEENPGPMYRMQLLSCIALSLALVTNSQVQLVESGGGLVKPGGSLRLSCAASGFTFSSYGMHWVRQAPGKGLEWVAFIRYDGSNKYYADSVKGRFTISRDNSKNTLYLQMNSLRAEDTAVYYCAKDRGLGDGTYFDYWGQGTTVTVSSGGGGSGGGGSGGGGSQSALTQPASVSGSPGQSITISCSGSSSNIGNNAVNWYQQLPGKAPKLLIYYDDLLPSGVSDRFSGSKSGTSAFLAISGLQSEDEADYYCAAWDDSLNGPVFGGGTKLTVLPSGQAGAAASESLFVSNHAYQVQLVQSGAEVKKPGASVKVSCKASGYSFTGYTMNWVRQAPGQGLEWMGLINPYNSDTNYAQKLQGRVTMTTDTSTSTAYMELRSLRSDDTAVYYCARVALRVALDYWGQGTLVTVSSGGGGSGGGGSGGGGSDIQMTQSPSSLSASVGDRVTITCKASQNVATHVGWYQQKPGKAPKRLIYSASYRYSGVPSRFSGSGSGTEFTLTISNLQPEDFATYYCQQYNRYPYTFGQGTKLEIKESKYGPPCPPCPAPEAAGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKYPYDVPDYAGLNAEGRGSLLTCGDVEENPGPMWLQSLLLLGTVACSISMRSSPGNMERIVICLMVIFLGTLVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKVSTLSFIEQKLISEEDLAVGQDTQEVIVVPHSLPFKVVVISAILALVVLTIISLIILIMLWQKKPRQLGSGATNFSLLKQAGDVEENPGPMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 232 - DNA, 5654 bp, Artificial Sequence
  Description of Artificial Sequence Synthetic polynucleotide
atggggtggtcaagcattattctgtttctggtcgctaccgctacaggcgtccatcagatccagctggtgcagtccggccccgagctgaagaagcccggcgagaccgtgaagatctcctgcaaggcctccggctacaccttcaccgactactccatcaactgggtgaagcgggcccccggcaagggcctgaagtggatgggctggatcaacaccgagacccgggagcccgcctacgcctacgacttccggggccggttcgccttctccctggagacctccgcctccaccgcctacctgcagatcaacaacctgaagtacgaggacaccgccacctacttctgcgccctggactactcctacgccatggactactggggccagggcacctccgtgaccgtgtcctccggtgggggcggctctggtggcggtggcagcggcggaggtggcagtgatattgttcttactcaatcacccccaagccttgcgatgtctcttggtaaacgagcgacaattagttgtagagcttctgaaagcgtaactattcttgggtcacatcttattcattggtatcaacaaaagccgggacaaccgcctacactcttgattcaactcgcgagcaatgttcaaacgggtgtccctgcacgcttttctgggagcggttcacgaacagattttactctcacgattgatccagtcgaagaagatgatgtcgctgtatattattgtctccaaagtaggacaataccaagaacttttggtggtggtacaaaattggaaattaaactcgagcccaaatcttgtgacaaaactcacacatgcccaccgtgcccggatcccaaaggtaccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgccacgtgggttctggagaaggacgcggttccttgttgacgtgtggcgatgtagaggaaaatccgggtccaatgtacaggatgcaactcctgtcttgcattgcactaagtcttgcacttgtcacaaacagtcaagtgcagctggttgaatccggtggcggtctggtcaagccgggcggctctttgcgtctgagctgtgccgcgtcgggttttaccttcagctcttatggtatgcattgggtgcgtcaggcgcctggcaaaggtctggagtgggttgcgttcatccgctacgatgggtctaacaaatattatgccgactcagtaaaaggacgcttcactattagccgcgacaatagcaaaaataccctgtacctgcaaatgaatagcctgcgcgccgaagataccgccgtttactattgcgctaaagatcgtggcctgggtgatggtacgtacttcgattactggggtcagggcaccaccgttaccgttagttcaggtgggggcggctctggtggcggtggcagcggcggaggtggcagtcagtcagcgcttacgcagccggcgtcggtgtcgggttccccgggtcagtcgatcacgatcagctgtagtgggagcagctccaacatcggtaacaacgcagtgaactggtatcagcaactgccgggaaaagcgccgaaactgctgatttactatgatgatttgctgccaagtggagttagtgaccgcttttccggcagtaaatcgggtacctcggcttttctggctatttcgggtctccagagcgaggatgaagctgattattattgcgccgcatgggatgatagcttaaatggcccagtttttggcggcggtactaaactgaccgtgctgccgagcggccaggcgggcgcggcggcatcggagtccctgtttgtgtcaaatcacgcctaccaggtgcagctggtgcagagcggcgccgaggtgaagaagcccggcgccagcgtgaaggtgagctgcaaggccagcggctacagcttcaccggctacaccatgaactgggtgcggcaggcccccggccagggcctggagtggatgggcctgatcaacccctacaacagcgacaccaactacgcccagaagctgcagggccgggtgaccatgaccaccgacaccagcaccagcaccgcctacatggagctgcggagcctgcggagcgacgacaccgccgtgtactactgcgcccgggtggccctgcgggtggccctggactactggggccagggcaccctggtgaccgtgagcagcggtgggggcggctctggtggcggtggcagcggcggaggtggcagtgacatccagatgacccagagccccagcagcctgagcgccagcgtgggcgaccgggtgaccatcacctgcaaggccagccagaacgtggccacccacgtgggctggtaccagcagaagcccggcaaggcccccaagcggctgatctacagcgccagctaccggtacagcggcgtgcccagccggttcagcggcagcggcagcggcaccgagttcaccctgaccatcagcaacctgcagcccgaggacttcgccacctactactgccagcagtacaaccggtacccctacaccttcggccagggcaccaagctggagatcaaggaaagcaaatatggcccgccgtgcccgccgtgcccggcgccggaagcggcgggcggcccgagcgtgtttctgtttccgccgaaaccgaaagataccctgatgattagccgcaccccggaagtgacctgcgtggtggtggatgtgagccaggaagatccggaagtgcagtttaactggtatgtggatggcgtggaagtgcataacgcgaaaaccaaaccgcgcgaagaacagtttcagagcacctatcgcgtggtgagcgtgctgaccgtgctgcatcaggattggctgaacggcaaagaatataaatgcaaagtgagcaacaaaggcctgccgagcagcattgaaaaaaccattagcaaagcgaaaggccagccgcgcgaaccgcaggtgtataccctgccgccgagccaggaagaaatgaccaaaaaccaggtgagcctgacctgcctggtgaaaggcttttatccgagcgatattgcggtggaatgggaaagcaacggccagccggaaaacaactataaaaccaccccgccggtgctggatagcgatggcagcttttttctgtatagccgcctgaccgtggataaaagccgctggcaggaaggcaacgtgtttagctgcagcgtgatgcatgaagcgctgcataaccattatacccagaaaagcctgagcctgagcctgggcaaatacccatacgatgttccagattacgctggtttaaacgccgagggcagaggaagtcttctaacatgcggtgacgtggaggagaatcccggccctgtcgacccaccatgtggctgcagagcctgctgctcttgggcactgtggcctgcagcatctctatgagatccagtcctggcaacatggagaggattgtcatctgtctgatggtcatcttcttggggacactggtccacaaatcaagctcccaaggtcaagatcgccacatgattagaatgcgtcaacttatagatattgttgatcagctgaaaaattatgtgaatgacttggtccctgaatttctgccagctccagaagatgtagagacaaactgtgagtggtcagctttttcctgctttcagaaggcccaactaaagtcagcaaatacaggaaacaatgaaaggataatcaatgtatcaattaaaaagctgaagaggaaaccaccttccacaaatgcagggagaagacagaaacacagactaacatgcccttcatgtgattcttatgagaaaaaaccacccaaagaattcctagaaagattcaaatcacttctccaaaaggtatctaccttaagtttcattgaacaaaaactcatctcagaagaggatctggctgtgggccaggacacgcaggaggtcatcgtggtgccacactccttgccctttaaggtggtggtgatctcagccatcctggccctggtggtgctcaccatcatctcccttatcatcctcatcatgctttggcagaagaagccacgtcaattgggcagcggcgccacaaacttctctctgctaaagcaagcaggtgatgttgaagaaaaccccgggcctatgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatg

SEQ ID NO: 233 - protein, 184 aa, Artificial Sequence [sequence omitted]

SEQ ID NO: 234 - DNA, 552 bp, Artificial Sequence
  Description of Artificial Sequence Synthetic polynucleotide
atgttgcagatggctgggcagtgctcccaaaatgaatattttgacagtttgttgcatgcttgcataccttgtcaacttcgatgttcttctaatactcctcctctaacatgtcagcgttattgtaatgcaagtgtgaccaattcagtgaaaggaacgaatgcgattctctggacctgtttgggactgagcttaataatttctttggcagttttcgtgctaatgtttttgctaaggaagataagctctgaaccattagaggacgagtttaaaaacacaggatcaggtctcctgggcatggctaacattgacctggaaaagagcaggactggtgatgaaattattcttccgagaggcctcgagtacacggtggaagaatgcacctgtgaagactgcatcaagagcaaaccgaaggtcgactctgaccattgctttccactcccagctatggaggaaggcgcaaccattcttgtcaccacgaaaacgaatgactattgcaagagcctgccagctgctttgagtgctacggagatagagaaatcaatttctgctagg

SEQ ID NO: 235 - protein, 345 aa, Artificial Sequence
  Description of Artificial Sequence Synthetic polypeptide
MYKDCIESTGDYFLLCDAEGPWGIILESLAILGIVVTILLLLAFLFLMRKIQDCSQWNVLPTQLLFLLSVLGLFGLAFAFIIELNQQTAPVRYFLFGVLFALCFSCLLAHASNLVKLVRGCVSFSWTTILCIAIGCSLLQIIIATEYVTLIMTRGMMFVNMTPCQLNVDFVVLLVYVLFLMALTFFVSKATFCGPCENWKQHGRLIFITVLFSIIIWVVWISMLLRGNPQFQRQPQWDDPVVCIALVTNAWVFLLLYIVPELCILYRSCRQECPLQGNACPVTAYQHSFQVENQELSRARDSDGAEEDVALTSYGTPIQPQTVDPTQECFIPQAKLSPQQDAGGV

SEQ ID NO: 236 - DNA, 1035 bp, Artificial Sequence
  Description of Artificial Sequence Synthetic polynucleotide
atgtataaagattgtattgagtctactggtgactatttcctgctgtgtgacgccgagggcccatggggaatcattctggaatctctggccatcctgggaatcgtggtgaccattttgctccttcttgcttttctgtttctgatgcgcaaaatccaggattgcagccagtggaatgtcctgcccactcagcttttgttcttgcttagcgttcttggcctgtttgggctggctttcgcctttattatcgaactgaaccagcaaacagcccccgtgcgctactttctcttcggcgtgctgtttgccctctgcttctcatgtcttctggcccatgccagtaacctggttaagctggtccggggatgtgtatcattttcatggactaccatcctgtgtatcgctataggctgtagtctgcttcagattatcatcgcgacagaatacgtgaccctgatcatgaccagaggcatgatgttcgtgaacatgactccatgccaactgaacgtagactttgttgtactccttgtgtacgttctgtttctgatggccctgactttcttcgtcagtaaagctacgttttgcggtccgtgcgagaattggaagcagcacggtcgcctgattttcattacggttctcttctctatcattatttgggtcgtgtggattagtatgctgctgcgcggaaatccacagtttcagcgccagccccagtgggacgatcctgtcgtttgtatcgctctcgtgactaacgcctgggttttcctgctcctgtacattgttccagagctgtgcattctctacagatcttgccgacaggaatgtcccctccaggggaacgcatgtcccgtgacagcctatcaacatagtttccaagtggaaaaccaggagctgtcacgggccagggactctgacggagccgaagaagatgtggcgctgacgagttatggcacacccatccaaccacagaccgtggatccgacccaagaatgcttcatcccacaggccaagctgagcccgcagcaggatgctggcggtgta

SEQ ID NO: 237 - protein, 557 aa, Artificial Sequence
  Description of Artificial Sequence Synthetic polypeptide
MYKDCIESTGDYFLLCDAEGPWGIILESLAILGIVVTILLLLAFLFLMRKIQDCSQWNVLPTQLLFLLSVLGLFGLAFAFIIELNQQTAPVRYFLFGVLFALCFSCLLAHASNLVKLVRGCVSFSWTTILCIAIGCSLLQIIIATEYVTLIMTRGMMFVNMTPCQLNVDFVVLLVYVLFLMALTFFVSKATFCGPCENWKQHGRLIFITVLFSIIIWVVWISMLLRGNPQFQRQPQWDDPVVCIALVTNAWVFLLLYIVPELCILYRSCRQECPLQGNACPVTAYQHSFQVENQELSRARDSDGAEEDVALTSYGTPIQPQTVDPTQECFIPQAKLSPQQDAGGVGLNHVGSGEGRGSLLTCGDVEENPGPQLMLQMAGQCSQNEYFDSLLHACIPCQLRCSSNTPPLTCQRYCNASVTNSVKGTNAILWTCLGLSLIISLAVFVLMFLLRKISSEPLEDEFKNTGSGLLGMANIDLEKSRTGDEIILPRGLEYTVEECTCEDCIKSKPKVDSDHCFPLPAMEEGATILVTTKTNDYCKSLPAALSATEIEKSISAR

SEQ ID NO: 238 - DNA, 1671 bp, Artificial Sequence
  Description of Artificial Sequence Synthetic polynucleotide
atgtataaagattgtattgagtctactggtgactatttcctgctgtgtgacgccgagggcccatggggaatcattctggaatctctggccatcctgggaatcgtggtgaccattttgctccttcttgcttttctgtttctgatgcgcaaaatccaggattgcagccagtggaatgtcctgcccactcagcttttgttcttgcttagcgttcttggcctgtttgggctggctttcgcctttattatcgaactgaaccagcaaacagcccccgtgcgctactttctcttcggcgtgctgtttgccctctgcttctcatgtcttctggcccatgccagtaacctggttaagctggtccggggatgtgtatcattttcatggactaccatcctgtgtatcgctataggctgtagtctgcttcagattatcatcgcgacagaatacgtgaccctgatcatgaccagaggcatgatgttcgtgaacatgactccatgccaactgaacgtagactttgttgtactccttgtgtacgttctgtttctgatggccctgactttcttcgtcagtaaagctacgttttgcggtccgtgcgagaattggaagcagcacggtcgcctgattttcattacggttctcttctctatcattatttgggtcgtgtggattagtatgctgctgcgcggaaatccacagtttcagcgccagccccagtgggacgatcctgtcgtttgtatcgctctcgtgactaacgcctgggttttcctgctcctgtacattgttccagagctgtgcattctctacagatcttgccgacaggaatgtcccctccaggggaacgcatgtcccgtgacagcctatcaacatagtttccaagtggaaaaccaggagctgtcacgggccagggactctgacggagccgaagaagatgtggcgctgacgagttatggcacacccatccaaccacagaccgtggatccgacccaagaatgcttcatcccacaggccaagctgagcccgcagcaggatgctggcggtgtaggtttaaaccacgtgggttctggagaaggacgcggttccttgttgacgtgtggcgatgtagaggaaaatccgggtccacaattgatgttgcagatggctgggcagtgctcccaaaatgaatattttgacagtttgttgcatgcttgcataccttgtcaacttcgatgttcttctaatactcctcctctaacatgtcagcgttattgtaatgcaagtgtgaccaattcagtgaaaggaacgaatgcgattctctggacctgtttgggactgagcttaataatttctttggcagttttcgtgctaatgtttttgctaaggaagataagctctgaaccattagaggacgagtttaaaaacacaggatcaggtctcctgggcatggctaacattgacctggaaaagagcaggactggtgatgaaattattcttccgagaggcctcgagtacacggtggaagaatgcacctgtgaagactgcatcaagagcaaaccgaaggtcgactctgaccattgctttccactcccagctatggaggaaggcgcaaccattcttgtcaccacgaaaacgaatgactattgcaagagcctgccagctgctttgagtgctacggagatagagaaatcaatttctgctagg

SEQ ID NO: 239 - protein, 665 aa, Artificial Sequence
  Description of Artificial Sequence Synthetic polypeptide
MEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKRGLNAEGRGSLLTCGDVEENPGPVDMTRLTVLALLAGLLASSRAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSQLGSGATNFSLLKQAGDVEENPGPMGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVV

SEQ ID NO: 240 - protein, 308 aa, Artificial Sequence
  Description of Artificial Sequence Synthetic polypeptide
MEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKR

SEQ ID NO: 241 - protein, 19 aa, Artificial Sequence
  Description of Artificial Sequence Synthetic peptide
ATNFSLLKQAGDVEENPGP

SEQ ID NO: 242 - DNA, 1995 bp, Artificial Sequence
  Description of Artificial Sequence Synthetic polynucleotide
atggaagtcaggcccgaggaacctctagtggtgaaggtggaagagggagataacgctgtgctgcagtgcctcaaggggacctcagatggccccactcagcagctgacctggtctcgggagtccccgcttaaacccttcttaaaactcagcctggggctgccaggcctgggaatccacatgaggcccctggccatctggcttttcatcttcaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagccaggacctcaccatggcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatggagacgggtctgttgttgccccgggccacagctcaagacgctggaaagtattattgtcaccgtggcaacctgaccatgtcattccacctggagatcactgctcggccagtactatggcactggctgctgaggactggtggctggaaggtctcagctgtgactttggcttatctgatcttctgcctgtgttcccttgtgggcattcttcatcttcaaagagccctggtcctgaggaggaaaagaggtttaaacgccgagggcagaggaagtcttctaacatgcggtgacgtggaggagaatcccggccctgtcgacatgacccgcctcacagtcctcgcgctcctcgccggactgctcgcctcttcacgggcaggaatacatgtattcattcttggatgtttcagtgccggtctcccgaaaaccgaagccaactgggtgaatgtgatcagtgaccttaagaagatagaggaccttatccagagcatgcacattgatgctacactctataccgaaagtgatgttcacccatcatgcaaagtaacagccatgaagtgctttcttctggagcttcaagttattagccttgagtccggcgatgccagtattcatgatactgtagaaaatctgattatccttgcgaacaacagtttgtccagcaatgggaatgttactgaatctgggtgtaaagaatgtgaggaacttgaggaaaagaatatcaaggagttcttgcagtcatttgtgcatattgtccaaatgttcataaataccagtcaattggggagtggagctaccaatttctcactcctgaaacaggcgggcgacgtcgaggagaatcctggccccatggggacatcccttctttgttggatggctctctgtttgctcggagccgaccacgctgacgcctgtccttactctaatccctctttgtgttcaggtggtggtgggtcagagttgcctacacaaggtacattctcaaatgttagtacaaatgtcagtcccgccaagccaacgacgacggcatgtccctactctaatccaagtttgtgttctggtggcggtggatctccagcccccagaccacccactcctgctcccactatcgcctctcaacccctctcactcagacctgaggcatgtagaccagccgctggtggggccgtacacacaagaggattggacttcgcctgtgacatctacatatgggcccctctcgctgggacgtgtggagtcttgctcctctccttggtaatcacactctactgtaatcaccggaatcgcaggagagtctgtaagtgtccaagacccgtagtc

SEQ ID NO: 243 - DNA, 924 bp, Artificial Sequence
  Description of Artificial Sequence Synthetic polynucleotide
atggaagtcaggcccgaggaacctctagtggtgaaggtggaagagggagataacgctgtgctgcagtgcctcaaggggacctcagatggccccactcagcagctgacctggtctcgggagtccccgcttaaacccttcttaaaactcagcctggggctgccaggcctgggaatccacatgaggcccctggccatctggcttttcatcttcaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagccaggacctcaccatggcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatggagacgggtctgttgttgccccgggccacagctcaagacgctggaaagtattattgtcaccgtggcaacctgaccatgtcattccacctggagatcactgctcggccagtactatggcactggctgctgaggactggtggctggaaggtctcagctgtgactttggcttatctgatcttctgcctgtgttcccttgtgggcattcttcatcttcaaagagccctggtcctgaggaggaaaaga

SEQ ID NO: 244 - DNA, 57 bp, Artificial Sequence
  Description of Artificial Sequence Synthetic oligonucleotide
gccgagggcagaggaagtcttctaacatgcggtgacgtggaggagaatcccggccct

SEQ ID NO: 245 - DNA, 57 bp, Artificial Sequence
  Description of Artificial Sequence Synthetic oligonucleotide
gctaccaatttctcactcctgaaacaggcgggcgacgtcgaggagaatcctggcccc

SEQ ID NO: 246 - protein, 819 aa, Artificial Sequence
  Description of Artificial Sequence Synthetic polypeptide
MGWSSIILFLVATATGVHQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSGGGGSGGGGSGGGGSDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKLEPKSCDKTHTCPPCPDPKGTFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGLNAEGRGSLLTCGDVEENPGPVDMTRLTVLALLAGLLASSRAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSQLGSGATNFSLLKQAGDVEENPGPMGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVV

SEQ ID NO: 247 - DNA, 2457 bp, Artificial Sequence
  Description of Artificial Sequence Synthetic polynucleotide
atggggtggtcaagcattattctgtttctggtcgctaccgctacaggcgtccatcagatccagctggtgcagtccggccccgagctgaagaagcccggcgagaccgtgaagatctcctgcaaggcctccggctacaccttcaccgactactccatcaactgggtgaagcgggcccccggcaagggcctgaagtggatgggctggatcaacaccgagacccgggagcccgcctacgcctacgacttccggggccggttcgccttctccctggagacctccgcctccaccgcctacctgcagatcaacaacctgaagtacgaggacaccgccacctacttctgcgccctggactactcctacgccatggactactggggccagggcacctccgtgaccgtgtcctccggtgggggcggctctggtggcggtggcagcggcggaggtggcagtgatattgttcttactcaatcacccccaagccttgcgatgtctcttggtaaacgagcgacaattagttgtagagcttctgaaagcgtaactattcttgggtcacatcttattcattggtatcaacaaaagccgggacaaccgcctacactcttgattcaactcgcgagcaatgttcaaacgggtgtccctgcacgcttttctgggagcggttcacgaacagattttactctcacgattgatccagtcgaagaagatgatgtcgctgtatattattgtctccaaagtaggacaataccaagaacttttggtggtggtacaaaattggaaattaaactcgagcccaaatcttgtgacaaaactcacacatgcccaccgtgcccggatcccaaaggtaccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggtttaaacgccgagggcagaggaagtcttctaacatgcggtgacgtggaggagaatcccggccctgtcgacatgacccgcctcacagtcctcgcgctcctcgccggactgctcgcctcttcacgggcaggaatacatgtattcattcttggatgtttcagtgccggtctcccgaaaaccgaagccaactgggtgaatgtgatcagtgaccttaagaagatagaggaccttatccagagcatgcacattgatgctacactctataccgaaagtgatgttcacccatcatgcaaagtaacagccatgaagtgctttcttctggagcttcaagttattagccttgagtccggcgatgccagtattcatgatactgtagaaaatctgattatccttgcgaacaacagtttgtccagcaatgggaatgttactgaatctgggtgtaaagaatgtgaggaacttgaggaaaagaatatcaaggagttcttgcagtcatttgtgcatattgtccaaatgttcataaataccagtcaattggggagtggagctaccaatttctcactcctgaaacaggcgggcgacgtcgaggagaatcctggccccatggggacatcccttctttgttggatggctctctgtttgctcggagccgaccacgctgacgcctgtccttactctaatccctctttgtgttcaggtggtggtgggtcagagttgcctacacaaggtacattctcaaatgttagtacaaatgtcagtcccgccaagccaacgacgacggcatgtccctactctaatccaagtttgtgttctggtggcggtggatctccagcccccagaccacccactcctgctcccactatcgcctctcaacccctctcactcagacctgaggcatgtagaccagccgctggtggggccgtacacacaagaggattggacttcgcctgtgacatctacatatgggcccctctcgctgggacgtgtggagtcttgctcctctccttggtaatcacactctactgtaatcaccggaatcgcaggagagtctgtaagtgtccaagacccgtagtc

SEQ ID NO: 248 - protein, 21 aa, Artificial Sequence
  Description of Artificial Sequence Synthetic peptide
GSGEGRGSLLTCGDVEENPGP

SEQ ID NO: 249 - DNA, 363 bp, Artificial Sequence
  Description of Artificial Sequence Synthetic polynucleotide
caagtgcagctggttgaatccggtggcggtctggtcaagccgggcggctctttgcgtctgagctgtgccgcgtcgggttttaccttcagctcttatggtatgcattgggtgcgtcaggcgcctggcaaaggtctggagtgggttgcgttcatccgctacgatgggtctaacaaatattatgccgactcagtaaaaggacgcttcactattagccgcgacaatagcaaaaataccctgtacctgcaaatgaatagcctgcgcgccgaagataccgccgtttactattgcgctaaagatcgtggcctgggtgatggtacgtacttcgactattggggacaagggaccacagttacagtcagctcc

SEQ ID NO: 250 - DNA, 687 bp, Artificial Sequence
  Description of Artificial Sequence Synthetic polynucleotide
gagtccaagtacggtcccccttgtcccccatgtcctgctcctgaggctgccggtggtccctcagtcttccttttcccacccaagcccaaggacacgctcatgatctctaggactcccgaggtcacttgtgtcgtcgttgacgtcagtcaagaggacccagaggtacaattcaactggtacgtcgatggagttgaggtccacaatgctaagacgaagcctcgcgaggagcaattccaatctacatacagagttgtctcagttttgactgttttgcaccaagactggctcaatggtaaggaatacaagtgtaaggtttccaataagggtttgccctctagtatcgagaagactatctccaaggccaagggacaacctagggagccacaagtctacactctcccaccaagtcaagaggagatgactaagaatcaagtatccctcacttgtttggtcaaggggttctacccaagtgacatagcagttgagtgggagtccaatgggcaaccagagaataattacaagacgactccgcccgttttggactccgacgggtctttcttcctctacagtaggcttacggttgacaagtcccgatggcaagaggggaatgtattctcttgttctgttatgcacgaggcccttcacaatcactacacacaaaagtctctctcactttcactcgggaag

SEQ ID NO: 251 - DNA, 57 bp, Artificial Sequence
  Description of Artificial Sequence Synthetic oligonucleotide
gcagaagggcgagggtctctcttgacttgtggagatgttgaagaaaaccctggtcct

SEQ ID NO: 252 - protein, 107 aa, Artificial Sequence
  Description of Artificial Sequence Synthetic polypeptide
DIQMTQSPSSLSASVGDRVTITCSASQDISNYLNWYQQKPGKAPKLLIYYTSNLHSGVPSRFSGSGSGTDFTLTISSLQPEDFATYYCQQYRKLPWTFGQGTKLEIK

(19) United States
(12) Patent Application Publication
(10) Pub. No.: US 2023/0331872 A1
(43) Pub. Date: Oct. 19, 2023

(54) BISPECIFIC ANTIBODY CAR CELL IMMUNOTHERAPY

(71) Applicant: CYTOIMMUNE THERAPEUTICS, INC., Wilmington, DE (US)

(72) Inventor: Lei Zhang, Wilmington, DE (US)

(21) Appl. No.: 18/022,741

(22) PCT Filed: Aug. 24, 2021
(86) PCT No.: PCT/US2021/047375

§ 371 Date: Feb. 22, 2023

Related U.S. Application Data
(60) Provisional application No. 63/070,219, filed on Aug. 25, 2020.
(60) Provisional application No. 63/075,750, filed on Sep. 8, 2020.
(60) Provisional application No. 63/142,426, filed on Jan. 27, 2021.
(60) Provisional application No. 63/193,024, filed on May 25, 2021.

(51) Int. Cl. C07K 16/46; C07K 14/725; A61P 35/00; C07K 14/54

(52) U.S. Cl. CPC C07K 16/468; C07K 14/7051; A61P 35/00; C07K 14/54; C07K 2319/03; C07K 2317/565; C07K 2317/52

(57) ABSTRACT

Provided is a polypeptide comprising, or consisting essentially of, or yet further consisting of a Chimeric Antigen Receptor (CAR) that recognizes and binds a (anti-TAA antigen binding sequence), a bispecific antibody recognizes and binds NKG2D and G Protein-Coupled Receptor Class C Group 5 Member D (GPRCSD), an optional cytokine and an optional suicide gene product. Also provided are related compositions, polynucleotide, vector, and methods.

### CROSS-REFERENCE TO RELATED APPLICATIONS

This application claims priority under 35 U.S.C. § 119(e) of U.S. Provisional Application Nos. 63/070,219, filed Aug. 25, 2020; 63/075,750, filed Sep. 8, 2020; 63/142,426, filed Jan. 27, 2021; and 63/193,024, filed May 25, 2021, the contents of each of which are hereby incorporated by reference into this application in their entireties.

### SEQUENCE LISTING

The instant application contains a Sequence Listing which has been submitted electronically in ASCII format and is hereby incorporated by reference in its entirety. Said ASCII copy, created on Aug. 23, 2021, is named 113086-9260_SL.txt and is 561,741 bytes in size.

### TECHNICAL FIELD

The present disclosure relates generally to the field of human immunology, specifically cancer immunotherapy.

### BACKGROUND

The following discussion of the background of the invention is merely provided to aid art to the present invention.

Current cancer treatment regimens including chemotherapies, immunomodulatory drugs, monoclonal antibodies, and autologous or allogeneic transplantation. These therapies often lead to remission, but nearly all patients eventually relapse and succumb to death due to return of the disease. Thus, there is an unmet need for new therapies, including new combination immunotherapies for relapsed and/or refractory disease. This disclosure addresses these limitations and provides related advantages as well.

### SUMMARY OF THE DISCLOSURE

The foregoing general description and following detailed description are exemplary and explanatory and are intended to provide further explanation of the disclosure as claimed. Other objects, advantages, and novel features will be readily apparent to those skilled in the art from the following brief description of the drawings and detailed description of the disclosure.

In one aspect, provided is a polypeptide or a polynucleotide encoding the polypeptide, wherein the polypeptide comprises, or consists essentially of, or yet further consists of (i) an amino acid sequence (also referred to herein as a peptide) of a Chimeric Antigen Receptor (CAR) and (ii) an amino acid sequence of a bispecific antibody. Further, the CAR peptide comprises, or consists essentially of, or yet further consists of (1) an antigen binding amino acid sequence (such as a single-chain variable fragment, i.e., an scFv) that recognizes and binds a tumor associated antigen (TAA) (anti-TAA antigen binding sequence), (2) a hinge domain, (3) a transmembrane domain, and (4) an intracellular domain. Additionally, the bispecific antibody comprises, or consists essentially of, or yet further consists of (1) an antigen binding sequence (such as an scFv) that recognizes and binds NKG2D (anti-NKG2D antigen binding sequence), and (2) an antigen binding sequence (such as an scFv) that recognizes and binds G Protein-Coupled Receptor Class C Group 5 Member D (GPRC5D) (anti-GPRC5D antigen binding sequence). In some embodiments, the TAA is B-cell maturation antigen (BCMA).

In some embodiments, the bispecific antibody comprises, or consists essentially of, or yet further consists of, from the N terminus to the C terminus, the anti-GPRC5D antigen binding sequence and the anti-NKG2D antigen binding sequence. In some embodiments, the bispecific antibody comprises, or consists essentially of, or yet further consists of, from the N terminus to the C terminus, the anti-NKG2D antigen binding sequence and the anti-GPRC5D antigen binding sequence.

In some embodiments, the polypeptide further comprises a cleavable peptide (such as a self-cleaving peptide) located between the CAR amino acid sequence and the amino acid sequence of the bispecific antibody. In further embodiments, the polypeptide can be cleaved by itself or by an enzyme, into two or more peptides, for example, the CAR peptide and the bispecific antibody. Accordingly, also provided is a composition comprising, or consisting essentially of, or yet further consisting of a CAR peptide and a bispecific antibody cleaved from a polypeptide. The polypeptide comprises, or consists essentially of, or yet further consists of an amino acid sequence of the CAR peptide, an amino acid sequence of the bispecific antibody, and a cleavable peptide located between the CAR sequence and the antibody sequence. In some embodiments, the composition further comprises the polypeptide which has not been cleaved.

In another aspect, provided is a polynucleotide comprising, or consisting essentially of, or yet further consisting of a nucleotide sequence encoding a CAR peptide as disclosed herein, a nucleotide sequence encoding a bispecific antibody as disclosed herein, and an internal ribosome entry site (IRES) located between the CAR coding sequence and the bispecific antibody coding sequence. In yet another aspect, provided is a polynucleotide comprising, or consisting essentially of, or yet further consisting of a nucleotide sequence encoding a CAR peptide, a first regulatory sequence directing the expression thereof, a nucleotide sequence encoding a bispecific antibody, and a second regulatory sequence directing the expression thereof. In some embodiments, the two regulatory sequences are different in their sequences or locations or both in the polynucleotide. In some embodiments, the first regulatory sequence and the second regulatory sequence constitutes, comprises, consists essentially of, or consists of, a bidirectional regulatory sequence. Accordingly, provided is a composition comprising, or consisting essentially of, or yet further consisting of a CAR peptide and a bispecific antibody, both of which are encoded by the polynucleotide.

In a further aspect, provided is a cell comprising a polypeptide comprising, or consisting essentially of, or yet further consisting of (i) an amino acid sequence of a Chimeric Antigen Receptor (CAR) as disclosed herein and (ii) an amino acid sequence of a bispecific antibody as disclosed herein, or a polynucleotide as disclosed herein, or both of the polypeptide and the polynucleotide. In some embodiments, the polypeptide is further processed, for example, cleaved by itself or by the cell, to yield the CAR peptide and the bispecific antibody. In some embodiments, the cell further comprises the CAR peptide and the bispecific antibody. In some embodiments, the cell comprises or further comprises the polynucleotide as disclosed herein and a regulatory sequence suitable for expressing or replicating the polynucleotide in the cell.

In yet a further aspect, provided is a cell comprising a CAR peptide as disclosed herein and a bispecific antibody as disclosed herein. In some embodiments, the cell comprises the CAR on the cell membrane and secretes the bispecific antibody out of the cell. Additionally, or alternatively, the CAR peptide and the bispecific antibody are produced, for example in the cell, via cleaving a polypeptide comprising, or consisting essentially of, or yet further consisting of (i) an amino acid sequence of the CAR, (ii) an amino acid sequence of the bispecific antibody, and a cleavable peptide located between (i) and (ii).

In some embodiment, the polypeptide as disclosed herein further comprises (iii) an amino acid sequence of a cytokine, or (iv) an amino acid sequence of a suicide gene product or a detectable marker or both, or both (iii) and (iv). In some embodiments, the polypeptide as provided comprises, or consists essentially of, or yet further consists of (i) an amino acid sequence of a Chimeric Antigen Receptor (CAR), (ii) an amino acid sequence of a bispecific antibody, and (iii) an amino acid sequence of a cytokine. In some embodiments, the polypeptide as provided comprises, or consists essentially of, or yet further consists of (i) an amino acid sequence of a Chimeric Antigen Receptor (CAR), (ii) an amino acid sequence of a bispecific antibody, and (iv) an amino acid sequence of a suicide gene product or a detectable marker or both. In some embodiments, the polypeptide as provided comprises, or consists essentially of, or yet further consists of (i) an amino acid sequence of a Chimeric Antigen Receptor (CAR), (ii) an amino acid sequence of a bispecific antibody, (iii) an amino acid sequence of a cytokine and (iv) an amino acid sequence of a suicide gene product or a detectable marker or both.

In some embodiments, the suicide gene product or a detectable marker or both is a truncated epidermal growth factor receptor (tEGFR). In some embodiments, the suicide gene product or a detectable marker or both is RQR8. In some embodiments, cytokine is selected from IL-15 or IL-21. In further embodiments, the amino acid sequence of the CAR comprises, or consists essentially of, or yet further consists of (1) an antigen binding amino acid sequence that recognizes and binds B-cell maturation antigen (BCMA) (anti-BCMA antigen binding sequence), (2) a hinge domain, (3) a transmembrane domain, and (4) an intracellular domain. In some embodiments, the amino acid sequence of the bispecific antibody comprises, or consists essentially of, or yet further consists of (1) an antigen binding sequence that recognizes and binds NKG2D (anti-NKG2D antigen binding sequence), and (2) an antigen binding sequence that recognizes and binds G Protein-Coupled Receptor Class C Group 5 Member D (GPRCSD) (anti-GPRCSD antigen binding sequence). In some embodiments, the polypeptide comprises, or consists essentially of, or yet further consists of one or more of the amino acid sequences as disclosed herein. In some embodiments, any two amino acid sequences in a polypeptide as disclosed herein, such as those selected from (i), (ii), (iii) and (iv), may be separated by a cleavable peptide, thereby permitting the amino acid sequences of the polypeptide to be cleaved into two or more peptide fragments.

Accordingly, provided is a cell comprising a polypeptide as described herein, or a polynucleotide encoding the polypeptide, or both. In some embodiments, the polypeptide is further processed, for example, cleaved by itself or by the cell, to yield any one or two or three or all four of the following: the CAR peptide, the bispecific antibody, the cytokine, and the suicide gene product or a detectable marker or both. In some embodiments, the cell further comprises any one or two or three or all four of the following: the CAR peptide, the bispecific antibody, the cytokine, and the suicide gene product or a detectable marker or both. In some embodiments, the cell expresses the CAR on the cell membrane and secretes the bispecific antibody out of the cell. In further embodiments, the cell secretes the cytokine out of the cell. Additionally or alternatively, the cell expresses the suicide gene product or a detectable marker or both, for example on the cell membrane. In some embodiments, the cell comprises the polynucleotide as disclosed herein and a regulatory sequence suitable for expressing or replicating the polynucleotide in the cell.

Also provided is a polynucleotide comprising, or consisting essentially of, or yet further consisting of: a nucleotide sequence encoding a CAR peptide as disclosed herein and a nucleotide sequence encoding a bispecific antibody as disclosed herein. In further embodiments, the polynucleotide further comprises a nucleotide sequence encoding a cytokine as disclosed herein. Additionally or alternatively, the polynucleotide further comprises a nucleotide sequence encoding a suicide gene product or a detectable marker or both as disclosed herein. In yet further embodiments, the polynucleotide further comprises a regulatory sequence directing the expression of any of the coding sequences. Additionally or alternatively, the polynucleotide further comprises an internal ribosome entry site (IRES) located between any two of the coding sequences. In some embodiments, the polynucleotide comprises more than one regulatory sequences. In further embodiments, each of the regulatory sequences are different in their sequences or locations or both in the polynucleotide. In some embodiments, the regulatory sequence constitutes a bidirectional regulatory sequence. In some embodiments, the polynucleotide comprises more than one IRESs. Accordingly, provided is a composition comprising, or consisting essentially of, or yet further consisting of at least two of the following: a CAR peptide, a bispecific antibody, a cytokine, a detectable marker, or a suicide gene product, each of which is encoded by the polynucleotide.

In some embodiments of any aspect as disclosed herein, the bispecific antibody comprises an anti-TAA antigen binding sequence which is anti-GPRC5D as disclosed herein or alternatively an anti-TAA antigen binding sequence other than anti-GPRC5D. In some embodiments, the anti-TAA antigen binding sequence of the CAR is anti-GPRC5D and the anti-TAA antigen binding sequence of the bispecific antibody is anti-BCMA. In other embodiments, the anti-TAA antigen binding sequence of the CAR is anti-BCMA and the anti-TAA antigen binding sequence of the bispecific antibody is anti-GPRC5D. Accordingly, in the bispecific antibody element, e.g., the anti-NKG2D antigen binding sequence, may be located at the N terminus side of the anti-TAA antigen binding sequence, or located at the C terminus side of the anti-TAA antigen binding sequence. In further embodiments, in the bispecific antibody, the anti-NKG2D antigen binding sequence is linked to the anti-TAA antigen binding sequence via a peptide linker, such as an HMA linker, or a fragment crystallizable (Fc) region of an immunoglobulin, a mutant thereof, or an equivalent thereof, or both a peptide linker and a Fc region of an immunoglobulin, a mutant thereof, or an equivalent thereof.

In some embodiments of any aspect as disclosed herein, either or both of the antigen binding sequence of the bispecific antibody is a single-chain variable fragment (scFv). In some embodiments, the anti-bispecific antibody comprises, or consists essentially of, or yet further consists of an anti-NKG2D scFv and an anti-GPRC5D scFv (or an scFv recognizing and binding to another TAA). In some embodiments, the NKG2D scFv, is located at the N terminus side of the anti-TAA scFv. In some embodiments, the anti-NKG2D scFv is located at the C terminus side of the anti-TAA scFv. In further embodiments, the anti-NKG2D scFv is linked to the anti-TAA scFv via a peptide linker, such as an HMA linker, or a fragment crystallizable (Fc) region of an immunoglobulin, a mutant thereof, or an equivalent thereof, or both a peptide linker and a Fc region of an immunoglobulin, a mutant thereof, or an equivalent thereof. In some embodiments, the anti-NKG2D scFv is replaced with an scFv that recognizes and binds to an antigen which is not NKG2D but also present on the cell surface of an immune cell as disclosed herein, such as a T cell, an NK cell, or an NKT cell. Suitable antigens are exemplified herein in the later paragraphs.

Additionally or alternatively, the bispecific antibody comprises an NKG2D ligand instead of or in addition to the anti-NKG2D antigen binding sequence, such as MEW class I polypeptide-related sequence A (MICA), MHC class I polypeptide-related sequence A (MICB), UL16-binding protein 1 (ULBP1), UL16-binding protein 2 (ULBP2), UL16-binding protein 3 (ULBP3), UL16-binding protein 4 (ULBP4), UL16-binding protein 5 (ULBP5), UL16-binding protein 6 (ULBP6), or any other suitable NKG2D ligand(s). In some embodiments of any aspect as disclosed herein, the anti-NKG2D antigen binding sequence in the bispecific antibody may be replaced with an antigen binding sequence or a ligand that recognizes and binds to an antigen which is not NKG2D but also present on the cell surface of an immune cell as disclosed herein, such as a T cell, an NK cell, or an NKT cell. Non-limiting examples of such antigen include CD3, CD28, OX40, 4-1BB, ICOS, NKp46, NKp44, NKp30, CD16, Integrin alpha-L (LFA-1), CD27, CD56 or cytotoxic and regulatory T-cell molecule (CRTAM). Therefore, the bispecific antibody brings a cancer cell within the proximity of an immune cell via binding to a TAA present on the cancer cell (such as GPRC5D) and binding to an antigen on the immune cell (such as NKG2D or another as disclosed herein). In further embodiments, binding of the antigen binding sequence or a ligand with the antigen which is not NKG2D but expressed on the cell surface of an immune cell activates the immune cell.

Without wishing to be bound by the theory, Applicant discovered that targeting BCMA and GPRC5D via either or both of a CAR or a bispecific antibody engaging an immune cell (such as an NK cell, an NKT cell or a T cell) optionally via targeting NKG2D expressed on the immune cell provides surprising more than additive (such as synergistic) effect(s) in killing cancer/tumor cells. In some embodiments, the effect(s) is or are observed in vivo. Additionally or alternatively, the effect(s) can also be observed in vitro or ex vivo, therefore leading to an effective therapeutic regimen in treating a cancer, such as multiple myeloma (MM). Accordingly, as disclosed herein, provided is a polypeptide comprising, or consisting essentially of, or yet further consisting of (i) an amino acid sequence of a Chimeric Antigen Receptor (CAR) comprising an anti-BCMA antigen binding sequence and (ii) an amino acid sequence of a bispecific antibody comprising an anti-GPRC5D antigen binding sequence. It would be understand by one of skill in the art that the disclosure herein also includes embodiments and aspects wherein the anti-GPRC5D antigen binding sequence is switched with the anti-BCMA antigen binding sequence if both are present.

In another aspect, provided is a Chimeric Antigen Receptor (CAR) comprising, or consisting essentially of, or yet further consisting of (1) an antigen binding amino acid sequence that recognizes and binds G Protein-Coupled Receptor Class C Group 5 Member D (GPRC5D) (anti-GPRC5D antigen binding sequence), (2) a hinge domain, (3) a transmembrane domain, and (4) an intracellular domain. In some embodiments, the CAR further comprises an antigen binding sequence that recognizes and binds B-cell maturation antigen (BCMA).

In yet another aspect, provided is an antibody (such as a bispecific antibody) or a fragment thereof that recognizes and binds both NKG2D and G Protein-Coupled Receptor Class C Group 5 Member D (GPRC5D). The bispecific antibody or a fragment thereof comprises, or consists essentially of, or yet further consists of (1) an antigen binding sequence that recognizes and binds NKG2D (anti-NKG2D antigen binding sequence), and (2) an antigen binding sequence that recognizes and binds GPRC5D (anti-GPRC5D antigen binding sequence).

In some embodiments relating to any aspect of the disclosure herein, the anti-GPRC5D antigen binding sequence comprises, or consists essentially of, or yet further consists of one or two or three or four or five or all six of the following CDRs: a CDRL1 comprising, or consisting essentially of, or yet further consisting of KASQNVATHVG (SEQ ID NO: 10) or an equivalent thereof; a CDRL2 comprising, or consisting essentially of, or yet further consisting of SASYRYS (SEQ ID NO: 11) or an equivalent thereof; a CDRL3 comprising, or consisting essentially of, or yet further consisting of QQYNRYPYT (SEQ ID NO: 12) or an equivalent thereof; a CDRH1 comprising, or consisting essentially of, or yet further consisting of GYSFTGY (SEQ ID NO: 13) or an equivalent thereof; a CDRH2 comprising, or consisting essentially of, or yet further consisting of NPYNSD (SEQ ID NO: 14) or an equivalent thereof; and a CDRH3 comprising, or consisting essentially of, or yet further consisting of VALRVALDY (SEQ ID NO: 15) or an equivalent thereof, with the proviso that the equivalents thereof recognizes and binds GPRCSD. In some embodiments, wherein the anti-GPRCSD antigen binding sequence comprises, or consists essentially of, or yet further consists of a light chain variable region comprising, or consisting essentially of, or yet further consisting of SEQ ID NO: 16, or an equivalent thereof; or a heavy chain variable region comprising, or consisting essentially of, or yet further consisting of SEQ ID NO: 17, or an equivalent thereof, or both the light chain variable region or an equivalent thereof and the heavy chain variable region or an equivalent thereof, with the proviso that the equivalents thereof recognizes and binds GPRCSD. In some embodiments, the anti-GPRCSD antigen binding sequence comprises, or consists essentially of, or consists of both the HC and the LC, or an equivalent of one or both thereof and wherein the equivalents thereof recognizes and binds GPRCSD.

In some embodiments relating to any aspect of the disclosure herein, the hinge domain comprises, or consists essentially of, or yet further consists of a CD8α hinge domain or an IgG1 hinge domain or both. In some embodiments relating to any aspect of the disclosure herein, the transmembrane domain comprises, or consists essentially of, or yet further consists of a CD8α transmembrane domain or a CD28 transmembrane domain or both. In some embodiments relating to any aspect of the disclosure herein, the intracellular domain comprises, or consists essentially of, or yet further consists of one or more (such as, two or more) costimulatory regions selected from a CD28 costimulatory signaling region, a 4-1BB costimulatory signaling region, an ICOS costimulatory signaling region, an OX40 costimulatory region, a DAP 10 costimulatory region, or a DAP12 costimulatory region. In some embodiments relating to any aspect of the disclosure herein, the intracellular domain comprises, or consists essentially of, or yet further consists of one costimulatory region. In further embodiments, the intracellular domain comprises, or consists essentially of, or yet further consists of a CD28 costimulatory signaling region. Additionally or alternatively, the intracellular domain comprises, or consists essentially of, or yet further consists of a CD3 zeta signaling domain. In some embodiments relating to any aspect of the disclosure herein, any one or more of a CAR, a bispecific antibody, a cytokine, a suicide gene product, a detectable marker, or a polypeptide as disclosed herein further comprises a signal peptide at the N terminus of each.

In a further aspect, provided is a polynucleotide encoding one or more of the following: a polypeptide as disclosed herein, a bispecific antibody or a fragment thereof as disclosed herein, or a CAR peptide as disclosed herein; or a polynucleotide that is complement or reverse or both complement and reverse thereto. Also provided is a vector comprising, or consisting essentially of, or yet further consisting of a polynucleotide as disclosed herein. Further provided is an isolated or engineered cell comprising one or more of the following: a polypeptide as disclosed herein, a bispecific antibody or a fragment thereof as disclosed herein, a CAR as disclosed herein, a polynucleotide as disclosed herein, or a vector as disclosed herein. In some embodiments, the cell is an immune cell, optionally a T-cell, a B cell, a NK cell, a NKT cell, a dendritic cell, a myeloid cell, a monocyte, or a macrophage. In further embodiments, the immune cell is derived from Hematopoietic stem cells (HSCs) or induced pluripotent stem cells (iPSCs). In some embodiments, the cell is a stem cell, such as an HSC or iPSC. In some embodiments, the cell comprises the CAR in the cell membrane and secretes the bispecific antibody out of the cell. In further embodiments, the cell secretes a cytokine as disclosed herein. Additionally or alternatively, the cell expresses a suicide gene product or a detectable marker or both as disclosed herein, for example, on the cell surface.

In a further aspect, provided is a vector system comprising, or consisting essentially of, or yet further consisting of more than one vectors. The vectors encodes (i) an amino acid sequence of a Chimeric Antigen Receptor (CAR) comprising, or consisting essentially of, or yet further consisting of: (1) an antigen binding amino acid sequence that recognizes and binds B-cell maturation antigen (BCMA) (anti-BCMA antigen binding sequence), (2) a hinge domain, (3) a transmembrane domain, and (4) an intracellular domain; (ii) an amino acid sequence of a bispecific antibody comprising, or consisting essentially of, or yet further consisting of (1) an antigen binding sequence that recognizes and binds NKG2D (anti-NKG2D antigen binding sequence), and (2) an antigen binding sequence that recognizes and binds G Protein-Coupled Receptor Class C Group 5 Member D (GPRC5D) (anti-GPRC5D antigen binding sequence); (iii) an optional amino acid sequence of a cytokine selected from IL15 or IL-21; and (iv) an optional amino acid sequence of a suicide gene product or a detectable marker or both, optionally a truncated epidermal growth factor receptor (tEGFR) or RQR8.

In yet a further aspect, provided is a composition comprising, or consisting essentially of, or yet further consisting of a carrier, optionally a pharmaceutically acceptable carrier, and one or more of the following: a polypeptide as disclosed herein, a bispecific antibody or a fragment thereof as disclosed herein, a CAR as disclosed herein, a polynucleotide as disclosed herein, a vector as disclosed herein, a vector system as disclosed herein, an isolated cell as disclosed herein, or a cell or cell population as disclosed herein.

Also provided is an isolated complex comprising, or consisting essentially of, or yet further consisting of one or more of the following: a polypeptide as disclosed herein bound to a cancer cell, a bispecific antibody or a fragment thereof as disclosed herein bound to a cancer cell, a CAR as disclosed herein bound to a cancer cell, an isolated or engineered cell bound to a cancer cell, or a cancer cell bound with an isolated or engineered cell and a bispecific antibody.

In one aspect, provided is a method of producing a cell expressing a CAR or secreting a bispecific antibody or both expressing a CAR and secreting a bispecific antibody. In some embodiments, the cell further secretes a cytokine, or expresses a suicide gene product (or a detectable marker or both), or both secretes a cytokine and expresses a suicide gene product or a detectable marker or both. The method comprises, or consists essentially of, or yet further consists of transducing a cell or a population thereof with a polynucleotide as disclosed herein, a vector as disclosed herein, or a vector system as disclosed herein. Additionally or alternatively, the method comprises, or consists essentially of, or yet further consists of culturing a cell or a cell population as disclosed herein.

In another aspect, provided is a method of inhibiting the growth of a cancer cell expressing a tumor associated antigen (TAA, such as GPRC5D or BCMA or both) or a tissue comprising the cancer cell. The method comprises, or consists essentially of, or yet further consists of contacting the cancer cell or the tissue with an isolated or engineered cell as disclosed herein. In some embodiments, the cell is an immune cell, optionally a T-cell, a B cell, a NK cell, a NKT cell, a dendritic cell, a myeloid cell, a monocyte, or a macrophage. In further embodiments, the immune cell is derived from Hematopoietic stem cells (HSCs) or induced pluripotent stem cells (iPSCs). The contacting can be in vitro or ex vivo or in vivo.

In yet another aspect, provided is a method for treating a cancer in a subject in need thereof. The method comprises, or consists essentially of, or yet further consists of administering for example an effective amount of, an isolated or engineered cell as disclosed herein to the subject. In some embodiments, the subject is selected for the treatment if a cancer cell of the subject expresses one or more of tumor associated antigen(s) (TAAs, such as GPRC5D or BCMA or both). In some embodiments, the cancer is Multiple Myeloma (MM).

In one aspect, provided is a method for treating a GPRC5D-expressing cancer in a subject. The method comprises, or consists essentially of, or yet further consists of administering either or both of a bispecific antibody or a fragment thereof as disclosed herein, or an isolated or engineered cell as disclosed herein to the subject.

Another anti-cancer therapy or a therapy that upregulates the expression of the tumor associated antigen(s) or both therapies may be combined with a method as disclosed herein.

Additionally provided is a kit comprising, or consisting essentially of, or yet further consisting of an optional instruction for use and one or more of the following: a polypeptide as disclosed herein, a bispecific antibody or a fragment thereof as disclosed herein, a CAR as disclosed herein, a polynucleotide as disclosed herein, a vector as disclosed herein, a vector system as disclosed herein, an isolated or engineered cell as disclosed herein, a composition as disclosed herein, or an isolated complex as disclosed herein.

The foregoing general description and following detailed description are exemplary and explanatory and are intended to provide further explanation of the disclosure as claimed. Other objects, advantages, and novel features will be readily apparent to those skilled in the art from the following brief description of the drawings and detailed description of the disclosure.

### BRIEF DESCRIPTION OF THE DRAWINGS

FIGS. 1A-1E provide flow analysis of BCMA, GPRC5D and NKG2D surface expression on Multiple Myeloma (MM) cell lines (FIG. 1A), primary B cell (FIG. 1B), NK cells (FIG. 1C) and T cells (FIG. 1D) along with a bar graph summarizing the same (FIG. 1E). MM1.S, OPM2, H929, and RPMI8226 are Multiple Myeloma (MM) cell lines and were cultured in complete RPMI 1640 media. The freshly isolated B cells, and activated NK cells and T cells were used in this study. Total 2×10⁵ cells were blocked using human TRUSTAIN FCX™ (Fc Receptor Blocking Solution) at 1:50 dilution, then stain BCMA, GPRC5D and NKG2D with their isotype controls. In FIG. 1E, the bars of each group (BCMA, GPRCSD or NKG2D) represent, from left to right, results from MM cell lines, primary B cells, activated NK cells and activated T cells. The data disclosed herein demonstrated that, the BCMA is highly expressed on the surface of all the four MM cell lines, but not primary B cells and activated NK and T cells. The G protein-coupled receptor class C group 5 member D (GPRCSD) were detected only on the surface of three MM cell lines MM1.S,OPM2 and H929, but excepted for all primary B cells, NK and T cells.

FIGS. 2A-2C provide schematic representations of empty control (FIG. 2A), anti-BCMA conventional CAR (FIG. 2B) and two anti GPRCSD and NKG2D BiTE/BiKE CAR retroviral vectors (FIG. 2C). There were two BiTE/BiKE-BCMA CAR constructs used in this study with differences of first scFv and second scFv in BiTE/BiKE components. All the four constructs were used to generate BiTE/BiKE CAR for use in an immune cell, such as a T cell or an NK cell. BCMA: B-cell maturation antigen; BiTE: Bispecific T-cell Engager; BiKE: Bispecific NK-cell Engager. GN1: a bispecific antibody comprising the anti-GPCRSD antigen binding sequence on the N terminus side to the anti-NKG2D antigen binding sequence. NG2: a bispecific antibody comprising the anti-GPCRSD antigen binding sequence on the C terminus side to the anti-NKG2D antigen binding sequence.

FIGS. 3A-3C provide diagraphs of BiTE/BiKE-BCMA CAR and controls used in this study (“(G4S)3” disclosed as SEQ ID NO: 9) (FIGS. 3A-3B) with an alignment of mouse anti human BCMA and humanized anti BCMA used in this study (SEQ ID NOS: 41, 252, and 44-45, respectively, in order of appearance) (FIG. 3C). FIG. 3A shows that the anti BCMA scFv was extracted from mouse anti human BCMA antibody C11D 5.3 (WO 2015/158671 A1), and bispecific T cell engager (BiTE) or bispecific NK cell engager (BiKE) was full human source: human anti human NKG2D antibody KYK-2.0 (US20110150870A1), and human anti human GPRC5D antibody GC5B596 (US Patent Application 20180037651). There were two BiTE/BiKE-BCMA CAR constructs used in this study with differences of first scFv and second scFv in BiTE/BiKE components. A three-site mutated human IgG4 Fc was used to stabilize the BiTE/BiKE secreted. IgG4-Fc had been mutated at three sites, F234A, L235A within hinge region and N297Q within CH2 region, substitutions in its Fc region were aimed to avoid the Fc receptors binding. All the above four inserts were cloned into retrovirus vectors PCIR between 5′ and 3′ LTR. The letter “m” means mouse source. The amino acids and nucleotide sequences for each components and whole sequences of above constructs are provided in Tables 1a, 1b, 1c, 1d and 1e. FIG. 3B shows that the anti BCMA scFv was extracted from humanized anti human BCMA antibody (U.S. Ser. No. 10/174,095 B2), and BiTE/BiKE was full human source: human anti human NKG2D antibody KYK-2.0 (US20110150870A1), and human anti human GPRC5D antibody GC5B596 (US Patent Application 20180037651). There were two BiTE/BiKE-BCMA CAR constructs used in this study with differences of first scFv and second scFv in BiTE/BiKE components. A three-site mutated human IgG4 Fc was used to stabilize the BiTE/BiKE secreted. IgG4-Fc had been mutated at three sites, F234A, L235A within hinge region and N297Q within CH2 region, substitutions in its Fc region were aimed to avoid the Fc receptors binding. All the above four inserts were cloned into retrovirus vectors PCIR between 5′ and 3′ LTR. The letter “hu” means humanized. The amino acids and nucleotide sequences for each components and whole sequences of above constructs are provided in Tables 1f, 1g, 1h and 1e. As shown in FIG. 3C, CDRs were designated via AbRSA (Antibody Numbering and CDRs Delimiting) with Chothia numbering scheme (Al-Lazikani et al.,(1997), JMB 273:214-218). The amino acid coordinates for C11D5.3 and humanized anti-BCMA were from U.S. Ser. No. 10/174,095 B2.

FIGS. 4A-4B provide transduction evaluation and purity detection by flow assay before and after enrichment. All PCIR plasmids were packaged with PEQ-PEM 3(e), enveloped with BaEVTR at 1.5:1.5:1 ratio and transfected 293 T cell via PEI MAX 40K (Polysciences, Inc, Cat #24765). The virus supernatant at 48h and 72 h were collected and titration was tested using HT1080 cell line. Four donors' T cells were isolated using MACSxpress Whole Blood Pan T Cell Isolation Kit (Miltenyi Biotec, human,130-098-193), and cultured with T Cell TRANSACT™ (Miltenyi Biotec, human,130-111-160), 50 IU/ml of IL-2 (Fisher Scientific, human, CTP0023), 0.5 ng/ml of IL-15 (Miltenyi Biotec, human,130-095-765), and 5% AB human serum (Fisher BioReagents, BP2525100). Using retronectin reagent (TakaRa, T100A/B) coated non-tissue culture plate, Applicant transduced above constructs (FIG. 3A) into activated T cells (2-5 days) with MOI 3. Flow analysis determine the transduction rates on the day 6 of post transduction using ALEXA FLUOR® 647 AffiniPure Goat Anti-Mouse IgG, F(ab′)2 fragment specific (Jackson ImmunoResearch 115-605-006) and PE anti-human CD19 Antibody (BioLegend, #302208) were used to detection the BCMA-CAR and truncated CD19 expression (FIG. 4A). For enrichment of all the three BCMA CAR-T with two BiTE/BiKE-BCMA CAR-T and one conventional BCMA-CAR-T, the cells were stained with the primary antibody Biotin-SP (long spacer) AffiniPure Goat Anti-Mouse IgG, F(ab′)2 Fragment Specific (115-065-072 Jackson Immunoresearch), and followed by a secondary staining with INVITROGEN™ DYNABEADS™ M-280 Streptavidin (Thermo Fisher, 11205D). Further, positive selection was performed on DYNAMAG™-15 Magnet (Thermo Fisher, 12301D). For enrichment of transduced T cell with truncated CD19 empty control, the REAlease CD19 MicroBead Kit (Miltenyi Biotec, human, 130-117-034) was used. Further, positive selection was performed with LS column and the cells were then proceeded to magnetic separation. Flow analysis demonstrated there were highly enriched CAR-T and empty vector control (FIG. 4B).

FIGS. 5A-5C provide results determining the expression and concentration of bispecific antibody secreted from T cells by Western Blotting and ELISA. As shown in FIG. 5A, ELISA analysis showed there were detectable BiTE/BiKE from non-concentrated supernatant of transduced BITE/BiKE-BCMA CAR-T cells cultured in 6 different commercial T cell expansion Media, AIM V™ (GIBCO™, 12055083), PRIME-XV T Cell CDM (FUJIFILM, 91154), X-VIVO 15 (Lonza, BE02-054Q), LymphoONE (Takara, WK552S), TEXMACS™ (Miltinenyi,130-097-196), and CTS™ OPTMIZER™ T Cell Expansion SFM (GIBCO™ A1048501). For each group, the bars from left to right represent data obtained for PCIR-BCGN1 IgG4-Fc, PCIR-BCNG2 IgG4-Fc, PCIR-tCD19, PCIR-BCMA only and NT. Data presented as an average from 4 independent donors as fold changes±SEM. As shown in FIG. 5B, the pool of T cell supernatant from Four donors cultured in EX-CELL® 620-HSF Serum-Free Medium for Hybridoma Cells (Sigma:14621C) were purified using PIERCE™ Anti-HA Agarose (Thermo 26182), five elusions were performed ELISA assay. As shown in FIG. 5C, total 10 μl of Unpurified Supernatant (U), Flow through (F), and Elution (E) from transduced T cells with “PCIR-BCGN1 IgG4-Fc”, “PCIR-BCNG2 IgG4-Fc”, “PCIR-Tcd19-EV”, and “PCIR-BCMA CAR only” were loaded onto the 4-20% MINI-PROTEAN® TGX™ Precast Protein Gels (Bio-Rad, #4561096). Primary antibody used was Rabbit-Anti-HA (CST, #3724) and the secondary antibody was LI-COR IRDYE® 800CW Goat anti-Rabbit IgG (H+L) (Li-COR, #926-32211). There were expected˜75 KD HA-Tagged BiTE/BiKE protein bands only in Elution from the “PCIR-BCGN1 IgG4-Fc” and “PCIR-BCNG2 IgG4-Fc” groups, but not from supernatant of samples of “PCIR-tCD19-EV” and “PCIR-BCMA CAR only”.

FIGS. 6A-6B provide results of safety, growth, and survival study. Cytokine independent growth experiment was performed to ensure CAR T cells are not transformed. Equal number cells (1.5×10⁴ cells were seeded in the 96 well plates with media X-VIVO 15 containing 5% human AB type serum at the condition “no cytokine” (FIG. 6A) and “cytokine plus” (IL-2 50 IU/ml; IL-15 0.5 ng/ml) (FIG. 6B). NovoCyte 3005 Flow Cytometry used to count the cell number. The fold changes of total DAPI negative live cells verses the starting 15000 cell number were used to evaluate the cell survival and growth. Data is presented as an average from 4 independent donors as fold changes±SEM. Without cytokines, all groups died on day 10, and the ranges of growth were 100-250 folds on day 10 with cytokines present, suggesting growth of all these transduced T cells was dependent on cytokines and not transformed. Importantly, there were significantly beneficial effects on survival and growth of BiTE/BiKE-BCMA CAR-T, particular “PCIR-BCGN1 IgG4-Fc” compared to “conventional PCIR-BCMA CAR only” in both condition of absent and present cytokines. P value here only demonstrated the GN1-BiTE/BiKE-BCMA CAR-T compared to Conventional BCMA-CAR-T. *: p<0.05; **: p<0.01

FIGS. 7A-7C show generation of BiTE/BiKE specific killing models in both homogenous and heterogeneous tumor targets environment with a mixture containing BCMA+/GPRCSD+cell lines MM1.S or OPM2, and BCMA+/GPRCSD-RPMI8226 as well as BCMA-/GPRCSD-MV411. As shown in FIG. 7A, flow analysis determined that the MM1.S and OPM2 are BCMA and GPRCSD positive MM cell lines while the RPMI8226 is BCMA positive but GPRCSD negative MINI cell line. The MV411 used in this study is BCMA and GPRC5D double negative AML cell line. For generation of the BCMA and GPRC5D targets homogenous and heterogeneous MINI tumor environment, Applicant mixed 75% of MM1.S or OPM2 with 25% of MV411, and 40% of MM1.S or OPM2 with 60% of MV411 (FIG. 7B) and the long-term 120 hours killing assay results are shown in FIGS. 8A-9D. For generation of GPRCSD target homogenous and heterogeneous MINI tumor environment, 75% of MM1.S or OPM2 with 25% of RPMI 8226, and 40% of MM1.S or OPM2 with 60% of RPMI 8226 (FIG. 7C) and the long-term 120 hours killing assay results are shown in FIGS. 10A-11D. BCMA negative, GPRCSD positive MM cell lines were made to generate BCMA target homogenous and heterogeneous MINI tumor environment.

FIGS. 8A-8D show that GN1-BiTE/BiKE BCMA-CAR-T cells outperform (for example, demonstrate significantly better efficacy in killing target cell) conventional BCMA-CAR-T cells in both BCMA+/GPRCSD+MM1.S and BCMA-/GPRCSD-MV411 homogenous and heterogeneous tumor target environment. Long-term killing assay was performed. BCMA+/GPRC5D+MM1.S and BCMA-/GPRCSD-MV411 cells were mixed at variable percentages, such as 100% MM1.S (FIG. 8A), 100% MV411 (FIG. 8B), 75% MM1.S plus 25% MV411 (FIG. 8C), and 40% MM1.S plus 60% MV411 (FIG. 8D). There were significantly tumor killing effects for both GN1-BiTE/BiKE BCMA-CAR (PCIR-BCGN1 IgG4-Fc) and conventional BCMA-CAR-T compared to empty control non-transduced T cells in homogenous and heterogeneous models (FIG. 7B). However, GN1-BiTE/BiKE BCMA-CAR demonstrated dramatically tumor killing effects compared to conventional BCMA-CAR (statistical significance P<0.0001). Data is presented as an average from 4 independent donors as tumor lysis±SEM. Two tails Student's T-Test was used to evaluate the significances between two groups, and the index for P value were: * p<0.05; ** p<0.01; *** p<0.001; **** p<0.0001.

FIGS. 9A-9D show that GN1-BiTE/BiKE BCMA-CAR-T outperform (for example, demonstrate significantly better efficacy in killing target cell) conventional BCMA-CAR-T in both BCMA+/GPRCSD+OPM2 and BCMA-/GPRCSD-MV411 homogenous and heterogeneous tumor target environment. Long-term killing assay was performed. BCMA+/GPRCSD+OPM2 and BCMA-/GPRCSD-MV411 cells were mixed at variable percentages, such as 100% OPM2 (FIG. 9A), 100% MV411 (FIG. 9B), 75% OPM2 plus 25% MV411 (FIG. 9C), and 40% OPM2 plus 60% MV411 (FIG. 9D). There were significantly tumor killing effects for GN1-BiTE/BiKE BCMA-CAR (PCIR-BCGN1 IgG4-Fc), NG2-BiTE/BiKE BCMA-CAR (PCIR-BCGN1 IgG4-Fc), and conventional BCMA-CAR-T compared to empty control non-transduced T cells in homogenous and heterogeneous models (FIG. 7B). However, GN1-BiTE/BiKE BCMA-CAR demonstrated dramatically tumor killing effects compared to conventional BCMA-CAR (statistical significance P<0.0001). Data is presented as an average from 4 independent donors as Tumor lysis±SEM. Two tails Student's T-Test was used to evaluate the significances between two groups, and the index for P value were: * p<0.05; ** p<0.01; *** p<0.001; **** p<0.0001.

FIGS. 10A-10D show that GN1-BiTE/BiKE BCMA-CAR-T outperform (for example, demonstrate significantly better efficacy in killing target cell) conventional BCMA-CAR-T in both BCMA+/GPRC5D+MM1.S and BCMA+/GPRC5D-RPMI8226 homogenous and heterogeneous tumor target environment. Long-term killing assay was performed. BCMA+/GPRC5D+MM1.S and BCMA+/GPRC5D-RPMI8226 cells were mixed at variable percentages, such as 100% MM1.S (FIG. 10A), 100% RPMI8226 (FIG. 10B), 75% MM1.S plus 25% RPMI8226 (FIG. 10C), and 40% MM1.S plus 60% RPMI8226 (FIG. 10D). There were significantly tumor killing effects for both GN1-BiTE/BiKE BCMA-CAR (PCIR-BCGN1 IgG4-Fc) and conventional BCMA-CAR-T compared to empty control non transduced T cells in homogenous and heterogeneous models (FIG. 7C). However, GN1-BiTE/BiKE BCMA-CAR demonstrated dramatically tumor killing effects compared to conventional BCMA-CAR (statistical significance P<0.0001). Data is presented as an average from 4 independent donors as Tumor lysis SEM. Two tails Student's T-Test was used to evaluate the significances between two groups, and the index for P value were: * p<0.05; ** p<0.01; *** p<0.001; **** p<0.0001.

FIGS. 11A-11D show that GN1-BiTE/BiKE BCMA-CAR-T outperform (for example, demonstrate significantly better efficacy in killing target cell) conventional BCMA-CAR-T in both BCMA+/GPRC5D+OPM2 and BCMA+/GPRC5D-RPMI8226 homogenous and heterogeneous tumor target environment. Long-term killing assay was performed. BCMA+/GPRC5D+OPM2 and BCMA+/GPRC5D-RPMI8226 were mixed at variable percentages, such as 100% OPM2 (FIG. 11A), 100% RPMI8226 (FIG. 11B), 75% OPM2 plus 25% RPMI8226 (FIG. 11C), and 40% OPM2 plus 60% RPMI8226 (FIG. 11D). There were significantly tumor killing effects for GN1-BiTE/BiKE BCMA-CAR (PCIR-BCGN1 IgG4-Fc), NG2-BiTE/BiKE BCMA-CAR (PCIR-BCGN1 IgG4-Fc), and conventional BCMA-CAR for use in an immune cell, such as a T cell, compared to empty control non transduced T cells in homogenous and heterogeneous models (FIG. 7C). However, GN1-BiTE/BiKE BCMA-CAR demonstrated dramatically tumor killing effects compared to conventional BCMA-CAR (statistical significance P<0.0001). Data is presented as an average from 4 independent donors as tumor lysis±SEM. Two tails Student's T-Test was used to evaluate the significances between two groups, and the index for P value were: * p<0.05; ** p<0.01; *** p<0.001; **** p<0.0001.

FIGS. 12A-12C provide schematic representation of empty control (FIG. 12A), anti-BCMA conventional CAR for use in an immune cell, such as an NK cell (FIG. 12B) and two anti GPRC5D and NKG2D BiTE/BiKE CAR (FIG. 12C) with soluble cytokines (IL-15) and suicide gene tEGFR modification retroviral vectors. There were two BiTE/BiKE-BCMA CAR constructs used in this study with differences of first scFv and second scFv in BiTE/BiKE components (FIG. 12C). Soluble cytokines (IL-15) and suicide gene tEGFR modification were introduced for CAR for use in an immune cell, such as an NK cell.

FIGS. 13A-13C provide schematic representations of empty control (FIG. 13A), anti-BCMA conventional CAR for use in an immune cell, such as an NK cell (FIG. 13B) and two anti GPRC5D and NKG2D BiTE/BiKE CAR (FIG. 13C) retroviral vectors with soluble IL-15 and receptor complex (SIL15C) and suicide gene tEGFR modification. There were two BiTE/BiKE-BCMA CAR constructs used in this study with differences of first scFv and second scFv in BiTE/BiKE components (FIG. 13C). Soluble IL-15 with receptor complex (SIL15C) and suicide gene tEGFR modification were introduced for CAR for use in an immune cell, such as an NK cell.

FIGS. 14A-14C provide schematic representations of empty control (FIG. 14A), anti-BCMA conventional CAR for use in an immune cell, such as an NK cell(FIG. 14B) and two anti-GPRC5D and anti-NKG2D BiTE/BiKE CAR (FIG. 14C) retroviral vectors with membrane bound IL-15 (mbIL-15) and suicide gene tEGFR modification. There were two BiTE/BiKE-BCMA CAR constructs used in this study with differences of first scFv and second scFv in BiTE/BiKE components (FIG. 14C). On-Board cytokines (IL-15) was bounded on cell membrane (mbIL-15) with PDGFRβ transmembrane domain and suicide gene tEGFR modification were introduced for CAR for use in an immune cell, such as an NK cell.

FIGS. 15A-15C provide schematic representations of empty control (FIG. 15A), anti-BCMA conventional CAR for use in an immune cell, such as an NK cell (FIG. 15B) and two anti GPRC5D and NKG2D BiTE/BiKE CAR (FIG. 15C) retroviral vectors membrane bound IL-21 (mblL-21) and suicide gene tEGFR modification. There were two BiTE/BiKE-BCMA CAR constructs used in this study with differences of first scFv and second scFv in BiTE/BiKE components (FIG. 15C). On-Board cytokine (IL-21) was bounded on cell membrane (mblL-21) with PDGFRβ transmembrane domain and suicide gene tEGFR modification were introduced for CAR for use in an immune cell, such as an NK cell.

FIGS. 16A-16C provide schematic representations of tumor antigens BCMA (FIG. 16A), GPRC5D (FIG. 16B), and dual GPRC5D and BCMA (FIG. 16C) retroviral vectors used for generation artificial cell lines. The BCMA protein sequences used in this study was: UniProtKB-Q02223 (TNR17 HUMAN) and the GPRC5D protein was UniProtKB-Q9NZD1 (GPC5D HUMAN). All the inserts were cloned into retrovirus vectors PCIR between 5′ and 3′ LTR.

FIGS. 17A-17F provide diagraphs of BiTE/BiKE-BCMA CAR and controls used in this study (“(G4S)3” disclosed as SEQ ID NO: 9) (FIGS. 17A-17E), along with diagraphs of tumor antigens BCMA, GPRC5D, and dual GPRC5D and BCMA retroviral vectors used for generation artificial cell lines (FIG. 17F). The anti BCMA scFv was extracted from mouse anti human BCMA antibody C11D 5.3 (WO 2015/158671 A1), and BiTE/BiKE was full human source: human anti human NKG2D antibody KYK-2.0 (US20110150870A1), and human anti human GPRC5D antibody GC5B596 (US Patent Application 20180037651). There were two BiTE/BiKE-BCMA CAR constructs used in this study with differences of first scFv and second scFv in BiTE/BiKE components. A three-site mutated human IgG4 Fc was used to stabilize the BiTE/BiKE secreted. IgG4-Fc had been mutated at three sites, F234A, L235A within hinge region and N297Q within CH2 region, substitutions in its Fc region were aimed to avoid the Fc receptors binding. All the inserts were cloned into retrovirus vectors PCIR between 5′ and 3′ LTR. The amino acids and nucleotide sequences for each components and whole sequences of the constructs as shown in FIG. 17A are provided in Tables 2a, b, c, d and e. As shown in FIG. 17B, soluble cytokines (IL-15) and suicide gene tEGFR modification were introduced for CAR for use in an immune cell, such as an NK cell. The amino acids and nucleotide sequences for each components and whole sequences of the constructs as shown in FIG. 17B are provided in Tables 2a, f, g, h and i. As shown in FIG. 17C, soluble IL-15 with receptor complex (SIL15C) and suicide gene tEGFR modification were introduced for CAR, such as those for use in NK cells or other immune cells. The amino acids and nucleotide sequences for each components and whole sequences of the constructs as shown in FIG. 17C are provided in Tables 2a, j, k,l and m. As shown in FIG. 17D, on-board cytokines (IL-15) was bounded on cell membrane (mbIL-15) with PDGFRβ transmembrane domain and suicide gene tEGFR modification were introduced for CAR, such as those for use in NK cells or other immune cells. The amino acids and nucleotide sequences for each components and whole sequences of the constructs as shown in FIG. 17D are provided in Tables 2a, n, o, p and q. As shown in FIG. 17E, on-board cytokines (IL-21) was bounded on cell membrane (mblL-21) with PDGFRβ transmembrane domain and suicide gene tEGFR modification were introduced for CAR, such as those for use in NK cells or other immune cells. The amino acid and nucleotide sequences for each components and whole sequences of the constructs as shown in FIG. 17E are provided in Tables 2a, r, s, t and u. FIG. 17F provides diagraphs of tumor antigens BCMA, GPRC5D, and dual GPRC5D and BCMA retroviral vectors used for generation artificial cell lines. The BCMA protein sequences used in this study was UniProtKB—Q02223 (TNR17 HUMAN), while the GPRC5D protein was UniProtKB—Q9NZD1 (GPC5D HUMAN). All the inserts were cloned into retrovirus vectors PCIR between 5′ and 3′ LTR. The amino acid and nucleotide sequences for each components and whole sequences of the constructs as shown in FIG. 17F are provided in Tables 2a, v, w, and x.

FIG. 18 shows data determining the secretion of bispecific antibody secreted from transduced NK cells by ELISA. Data presented as average±SEM from 4 independent donors. ELISA analysis as shown detected BiTE/BiKE from non-concentrated supernatant of transduced BiTE/BIKE-BCMA CAR-NK cells cultures in all 5 groups CAR-NK with or without onboard cytokine modification. The bars within each group represent, from left to right, data obtained via using the conventional mBCMA-CAR, GN1-BiTE/BiKE mBCMA-CAR, NG2-BiTE/BiKE mBCMA-CAR, empty control-tCD19 and non-transduced cells.

FIGS. 19A-19C show effector cells (such as CAR expressing immune cells) and heterogeneous tumor model used for RTCA (Real Time cytotoxicity Assay) in this study. xCELLigence MP system instruments and RTCA Software Pro 2.3 (ACEA Biosciences, Agilent Technologies, Inc.) were used to perform and analyze the RTCA. Effector cell groups and transduction evaluation used in RTCA are illustrated in FIGS. 19A-19B. Artificial over expression GPRC5D and double BCMA/GPRC5D gene were transduced into 293T using modified retrovirus gene delivery system and served as a target cell, which is also referred to herein as a Target. With mixtures of two parts of 293T-GPRC5D (76.74%) and three parts of 293T-GPRC5D/BCMA (70.62% double positive, and 21.97% BCMA+), Applicant generated heterogeneous tumor model to mimic the Multiple Myeloma tumor environment (FIG. 19C). Total 5000 targets were seeded on E-Plate 96 PET (Agilent REF 300600910) and incubated at 37 degree with 5% CO₂ for about 20 h, then total 500 effectors were added into the wells with targets. The ratio of Effectors (immune cells, such as NK cells, transduced with the vector as disclosed herein) and Targets (293 T cells infected with GPRC5D or both BCMA and GPRC5D) used in this study was 1:10.

FIG. 20 provides an RTCA assay for BiTE/BiKE-CAR with soluble cytokines (IL-15) and suicide gene tEGFR modification expressed in an immune cell such as NK cell. Data presented as % cytolysis from 2 independent donors. The cells were real-time monitored at 37° C. in humidified 5% CO₂ atm. Cell Index (CI) data was recorded every 15 min for 120 h. The selected normalization time point was 20 h after seeding. The time to reach maximum Cell Index for targets alone group get was ˜80h, so the % cytolysis was calculated and compared at ˜60h with addition of effectors.

FIG. 21 provides an RTCA assay for BiTE/BiKE-CAR with soluble IL-15 and IL-15 receptor complex (SIL15C) and suicide gene tEGFR modification expressed in an immune cell such as NK cell. Data presented as % cytolysis from 2 independent donors. The cells were real-time monitored at 37° C. in humidified 5% CO₂ atm. Cell Index (CI) data was recorded every 15 min for 120 h. The selected normalization time point was 20 h after seeding. The time to reach maximum Cell Index for targets alone group get was ˜80h, so the % Cytolysis was calculated and compared at ˜60h with addition of effectors.

FIG. 22 provides an RTCA assay for BiTE/BiKE-CAR with membrane bound IL-15 and suicide gene tEGFR modification expressed in an immune cell such as NK cell. Data presented as % cytolysis from 2 independent donors. The cells were real-time monitored at 37° C. in humidified 5% CO₂ atm. Cell Index (CI) data was recorded every 15 min for 120 h. The selected normalization time point was 20 h after seeding. The time to reach maximum Cell Index for targets alone group get was ˜80h, so the % cytolysis was calculated and compared at ˜60h with addition of effectors.

FIG. 23 provides an RTCA assay for BiTE/BiKE-CAR with membrane bound IL-21 and suicide gene tEGFR modification expressed in an immune cell such as NK cell. Data presented as % cytolysis from 2 independent donors. The cells were real-time monitored at 37° C. in humidified 5% CO₂ atm. Cell Index (CI) data was recorded every 15 min for 120 h. The selected normalization time point was 20 h after seeding. The time to reach maximum Cell Index for targets alone group get was ˜80h, so the % cytolysis was calculated and compared at ˜60h with addition of effectors.

FIG. 24 provides an RTCA assay for BiTE/BiKE-CAR without modification expressed in an immune cell such as NK cell. Data presented as % cytolysis from 2 independent donors. The cells were real-time monitored at 37° C. in humidified 5% CO₂ atm. Cell Index (CI) data was recorded every 15 min for 120 h. The selected normalization time point was 20 h after seeding. The time to reach maximum Cell Index for targets alone group get was ˜80h, so the % cytolysis was calculated and compared at ˜60h with addition of effectors.

FIG. 25 shows increased cytolysis of NKG2D and GPRCSD BiTE/BiKE-BCMA CAR expressed in an immune cell such as NK cell compared to conventional BCMA-CAR determined by RTCA. Data presented as an average from 2 independent donors as tumor lysis±SD at reaction of 60h.

FIGS. 26A-26C provide schematic representations of an empty control (FIG. 26A), anti-BCMA conventional CAR-NK (FIG. 26B) and two anti GPRCSD and NKG2D BiKE CAR (FIG. 26C) with soluble cytokines (IL-15) and suicide gene RQR8 modification retroviral vectors. As shown in FIG. 26C, there were two BiKE-BCMA CAR constructs used in this study with differences of first scFv and second scFv in BiKE components. Soluble cytokines (IL-15) and suicide gene RQR8 modification were introduced into the CAR-NK. *: The efficacies of two kinds of BiKE with IL-2 signal peptide or azurocidin signal peptide were investigated based on the feasibility of virus production, transduction of cold blood derived NK cells, secretion of BiKE, and specific tumor killing abilities.

FIG. 27 shows optimizing of constructs “Two in One” with highly compact sort-suicide reporter RQR8 and strong azurocidin signal peptide for BiKE secretion. RQR8 is a dual role reporter for both selection/tracking and suicide gene with highly compact size 136 aa compared to the 336 aa tEGFR. azurocidin signal peptide with proven evidences in other constructs as a strong signal peptide for soluble BiTE/BiKE secretion and used to replace the previous IL 2 signal peptide for BiTE/BiKE. The goals of these modifications are improving the transgenes expression and efficacy of antitumor.

FIG. 28 provides a diagraph of BiKE-BCMA CAR and controls, such as P67 (PCIR-mBCMA-Az-SIL-15-RQR8), P61 (PCIR-GN1-BiTE/BiKE-Az-SIL-15-RQR8), P62 (PCIR-NG2-BiTE/BiKE-Az-SIL-15-RQR8), P72 (PCIR-NG2-BiTE/BiKE-Az-SIL-15-RQR8), and P68 (PCIR-tDC19-Az-SIL-15-RQR8). The amino acid and nucleotide sequences for each components and whole sequences of the constructs are provided in Table 3. “(G4S)3” disclosed as SEQ ID NO: 9.

FIGS. 29A-29B provide an assessment of transgene expression of “Two in One” BCMA-CAR-NK secretion BiKE. It provides the feasibility of “Two in One” virus production and transduction of cold blood derived NK cells. NK cells from 4 donors (UCBV-033, UCBV-034, UCOSU-018 and UCSC-711) were co-cultured with a modified IRR K562 feeder cells at a ratio of 1:2 using SCGM media with 50 IU/ml of IL-2. NK cells were transduced with retrovirus expressing soluble IL-15 and sort/suicide marker RQR8 at day 6 at an MOI of 3. FIG. 29A provides representative flow diagrams of the transduction efficiency of NK cells on Day 6 assessed by using anti human CD34 with the flow antibody QBEND/10 (R&D Systems, Inc. FAB7227A). FIG. 29B shows data presented as positive RQR8 population with mean±SEM.

FIGS. 30A-30C provide efficacy of tumor killing with Flow Cytometry of “Two in One” BCMA-CAR-NK with secretion of IL2 SP GPRCSD-NKG2D BiKE. It provides efficacy of targeting BCMA with secretion of bispecific antibody NKG2D-CS1 or NKG2D-GPRCSD for MINI cell line MM1.S in vitro long-term killing. FIG. 30A provides samples statistics of Flow Cytotoxicity used in CAR-NK study. Flow Cytometer NovoCyte 451181231028 and software NovoExpress 1.5.1 (Acea) were used for data collection and analysis. Equal update volume 100 μl of samples was collected. Multiple Myeloma cell lines, MM1.S was infected with Luc-ZsGreen plasmid in advance, and parameters were used to discriminate the effectors from target cells. The cell counts of gate Q4-4 (ZsGreen positive, marked in red) from DAPI negative live singlet were used to evaluate the live MINI cells. The percentage of specific lysis % is calculated using the formula: Specific lysis %=100*(Targets only-targets in mix)/Targets only. FIG. 30B shows tumor antigens BCMA, GPRCSD, expressed on MM cell line, MM1.S. FIG. 30C shows effector CAR-NK cells and their mock empty vectors control (FIG. 27) were co-culture with Multiple Myeloma cell line, MM1.S at the transduced CAR-NK: tumor ratio 1:10 for 96h. Data presented as an average from 4 independent donors as Tumor lysis±SEM.

FIG. 31 shows efficacy of tumor killing with Real Time Cytotoxicity Assay (RTCA) of “Two in One” BCMA CAR-NK with secretion of IL2 SP GPRC5D-NKG2D BiKE. An RTCA assay was performed for BiKE-CAR-NK with soluble cytokines (IL-15) and suicide gene RQR8 modification. The transduced CAR-NK: tumor ratio was 1:5, and the cells were real-time monitored at 37° C. in humidified 5% CO₂ atm. Cell Index (CI) data were recorded every 15 min for 120 h. The selected normalization time point was 30 h after seeding. The time to reach maximum Cell Index for targets alone group get was ˜80h, so the % cytolysis was calculated and compared at ˜60h with addition of effectors.

FIG. 32 shows “CAR-NK” combination with “Bispecific Antibody” outperform “CAR-NK” based products in killing assays. RTCA (Real Time Cytolysis Assay) demonstrated that there were enhanced killing efficacy of products “BCMA-CAR-NK” combined with BiKE-GPRCSD-NKG2D (GN1) or BiKE-NKG2D-GPRCSD (NG2), compared to the “CAR-NK” based products (conventional single BCMA-CAR-NK, GPRCSD-CAR-NK, as well as tandem BCMA-GPRCSD CAR-NK). Non-transduced NK cell and empty vector mock infection served as negative control for both products. The HT1080 cell line with forced GPRCSD and BCMA antigens expression was used as targets, and the ratio of transduced NK vs tumor target was 1:10 (total NK vs tumor was 1:2) in each group. Data presented as an average from 2 independent donors as tumor lysis±SD at reaction 44 h with addition of effectors.

FIGS. 33A-33B show efficacy of in vitro killing activity of “CAR-NK” combination with “Bispecific Antibody.” RTCA (Real Time Cytolysis Assay) demonstrated that there were enhanced killing efficacy for both strategy 1 and 2 products “BCMA-CAR-NK combined with BiKE-GPRCSD-NKG2D (GN1) or BiKE-NKG2D-GPRCSD (NG2), compared to the “CAR-NK” alone product. Non transduced NK cell and empty vector mock infection served as negative control for both products. The HT1080 cell line with forced GPRCSD and BCMA antigens expression was used as targets, and the ratio of transduced NK vs tumor target was 1:10 (total NK vs tumor was 1:2) in each group. Data presented as an average from 3 independent donors as tumor lysis±SEM at reaction 44 h with addition of effectors. Paired T test is used to evaluate the differences of conventional BCMA group with the other groups. FIG. 33A shows strategy 1 “Two in One” while FIG. 33B shows strategy 2 “Virus Mix.”

FIGS. 34A-34B show two strategies of “CAR-NK” combination with “Bispecific Antibody.” Schematic representation was provided of original strategy 1 “anti-BCMA conventional CAR-NK and two anti GPRC5D and NKG2D BiKE CAR with soluble cytokines (IL-15) and suicide gene tEGFR modification retroviral vectors” and strategy 2 “Virus Mixture”. There were two BiKE-BCMA CAR constructs used in this study with differences of first scFv and second scFv in BiKE components. Soluble cytokines (IL-15) and suicide gene tEGFR modification were introduced for CAR-NK. FIG. 34A shows strategy 1 “Two in One”, while FIG. 34B shows virus mixture strategy 2 “One plus One”.

FIG. 35 shows optimizing of constructs “Two in One” with highly compact sort-suicide reporter RQR8 and strong azurocidin signal peptide for BiKE secretion. Original construct “Two in One” was optimized with highly compact sort-suicide reporter RQR8 and strong Azurocidin signal peptide for BiKE. RQR8 is a dual role reporter for both selection/tracking and suicide gene with highly compact size 136 aa compared to the 336 aa tEGFR. azurocidin signal peptide with proven evidences in other constructs as a strong signal peptide for soluble BiTE/BiKE secretion and used to replace the previous IL 2 signal peptide for BiKE. The goals of these modifications are improving the transgenes expression and efficacy of antitumor.

FIGS. 36A-36D provide in vivo efficacy of the CAR cells. FIG. 36A provides a scheme of the in vivo experimental model and representative images showing tumor loads of mice treated with different therapies as labeled (including: mice injected with saline serving as a control, mice injected with empty control transfected/transduced T cells serving as a control; mice injected with bispecific antibody expressing T cells, mice injected with anti-BCMA CAR expressing T cells, mice injected sequentially with bispecific antibody expressing T cells and CAR expressing T cells, and mice injected with CAR and bispecific antibody expressing T cells) and at various time points as labeled. Each time point was indicated from two perspectives: the larger day number provides days after tumor implantation, while the smaller day number provides days after treatment. FIG. 36B shows exemplified flowy cytometry plots of engineered T cells in a sample isolated from treated mice. FIG. 36C plots total PBL cell numbers, Fab+percentages and Fab+cell numbers in a sample isolated from treated mice. FIG. 36D provides a survival curve of mice treated with different therapies as labeled.

FIGS. 37A-37C provide schematic representations of an empty control (FIG. 37A), an anti-BCMA conventional CAR (FIG. 37B) and two anti NKG2D and GPRC5D BiKE CAR (FIG. 37C) with soluble cytokines (IL-15) and suicide gene RQR8 modification retroviral vectors. There were two BiKE-BCMA CAR constructs used in this study with differences of first scFv and second scFv in BiTE/BiKE components. Soluble cytokines (IL-15) and suicide gene RQR8 modification were introduced into CAR-NK.

FIG. 38 provides a diagraph of BiKE-BCMA CAR and controls (“(G45)3” disclosed as SEQ ID NO: 9).. All the four inserts were cloned into retrovirus vectors PCIR between 5′ and 3′ LTR. The amino acids and nucleotide sequences for each components and whole sequences of above constructs were provided in Table 3. P68: Empty Control-Truncated CD19-SIL-15-RQR8; P67: Conventional BCMA-CAR-SIL-15-RQR8; P61: GPRC5D-NKG2D BiKE-BCMA CAR-NK; P62: NKG2D-GPRC5D BiKE-BCMA CAR-NK.

FIGS. 39A-39B show determination of the transgene expression of BCMA CAR NK, and BCMA CAR NK with secretion of NKG2D and GPRC5D BiKE by flow cytometry analysis before cytotoxicity. Flow antibodies against human CD34 Antibody (QBEnd/10) (Allophycocyanin) (Novus Biologicals, LLC, #FAB7227A) was used to detect reported gene RQR8. FIG. 39A shows NovoCyte 3005 Flow Cytometry analysis demonstrated transduction efficiency for Gamma Retrovirus vector PCIR/BaEVTR engineered BCMA-CAR-NK from four donors. FIG. 39B presents data as average percentage of RQR8 positive population from four independent donors as mean±SEM.

FIGS. 40A-40B show determination of the expression and concentration of Bispecific antibody secreted from NK cells by ELISA. The secretion of soluble IL-15 and BiKE secreted from transduced NK cells were determined by ELISA. 0.5-1×10⁶ transduced NK cells (FIG. 39) were seeded and cultured in serum free media for 72 hours. Supernatant was subsequently collected, centrifuged, and immediately added to IL-15 pre-coated ELISA plate (R&D Systems S1500) and home developed BiKE detection ELISA with NKG2D-fusion protein coated plate. ELISA analysis showed there were detectable and soluble IL-15 in each group (FIG. 40A) and IL2 SP BiKE from non-concentrated supernatant of transduced BIKE-BCMA CAR-NK cells cultures in all 5 groups at culture 72 hours (FIG. 40B). Data present here was as average concentration from four independent donors as mean±SD.

FIG. 41 shows RTCA (Real Time cytotoxicity Assay) assessment and % cytolysis monitored between and among groups of different modification of CAR-NK illustrated with independent assays. RTCA assay was performed for BiKE-CAR-NK with soluble cytokines (IL-15) and report gene RQR8 modification. xCELLigence MP system instruments and RTCA Software Pro 2.3 (ACEA Biosciences, Agilent Technologies, Inc.) were used to perform and analyze the RTCA. Effector cells groups and transduction evaluation used in RTCA were illustrated in FIG. 39, and the primary NK cells from cord blood cell were used in this study. Forced expression GPRCSD and BCMA gene on HT1080 were used as tumor antigen targets in RTCA assay. Total 5000 targets were seeded on E-Plate 96 PET (Agilent REF 300600910) and incubated at 37 degree with 5% CO₂ for 23 h, then added total 5000 effectors into the wells with targets. Equal 1250 transduced NK used in each group by adjusting with non-transduced NK form same donor according to transgene expression. The ratio of transduced NK and Targets used in this study was 1:4. Cell Index (CI) data were recorded every 15 min for 120 h. The time to reach maximum Cell Index for targets alone group get was ˜56h, so the % cytolysis was calculated and compared at ˜26h with addition of effectors. The data demonstrated there were enhanced tumor killing efficacy of GN1 BiKE BCMA CAR NK compared to other groups.

FIGS. 42A-42B provide data accessing long term in vitro cytotoxicity by Flow Cytometry. Efficacy was evaluated of targeting BCMA with secretion of bispecific antibody GPRCSD-NKG2D for MM cell lines in vitro long-term killing. FIG. 42A provides diagrams of BCMA and GPRCSD surface expression on Multiple Myeloma (MM) cell lines, MM1.S, OPM2, H929 and RPMI8226 assessed by using the flow antibody anti human BCMA (BioLegend, Cat #357506) and GPRCSD (R&D Systems, FAB6300P-100) with their isotype controls. The data demonstrated that, there are broad BCMA and GPRCSD on the surface of MM cell. FIG. 42B provides effectors CAR-NK cells and their mock empty vectors (Tcd19) control as well as non-transduced NK were co-culture with four Multiple Myeloma cell lines, MM1.S, OPM2,H929, and RPMI 8226 at the transduced CAR-NK: tumor ratio 1:8 (Total NK: Tumor 1:2) for 96h. Data presented as an average from 4 independent donors as Tumor lysis±SEM. Two tails Student's T-Test were used to evaluate the significances between two groups, and the index for P value were: * p<0.05; ** p<0.01. There are dramatically tumor killing activity of BCMA-CAR-NK with secretion bispecific antibody GPRCSD-NKG2D (GN1-BiKE) compared to conventional BCMA-CAR-NK, and others.

### DETAILED DESCRIPTION

As it would be understood, the section or subsection headings as used herein is for organizational purposes only and are not to be construed as limiting and/or separating the subject matter described.

Unless defined otherwise, all technical and scientific terms used herein have the same meanings as commonly understood by one of ordinary skill in the art to which this disclosure belongs. Although any methods and materials similar or equivalent to those described herein can be used in the practice or testing of the present disclosure, the preferred methods, devices, and materials are now described. All technical and patent publications cited herein are incorporated herein by reference in their entirety. Nothing herein is to be construed as an admission that the disclosure is not entitled to antedate such disclosure by virtue of prior disclosure.

The practice of the present disclosure will employ, unless otherwise indicated, conventional techniques of tissue culture, immunology, molecular biology, microbiology, cell biology and recombinant DNA, which are within the skill of the art. See, e.g., Sambrook and Russell eds. (2001) Molecular Cloning: A Laboratory Manual, 3rd edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); Herzenberg et al. eds (1996) Weir's Handbook of Experimental Immunology; Manipulating the Mouse Embryo: A Laboratory Manual, 3rd edition (Cold Spring Harbor Laboratory Press (2002)); Sohail (ed.) (2004) Gene Silencing by RNA Interference: Technology and Application (CRC Press).

All numerical designations, e.g., pH, temperature, time, concentration, and molecular weight, including ranges, are approximations which are varied (+) or (−) by increments of 0.1 or 1.0, where appropriate. It is to be understood, although not always explicitly stated that all numerical designations are preceded by the term “about.” It also is to be understood, although not always explicitly stated, that the reagents described herein are merely exemplary and that equivalents of such are known in the art.

The term “about,” as used herein when referring to a measurable value such as an amount or concentration and the like, is meant to encompass variations of 20%, 10%, 5%, 1%, 0.5%, or even 0.1% of the specified amount.

As used in the specification and claims, the singular form “a”, “an” and “the” include plural references unless the context clearly dictates otherwise. For example, the term “a cell” includes a plurality of cells, including mixtures thereof.

As used herein, the term “comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude trace contaminants from the isolation and purification method and pharmaceutically acceptable carriers, such as phosphate buffered saline, preservatives and the like. “Consisting of” shall mean excluding more than trace elements of other ingredients and substantial method steps for administering the compositions of this disclosure or process steps to produce a composition or achieve an intended result. Embodiments defined by each of these transition terms are within the scope of this disclosure.

“Optional” or “optionally” means that the subsequently described circumstance may or may not occur, so that the description includes instances where the circumstance occurs and instances where it does not.

As used herein, “and/or” refers to and encompasses any and all possible combinations of one or more of the associated listed items, as well as the lack of combinations when interpreted in the alternative (“or”).

“Substantially” or “essentially” means nearly totally or completely, for instance, 95% or greater of some given quantity. In some embodiments, “substantially” or “essentially” means 95%, 96%, 97%, 98%, 99%, 99.5%, or 99.9%.

In some embodiments, the terms “first” “second” “third” “fourth” or similar in a component name are used to distinguish and identify more than one components sharing certain identity in their names. For example, “first TAA” and “second TAA” are used to distinguishing two TAAs.

The term “isolated” as used herein with respect to nucleic acids, such as DNA or RNA, refers to molecules separated from other DNAs or RNAs, respectively that are present in the natural source of the macromolecule. The term “isolated nucleic acid” is meant to include nucleic acid fragments which are not naturally occurring as fragments and would not be found in the natural state. The term “isolated” is also used herein to refer to polypeptides, proteins and/or host cells that are isolated from other cellular proteins and is meant to encompass both purified and recombinant polypeptides. In other embodiments, the term “isolated” means separated from constituents, cellular and otherwise, in which the cell, tissue, polynucleotide, peptide, polypeptide, protein, antibody or fragment(s) thereof, which are normally associated in nature. For example, an isolated cell is a cell that is separated form tissue or cells of dissimilar phenotype or genotype. As is apparent to those of skill in the art, a non-naturally occurring polynucleotide, peptide, polypeptide, protein, antibody or fragment(s) thereof, does not require “isolation” to distinguish it from its naturally occurring counterpart.

In some embodiments, the term “engineered” or “recombinant” refers to having at least one modification not normally found in a naturally occurring protein, polypeptide, polynucleotide, strain, wild-type strain or the parental host strain of the referenced species. In some embodiments, the term “engineered” or “recombinant” refers to being synthetized by human intervention. As used herein, the term “recombinant protein” refers to a polypeptide which is produced by recombinant DNA techniques, wherein generally, DNA encoding the polypeptide is inserted into a suitable expression vector which is in turn used to transform a host cell to produce the heterologous protein.

The terms “polynucleotide”, “nucleic acid” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any embodiment of this disclosure that is a polynucleotide encompasses both the double-stranded form and each of two complementary single-stranded forms known or predicted to make up the double-stranded form.

A polynucleotide is composed of a specific sequence of four nucleotide bases: adenine (A); cytosine (C); guanine (G); thymine (T); and uracil (U) for thymine when the polynucleotide is RNA. Thus, the term “polynucleotide sequence” is the alphabetical representation of a polynucleotide molecule. This alphabetical representation can be input into databases in a computer having a central processing unit and used for bioinformatics applications such as functional genomics and homology searching.

The expression “amplification of polynucleotides” includes methods such as PCR, ligation amplification (or ligase chain reaction, LCR) and amplification methods. These methods are known and widely practiced in the art. See, e.g., U.S. Pat. Nos. 4,683,195 and 4,683,202 and Innis et al., 1990 (for PCR); and Wu et al. (1989) Genomics 4:560-569 (for LCR). In general, the PCR procedure describes a method of gene amplification which is comprised of (i) sequence-specific hybridization of primers to specific genes within a DNA sample (or library), (ii) subsequent amplification involving multiple rounds of annealing, elongation, and denaturation using a DNA polymerase, and (iii) screening the PCR products for a band of the correct size. The primers used are oligonucleotides of sufficient length and appropriate sequence to provide initiation of polymerization, i.e. each primer is specifically designed to be complementary to each strand of the genomic locus to be amplified.

Reagents and hardware for conducting PCR are commercially available. Primers useful to amplify sequences from a particular gene region are preferably complementary to, and hybridize specifically to sequences in the target region or its flanking regions. Nucleic acid sequences generated by amplification may be sequenced directly. Alternatively, the amplified sequence(s) may be cloned prior to sequence analysis. A method for the direct cloning and sequence analysis of enzymatically amplified genomic segments is known in the art.

A “gene” refers to a polynucleotide containing at least one open reading frame (ORF) that is capable of encoding a particular polypeptide or protein after being transcribed and translated.

The term “express” refers to the production of a gene product, such as mRNA, peptides, polypeptides or proteins. As used herein, “expression” refers to the process by which polynucleotides are transcribed into mRNA or the process by which the transcribed mRNA is subsequently being translated into peptides, polypeptides, or proteins. If the polynucleotide is derived from genomic DNA, expression may include splicing of the mRNA in a eukaryotic cell.

A “gene product” or alternatively a “gene expression product” refers to the amino acid (e.g., peptide or polypeptide) generated when a gene is transcribed and translated. In some embodiments, the gene product may refer to an mRNA or other RNA, such as an interfering RNA, generated when a gene is transcribed.

The term “encode” as it is applied to polynucleotides refers to a polynucleotide which is said to “encode” a polypeptide if, in its native state or when manipulated by methods well known to those skilled in the art, it can be transcribed to produce the mRNA for the polypeptide or a fragment thereof, and optionally translated to produce the polypeptide or a fragment thereof. The antisense strand is the complement of such a nucleic acid, and the encoding sequence can be deduced therefrom. Further, as used herein an amino acid sequence coding sequence refers to a nucleotide sequence encoding the amino acid sequence.

“Under transcriptional control”, which is also used herein as “directing expression of”, is a term well understood in the art and indicates that transcription of a polynucleotide sequence, usually a DNA sequence, depends on its being operatively linked to an element which contributes to the initiation of, or promotes, transcription. “Operatively linked” intends the polynucleotides are arranged in a manner that allows them to function in a cell.

The term “a regulatory sequence”, “an expression control element” or “promoter” as used herein, intends a polynucleotide that is operatively linked to a target polynucleotide to be transcribed or replicated, and facilitates the expression or replication of the target polynucleotide. A promoter is an example of an expression control element or a regulatory sequence. Promoters can be located 5′ or upstream of a gene or other polynucleotide, that provides a control point for regulated gene transcription. Polymerase II and III are examples of promoters. In some embodiments, a regulatory sequence is bidirectional, i.e., acting as a regulatory sequence for the coding sequences on both sides of the regulatory sequence. Such bidirectional regulatory sequence may comprises, or consists essentially of, or consists of a bidirectional promoter (see for example Trinklein N D, et al. An abundance of bidirectional promoters in the human genome. Genome Res. 2004 Jan;14(1):62-6).

The term “promoter” as used herein refers to any sequence that regulates the expression of a coding sequence, such as a gene. Promoters may be constitutive, inducible, repressible, or tissue-specific, for example. A “promoter” is a control sequence that is a region of a polynucleotide sequence at which initiation and rate of transcription are controlled. It may contain genetic elements at which regulatory proteins and molecules may bind such as RNA polymerase and other transcription factors. Non-limiting examples of promoters include the EFlalpha promoter and the CMV promoter. The EFlalpha sequence is known in the art (see, e.g., addgene.org/11154/sequences/;

ncbi.nlm.nih.gov/nuccore/J04617, each last accessed on Mar. 13, 2019, and Zheng and Baum (2014) Int′ I. J. Med. Sci. 11(5):404-408). The CMV promoter sequence is known in the art (see, e.g., snapgene.com/resources/plasmid-files/?set=basic cloning vectors&plasmid=CMVpromoter, last accessed on Mar. 13, 2019 and Zheng and Baum (2014), supra.).

An enhancer is a regulatory element that increases the expression of a target sequence. A “promoter/enhancer” is a polynucleotide that contains sequences capable of providing both promoter and enhancer functions. For example, the long terminal repeats of retroviruses contain both promoter and enhancer functions. The enhancer/promoter may be “endogenous” or “exogenous” or “heterologous.” An “endogenous” enhancer/promoter is one which is naturally linked with a given gene in the genome. An “exogenous” or “heterologous” enhancer/promoter is one which is placed in juxtaposition to a gene by means of genetic manipulation (i.e., molecular biological techniques) such that transcription of that gene is directed by the linked enhancer/promoter.

As used herein, the term “enhancer”, as used herein, denotes sequence elements that augment, improve or ameliorate transcription of a nucleic acid sequence irrespective of its location and orientation in relation to the nucleic acid sequence to be expressed. An enhancer may enhance transcription from a single promoter or simultaneously from more than one promoter. As long as this functionality of improving transcription is retained or substantially retained (e.g., at least 70%, at least 80%, at least 90% or at least 95% of wild-type activity, that is, activity of a full-length sequence), any truncated, mutated or otherwise modified variants of a wild-type enhancer sequence are also within the above definition.

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PCR reaction, or the enzymatic cleavage of a polynucleotide by a ribozyme.

As used herein, “complementary” sequences refer to two nucleotide sequences which, when aligned anti-parallel to each other, contain multiple individual nucleotide bases which pair with each other. Paring of nucleotide bases forms hydrogen bonds and thus stabilizes the double strand structure formed by the complementary sequences. It is not necessary for every nucleotide base in two sequences to pair with each other for sequences to be considered “complementary”. Sequences may be considered complementary, for example, if at least 30%, 40%, 50%, 55%, 60%, 65%, 70%, 75%, 80%, 85%, 90%, 95%, 98%, 99%, or 100% of the nucleotide bases in two sequences pair with each other. In some embodiments, the term complementary refers to 100% of the nucleotide bases in two sequences pair with each other. In addition, sequences may still be considered “complementary” when the total lengths of the two sequences are significantly different from each other. For example, a primer of 15 nucleotides may be considered “complementary” to a longer polynucleotide containing hundreds of nucleotides if multiple individual nucleotide bases of the primer pair with nucleotide bases in the longer polynucleotide when the primer is aligned anti-parallel to a particular region of the longer polynucleotide. Nucleotide bases paring is known in the field, such as in DNA, the purine adenine (A) pairs with the pyrimidine thymine (T) and the pyrimidine cytosine (C) always pairs with the purine guanine (G); while in RNA, adenine (A) pairs with uracil (U) and guanine (G) pairs with cytosine (C). Further, the nucleotide bases aligned anti-parallel to each other in two complementary sequences, but not a pair, are referred to herein as a mismatch.

Hybridization reactions can be performed under conditions of different “stringency”. In general, a low stringency hybridization reaction is carried out at about 40° C. in 10×SSC or a solution of equivalent ionic strength/temperature. A moderate stringency hybridization is typically performed at about 50° C. in 6×SSC, and a high stringency hybridization reaction is generally performed at about 60° C. in 1×SSC. Hybridization reactions can also be performed under “physiological conditions” which is well known to one of skill in the art. A non-limiting example of a physiological condition is the temperature, ionic strength, pH and concentration of Mg′ normally found in a cell.

When hybridization occurs in an antiparallel configuration between two single-stranded polynucleotides, the reaction is called “annealing” and those polynucleotides are described as “complementary.” A double-stranded polynucleotide can be “complementary” or “homologous” to another polynucleotide, if hybridization can occur between one of the strands of the first polynucleotide and the second. “Complementarity” or “homology” (the degree that one polynucleotide is complementary with another) is quantifiable in terms of the proportion of bases in opposing strands that are expected to form hydrogen bonding with each other, according to generally accepted base-pairing rules.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, or alternatively less than 25% identity, with one of the sequences of the present disclosure.

A polynucleotide or polynucleotide region (or a polypeptide or polypeptide region) has a certain percentage (for example, 70%, 75%, 80%, 85%, 90%, 95%, 98% or 99%) of “sequence identity” to another sequence means that, when aligned, that percentage of bases (or amino acids) are the same in comparing the two sequences. This alignment and the percent homology or sequence identity can be determined using software programs known in the art, for example, those described in Ausubel et al. eds. (2007) Current Protocols in Molecular Biology. Preferably, default parameters are used for alignment. One alignment program is BLAST, using default parameters. In particular, programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: www.ncbi.nlm.nih.gov/cgi-bin/BLAST. In another embodiment, the program is any one of: Clustal Omega accessible at www.ebi.ac.uk/Tools/msa/clustalo/, Needle (EMBOSS) accessible at www.ebi.ac.uk/Tools/psa/emboss_needle/, Stretcher (EMBOSS) accessible at www.ebi.ac.uk/Tools/psa/emboss_stretcher/, Water (EMBOSS) accessible at www.ebi.ac.uk/Tools/psa/emboss_water/, Matcher (EMBOSS) accessible at www.ebi.ac.uk/Tools/psa/emboss_matcher/, LALIGN accessible at www.ebi.ac.uk/Tools/psa/lalign/. In further embodiments, the default setting is used.

In some embodiments, the polynucleotide as disclosed herein is a RNA. In some embodiments, the polynucleotide as disclosed herein is a DNA. In some embodiments, the polynucleotide as disclosed herein is a hybrid of DNA and RNA.

In some embodiments, an equivalent to a reference nucleic acid, polynucleotide or oligonucleotide encodes the same sequence encoded by the reference. In some embodiments, an equivalent to a reference nucleic acid, polynucleotide or oligonucleotide hybridizes to the reference, a complement reference, a reverse reference, or a reverse-complement reference, optionally under conditions of high stringency.

Additionally or alternatively, an equivalent nucleic acid, polynucleotide or oligonucleotide is one having at least 70% sequence identity, or at least 75% sequence identity, or at least 80% sequence identity, or alternatively at least 85% sequence identity, or alternatively at least 90% sequence identity, or alternatively at least 92% sequence identity, or alternatively at least 95% sequence identity, or alternatively at least 97% sequence identity, or alternatively at least 98% sequence, or alternatively at least 99% sequence identity to the reference nucleic acid, polynucleotide, or oligonucleotide, or alternatively an equivalent nucleic acid hybridizes under conditions of high stringency to a reference polynucleotide or its complement. In one aspect, the equivalent must encode the same protein or a functional equivalent of the protein that optionally can be identified through one or more assays described herein. In addition or alternatively, the equivalent of a polynucleotide would encode a protein or polypeptide of the same or similar function as the reference or parent polynucleotide.

The term “transduce” or “transduction” as it is applied to the production of cells, such as chimeric antigen receptor cells, refers to the process whereby a foreign nucleotide sequence is introduced into a cell. In some embodiments, this transduction is done via a vector, viral or non-viral.

As used herein, a restriction enzyme is an enzyme that cleaves DNA into fragments at or near specific recognition sites within molecules known as restriction sites. They are used to assist insertion of a polynucleotide, such as a gene, into a vector (e.g., plasmid vectors) during gene cloning and protein production experiments. For optimal use, plasmids, for example those encoding viral vector genomes, that are commonly used for gene cloning are modified to include a short polylinker sequence (called the multiple cloning site, or MCS) rich in restriction enzyme recognition sequences. This allows flexibility when inserting gene fragments into the plasmid vector; restriction sites contained naturally within genes influence the choice of endonuclease for digesting the DNA, since it is necessary to avoid restriction of wanted DNA while intentionally cutting the ends of the DNA. To clone a gene fragment into a vector, both plasmid DNA and gene insert are typically cut with the same restriction enzymes, and then glued together with the assistance of an enzyme known as a DNA ligase.

The term “protein”, “peptide” and “polypeptide” are used interchangeably and in their broadest sense to refer to a compound of two or more subunit amino acids, amino acid analogs or peptidomimetics. The subunits (which are also referred to as residues) may be linked by peptide bonds. In another embodiment, the subunit may be linked by other bonds, e.g., ester, ether, etc. A protein or peptide must contain at least two amino acids and no limitation is placed on the maximum number of amino acids which may comprise a protein's or peptide's sequence. As used herein the term “amino acid” refers to either natural and/or unnatural or synthetic amino acids, including glycine and both the D and L optical isomers, amino acid analogs and peptidomimetics.

In some embodiments, an antigen binding amino acid sequence as used herein refers to an antibody or a fragment thereof. As used herein, the term “antibody” collectively refers to immunoglobulins or immunoglobulin-like molecules including by way of example and without limitation, IgA, IgD, IgE, IgG and IgM, combinations thereof, and similar molecules produced during an immune response in any vertebrate, for example, in mammals such as humans, goats, rabbits and mice, as well as non-mammalian species, such as shark immunoglobulins. Unless specifically noted otherwise, the term “antibody” includes intact immunoglobulins and “antibody fragments” or “antigen binding fragments” that specifically bind to a molecule of interest (or a group of highly similar molecules of interest) to the substantial exclusion of binding to other molecules (for example, antibodies and antibody fragments that have a binding constant for the molecule of interest that is at least 10³ M⁻¹ greater, at least 10⁴ M⁻¹ greater or at least 10⁵ M⁻¹ greater than a binding constant for other molecules in a biological sample). The term “antibody” also includes genetically engineered forms such as chimeric antibodies (for example, murine or humanized non-primate antibodies), heteroconjugate antibodies (such as, bispecific antibodies). See also, Pierce Catalog and Handbook, 1994-1995 (Pierce Chemical Co., Rockford, Ill.); Owen et al., Kuby Immunology, 7th Ed., W.H. Freeman & Co., 2013; Murphy, Janeway's Immunobiology, 8th Ed., Garland Science, 2014; Male et al., Immunology (Roitt), 8th Ed., Saunders, 2012; Parham, The Immune System, 4th Ed., Garland Science, 2014. In some embodiments, the term “antibody” refers to a single-chain variable fragment (scFv, or ScFV). In some embodiments, the term “antibody” refers to more than one single-chain variable fragments (scFv, or ScFV) linked with each other, optionally via a peptide linker or another suitable component as disclosed herein. In some embodiments, an antibody is a monoclonal antibody. In some embodiments, an antibody is a monospecific antibody or a multispecific antibody, such as a bispecific antibody or a trispecific antibody. The species of the antibody can be a human or non-human, e.g., mammalian.

As used herein, an epitope refers to contiguous or non-contiguous amino acid residues in an antigen, such as those adjacent to each other in a three-dimensional structure of the antigen, wherein those residues are recognized and bound by an antibody or another component of the immune system.

As used herein, the term “multispecific” refers to capability of binding to more than one epitopes or antigens which are different from each other. In some embodiments, the term “multispecific” refers to comprising, or consisting essentially of, or consisting of more than one antigen binding sequences or antigen ligands, optionally linked together by a peptide linker or another component as disclosed herein. In further embodiments, the term “multispecific” refers to comprising, or consisting essentially of, or consisting of more than one antigen binding sequences (such as scFv), optionally linked together by a peptide linker or another component as disclosed herein. In some embodiments, the more than one (such as two) epitopes are located in the same antigen. Alternatively, the more than one (such as two) epitopes are from at least two antigens. In some embodiments, the ligand refers a ligand of the antigen. In some embodiments, a multispecific antibody comprises, or consists essentially of, or consists of at least two antigen binding sequences. In some embodiments, a multispecific antibody comprises, or consists essentially of, or consists of at least one antigen binding sequence and at least one ligand.

Accordingly, a bispecific antibody (abbreviated as BsAb) refers to an antibody capable of binding to two epitopes or antigens which are different from each other. In some embodiments, a bispecific antibody comprises, or consists essentially of, or consists of two antigen binding sequences or antigen ligands, optionally linked together by a peptide linker or another component as disclosed herein. In further embodiments, a bispecific antibody comprises, or consists essentially of, or consists of two antigen binding sequences (such as scFv), optionally linked together by a peptide linker or another component as disclosed herein. In some embodiments, a bispecific antibody comprises, or consists essentially of, or consists of one antigen binding sequence recognizing and binding the first epitope and one ligand recognizing and binding the antigen comprising the second epitope. In some embodiments, the two epitopes are located in the same antigen. Alternatively, the two epitopes are from two antigens which are different from each other. In some embodiments, the ligand refers to a ligand of the antigen. In some embodiments, a bispecific antibody comprises, or consists essentially of, or consists of at least two antigen binding sequences. In some embodiments, a bispecific antibody comprises, or consists essentially of, or consists of at least one antigen binding sequence and at least one ligand.

As used herein, the term BiKE is an abbreviation for bispecific NK-cell engager, referring to a bispecific antibody, an antigen binding fragment thereof, or an equivalent of each thereof, recognizing and binding a first epitope or antigen of an NK cell and a second epitope or antigen of a non-NK target (such as a cancer cell). Accordingly, a BiKE brings the non-NK target (such as a cancer cell) within the proximity of the NK cell via binding to an epitope or antigen on the target and binding to an epitope or antigen on the NK cell. In some embodiments, the second epitope or antigen is of a cancer cell. In some embodiments, the second epitope or antigen is of a TAA. Additionally or alternatively, the second epitope or antigen is not present on an NK cell, or is present on an NK cell but at a lower level (such as 10% or lower, or 1% or lower), for example, compared to on a desirable target (such as a cancer cell), thereby the BiKE may still recognize and bind an NK cell and the desirable target at the same time and bring the desirable target within the proximity of the NK cell.

As used herein, the term BiTE is an abbreviation for bispecific T-cell engager, referring to a bispecific antibody, an antigen binding fragment thereof, or an equivalent of each thereof, recognizing and binding a first epitope or antigen of a T cell and a second epitope or antigen of a non-T target (such as a cancer cell). Accordingly, a BiTE brings the non-T target (such as a cancer cell) within the proximity of the T cell via binding to an epitope or antigen on the target and binding to an epitope or antigen on the T cell. In some embodiments, the second epitope or antigen is of a cancer cell. In some embodiments, the second epitope or antigen is of a TAA. Additionally or alternatively, the second epitope or antigen is not present on a T cell, or is present on a T cell but at a lower level (such as 10% or lower, or 1% or lower), for example, compared to on a desirable target (such as a cancer cell), thereby the BiTE may still recognize and bind a T cell and the desirable target at the same time and bring the desirable target within the proximity of the T cell.

In some embodiments, NKG2D expresses on both NK cells and T cells and accordingly anti-NKG2D BiTE and anti-NKG2D BiKE can be used interchangeably.

As used herein, the language of “the proximity” of an immune cell, such as a T cell or an NK cell, refers to any location around the immune cell, a target of the immune cell (such as a cancer cell) present in which location can be recognized by the immune cell, such as via binding of a molecule present on the immune cell to another molecule present on the target, and optionally damaged or killed by the immune cell. Therefore, the action of bringing to the proximity of an immune cell is also referred to herein as engaging an immune cell.

As used herein relating to a cytotoxicity test, an effector refers to an immune cell, such as a T cell or an NK cell, which damages or kills a target cell, such as a cancer cell, in the cytotoxicity test; while a target refers to a target cell, such as a cancer cell, which is recognized, damaged or killed by the effector in the cytotoxicity test. In some embodiments, an effector is an isolated or engineered cell as disclosed herein. Additionally or alternatively, a target is a cancer cell expressing one or more TAA(s) as disclosed herein.

As used herein, the term “monoclonal antibody” refers to an antibody produced by a single clone of B-lymphocytes or by a cell into which the light and heavy chain genes of a single antibody have been transfected. Monoclonal antibodies are produced by methods known to those of skill in the art, for instance by making hybrid antibody-forming cells from a fusion of myeloma cells with immune spleen cells. Monoclonal antibodies include humanized monoclonal antibodies.

In terms of antibody structure, an immunoglobulin has heavy (H) chains and light (L) chains interconnected by disulfide bonds. There are two types of light chain, lambda (λ) and kappa (κ). There are five main heavy chain classes (or isotypes) which determine the functional activity of an antibody molecule: IgM, IgD, IgG, IgA and IgE. Each heavy and light chain contains a constant region and a variable region, (the regions are also known as “domains”). In combination, the heavy and the light chain variable regions specifically bind the antigen. Light and heavy chain variable regions contain a “framework” region interrupted by three hypervariable regions, also called “complementarity-determining regions” or “CDRs”. The extent of the framework region and CDRs have been defined (see, Kabat et al., Sequences of Proteins of Immunological Interest, U.S. Department of Health and Human Services, 1991, which is hereby incorporated by reference). The Kabat database is now maintained online. The sequences of the framework regions of different light or heavy chains are relatively conserved within a species. The framework region of an antibody, that is the combined framework regions of the constituent light and heavy chains, largely adopts a β-sheet conformation and the CDRs form loops which connect, and in some cases form part of, the β-sheet structure. Thus, framework regions act to form a scaffold that provides for positioning the CDRs in correct orientation by inter-chain, non-covalent interactions.

The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located (heavy chain regions labeled CDRH and light chain regions labeled CDRL). Thus, a CDRH3 is the CDR3 from the variable domain of the heavy chain of the antibody in which it is found, whereas a CDRL1 is the CDR1 from the variable domain of the light chain of the antibody in which it is found. For example, an anti-BCMA antibody will have a specific VH region and the VL region sequence unique to the BCMA relevant antigen, and thus specific CDR sequences. Antibodies with different specificities (i.e., different combining sites for different antigens) have different CDRs. Although it is the CDRs that vary from antibody to antibody, only a limited number of amino acid positions within the CDRs are directly involved in antigen binding. These positions within the CDRs are called specificity determining residues (SDRs).

As used herein, a single-chain variable fragment (scFv or ScFV), also referred to herein as a fragment of an antibody, and is a fusion protein of the variable regions of the heavy (VH) and light chains (VL) of immunoglobulins, optionally connected with a short linker peptide of about 10 to about 25 amino acids. The linker is usually rich in glycine for flexibility, as well as serine or threonine for solubility, and can either connect the N-terminus of the VH with the C-terminus of the VL, or vice versa. This protein retains the specificity of the original immunoglobulin, despite removal of the constant regions and the introduction of the linker.

As used herein, a fragment crystallizable (Fc) region refers to the tail region of an antibody that stabilizes the antibody, such as a bispecific antibody, and optionally interacts with (such as binds) an Fc receptor on an immune cell or on a platelet or that binds a complement protein. In some embodiments, a Fc mutant may be used, such as comprising one or two or all three mutations of F234A, L235A and N297Q of human IgG4 Fc region in a Fc or an equivalent thereof at positions corresponding to those of human IgG4 Fc region, such as for SEQ ID NO: 18 or 19, the corresponding positions are amino acid (aa) 16, aa 17 and aa 79 of SEQ ID NO: 18 or 19. As shown in Wang et al. Protein Cell. 2018 January;9(1):63-73. Epub 2017 Oct 6 and other publications, one of skill in the art would engineers an Fc region according to the use, such as reducing inflammatory cytokine release etc.

The polypeptide or an equivalent thereof, can be followed by an additional 50 amino acids, or alternatively about 40 amino acids, or alternatively about 30 amino acids, or alternatively about 20 amino acids, or alternatively about 10 amino acids, or alternatively about 5 amino acids, or alternatively about 4, or 3, or 2 or 1 amino acids at the carboxy-terminus (C-terminus). Additionally or alternatively, the polypeptide or an equivalent thereof can further comprises an additional 50 amino acids, or alternatively about 40 amino acids, or alternatively about 30 amino acids, or alternatively about 20 amino acids, or alternatively about 10 amino acids, or alternatively about 5 amino acids, or alternatively about 4, or 3, or 2 or 1 amino acids at the amine-terminus (N-terminus).

An equivalent of a reference polypeptide comprises, consists essentially of, or alternatively consists of an polypeptide having at least 80% amino acid identity to the reference polypeptide, such as the CAR as disclosed herein, or a polypeptide that is encoded by a polynucleotide that hybridizes under conditions of high stringency to the complement of a polynucleotide encoding the reference polypeptide, such as a CAR as disclosed herein, wherein conditions of high stringency comprises incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water.

Alternative embodiments include one or more of the CDRs (e.g., CDR1, CDR2, CDR3) from the LC variable region substituted with appropriate CDRs from other antibody CDRs, or an equivalent of each thereof. Accordingly, and as an example, the CDR1 and CDR2 from the LC variable region can be combined with the CDR3 of another antibody's LC variable region, and in some aspects, can include an additional 50 amino acids, or alternatively about 40 amino acids, or alternatively about 30 amino acids, or alternatively about 20 amino acids, or alternatively about 10 amino acids, or alternatively about 5 amino acids, or alternatively about 4, or 3, or 2 or 1 amino acids at the carboxy-terminus.

In some embodiments, the term “equivalent” or “biological equivalent” of an antibody means the ability of the antibody to selectively bind its epitope protein or a fragment thereof as measured by ELISA or other suitable methods is substantively maintained, for example, at a level of at least 50%, or at least 55%, or at least 60%, or at least 65%, or at least 70%, or at least 75%, or at least 80%, or at least 85%, or at least 90%, or at least 95%, or at least 99%, or more. Biologically equivalent antibodies include, but are not limited to, those antibodies, peptides, antibody fragments, antibody variant, antibody derivative and antibody mimetics that bind to the same epitope as the reference antibody. Additionally or alternatively, the equivalent and the reference antibody shares the same set of CDRs but other amino acids are modified.

It is to be inferred without explicit recitation and unless otherwise intended, that when the present disclosure relates to a polypeptide, protein, polynucleotide or antibody, an equivalent or a biologically equivalent of such is intended within the scope of this disclosure. As used herein, the term “biological equivalent thereof” is intended to be synonymous with “equivalent thereof” when referring to a reference protein, antibody, polypeptide or nucleic acid, intends those having minimal homology while still maintaining desired structure or functionality. Unless specifically recited herein, it is contemplated that any polynucleotide, polypeptide or protein mentioned herein also includes equivalents thereof. For example, an equivalent intends at least about 70% homology or identity, or at least 80% homology or identity, or at least about 85% homology or identity, or alternatively at least about 90% homology or identity, or alternatively at least about 95% homology or identity, or alternatively 98% homology or identity and exhibits substantially equivalent biological activity to the reference protein, polypeptide or nucleic acid. Alternatively, when referring to polynucleotides, an equivalent thereof is a polynucleotide that hybridizes under stringent conditions to the reference polynucleotide or its complement.

The term “antibody variant” intends to include antibodies produced in a species other than a mouse. It also includes antibodies containing post-translational modifications to the linear polypeptide sequence of the antibody or a fragment thereof. It further encompasses fully human antibodies.

The term “antibody derivative” is intended to encompass molecules that bind an epitope as defined above and which are modifications or derivatives of a native monoclonal antibody of this disclosure. Derivatives include, but are not limited to, for example, bispecific, multi specific, heterospecific, tri specific, tetraspecific, multi specific antibodies, diabodies, chimeric, recombinant and humanized.

As used herein, the term “specific binding” or “binding” means the contact between an antibody and an antigen with a binding affinity of at least 10⁻⁶ M. In certain embodiments, antibodies bind with affinities of at least about 10⁻⁷ M, and preferably at least about 10⁻⁸ M, at least about 10⁻⁹ M, at least about 10⁻¹⁰ M, at least about 10⁻¹¹ M, or at least about 10⁻¹² M.

As used herein, the term “antigen” refers to a compound, composition, or substance that may be specifically bound by the products of specific humoral or cellular immunity, such as an antibody molecule or T-cell receptor. Antigens can be any type of molecule including, for example, haptens, simple intermediary metabolites, sugars (e.g., oligosaccharides), lipids, and hormones as well as macromolecules such as complex carbohydrates (e.g., polysaccharides), phospholipids, and proteins. Common categories of antigens include, but are not limited to, viral antigens, bacterial antigens, fungal antigens, protozoa and other parasitic antigens, tumor antigens, antigens involved in autoimmune disease, allergy and graft rejection, toxins, and other miscellaneous antigens.

In some embodiments, antigen of a binding moiety, such as an antibody, an antigen binding fragment thereof, or a CAR, may be provided herein in a format of “antigen” followed by the binding moiety (such as a BCMA CAR), or having “anti” or “anti-” before the antigen and the binding moiety after the antigen (such as an anti-BCMA antibody), or the binding moiety followed by “to” or “directed to” and then the antigen (such as an antibody to BCMA).

As used herein, the terms tumor associated antigen (TAA), cancer antigen, tumor antigen, cancer relevant antigen, and tumor relevant antigen are used interchangeably herein, referring to antigenic substance of a cancer or tumor cells. In some embodiments, a TAA presents on some tumor or cancer cells and also on some normal cells, optionally at a lower level. In some embodiments, a TAA only presents on a tumor or cancer cell but not on a normal cell. In some embodiments, a TAA refers to a TAA recognized and bound by a CAR as disclosed herein. In some embodiments, a TAA refers to a TAA recognized and bound by an antibody as disclosed herein. In some embodiments, a TAA is selected from BCMA, GPRC5D, FLT3, CD19, mesothelin, human epidermal growth factor receptor 2 (HER2), prostate stem cell antigen (PSCA), carcinoembryonic antigen (CEA), CD33, GTPase-activating protein (GAP), ganglioside G2 (GD2), CD5, prostate specific membrane antigen (PSMA), receptor tyrosine kinase-like orphan receptor 1 (ROR1), CD123, CD70, CD38, mucin 1, (Muc1), ephrin type-A receptor 2 precursor (EphA2), epidermal growth factor receptor variant III (EGFRVIII), interleukin 13 receptor alpha 2 (IL13Ra2), CD133, glypican 3 (GPC3), epithelial cell adhesion molecule precursor (EpCam), fibroblast activation protein alpha (FAP), vascular endothelial growth factor receptor 2 (VEGFR2), cancer/testis (CT), guanylyl cyclase C (GUCY2C), tumor-associated glycoprotein-72 (TAG-72), thymidine kinase 1 (TK1), or hypoxanthine guanine phosphoribosyltransferase (HPRT1).

As used herein the term “NKG2D” refers to a transmembrane protein belonging to the CD94/NKG2 family of C-type lectin-like receptors and encoded by the gene KLRK1 gene, which is located in the NK-gene complex or a biological equivalent thereof. Non-limiting exemplary sequences of this protein or the underlying gene may be found under Gene Cards ID: GC12M011728, HGNC: 18788, Entrez Gene: 22914, Ensembl: ENSG00000213809, OMIM: 611817, or UniProtKB: P26718, each of which is incorporated by reference herein in its entirety.

As used herein the terms “BCMA” and “B-cell maturation antigen” are used interchangeably to refer to a protein belonging to the TNF superfamily which recognizes B-cell activating factor (BAFF) and encoded by the TNFRSF17 gene. Non-limiting exemplary sequences of this protein or the underlying gene may be found under Gene Cards ID: GC16P012058, HGNC: 11913, Entrez Gene: 608, Ensembl: ENSG00000048462, OMIM: 109545, or UniProtKB: Q02223, each of which is incorporated by reference herein in its entirety.

As used herein, the term “antigen binding domain” refers to any protein or polypeptide domain that can specifically bind to an antigen target.

As used herein, the term “autologous,” in reference to cells refers to cells that are isolated and infused back into the same subject (recipient or host). “Allogeneic” refers to non-autologous cells.

The term “chimeric antigen receptor” (CAR), as used herein, refers to a fused protein comprising an extracellular domain capable of binding to an antigen, a transmembrane domain derived from a polypeptide different from a polypeptide from which the extracellular domain is derived, and at least one intracellular domain. The “chimeric antigen receptor (CAR)” is sometimes called a “chimeric receptor”, a “T-body”, or a “chimeric immune receptor (CIR).” The “extracellular domain capable of binding to an antigen” means any oligopeptide or polypeptide that can bind to a certain antigen. The “intracellular domain” or “intracellular signaling domain” means any oligopeptide or polypeptide known to function as a domain that transmits a signal to cause activation or inhibition of a biological process in a cell, such as an immune cell. In certain embodiments, the intracellular domain may comprise, alternatively consist essentially of, or yet further consist of one or more costimulatory signaling domains in addition to the primary signaling domain. The “transmembrane domain” means any oligopeptide or polypeptide known to span the cell membrane and that can function to link the extracellular and signaling domains.

A chimeric antigen receptor may optionally comprise a “hinge domain” which serves as a linker between the extracellular and transmembrane domains. Non limiting examples of such domains are provided herein, e.g.: Hinge domain: IgG1 heavy chain hinge coding sequence: SEQ ID NO: 82 or a IgG1 hinge amino acid sequence comprising, or consisting essentially of, or yet further consisting of LEPKSCDKTHTCPPCP (SEQ ID NO: 83), or LEPKSCDKTHTCPPCPDPKGT (SEQ ID NO: 47) or an equivalent of each thereof. As used herein, the term IgG1 hinge domain also refers to a specific protein fragment associated with this name or any other molecules that have analogous biological function that share at least about 70%, or alternatively at least 80% amino acid sequence identity, preferably at least about 90% sequence identity, more preferably at least about 95% sequence identity with the IgG1 hinge domain sequence as shown herein. Additional example sequences of IgG1 hinge domain are provided in, e.g., US20180273642A1 and Dall'Acqua WF, Cook KE, Damschroder MM, Woods RM, Wu H. Modulation of the effector functions of a human IgG1 through engineering of its hinge region. J Immunol. 2006 Jul. 15; 177(2):1129-38. Additional non-limiting example of a hinge domain includes those of another immunoglobulin, such as an IgG4 hinge region, and an IgD hinge domain. See, for example, US20180273642A1. Another example is a CD8 hinge domain, such as a CD8α hinge domain, as known in the art.

Further embodiments of each exemplary domain component include other proteins, or a fragment thereof, that have analogous biological function that share at least about 70%, or alternatively at least about 80% amino acid sequence identity, preferably at least about 90% sequence identity, more preferably at least about 95% sequence identity with a domain as disclosed herein, such as domains of the proteins encoded by the nucleic acid sequences as disclosed herein. Further, non-limiting examples of such domains are provided herein.

As used herein, the term “CD8α hinge domain” also refers to a specific protein fragment associated with this name or any other molecules that have analogous biological function that share at least about 70%, or alternatively at least 80% amino acid sequence identity, preferably at least about 90% sequence identity, more preferably at least about 95% sequence identity with the CD8α hinge domain sequence as shown herein. The example sequences of CD8α hinge domain for human, mouse, and other species are provided in Pinto, R. D. et al. (2006) Vet. Immunol. Immunopathol. 110:169-177. The sequences associated with the CD8α hinge domain are also provided in Pinto, R. D. et al. (2006) Vet. Immunol. Immunopathol. 110:169-177. Non-limiting examples of such include: Human CD8 alpha hinge domain: SEQ ID NO: 84. Mouse CD8 alpha hinge domain: SEQ ID NO: 85. Cat CD8 alpha hinge domain: SEQ ID NO: 86.

As used herein, the term “CD8α transmembrane domain” refers to a specific protein fragment associated with this name or any other molecules that have analogous biological function that share at least about 70%, or alternatively at least about 80% amino acid sequence identity, preferably at least about 90% sequence identity, more preferably at least about 95% sequence identity with the CD8α transmembrane domain sequence as shown herein. The fragment sequences associated with the amino acid positions 183 to 203 of the human T-cell surface glycoprotein CD8 alpha chain (GenBank Accession No: NP_001759.3), or the amino acid positions 197 to 217 of the mouse T-cell surface glycoprotein CD8 alpha chain (GenBank Accession No: NP_001074579.1), or the amino acid positions 190 to 210 of the rat T-cell surface glycoprotein CD8 alpha chain (GenBank Accession No: NP_113726.1) provide additional example sequences of the CD8α transmembrane domain. The sequences associated with each of the listed accession numbers are provided as follows: human CD8 alpha transmembrane domain: IYIWAPLAGTCGVLLLSLVIT (SEQ ID NO: 87); mouse CD8 alpha transmembrane domain: IWAPLAGICVALLLSLIITLI (SEQ ID NO: 88); and rat CD8 alpha transmembrane domain: IWAPLAGICAVLLLSLVITLI (SEQ ID NO: 89).

As used herein, the term “CD28 transmembrane domain” refers to a specific protein fragment associated with this name or any other molecules that have analogous biological function that share at least about 70%, or alternatively at least about 80% amino acid sequence identity, or alternatively at least about 90% sequence identity, or alternatively at least about 95% sequence identity with the CD28 transmembrane domain sequence as shown herein. The fragment sequences associated with the GenBank Accession Nos: XM_006712862.2 or XM_009444056.1 provide additional, non-limiting, exemplified sequences of the CD28 transmembrane domain. The sequences associated with each of the listed accession numbers are provided herein, for example, transmembrane domain: CD28 transmembrane region coding sequence: SEQ ID NO: 90 or a CD28 transmembrane region amino acid sequence comprising, consisting essentially of, or consisting of SEQ ID NO: 91 or an equivalent thereof.

As used herein, the term “4-1BB costimulatory signaling region” or “4-1BB costimulatory region” refers to a specific protein fragment associated with this name or any other molecules that have analogous biological function that share at least about 70%, or alternatively at least about 80% amino acid sequence identity, preferably at least about 90% sequence identity, more preferably at least about 95% sequence identity with the 4-1BB costimulatory signaling region sequence as shown herein. Non-limiting example sequences of the 4-1BB costimulatory signaling region are provided in U.S. Publication 20130266551A1, such as the exemplary sequence provided below: 4-1BB costimulatory signaling region: SEQ ID NO: 92; and Intracellular domain: 4-1BB co-stimulatory signaling region coding sequence: SEQ ID NO: 93.

As used herein, the term “CD28 costimulatory signaling region” or “CD28 costimulatory region” refers to a specific protein fragment associated with this name or any other molecules that have analogous biological function that share at least about 70%, or alternatively at least about 80% amino acid sequence identity, preferably at least about 90% sequence identity, more preferably at least about 95% sequence identity with the CD28 costimulatory signaling region sequence shown herein. The example sequences CD28 costimulatory signaling domain are provided in U.S. Pat. No. 5,686,281; Geiger, T. L. et al., Blood 98: 2364-2371 (2001); Hombach, A. et al., J Immunol 167: 6123-6131 (2001); Maher, J. et al. Nat Biotechnol 20: 70-75 (2002); Haynes, N. M. et al., J Immunol 169: 5780-5786 (2002); or Haynes, N. M. et al., Blood 100: 3155-3163 (2002). Non-limiting examples include residues 114-220 of the CD28 Sequence: SEQ ID NO: 94, and equivalents thereof. In some embodiments, a CD28 costimulatory signaling region comprises, or consists essentially of, or consists of SEQ ID NO: 95 or an equivalent thereof. In further embodiments, a CD28 co-stimulatory signaling region coding sequence comprises, or consists essentially of, or consists of SEQ ID NO: 96.

As used herein, the term “ICOS costimulatory signaling region” or “ICOS costimulatory region” refers to a specific protein fragment associated with this name or any other molecules that have analogous biological function that share at least about 70%, or alternatively at least about 80% amino acid sequence identity, preferably at least about 90% sequence identity, more preferably at least about 95% sequence identity with the ICOS costimulatory signaling region sequence as shown herein. Non-limiting example sequences of the ICOS costimulatory signaling region are provided in U.S. Publication 2015/0017141A1 as well as ICOS costimulatory signaling region coding sequence: SEQ ID NO: 97 or an equivalent thereof.

As used herein, the term “OX40 costimulatory signaling region” or “OX40 costimulatory region” refers to a specific protein fragment associated with this name or any other molecules that have analogous biological function that share at least about 70%, or alternatively at least about 80% amino acid sequence identity, or alternatively at least about 90% sequence identity, or alternatively at least about 95% sequence identity with the OX40 costimulatory signaling region sequence as shown herein. Non-limiting example sequences of the OX40 costimulatory signaling region are disclosed in U.S. Publication 2012/20148552A1, and include the exemplary sequence provided below: OX40 costimulatory signaling region coding sequence: SEQ ID NO: 98, and equivalents thereof.

As used herein, the term “DAP10 costimulatory signaling region” or “DAP10 costimulatory region” refers to a specific protein fragment associated with this name or any other molecules that have analogous biological function that share at least about 70%, or alternatively at least about 80% amino acid sequence identity, or alternatively at least about 90% sequence identity, or alternatively at least about 95% sequence identity with the DAP10 costimulatory signaling region sequence as shown herein. Non-limiting example sequences of the DAP10 costimulatory signaling region are disclosed in U.S. Pat. No. 9,587,020B2, and include the exemplary sequence provided below: RPRRSPAQDGKVYINMPGRG (SEQ ID NO: 99), or equivalents thereof.

As used herein, the term “DAP12 costimulatory signaling region” or “DAP12 costimulatory region” refers to a specific protein fragment associated with this name or any other molecules that have analogous biological function that share at least about 70%, or alternatively at least about 80% amino acid sequence identity, or alternatively at least about 90% sequence identity, or alternatively at least about 95% sequence identity with the DAP12 costimulatory signaling region sequence as shown herein U.S. Pat. No. 9,587,020B2. Non-limiting example sequences of the DAP12 costimulatory signaling region are disclosed in U, and include the exemplary sequence provided below: ESPYQELQGQRSDVYSDLNTQ (SEQ ID NO: 100), or equivalents thereof.

As used herein, the term “CD3 zeta signaling domain” refers to a specific protein fragment associated with this name or any other molecules that have analogous biological function that share at least about 70%, or alternatively at least about 80% amino acid sequence identity, preferably at least about 90% sequence identity, more preferably at least about 95% sequence identity with the CD3 zeta signaling domain sequence as shown herein. Non-limiting example sequences of the CD3 zeta signaling domain are provided in U.S. Publication 20130266551A1, e.g.: SEQ ID NO: 101; and Intracellular domain: CD3 zeta signaling region coding sequence: SEQ ID NO: 102.

As used herein, cytokines are a large group of proteins, peptides or glycoproteins that are secreted by specific cells of the immune system and signaling molecules that mediate and regulate immunity, inflammation and hematopoiesis. In some embodiments, they are about 5 kDa to about 20 kDa. Cytokines comprise chemokines, interferons, interleukins, lymphokines, and tumour necrosis factors, but generally not hormones or growth factors. In some embodiments, the cytokine(s) as used herein promote one or more of the development, differentiation, activation, or expansion of immune cells, such as T cells, NK cells, NKT cells, or any combination thereof.

As used herein, interleukin (IL) refers to cytokines that was first seen to be expressed by white blood cells (leukocytes). The function of the immune system depends in a large part on interleukins. The majority of interleukins are synthesized by helper CD4 T lymphocytes, as well as through monocytes, macrophages, and endothelial cells. They promote the development and differentiation of T and B lymphocytes, NK cells, NKT cells, and hematopoietic cells. As used herein, an interleukin can be a soluble cytokine secreted out of a cell, or a membrane bound (mb) cytokine expressed on a cell surface. A soluble form and a membrane bound form of a cytokine can be converted by one of skill in the art, such as by a method comprising, consisting essentially of, or consisting of engineering a transmembrane domain (such as a platelet-derived growth factor receptor beta (PDGFRβ) transmembrane domain) or a signal peptide or both a transmembrane domain and a signal peptide to a cytokine.

In some embodiments, the cytokine as used herein comprises, or consists essentially of, or yet further consists of an IL-15. As used herein, the terms “IL15”, “IL 15”, “IL-15” and “Interleukin-15” are used interchangeably to refer to a cytokine that regulates T and natural killer cell activation and proliferation and encoded by the IL15 gene. Non-limiting exemplary sequences of this protein or the underlying gene may be found under Gene Cards ID: GC04P141636, HGNC: 5977, Entrez Gene: 3600, Ensembl: ENSG00000164136, OMIM: 600554, or UniProtKB: P40933, each of which is incorporated by reference herein by its entirety.

In some embodiments, the IL-15 is a soluble IL-15. In further embodiments, the soluble IL-15 comprises, or consists essentially of, or yet further consists of an amino acid sequence selected from SEQ ID NO: 103, SEQ ID NO: 106, or an amino acid sequence of SEQ ID NO: 104 (each of which may be referred to herein as a wildtype IL15) or an equivalent thereof. In some embodiments, the IL15 equivalent has at least about 10%, or about 20%, or about 30%, or about 40%, or about 50%, or about 60%, or about 70%, or about 80%, or about 90%, or about 95%, or about 100%, or about 1.5 fold, or about 2 folds, or about 3 folds, or about 5 folds, or about 10 folds, or more binding affinity to IL15RA compared to the wildtype IL15. In some embodiments, the IL-15 equivalent is a IL-15 comprising one or more mutations selected from L45D, L45E, S51D, L52D, N72D, N72E, N72A, N72S, N72Y and N72P, wherein the first letter indicates the original amino acid residue, the last letter indicates the mutated amino acid residue and the number in the center indicates the amino acid residue number in or aligned to the sequence of SEQ ID NO: 105.

In some embodiments, used herein as a cytokine is a soluble complex (SIL15C) comprising, or consisting essentially of, or consisting of an IL-15 and an IL15 receptor. In further embodiments, the IL-15 comprises, or consists essentially of, or yet further consists of an amino acid sequence of SEQ ID NO: 106, or an amino acid sequence of SEQ ID NO: 104 or an equivalent of each thereof.

In further embodiments, the IL15 receptor comprises, or consists essentially of, or yet further consists of an IL15 receptor subunit alpha (IL15RA) or an equivalent thereof. Additionally or alternatively, the IL15 receptor comprises, or consists essentially of, or yet further consists of an IL15 receptor subunit beta (IL15RB, i.e., IL2 receptor subunit beta). In some embodiments, the IL15RA equivalent has at least about 10%, or about 20%, or about 30%, or about 40%, or about 50%, or about 60%, or about 70%, or about 80%, or about 90%, or about 95%, or about 100%, or about 1.5 fold, or about 2 folds, or about 3 folds, or about 5 folds, or about 10 folds, or more binding affinity to IL15 compared to the wildtype IL15RA. The binding affinity can be determined by a method as provided in the Examples as well as in the art, such as Wei et al, J. Immunol, vol. 167(1), p:277-282, 2001. In some embodiments, the IL15RA equivalent comprises, or consists essentially of, or yet further consists of a sushi domain of the IL15RA. In further embodiments, the IL15RA equivalent is a fragment of the wildtype IL15RA comprising, or consisting essentially of, or yet further consisting of a sushi domain of the IL15RA. In some embodiments, the IL15 receptor comprises, or consists essentially of, or consists of SEQ ID NO: 107 or an equivalent thereof.

In further embodiments, the SIL15C comprises, or consists essentially of, or yet further consists of an amino acid sequence of SEQ ID NO: 106 or an equivalent thereof and an amino acid sequence of SEQ ID NO: 107 or an equivalent thereof. In some embodiments, the IL15 and the IL15 receptor are linked by a peptide linker. In some embodiments, the linker comprises SGGGSGGGGSGGGGSGGGGSGGGSLQ (SEQ ID NO: 108) or SEQ ID NO: 9, or an equivalent of each thereof. In some embodiments, the SIL15C comprises, or consists essentially of, or yet further consists of ALT-803 (IL15N72DIL15RαSu/IgG1 Fc complex). ALT-803 is an IL-15 superagonist complex IL-15N72D:IL-15RaSu/Fc that comprises, or consists essentially of, or yet further consists of an IL-15 mutant (IL-15N72D) and a dimeric IL-15 receptor a sushi domain-IgG1 Fc fusion protein.

As used herein, the IL15RA sushi domain refers to a consensus sequence of CPX₁PX₂SVEHADIX₃VKSYSLX₄SRERYX₅CNSGFKRKAGTSSLTECVLNKATNX₆AX₇WTTPSLKC, wherein X₁ is P or A, X₂ is M or V, X₃ is W, R or Q, X₄ is Y or H, X₅ is I or V, X₆ iS V or A, and X₇ iS V or A (SEQ ID NO: 109).

As used herein, the terms “IL15RA”, “IL 15 RA”, “IL-15 RA” and “IL 15 Receptor Subunit Alpha” are used interchangeably to refer to a cytokine receptor that specifically binds interleukin 15 (IL15) with high affinity and encoded by the IL15RA gene or a fragment thereof. IL15RA can signal both in cis and trans where IL15 receptor (IL15R) from one subset of cells presents IL15 to neighboring IL2RG-expressing cells. Non-limiting exemplary sequences of this protein or the underlying gene or functions thereof may be found under Gene Cards ID: GC10M005943, HGNC: 5978, Entrez Gene: 3601, Ensembl: ENSG00000134470, OMIM: 601070, or UniProtKB: Q13261, each of which is incorporated by reference herein in its entirety. In some embodiments, the IL15RA comprises, or consists essentially of, or yet further consists of an amino acid sequence of SEQ ID NO: 107.

As used herein, IL2RB and IL15RB are used interchangeably to refer to a receptor for IL2 and in association with IL15RA, involved in the stimulation of neutrophil phagocytosis by IL15. See, for example, Ratthe et al. J. Leukoc. Biol. 76:162-168(2004). Non-limiting exemplary sequences of this protein or the underlying gene or functions thereof may be found under Gene Cards ID: GC22M037125, HGNC: 6009, Entrez Gene: 3560, Ensembl: ENSG00000100385, OMIM: 146710, or UniProtKB: P14784, each of which is incorporated by reference herein in its entirety.

In other embodiments, the IL-15 is a membrane bound IL-15 (mbIL-15), such as a soluble IL-15 conjugated with a transmembrane domain optionally further comprising a peptide linker or one or more random amino acid residues, and thus bounded on cell membrane. In further embodiments, the mbIL-15 is IL15 conjugated with a PDGFRβ transmembrane domain or an equivalent thereof.

In some embodiments, the cytokine as used herein comprises, or consists essentially of, or yet further consists of an IL-21, As used herein, the terms “IL21”, “IL 21”, “IL-21” and “Interleukin-21” are used interchangeably to refer to a cytokine with immunoregulatory activity and encoded by the IL21 gene. Non-limiting exemplary sequences of this protein or the underlying gene may be found under Gene Cards ID: C04M122612, HGNC: 6005, Entrez Gene: 59067, Ensembl: ENSG00000138684, OMIM: 605384, or UniProtKB: Q9HBE4, each of which is incorporated by reference herein in its entirety. UniProtKB/Swiss-Prot entry Q9HBE4 (IL21 HUMAN) describes 2 isoforms produced by alternative splicing: isoform 1 at 18 kDa, isoform 2 at 17 kDa, and there are 146 aa common sequences between the two isoforms by alignment. Without wishing to be bound by the theory and according to structure analysis for IL-21, a minimal sequence of IL-21 is 146 aa long, for example the first 146 aa of isoform 1 or isoform 2. Accordingly, in some embodiments, an IL-21 comprises, or consists essentially of, or consists of the common sequences shared by IL-21 isoform 1 and isoform 2, with or without an IL-21 signal peptide. Non-limiting example of the common sequence comprises, or consists essentially of, or consists of SEQ ID NO: 110, or SEQ ID NO: 111, or SEQ ID NO: 112. In some embodiments, an IL-21 comprises, or consists essentially of, or consists of an IL-21 isoform 1, with or without an IL-21 signal peptide. Non-limiting example of an IL-21 isoform 1 sequence comprises, or consists essentially of, or consists of SEQ ID NO: 113, or SEQ ID NO: 114, or SEQ ID NO: 115. In some embodiments, an IL-21 comprises, or consists essentially of, or consists of an IL-21 isoform 2, with or without an IL-21 signal peptide. Non-limiting example of an IL-21 isoform 2 sequence comprises, or consists essentially of, or consists of SEQ ID NO: 116, or SEQ ID NO: 117, or SEQ ID NO: 118. In some embodiments, the IL21 is a soluble IL21. In further embodiments, the IL21 comprises, or consists essentially of, or yet further consists of an amino acid sequence of SEQ ID NO: 116 or SEQ ID NO: 117, or SEQ ID NO: 118 or an equivalent of each thereof. In other embodiments, the IL21 is a membrane bound IL-21 (mblL-21), such as a soluble IL-21 conjugated with a transmembrane domain optionally further comprising a peptide linker or one or more random amino acid residues, and thus bounded on cell membrane. In further embodiments, the mblL-21 is IL21 conjugated with a PDGFRβ transmembrane domain or an equivalent thereof.

As used herein, the term “transmembrane domain” refers to a protein region that is hydrophobic, so that it prefers to be inserted into the cell membrane such that the parts of the protein on either side of the domain are on opposite sides of the membrane. In some embodiments, the transmembrane domain comprises, or consists essentially of, or yet further consists of a transmembrane segment of single alpha helix of a transmembrane protein. Additionally or alternatively, a transmembrane domain comprises, or consists essentially of, or yet further consists of predominantly of nonpolar amino acid residues and may traverse the membrane bilayer once or several times. In a further embodiment, the transmembrane domain comprises, or consists essentially of, or yet further consists of a PDGFRβ transmembrane domain. In yet a further embodiment, the PDGFRβ transmembrane domain comprises, or consists essentially of, or yet further consists of an amino acid sequence of

(SEQ ID NO: 119)
AVGQDTQEVIVVPHSLPFKVVVISAILALVVLTIISLIILIMLWQKKPR.

As used herein, the terms “PDGFRβ” and “platelet-derived growth factor receptor beta” are used interchangeably to refer to a cell surface tyrosine kinase receptor for members of the platelet-derived growth factor family and encoded by the PDGFRB gene. Non-limiting exemplary sequences of this protein or the underlying gene may be found under Gene Cards ID: GC05M150113, HGNC: 8804, Entrez Gene: 5159, Ensembl: ENSG00000113721, OMIM: 173410, or UniProtKB: P09619, each of which is incorporated herein by reference herein in its entirety.

As used herein, the term “suicide gene” refers to any gene that expresses a product (optionally with presence of another agent, such as an antibody) that is fatal to the cell expressing the suicide gene. Transcription or expression of such gene, i.e., presence of its gene product, in a cell alone or together with other agents causing the cell to kill itself, for example through apoptosis. It provides a possible strategy of eliminating a cell, for example, a therapeutic cell expressing CAR or bispecific antibody or both, after it performs its desired function, such as treating a cancer. In further embodiments, the suicide gene product is selected from one or more of: HSV-TK (Herpes simplex virus thymidine kinase), cytosine deaminase, nitroreductase, carboxylesterase, cytochrome P450 or PNP (Purine nucleoside phosphorylase), truncated EGFR (tEGFR), or inducible caspase (“iCasp”). In yet further embodiments, exemplified suicide strategy includes the thymidine kinase/ganciclovir system, the cytosine deaminase/5-fluorocytosine system, the nitroreductase/CB1954 system, carboxypeptidase G2/Nitrogen mustard system, cytochrome P450/oxazaphosphorine system, purine nucleoside phosphorylase/6-methylpurine deoxyriboside (PNP/MEP), the horseradish peroxidase/indole-3-acetic acid system (HRP/IAA), and the carboxylesterase/irinotecan (CE/irinotecan) system, the truncated EGFR (tEGFR), inducible caspase (“iCasp”), the E. coli gpt gene, the E. coli Deo gene and nitroreductase. See, more details at Karjoo, Z. et al. 2016. Adv. Drug Deliv. Rev. 99 (Pt. A):123-128.

A protein expressed on cell surface may be used as a marker (such as for purification or detection or tracking) or to provide a suicide switch of a CAR expressing cell as disclosed herein. Such protein is referred to herein as a suicide gene product or a detectable marker or both. A portion of or the whole cytoplasmic region of such protein is usually truncated so that the native function of the protein is reduced or even abolished. Thus, such a protein is also referred to herein as a truncated protein marker. In some embodiments, when used as a suicide switch of the CAR expressing cell, the truncated protein marker does not express or is expressed at a substantially lower level on a normal cell or a normal cell adjacent to the CAR expressing cell in the subject. Accordingly, upon removal of the CAR expressing cell (for example, by administering an antibody specially recognizing and binding the truncated protein marker, or by administering a toxin conjugated to a moiety directing the toxin to the truncated protein marker), a normal cell of the subject would not be jeopardized. Accordingly, in some embodiments, a method as disclosed herein can further comprise administering the subject an agent reducing or abolishing the CAR expressing cell in the subject. In further embodiments, the agent reducing or abolishing the CAR expressing cell in the subject comprises, or consists essentially of, or yet further consists of an antibody or a fragment thereof specifically recognizing and binding to the suicide gene product, such as tEGFR or RQR8. Additionally or alternatively, the administration of the agent reducing or abolishing the CAR expressing cell in the subject is about 1 day, about 3 days, about 1 week, about 2 weeks, about 3 weeks, about 4 weeks, about 5 weeks, about 6 weeks, about 7 weeks, about 8 weeks, about 9 weeks, about 10 weeks, about 3 months, about 4 months, about 5 months, about 6 months, about 1 year, about 1.5 years, about 2 years, or longer post the administration of a cell as disclosed herein.

In some embodiments, the tEGFR comprises, or consists essentially of, or yet further consists of an amino acid sequence of SEQ ID NO: 120. Expression of tEGFR on a cell permits recognition of the cell by an anti-EGFR antibody and thus eliminating the cell. See, for example, Wu, et al., Mol Ther. 2017 Oct. 4; 25(10):2270-2279. doi: 10.1016/j.ymthe.2017.06.026. Epub 2017 Jul 3.

As used herein, target epitopes from both CD34 and CD20 antigens can be combined and used herein as a suicide gene product or a detectable marker or both, for example, RQR8. RQR8 is an epitope-based marker/suicide gene that enables clinical selection, cell tracking, and deletion in case of toxicity. See, for example, Philip et al. Blood. 2014 Aug. 21; 124(8):1277-87, and U.S. Pat. No. 10,925,943. In some embodiments, RQR8 comprises, or consists essentially of, or yet further consists of SEQ ID NO: 121.

A signal peptide, as used herein, (sometimes referred to as signal sequence, targeting signal, localization signal, localization sequence, transit peptide, leader sequence or leader peptide) is a short peptide (usually 16-30 amino acids long) present at the N-terminus of the majority of newly synthesized proteins that are destined toward the secretory pathway. In some embodiments, the signal peptide comprises, or consists essentially of, or yet further consists of MYRMQLLSCIALSLALVTNS (SEQ ID NO: 20), or MWLQSLLLLGTVACSIS (SEQ ID NO: 122), or MRISKPHLRSISIQCYLCLLLNSHFLTEA (SEQ ID NO: 123), or MRSSPGNMERIVICLMVIFLGTLV (SEQ ID NO: 124), or MGWSSIILFLVATATGVH (SEQ ID NO: 21), or MLLLVTSLLLCELPHPAFLLIP (SEQ ID NO: 125) or an equivalent of each thereof. In one embodiment, the signal peptide comprises, or consists essentially of, or yet further consists of MYRMQLLSCIALSLALVTNS (SEQ ID NO: 20), or MWLQSLLLLGTVACSIS (SEQ ID NO: 122), or MRISKPHLRSISIQCYLCLLLNSHFLTEA (SEQ ID NO: 123), or MRSSPGNMERIVICLMVIFLGTLV (SEQ ID NO: 124), or an equivalent of each thereof. In another embodiments, the signal peptide comprises, or consists essentially of, or yet further consists of MGWSSIILFLVATATGVH (SEQ ID NO: 21), or MLLLVTSLLLCELPHPAFLLIP (SEQ ID NO: 125) or an equivalent of each thereof. In some embodiments, the signal peptide is an Azurocidin signal peptide. In further embodiments, the Azurocidin signal peptide comprises, or consists essentially of, or yet further consists of MTRLTVLALLAGLLASSRA (SEQ ID NO: 126). In some embodiments, the Azurocidin signal peptide can be encoded by ATGACTAGGTTGACAGTCCTCGCCTTGCTTGCTGGATTGCTTGCCAGTTCTCGAG CC (SEQ ID NO: 127). In one embodiment, the signal peptide is a secretary signal.

A secretary signal intends a secretory signal peptide that allows the export of a protein from the cytosol into the secretory pathway. Proteins can exhibit differential levels of successful secretion and often certain signal peptides can cause lower or higher levels when partnered with specific proteins. In eukaryotes, the signal peptide is a hydrophobic string of amino acids that is recognized by the signal recognition particle (SRP) in the cytosol of eukaryotic cells. After the signal peptide is produced from an mRNA-ribosome complex, the SRP binds the peptide and stops protein translation. The SRP then shuttles the mRNA/ribosome complex to the rough endoplasmic reticulum where the protein is translated into the lumen of the endoplasmic reticulum. The signal peptide is then cleaved off the protein to produce either a soluble, or membrane tagged (if a transmembrane region is also present), protein in the endoplasmic reticulum. These are known in the art, and commercially available from vendors, e.g., Oxford Genetics.

As used herein, a cleavable peptide, which is also referred to as a cleavable linker, means a peptide that can be cleaved, for example, by an enzyme. One translated polypeptide comprising such cleavable peptide can produce two final products, therefore, allowing expressing more than one polypeptides from one open reading frame. One example of cleavable peptides is a self-cleaving peptide, such as a 2A self-cleaving peptide. 2A self-cleaving peptides, is a class of 18-22 aa-long peptides, which can induce the cleaving of the recombinant protein in a cell. In some embodiments, the 2A self-cleaving peptide is selected from P2A, T2A, E2A, F2A and BmCPV2A. See, for example, Wang Y, et al. 2A self-cleaving peptide-based multi-gene expression system in the silkworm Bombyx mori. Sci Rep. 2015; 5:16273. Published 2015 Nov 5.

As used herein, the terms “T2A” and “2A peptide” are used interchangeably to refer to any 2A peptide or fragment thereof, any 2A-like peptide or fragment thereof, or an artificial peptide comprising the requisite amino acids in a relatively short peptide sequence (on the order of 20 amino acids long depending on the virus of origin) containing the consensus polypeptide motif D-V/I-E-X-N-P-G-P, wherein X refers to any amino acid generally thought to be self-cleaving (SEQ ID NO: 128).

As used herein the terms “linker sequence” “linker peptide” and “linker polypeptide” are used interchangeably, relating to any amino acid sequence comprising from 1 to 10, or alternatively 8 amino acids, or alternatively 6 amino acids, or alternatively 5 amino acids that may be repeated from 1 to 10, or alternatively to about 8, or alternatively to about 6, or alternatively to about 5, or alternatively, to about 4, or alternatively to about 3, or alternatively to about 2 times. For example, the linker may comprise up to 15 amino acid residues consisting of a pentapeptide repeated three times. In one embodiment, the linker sequence is a (Glycine4Serine)3 (SEQ ID NO: 9) flexible polypeptide linker comprising three copies of gly-gly-gly-gly-ser (SEQ ID NO: 129). In some embodiments, the linker sequence is a (G4S)n, wherein n is 1, or 2, or 3, or 4, or 5, or 6, or 7, or 8, or 9, or 10, or 11, or 12, or 13, or 14, or 15 (SEQ ID NO: 130). In some embodiments, the linker is a human muscle aldolase (HMA) linker. In further embodiments, the HMA linker comprises, or consists essentially of, or yet further consists of PSGQAGAAASESLFVSNHAY (SEQ ID NO: 81). In some embodiments, the linker is a cleavable peptide as disclosed herein. In some embodiments, the peptide linker comprises, or consists essentially of, or consists of

(SEQ ID NO: 108)
SGGGSGGGGSGGGGSGGGGSGGGSLQ.

The term “internal ribosome entry site” or “IRES” as used herein interchangeably refers to a polynucleotide that directly promotes ribosome binding and mRNA translation and thereby permits initiation of translation in cap-independent manner. In some embodiments, an IRES refers an RNA sequence on a messenger RNA (mRNA). Additionally or alternatively, an IRES also refers to a polynucleotide sequence (such as an RNA sequence, a DNA sequence or a hybrid thereof) complementary, or reverse, or both complementary and reverse to an IRES RNA sequence. Non-limiting examples of IRES can be found in Hellen CU and Sarnow P. Internal ribosome entry sites in eukaryotic mRNA molecules. Genes Dev. 2001 Jul. 1; 15(13):1593-612.

“Detectable label”, “label”, “detectable marker” or “marker” are used interchangeably, including, but not limited to radioisotopes, fluorochromes, chemiluminescent compounds, dyes, and proteins, including enzymes. Detectable labels can also be attached to a polynucleotide, polypeptide, antibody or composition described herein.

As used herein, the term “label” or a detectable label intends a directly or indirectly detectable compound or composition that is conjugated directly or indirectly to the composition to be detected, e.g., N-terminal histidine tags (N-His), magnetically active isotopes, e.g., ¹¹⁵Sn, ¹¹⁷Sn and ¹¹⁹Sn, a non-radioactive isotopes such as ¹³C and ¹⁵N, polynucleotide or protein such as an antibody so as to generate a “labeled” composition. The term also includes sequences conjugated to the polynucleotide that will provide a signal upon expression of the inserted sequences, such as green fluorescent protein (GFP) and the like. The label may be detectable by itself (e.g., radioisotope labels or fluorescent labels) or, in the case of an enzymatic label, may catalyze chemical alteration of a substrate compound or composition which is detectable. The labels can be suitable for small scale detection or more suitable for high-throughput screening. As such, suitable labels include, but are not limited to magnetically active isotopes, non-radioactive isotopes, radioisotopes, fluorochromes, chemiluminescent compounds, dyes, and proteins, including enzymes. The label may be simply detected, or it may be quantified. A response that is simply detected generally comprises a response whose existence merely is confirmed, whereas a response that is quantified generally comprises a response having a quantifiable (e.g., numerically reportable) value such as an intensity, polarization, or other property. In luminescence or fluorescence assays, the detectable response may be generated directly using a luminophore or fluorophore associated with an assay component actually involved in binding, or indirectly using a luminophore or fluorophore associated with another (e.g., reporter or indicator) component. Examples of luminescent labels that produce signals include, but are not limited to bioluminescence and chemiluminescence. Detectable luminescence response generally comprises a change in, or an occurrence of a luminescence signal. Suitable methods and luminophores for luminescently labeling assay components are known in the art and described for example in Haugland, Richard P. (1996) Handbook of Fluorescent Probes and Research Chemicals (6th ed). Examples of luminescent probes include, but are not limited to, aequorin and luciferases.

As used herein, the term “immunoconjugate” comprises an antibody or an antibody derivative associated with or linked to a second agent, such as a cytotoxic agent, a detectable agent, a radioactive agent, a targeting agent, a human antibody, a humanized antibody, a chimeric antibody, a synthetic antibody, a semisynthetic antibody, or a multispecific antibody.

Examples of suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade Blue™, and Texas Red. Other suitable optical dyes are described in the Haugland, Richard P. (1996) Handbook of Fluorescent Probes and Research Chemicals (6th ed.).

In some embodiments, the fluorescent label is functionalized to facilitate covalent attachment to a cellular component present in or on the surface of the cell or tissue such as a cell surface marker. Suitable functional groups, include, but are not limited to, isothiocyanate groups, amino groups, haloacetyl groups, maleimides, succinimidyl esters, and sulfonyl halides, all of which may be used to attach the fluorescent label to a second molecule. The choice of the functional group of the fluorescent label will depend on the site of attachment to either a linker, the agent, the marker, or the second labeling agent.

As used herein, a purification label or maker refers to a label that may be used in purifying the molecule or component that the label is conjugated to, such as an epitope tag (including but not limited to a Myc tag, a human influenza hemagglutinin (HA) tag, a FLAG tag), an affinity tag (including but not limited to a glutathione-S transferase (GST), a poly-Histidine (His) tag, Calmodulin Binding Protein (CBP), or Maltose-binding protein (MBP)), or a fluorescent tag.

As used herein, an amino acid (aa) or nucleotide (nt) residue position in a sequence of interest “corresponding to” an identified position in a reference sequence refers to that the residue position is aligned to the identified position in a sequence alignment between the sequence of interest and the reference sequence. Various programs are available for performing such sequence alignments, such as Clustal Omega and BLAST.

The term “consensus sequence” as used herein refers to an amino acid or nucleic acid sequence that is determined by aligning a series of multiple sequences and that defines an idealized sequence that represents the predominant choice of amino acid or base at each corresponding position of the multiple sequences. Depending on the sequences of the series of multiple sequences, the consensus sequence for the series can differ from each of the sequences by zero, one, a few, or more substitutions. Also, depending on the sequences of the series of multiple sequences, more than one consensus sequence may be determined for the series. The generation of consensus sequences has been subjected to intensive mathematical analysis. Various software programs can be used to determine a consensus sequence.

In some embodiments, the term “vector” intends a recombinant vector that retains the ability to infect or transduce non-dividing or dividing cells, such as slowly-dividing cells, and optionally integrate into the target cell's genome. In some embodiments, the vector is a non-viral vector, such as a plasmid. In some embodiments, the vector is a viral vector.

A “plasmid” is an extra-chromosomal DNA molecule separate from the chromosomal DNA which is capable of replicating independently of the chromosomal DNA. In many cases, it is circular and double-stranded. Plasmids provide a mechanism for horizontal gene transfer within a population of microbes and typically provide a selective advantage under a given environmental state. Plasmids may carry genes that provide resistance to naturally occurring antibiotics in a competitive environmental niche, or alternatively the proteins produced may act as toxins under similar circumstances. Many plasmids are commercially available for such uses. The gene to be replicated is inserted into copies of a plasmid containing genes that make cells resistant to particular antibiotics and a multiple cloning site (MCS, or polylinker), which is a short region containing several commonly used restriction sites allowing the easy insertion of DNA fragments at this location. In some embodiments, one or more plasmids are used in producing a viral vector or a viral genome. In some embodiments, a plasmid is used for replicating or amplifying a polynucleotide. Another major use of plasmids is to make large amounts of proteins. In this case, researchers grow bacteria containing a plasmid harboring the gene of interest. Just as the bacterium produces proteins to confer its antibiotic resistance, it can also be induced to produce large amounts of proteins from the inserted gene. This is a cheap and easy way of mass-producing a gene or the protein it then codes for.

A “viral vector” is defined as a recombinantly produced virus or viral particle that comprises a polynucleotide (a viral genome) to be delivered into a host cell, either in vivo, ex vivo or in vitro or ex vivo. As is known to those of skill in the art, there are 6 classes of viruses. The DNA viruses constitute classes I and II. The RNA viruses and retroviruses make up the remaining classes. Class III viruses have a double-stranded RNA genome. Class IV viruses have a positive single-stranded RNA genome, the genome itself acting as mRNA Class V viruses have a negative single-stranded RNA genome used as a template for mRNA synthesis. Class VI viruses have a positive single-stranded RNA genome but with a DNA intermediate not only in replication but also in mRNA synthesis. Retroviruses carry their genetic information in the form of RNA; however, once the virus infects a cell, the RNA is reverse-transcribed into the DNA form which optionally integrates into the genomic DNA of the infected cell. The integrated DNA form is called a provirus.

Examples of viral vectors include retroviral vectors, lentiviral vectors, adenovirus vectors, adeno-associated virus vectors, alphavirus vectors and the like. Alphavirus vectors, such as Semliki Forest virus-based vectors and Sindbis virus-based vectors, have also been developed for use in gene therapy and immunotherapy. See, Schlesinger and Dubensky (1999) Curr. Opin. Biotechnol. 5:434-439 and Ying, et al. (1999) Nat. Med. 5(7):823-827.

In several embodiments, the vector is derived from or based on a wild-type virus. In further embodiments, the vector is derived from or based on one or more of a wild-type adenovirus, an adeno-associated virus, or a retrovirus such as a gammaretrovirus or a lentivirus. As used herein, the vector may be a gammaretroviral vector (PCIR). Examples of retrovirus include without limitation, moloney murine leukemia virus (MMLV), murine stem cell virus (MSCV), or friend murine embryonic stem cell virus (FMEV), human immunodeficiency virus (HIV), equine infectious anaemia virus (EIAV), simian immunodeficiency virus (SIV) and feline immunodeficiency virus (Hy). The viral vector may comprise components derived from two or more different viruses, and may also comprise synthetic components. Vector components can be manipulated to obtain desired characteristics such as target cell specificity.

The recombinant vectors of this disclosure may be derived from primates and non-primates. Examples of primate lentiviruses include the human immunodeficiency virus (HIV), the causative agent of human acquired immunodeficiency syndrome (AIDS), and the simian immunodeficiency virus (SIV). The non-primate lentiviral group includes the prototype “slow virus” visna/maedi virus (VMV), as well as the related caprine arthritis-encephalitis virus (CAEV), equine infectious anaemia virus (EIAV), the more recently described feline immunodeficiency virus (FIV), and bovine immunodeficiency virus (BIV). Prior art recombinant lentiviral vectors are known in the art, e.g., see U.S. Pat. Nos. 6,924,123; 7,056,699; 7,419,829 and 7,442,551, incorporated herein by reference. In some embodiments, the lentiviral vector is a self-inactivating lentiviral vector. In further embodiments, the lentiviral vector has a U3 region lacking a TATA box. Additionally or alternatively, the lentiviral vector has a U3 region lacking one or more of transcription factor binding site(s).

A retrovirus such as a gammaretrovirus or a lentivirus comprises (a) envelope comprising lipids and glycoprotein, (b) a vector genome, which is a RNA (usually a dimer RNA comprising a cap at the 5′ end and a polyA tail at the 3′ end flanked by LTRs) delivered to the target cell, (c) a capsid, and (d) other proteins, such as a protease. U.S. Pat. No. 6,924,123 discloses that certain retroviral sequence facilitate integration into the target cell genome. This patent teaches that each retroviral genome comprises genes called gag, pol and env which code for virion proteins and enzymes. These genes are flanked at both ends by regions called long terminal repeats (LTRs). The LTRs are responsible for proviral integration, and transcription. They also serve as enhancer-promoter sequences. In other words, the LTRs can control the expression of the viral genes. Encapsidation of the retroviral RNAs occurs by virtue of a psi sequence located at the 5′ end of the viral genome. The LTRs themselves are identical sequences that can be divided into three elements, which are called U3, R and U5. U3 is derived from the sequence unique to the 3′ end of the RNA. R is derived from a sequence repeated at both ends of the RNA, and U5 is derived from the sequence unique to the 5′end of the RNA. The sizes of the three elements can vary considerably among different retroviruses. For the viral genome and the site of poly (A) addition (termination) is at the boundary between R and U5 in the right hand side LTR. U3 contains most of the transcriptional control elements of the provirus, which include the promoter and multiple enhancer sequences responsive to cellular and in some cases, viral transcriptional activator proteins.

With regard to the structural genes gag, pol and env themselves, gag encodes the internal structural protein of the virus. Gag protein is proteolytically processed into the mature proteins MA (matrix), CA (capsid) and NC (nucleocapsid). The pol gene encodes the reverse transcriptase (RT), which contains DNA polymerase, associated RNase H and integrase (IN), which mediate replication of the genome.

For the production of viral vector particles, the vector genome (such as an RNA vector genome) is expressed from a DNA construct (such as a plasmid) encoding it, in a host cell. The components of the particles not encoded by the vector genome are provided in trans by additional nucleic acid sequences (the “packaging system”, which usually includes either or both of the gag/pol and env genes) expressed in the host cell. The set of sequences required for the production of the viral vector particles may be introduced into the host cell by transient transfection, or they may be integrated into the host cell genome, or they may be provided in a mixture of ways. The techniques involved are known to those skilled in the art.

In embodiments where gene transfer is mediated by a lentiviral vector, a vector construct refers to the polynucleotide comprising the lentiviral genome or part thereof, and a therapeutic gene. As used herein, “lentiviral mediated gene transfer” or “lentiviral transduction” carries the same meaning and refers to the process by which a gene or nucleic acid sequences are stably transferred into the host cell by virtue of the virus entering the cell and integrating its genome into the host cell genome. The virus can enter the host cell via its normal mechanism of infection or be modified such that it binds to a different host cell surface receptor or ligand to enter the cell. Retroviruses carry their genetic information in the form of RNA; however, once the virus infects a cell, the RNA is reverse-transcribed into the DNA form which integrates into the genomic DNA of the infected cell. The integrated DNA form is called a provirus. As used herein, lentiviral vector refers to a viral particle capable of introducing exogenous nucleic acid into a cell through a viral or viral-like entry mechanism. A “lentiviral vector” is a type of retroviral vector well-known in the art that has certain advantages in transducing nondividing cells as compared to other retroviral vectors. See, Trono D. (2002) Lentiviral vectors, New York: Spring-Verlag Berlin Heidelberg.

Lentiviral vectors of this disclosure are based on or derived from oncoretroviruses (the sub-group of retroviruses containing MLV), and lentiviruses (the sub-group of retroviruses containing HIV). Examples include ASLV, SNV and RSV all of which have been split into packaging and vector components for lentiviral vector particle production systems. The lentiviral vector particle according to the disclosure may be based on a genetically or otherwise (e.g. by specific choice of packaging cell system) altered version of a particular retrovirus.

The term “adeno-associated virus” or “AAV” as used herein refers to a member of the class of viruses associated with this name and belonging to the genus dependoparvovirus, family Parvoviridae. Multiple serotypes of this virus are known to be suitable for gene delivery; all known serotypes can infect cells from various tissue types. At least 11 sequentially numbered, AAV serotypes are known in the art. Non-limiting exemplary serotypes useful in the methods disclosed herein include any of the 11 serotypes, e.g., AAV2, AAV8, AAV9, or variant or synthetic serotypes, e.g., AAV-DJ and AAV PHP.B. The AAV particle comprises, alternatively consists essentially of, or yet further consists of three major viral proteins: VP1, VP2 and VP3. In one embodiment, the AAV refers to of the serotype AAV1, AAV2, AAV4, AAV5, AAV6, AAV7, AAV8, AAV9, AAV10, AAV11, AAV12, AAV13, AAV PHP.B, or AAV rh74. These vectors are commercially available or have been described in the patent or technical literature.

That the vector particle according to the disclosure is “based on” a particular retrovirus means that the vector is derived from that particular retrovirus. The genome of the vector particle comprises components from that retrovirus as a backbone. The vector particle contains essential vector components compatible with the genome, such as an RNA genome, including reverse transcription and integration systems. Usually these will include gag and pol proteins derived from the particular retrovirus. Thus, the majority of the structural components of the vector particle will normally be derived from that retrovirus, although they may have been altered genetically or otherwise so as to provide desired useful properties. However, certain structural components and in particular the env proteins, may originate from a different virus. The vector host range and cell types infected or transduced can be altered by using different env genes in the vector particle production system to give the vector particle a different specificity.

As used herein, a cell may be a prokaryotic or a eukaryotic cell. In further embodiments, the cell is an immune cell.

As used herein, “Immune cells” includes, e.g., white blood cells (leukocytes, such as granulocytes (neutrophils, eosinophils, and basophils), monocytes, and lymphocytes (T cells, B cells, natural killer (NK) cells and NKT cells)) which may be derived from hematopoietic stem cells (HSC) produced in the bone marrow, lymphocytes (T cells, B cells, natural killer (NK) cells, and NKT cells) and myeloid-derived cells (neutrophil, eosinophil, basophil, monocyte, macrophage, dendritic cells). In some embodiments, the immune cell is derived from one or more of the following: progenitor cells, embryonic stem cells, embryonic stem cell derived cells, embryonic germ cells, embryonic germ cell derived cells, stem cells, stem cell derived cells, pluripotent stem cells, induced pluripotent stem cells (iPSc), haematopoietic stem cells (HSCs), or immortalized cells. In some embodiments, the HSC are derived from umbilical cord blood of a subject, peripheral blood of a subject, or bone marrow of a subject.

“Host cell” refers not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

An “enriched population” of cells intends a substantially homogenous population of cells having certain defined characteristics. The cells are greater than 70%, or alternatively greater than 75%, or alternatively greater than 80%, or alternatively greater than 85%, or alternatively greater than 90%, or alternatively greater than 95%, or alternatively greater than 98% identical in the defined characteristics.

The term “propagate” means to grow a cell or population of cells. The term “growing” also refers to the proliferation of cells in the presence of supporting media, nutrients, growth factors, support cells, or any chemical or biological compound necessary for obtaining the desired number of cells or cell type.

The term “culturing” refers to the in vitro or ex vivo propagation of cells or organisms on or in media of various kinds. It is understood that the descendants of a cell grown in culture may not be completely identical (i.e., morphologically, genetically, or phenotypically) to the parent cell.

As used herein, the term “NK cell,” also known as natural killer cell, refers to a type of lymphocyte that originates in the bone marrow and play a critical role in the innate immune system. NK cells provide rapid immune responses against viral-infected cells, tumor cells or other stressed cell, even in the absence of antibodies and major histocompatibility complex on the cell surfaces. NK cells may either be isolated or obtained from a commercially available source. Non-limiting examples of commercial NK cell lines include lines NK-92 (ATCC® CRL-2407™), NK-92MI (ATCC® CRL-2408™). Further examples include but are not limited to NK lines HANK1, KHYG-1, NKL, NK-YS, NOI-90, and YT. Non-limiting exemplary sources for such commercially available cell lines include the American Type Culture Collection, or ATCC (www.atcc.org/) and the German Collection of Microorganisms and Cell Cultures (www.dsmz.de/).

As used herein, the term “T cell,” refers to a type of lymphocyte that matures in the thymus. T cells play an important role in cell-mediated immunity and are distinguished from other lymphocytes, such as B cells, by the presence of a T-cell receptor on the cell surface. T-cells may either be isolated or obtained from a commercially available source. “T cell” includes all types of immune cells expressing CD3 including T-helper cells (CD4+ cells), cytotoxic T-cells (CD8+ cells), natural killer T-cells, T-regulatory cells (Treg) and gamma-delta T cells. A “cytotoxic cell” includes CD8+T cells, natural-killer (NK) cells, and neutrophils, which cells are capable of mediating cytotoxicity responses. Non-limiting examples of commercially available T-cell lines include lines BCL2 (AAA) Jurkat (ATCC® CRL-2902™), BCL2 (S70A) Jurkat (ATCC® CRL-2900™), BCL2 (S87A) Jurkat (ATCC® CRL-2901™), BCL2 Jurkat (ATCC® CRL-2899™), Neo Jurkat (ATCC® CRL-2898™), TALL-104 cytotoxic human T cell line (ATCC #CRL-11386). Further examples include but are not limited to mature T-cell lines, e.g., such as Deglis, EBT-8, HPB-MLp-W, HUT 78, HUT 102, Karpas 384, Ki 225, My-La, Se-Ax, SKW-3, SMZ-1 and T34; and immature T-cell lines, e.g., ALL-SIL, Be13, CCRF-CEM, CML-T1, DND-41, DU.528, EU-9, HD-Mar, HPB-ALL, H-SB2, HT-1, JK-T1, Jurkat, Karpas 45, KE-37, KOPT-K1, K-T1, L-KAW, Loucy, MAT, MOLT-1, MOLT 3, MOLT-4, MOLT 13, MOLT-16, MT-1, MT-ALL, P12/Ichikawa, Peer, PER0117, PER-255, PF-382, PFI-285, RPMI-8402, ST-4, SUP-T1 to T14, TALL-1, TALL-101, TALL-103/2, TALL-104, TALL-105, TALL-106, TALL-107, TALL-197, TK-6, TLBR-1, -2, -3, and-4, CCRF-HSB-2 (CCL-120.1), J.RT3-T3.5 (ATCC TIB-153), J45.01 (ATCC CRL-1990), J.CaM1.6 (ATCC CRL-2063), RS4;11 (ATCC CRL-1873), CCRF-CEM (ATCC CRM-CCL-119); and cutaneous T-cell lymphoma lines, e.g., HuT78 (ATCC CRM-TIB-161), MJ[G11] (ATCC CRL-8294), HuT102 (ATCC TIB-162). Null leukemia cell lines, including but not limited to REH, NALL-1, KM-3, L92-221, are another commercially available source of immune cells, as well as cell lines derived from other leukemias and lymphomas, such as K562 erythroleukemia, THP-1 monocytic leukemia, U937 lymphoma, HEL erythroleukemia, HL60 leukemia, HMC-1 leukemia, KG-1 leukemia, U266 myeloma. Non-limiting exemplary sources for such commercially available cell lines include the American Type Culture Collection, or ATCC (www.atcc.org/) and the German Collection of Microorganisms and Cell Cultures (www.dsmz.de/).

The term “stem cell” refers to a cell that is in an undifferentiated or partially differentiated state and has the capacity for self-renewal or to generate differentiated progeny or both. Self-renewal is defined as the capability of a stem cell to proliferate and give rise to more such stem cells, while maintaining its developmental potential (i.e., totipotent, pluripotent, multipotent, etc.). The term “somatic stem cell” is used herein to refer to any stem cell derived from non-embryonic tissue, including fetal, juvenile, and adult tissue. Natural somatic stem cells have been isolated from a wide variety of adult tissues including blood, bone marrow, brain, olfactory epithelium, skin, pancreas, skeletal muscle, and cardiac muscle. Exemplary naturally occurring somatic stem cells include, but are not limited to, mesenchymal stem cells (MSCs) and neural or neuronal stem cells (NSCs). In some embodiments, the stem or progenitor cells can be embryonic stem cells or an induced pluripotent stem cell (iPSC). In some embodiments, the stem or progenitor cells are hematopoietic stem cells (HSCs). As used herein, “embryonic stem cells” refers to stem cells derived from tissue formed after fertilization but before the end of gestation, including pre-embryonic tissue (such as, for example, a blastocyst), embryonic tissue, or fetal tissue taken any time during gestation, typically but not necessarily before approximately 10-12 weeks gestation. Most frequently, embryonic stem cells are pluripotent cells derived from the early embryo or blastocyst. Embryonic stem cells can be obtained directly from suitable tissue, including, but not limited to human tissue, or from established embryonic cell lines. “Embryonic-like stem cells” refer to cells that share one or more, but not all characteristics, of an embryonic stem cell.

“Differentiation” describes the process whereby an unspecialized cell acquires the features of a specialized cell such as a heart, liver, immune or muscle cell. “Directed differentiation” refers to the manipulation of stem cell culture conditions to induce differentiation into a particular cell type. “Dedifferentiated” defines a cell that reverts to a less committed position within the lineage of a cell. As used herein, the term “differentiates or differentiated” defines a cell that takes on a more committed (“differentiated”) position within the lineage of a cell.

As used herein, the term “differentiates or differentiated” defines a cell that takes on a more committed (“differentiated”) position within the lineage of a cell. “Dedifferentiated” defines a cell that reverts to a less committed position within the lineage of a cell. Induced pluripotent stem cells are examples of dedifferentiated cells.

As used herein, the “lineage” of a cell defines the heredity of the cell, i.e. its predecessors and progeny. The lineage of a cell places the cell within a hereditary scheme of development and differentiation.

A “multi-lineage stem cell” or “multipotent stem cell” refers to a stem cell that reproduces itself and at least two further differentiated progeny cells from distinct developmental lineages. The lineages can be from the same germ layer (i.e. mesoderm, ectoderm or endoderm), or from different germ layers.

A “precursor” or “progenitor cell” intends to mean cells that have a capacity to differentiate into a specific type of cell. A progenitor cell may be a stem cell. A progenitor cell may also be more specific than a stem cell. A progenitor cell may be unipotent or multipotent. Compared to adult stem cells, a progenitor cell may be in a later stage of cell differentiation. An example of progenitor cell includes, without limitation, a progenitor nerve cell.

As used herein, a “pluripotent cell” defines a less differentiated cell that can give rise to at least two distinct (genotypically or phenotypically or both) further differentiated progeny cells. In another aspect, a “pluripotent cell” includes an Induced Pluripotent Stem Cell (iPSC) which is an artificially derived stem cell from a non-pluripotent cell, typically an adult somatic cell, that has historically been produced by inducing expression of one or more stem cell specific genes. Such stem cell specific genes include, but are not limited to, the family of octamer transcription factors, i.e. Oct-3/4; the family of Sox genes, i.e., Sox1, Sox2, Sox3, Sox 15 and Sox 18; the family of Klf genes, i.e. Klf1, Klf2, Klf4 and Klf5; the family of Myc genes, i.e. c-myc and L-myc; the family of Nanog genes, i.e., OCT4, NANOG and REX1; or LIN28. Examples of iPSCs are described in Takahashi et al. (2007) Cell advance online publication 20 Nov. 2007; Takahashi & Yamanaka (2006) Cell 126:663-76; Okita et al. (2007) Nature 448:260-262; Yu et al. (2007) Science advance online publication 20 Nov. 2007; and Nakagawa et al. (2007) Nat. Biotechnol. Advance online publication 30 Nov. 2007.

An “induced pluripotent cell” intends embryonic-like cells reprogrammed to the immature phenotype from adult cells. Various methods are known in the art, e.g., “A simple new way to induce pluripotency: Acid.” Nature, 29 Jan. 2014 and available at sciencedaily.com/releases/2014/01/140129184445, last accessed on Feb. 5, 2014 and U.S. Patent Application Publication No. 2010/0041054. Human iPSCs also express stem cell markers and are capable of generating cells characteristic of all three germ layers.

A “parthenogenetic stem cell” refers to a stem cell arising from parthenogenetic activation of an egg. Methods of creating a parthenogenetic stem cell are known in the art. See, for example, Cibelli et al. (2002) Science 295(5556):819 and Vrana et al. (2003) Proc. Natl. Acad. Sci. USA 100(Suppl. 1)11911-6.

As used herein, the term “pluripotent gene or marker” intends an expressed gene or protein that has been correlated with an immature or undifferentiated phenotype, e.g., Oct Sox2, Nanog, c-Myc and LIN-28. Methods to identify such are known in the art and systems to identify such are commercially available from, for example, EMD Millipore (MILLIPLEX® Map Kit).

As used herein, hematopoietic stem cells (HSCs) are cells, such as stem cells, that give rise to all types of blood cells, including but not limited to white blood cells, red blood cells, and platelets. Hematopoietic stem cells can be found in the peripheral blood and the bone marrow. In some embodiments, an immune cell as disclosed herein is derived from an HSC.

The term “phenotype” refers to a description of an individual's trait or characteristic that is measurable and that is expressed only in a subset of individuals within a population. In one aspect of the disclosure, an individual's phenotype includes the phenotype of a single cell, a substantially homogeneous population of cells, a population of differentiated cells, or a tissue comprised of a population of cells.

In some embodiments, a population of cells intends a collection of more than one cell that is identical (clonal) or non-identical in phenotype or genotype or both. The population can be purified, highly purified, substantially homogenous or heterogeneous as described herein.

The terms effective period (or time) and effective conditions refer to a period of time or other controllable conditions (e.g., temperature, humidity for in vitro or ex vivo methods), necessary or preferred for an agent or composition to achieve its intended result, e.g., the differentiation or dedifferentiation of cells to a pre-determined cell type.

“Substantially homogeneous” describes a population of cells in which more than about 50%, or alternatively more than about 60%, or alternatively more than 70%, or alternatively more than 75%, or alternatively more than 80%, or alternatively more than 85%, or alternatively more than 90%, or alternatively more than 95%, of the cells are of the same or similar phenotype. Phenotype can be determined by a pre-selected cell surface marker or other marker.

The terms or “acceptable,” “effective,” or “sufficient” when used to describe the selection of any components, ranges, dose forms, etc. disclosed herein intend that said component, range, dose form, etc. is suitable for the disclosed purpose.

As used herein, the terms “treating,” “treatment” and the like are used herein to mean obtaining a desired pharmacologic or physiologic effect. In some embodiments, the effect can be prophylactic in terms of completely or partially preventing a disorder or sign or symptom thereof, or can be therapeutic in terms of a partial or complete cure for a disorder or adverse effect attributable to the disorder. Examples of “treatment” include but are not limited to: preventing a disorder from occurring in a subject that may be predisposed to a disorder, but has not yet been diagnosed as having it; inhibiting a disorder, i.e., arresting its development; or relieving or ameliorating the symptoms of disorder. In some embodiments, treatment is the arrestment of the development of symptoms of the disease or disorder, e.g., a cancer. In some embodiments, they refer to (1) preventing the symptoms or disease from occurring in a subject that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of the present technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable. When the disease is cancer, the following clinical end points are non-limiting examples of treatment: reduction in tumor burden, slowing of tumor growth, longer overall survival, longer time to tumor progression, inhibition of metastasis or a reduction in metastasis of the tumor. In some embodiments where the disease is an immune cell cancer, such as multiple myeloma (MM) or an acute myeloid leukemia (AML), reduction in an immunoglobulin (such as IgG) level, or residual cancer cells (for example as measured by flow cytometry, RT-PCR, or other conventional clinical methods), or both, in a biological sample of a subject, such as peripheral blood, plasma or serum, may be used as a clinical end point. In some embodiments where the disease is a cancer or tumor, reduction in circulating tumor cells (CTCs, which refers to a cell that is shed into the vasculature or lymphatics and is carried around the subject body in the blood circulation) in a biological sample of a subject (for example as measured by PCR or other suitable clinical methods), such as peripheral blood, plasma or serum, may be used as a clinical end point. In some embodiments, treatment excludes prophylaxis. In one aspect, treatment excludes prophylaxis.

As used herein, the term “sample” and “biological sample” are used interchangeably, referring to sample material derived from a subject. Biological samples may include tissues, cells, protein or membrane extracts of cells, and biological fluids (e.g., ascites fluid or cerebrospinal fluid (CSF)) isolated from a subject, as well as tissues, cells and fluids present within a subject. Biological samples may include, but are not limited to, samples taken from breast tissue, renal tissue, the uterine cervix, the endometrium, the head or neck, the gallbladder, parotid tissue, the prostate, the brain, the pituitary gland, kidney tissue, muscle, the esophagus, the stomach, the small intestine, the colon, the liver, the spleen, the pancreas, thyroid tissue, heart tissue, lung tissue, the bladder, adipose tissue, lymph node tissue, the uterus, ovarian tissue, adrenal tissue, testis tissue, the tonsils, thymus, blood, hair, buccal, skin, serum, plasma, CSF, semen, prostate fluid, seminal fluid, urine, feces, sweat, saliva, sputum, mucus, bone marrow, lymph, and tears. In some embodiments, a biological sample is selected from peripheral blood, plasma or serum. In some embodiment, the sample is a tumor biopsy.

As used herein, a therapeutic protein or polypeptide refers to a protein or a polypeptide suitable for a treatment, including but not limited to an antibody or a fragment thereof, an enzyme, a ligand or a receptor. Such therapeutic protein or polypeptide may be chose by a physician or one of skill in the art, based on the disease to be treated. For example, for treating a cancer, an antibody to an immune checkpoint receptor or a ligand thereof may be used, such as an anti-PD-1 antibody or an anti-PD-L1 antibody or both.

In one embodiment, the term “disease” or “disorder” as used herein refers to a cancer, a status of being diagnosed with a cancer, a status of being suspect of having a cancer, or a status of at high risk of having a cancer.

As used herein, a “cancer” is a disease state characterized by the presence in a subject of cells demonstrating abnormal uncontrolled replication and in some aspects, the term may be used interchangeably with the term “tumor.” The term “cancer or tumor antigen” refers to an antigen known to be associated and expressed on the surface with a cancer cell or tumor cell or tissue, and the term “cancer or tumor targeting antibody” refers to an antibody that targets such an antigen. In some embodiments, the term “cancer” as used herein refers to multiple myeloma (MM). In some embodiments, the term “cancer” as used herein refers to acute myeloid leukemia (AML). Additionally or alternatively, the cancer as used herein expresses one or both of BCMA or GPRC5D.

A “solid tumor” is an abnormal mass of tissue that usually does not contain cysts or liquid areas. Solid tumors can be benign or malignant, metastatic or non-metastatic. Different types of solid tumors are named for the type of cells that form them. Examples of solid tumors include sarcomas, carcinomas, and lymphomas.

A “composition” is intended to mean a combination of active agent and another compound or composition, inert (for example, a detectable agent or label) or active, such as an adjuvant, diluent, binder, stabilizer, buffers, salts, lipophilic solvents, preservative, adjuvant or the like and include pharmaceutically acceptable carriers.

Carriers also include pharmaceutical excipients and additives proteins, peptides, amino acids, lipids, and carbohydrates (e.g., sugars, including monosaccharides, di-, tri, tetra-oligosaccharides, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid/antibody components, which can also function in a buffering capacity, include alanine, arginine, glycine, arginine, betaine, histidine, glutamic acid, aspartic acid, cysteine, lysine, leucine, isoleucine, valine, methionine, phenylalanine, aspartame, and the like. Carbohydrate excipients are also intended within the scope of this technology, examples of which include but are not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol.

A “pharmaceutical composition” is intended to include the combination of an active polypeptide, polynucleotide, antibody, or cell with a carrier, inert or active such as a solid support, making the composition suitable for diagnostic or therapeutic use in vitro, in vivo or ex vivo.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin (1975) Remington's Pharm. Sci., 15th Ed. (Mack Publ. Co., Easton). The term pharmaceutically acceptable carrier (or medium), which may be used interchangeably with the term biologically compatible carrier or medium, refers to reagents, cells, compounds, materials, compositions, or dosage forms, or any combination thereof, that are not only compatible with the cells and other agents to be administered therapeutically, but also are, within the scope of sound medical judgment, suitable for use in contact with the tissues of human beings and animals without excessive toxicity, irritation, allergic response, or other complication commensurate with a reasonable benefit to risk ratio. Pharmaceutically acceptable carriers suitable for use in the present disclosure include liquids, semi-solid (e.g., gels) and solid materials (e.g., cell scaffolds and matrices, tubes sheets and other such materials as known in the art and described in greater detail herein). These semi-solid and solid materials may be designed to resist degradation within the body (non-biodegradable) or they may be designed to degrade within the body (biodegradable, bioerodible). A biodegradable material may further be bioresorbable or bioabsorbable, i.e., it may be dissolved and absorbed into bodily fluids (water-soluble implants are one example), or degraded and ultimately eliminated from the body, either by conversion into other materials or breakdown and elimination through natural pathways.

“Pharmaceutically acceptable carriers” refers to any diluents, excipients, or carriers that may be used in the compositions disclosed herein. Pharmaceutically acceptable carriers include ion exchangers, alumina, aluminum stearate, lecithin, serum proteins, such as human serum albumin, buffer substances, such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Suitable pharmaceutical carriers are described in Remington's Pharmaceutical Sciences, Mack Publishing Company, a standard reference text in this field. They may be selected with respect to the intended form of administration, that is, oral tablets, capsules, elixirs, syrups and the like, and consistent with conventional pharmaceutical practices.

The compositions used in accordance with the disclosure can be packaged in dosage unit form for ease of administration and uniformity of dosage. The term “unit dose” or “dosage” refers to physically discrete units suitable for use in a subject, each unit containing a predetermined quantity of the composition calculated to produce the desired responses in association with its administration, i.e., the appropriate route and regimen. The quantity to be administered, both according to number of treatments and unit dose, depends on the result or protection or both desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition. Upon formulation, solutions are administered in a manner compatible with the dosage formulation and in such amount as is therapeutically or prophylactically effective. The formulations are easily administered in a variety of dosage forms, such as the type of injectable solutions described herein.

As used herein, the term “contacting” means direct or indirect binding or interaction between two or more molecules or other entities. A particular example of direct interaction is binding. A particular example of an indirect interaction is where one entity acts upon an intermediary molecule, which in turn acts upon the second referenced entity. Contacting as used herein includes in solution, in solid phase, in vitro, ex vivo, in a cell and in vivo. Contacting in vivo can be referred to as administering, or administration.

“Administration” or “delivery” of a cell or vector or other agent and compositions containing same can be performed in one dose, continuously or intermittently throughout the course of treatment. Methods of determining the most effective means and dosage of administration are known to those of skill in the art and will vary with the composition used for therapy, the purpose of the therapy, the target cell being treated, and the subject being treated. Single or multiple administrations can be carried out with the dose level and pattern being selected by the treating physician or in the case of animals, by the treating veterinarian. Suitable dosage formulations and methods of administering the agents are known in the art. Route of administration can also be determined and method of determining the most effective route of administration are known to those of skill in the art and will vary with the composition used for treatment, the purpose of the treatment, the health condition or disease stage of the subject being treated, and target cell or tissue. Non-limiting examples of route of administration include oral administration, intraperitoneal, infusion, nasal administration, inhalation, injection, and topical application. In some embodiments, the administration is an intratumoral administration, or administration to a tumor microenvironment, or both. In some embodiments, the administration is an infusion (for example to peripheral blood of a subject) over a certain period of time, such as about 30 minutes, about 1 hour, about 2 hours, about 3 hours, about 4 hours, about 5 hours, about 6 hours, about 7 hours, about 8 hours, about 9 hours, about 10 hours, about 11 hours, about 12 hours, about 24 hours or longer.

The term administration shall include without limitation, administration by oral, parenteral (e.g., intramuscular, intraperitoneal, intravenous, intracerebroventricular (ICV), intrathecal, intracisternal injection or infusion, subcutaneous injection, or implant), by inhalation spray nasal, vaginal, rectal, sublingual, urethral (e.g., urethral suppository) or topical routes of administration (e.g., gel, ointment, cream, aerosol, etc.) and can be formulated, alone or together, in suitable dosage unit formulations containing conventional non-toxic pharmaceutically acceptable carriers, adjuvants, excipients, and vehicles appropriate for each route of administration. The disclosure is not limited by the route of administration, the formulation or dosing schedule.

“Administration” can be performed in one dose, continuously or intermittently throughout the course of treatment. Methods of determining the most effective means and dosage of administration are known to those of skill in the art and will vary with the composition used for therapy, the purpose of the therapy, the target cell being treated, and the subject being treated. Single or multiple administrations can be carried out with the dose level and pattern being selected by the treating physician. Suitable dosage formulations and methods of administering the agents are known in the art. Route of administration can also be determined and method of determining the most effective route of administration are known to those of skill in the art and will vary with the composition used for treatment, the purpose of the treatment, the health condition or disease stage of the subject being treated, and target cell or tissue. In some embodiments, 1×10⁴ to 1×10¹⁵ or ranges in between of cells as disclosed herein are administrated to a subject, such as 1×10⁷ to 1×10¹⁰. In some embodiments, administering or a grammatical variation thereof also refers to more than one doses with certain interval. In some embodiments, the interval is 1 day, 2 days, 3 days, 4 days, 5 days, 6 days, 1 week, 10 days, 2 weeks, 3 weeks, 1 month, 2 months, 3 months, 4 months, 5 months, 6 months, 1 year or longer. In some embodiments, one dose is repeated for once, twice, three times, four times, five times, six times, seven times, eight times, nine times, ten times or more. For example, cells as disclosed herein may be administered to a subject weekly and for up to four weeks. The compositions and therapies can be combined with other therapies, e.g., lymphodepletion chemotherapy followed by infusions (e.g., four weekly infusions) of the therapy, defining one cycle, followed by additional cycles until a partial or complete response is seen or alternatively utilized as a “bridging” therapy to another modality, such as hematopoietic stem cell transplantation or CAR T cell therapy.

An agent of the present disclosure can be administered for therapy by any suitable route of administration. It will also be appreciated that the optimal route will vary with the condition and age of the recipient, and the disease being treated.

A “subject,” “individual” or “patient” is used interchangeably herein, and refers to a vertebrate, preferably a mammal, more preferably a human. Mammals include, but are not limited to, murines, rats, rabbit, simians, bovines, ovine, porcine, canines, feline, farm animals, sport animals, pets, equine, and primate, particularly human. Besides being useful for human treatment, the present disclosure is also useful for veterinary treatment of companion mammals, exotic animals and domesticated animals, including mammals, rodents. In one embodiment, the mammals include horses, dogs, and cats. In another embodiment of the present disclosure, the human is a fetus, an infant, a pre-pubescent subject, an adolescent, a pediatric patient, or an adult. In one aspect, the subject is pre-symptomatic mammal or human. In another aspect, the subject has minimal clinical symptoms of the disease. The subject can be a male or a female, adult, an infant or a pediatric subject. In an additional aspect, the subject is an adult. In some instances, the adult is an adult human, e.g., an adult human greater than 18 years of age.

The term “suffering” as it related to the term “treatment” refers to a patient or individual who has been diagnosed with or is predisposed to a disease as disclosed herein. This patient has not yet developed characteristic disease pathology.

An “effective amount” is an amount sufficient to effect beneficial or desired results. An effective amount can be administered in one or more administrations, applications or dosages. Such delivery is dependent on a number of variables including the time period for which the individual dosage unit is to be used, the bioavailability of the therapeutic agent, the route of administration, etc. It is understood, however, that specific dose levels of the therapeutic agents of the present disclosure for any particular subject depends upon a variety of factors including the activity of the specific compound employed, the age, body weight, general health, sex, and diet of the subject, the time of administration, the rate of excretion, the drug combination, and the severity of the particular disorder being treated and form of administration. Treatment dosages generally may be titrated to optimize safety and efficacy. Typically, dosage-effect relationships from in vitro, or ex vivo, or in vivo tests (or any combination thereof) initially can provide useful guidance on the proper doses for patient administration. In general, one will desire to administer an amount of the agent as disclosed herein (such as a cell) that is effective to achieve a serum level commensurate with the concentrations found to be effective in vitro or ex vivo. Determination of these parameters is well within the skill of the art. These considerations, as well as effective formulations and administration procedures are well known in the art and are described in standard textbooks.

“Therapeutically effective amount” of a drug or an agent refers to an amount of the drug or the agent (such as a cell as disclosed herein) that is an amount sufficient to obtain a pharmacological response; or alternatively, is an amount of the drug or agent that, when administered to a patient with a specified disorder or disease, is sufficient to have the intended effect, e.g., treatment, alleviation, amelioration, palliation or elimination of one or more manifestations of the specified disorder or disease in the patient. A therapeutic effect does not necessarily occur by administration of one dose, and may occur only after administration of a series of doses, as needed to induce a partial or complete effect. Thus, a therapeutically effective amount may be administered in one or more administrations. In some embodiments, a therapeutically effective amount of cells as disclosed herein is 1×10⁴ to 1×10¹⁵ or ranges, such as 1×10⁷ to 1×10¹⁰.

In some embodiments, a treatment, such as an immune cell comprising a polypeptide as disclosed herein, is administered to a subject as disclosed herein in an effective amount. In further embodiments, a treatment, such as an immune cell comprising a polypeptide as disclosed herein, is administered to a subject as disclosed herein in a therapeutically effective amount.

An “anti-cancer therapy,” as used herein, includes but is not limited to surgical resection, chemotherapy, cryotherapy, radiation therapy, immunotherapy and targeted therapy. Agents that act to reduce cellular proliferation are known in the art and widely used. Chemotherapy drugs that kill cancer cells only when they are dividing are termed cell-cycle specific. These drugs include agents that act in S-phase, including topoisomerase inhibitors and anti-metabolites.

Topoisomerase inhibitors are drugs that interfere with the action of topoisomerase enzymes (topoisomerase I and II). During the process of chemo treatments, topoisomerase enzymes control the manipulation of the structure of DNA necessary for replication and are thus cell cycle specific. Examples of topoisomerase I inhibitors include the camptothecan analogs listed above, irinotecan and topotecan. Examples of topoisomerase II inhibitors include amsacrine, etoposide, etoposide phosphate, and teniposide.

Antimetabolites are usually analogs of normal metabolic substrates, often interfering with processes involved in chromosomal replication. They attack cells at very specific phases in the cycle. Antimetabolites include folic acid antagonists, e.g., methotrexate; pyrimidine antagonist, e.g., 5-fluorouracil, foxuridine, cytarabine, capecitabine, and gemcitabine; purine antagonist, e.g., 6-mercaptopurine and 6-thioguanine; adenosine deaminase inhibitor, e.g., cladribine, fludarabine, nelarabine and pentostatin; and the like.

Plant alkaloids are derived from certain types of plants. The vinca alkaloids are made from the periwinkle plant (Catharanthus rosea). The taxanes are made from the bark of the Pacific Yew tree (taxus). The vinca alkaloids and taxanes are also known as antimicrotubule agents. The podophyllotoxins are derived from the May apple plant. Camptothecan analogs are derived from the Asian “Happy Tree” (Camptotheca acuminata). Podophyllotoxins and camptothecan analogs are also classified as topoisomerase inhibitors. The plant alkaloids are generally cell-cycle specific.

Examples of these agents include vinca alkaloids, e.g., vincristine, vinblastine and vinorelbine; taxanes, e.g., paclitaxel and docetaxel; podophyllotoxins, e.g., etoposide and tenisopide; and camptothecan analogs, e.g., irinotecan and topotecan.

In some embodiments where the cancer is an immune cell cancer, an anti-cancer therapy may comprises, or consists essentially of, or consists of a hematopoietic stem cell transplantation.

In some embodiments, a therapeutic agent, such as a cell as disclosed herein, may be combined in treating a cancer with another anti-cancer therapy or a therapy depleting an immune cell. For example, lymphodepletion chemotherapy is performed followed by administration of a cell as disclosed herein, such as four weekly infusions. In further embodiments, these steps may be repeated for once, twice, three or more times until a partial or complete effect is observed or a clinical end point is achieved.

Cryotherapy includes, but is not limited to, therapies involving decreasing the temperature, for example, hypothermic therapy.

Radiation therapy includes, but is not limited to, exposure to radiation, e.g., ionizing radiation, UV radiation, as known in the art. Exemplary dosages include, but are not limited to, a dose of ionizing radiation at a range from at least about 2 Gy to not more than about 10 Gy or a dose of ultraviolet radiation at a range from at least about 5 J/m² to not more than about 50 J/m², usually about 10 J/m².

The phrase “first line” or “second line” or “third line” refers to the order of treatment received by a patient. First line therapy regimens are treatments given first, whereas second or third line therapy are given after the first line therapy or after the second line therapy, respectively. The National Cancer Institute defines first line therapy as “the first treatment for a disease or condition”. In patients with cancer, primary treatment can be surgery, chemotherapy, radiation therapy, or a combination of these therapies. First line therapy is also referred to those skilled in the art as “primary therapy and primary treatment.” See National Cancer Institute website at www.cancer.gov, last visited on May 1, 2008. Typically, a patient is given a subsequent chemotherapy regimen because the patient did not show a positive clinical or sub-clinical response to the first line therapy or the first line therapy has stopped.

### MODES FOR CARRYING OUT THE DISCLOSURE

Without wishing to be bound by the theory, it is discovered that targeting BCMA and GPRC5D via either or both of a CAR or a bispecific antibody engaging an immune cell (such as an NK cell, an NKT cell or a T cell) optionally via targeting NKG2D expressed on the immune cell provides surprising more than additive (such as synergistic) effects in killing cancer cells in vivo, ex vivo, or in vivo, therefore providing an effective therapeutic regimen in inhibiting the growth of cancer cells or treating a cancer, such as multiple myeloma (MM). Additionally or alternatively, such expression of one or more of a CAR, a bispecific antibody, or another component as disclosed herein (such as a cytokine) benefits survival or growth of the immune cell in vivo, ex vivo, or in vitro.

Accordingly, as disclosed herein, provided is a polypeptide comprising, or consisting essentially of, or yet further consisting of (i) an amino acid sequence of a Chimeric Antigen Receptor (CAR) comprising an anti-BCMA antigen binding sequence and (ii) an amino acid sequence of a bispecific antibody comprising an anti-GPRC5D antigen binding sequence. It would be understand by one of skill in the art that the disclosure herein also includes embodiments and aspects wherein the anti-GPRC5D antigen binding sequence is switched with the anti-BCMA antigen binding sequence if both are present.

In one aspect, provided is a polypeptide comprising, or consisting essentially of, or yet further consisting of (i) an amino acid sequence of a Chimeric Antigen Receptor (CAR) and (ii) an amino acid sequence of a bispecific antibody.

In some embodiments, the bispecific antibody is an immune cell engager, optionally via specifically recognizing and binding an immune cell. In further embodiments, the bispecific antibody further recognizes and binds a TAA. Therefore, the bispecific antibody brings a cancer cell within the proximity of an immune cell via binding to a TAA present on the cancer cell (such as GPRC5D) and binding to an antigen on the immune cell (such as NKG2D or another as disclosed herein). In some embodiments, the bispecific antibody engages a T cell or an NKT cell (thus referred to herein as a bispecific T cell engager, BiTE) optionally via specifically recognizing and binding to CD3, NKG2D, CD28, OX40, 4-1BB, ICOS, CD27, Integrin alpha-L (LFA-1), CD56, cytotoxic and regulatory T-cell molecule (CRTAM) or CD2. In other embodiments, the bispecific antibody engages an NK cell or an NKT cell (thus referred to herein as a bispecific NK cell engager, BiKE) optionally by or via specifically recognizing and binding to CD3, NKG2D, CD16, SLAM, NKp30, NKp44 or NKp46, or CD56. In some embodiments, the bispecific antibody is a BiTE or a BiKE or both, that acts or act by or via specifically recognizing and binding NKG2D. In further embodiments, binding of the antigen binding sequence or a ligand with the antigen which is not NKG2D but expressed on the cell surface of an immune cell activates the immune cell. In further embodiments, the bispecific antibody further comprises an antigen binding amino acid sequence that recognizes and binds a tumor associated antigen (TAA) (anti-TAA antigen binding sequence) or an equivalent thereof with the proviso that the equivalent recognizes and binds the TAA. Additionally or alternatively, the bispecific antibody may comprise an NKG2D ligand instead of or in addition to the anti-NKG2D antigen binding sequence, such as MHC class I polypeptide-related sequence A (MICA), MEW class I polypeptide-related sequence A (MICB), UL16-binding protein 1 (ULBP1), UL16-binding protein 2 (ULBP2), UL16-binding protein 3 (ULBP3), UL16-binding protein 4 (ULBP4), UL16-binding protein 5 (ULBP5), UL16-binding protein 6 (ULBP6), or any other suitable NKG2D ligand(s). In further embodiments, the TAA of the bispecific antibody recognizes and binds G Protein-Coupled Receptor Class C Group 5 Member D (GPRC5D) or B-cell maturation antigen (BCMA) or an equivalent thereof and wherein the equivalent recognizes and binds GPRC5D or BCMA, respectively. In further embodiments, in the bispecific antibody, the anti-NKG2D antigen binding sequence is linked to the anti-TAA antigen binding sequence via a peptide linker, such as an HMA linker, or a fragment crystallizable (Fc) region of an immunoglobulin, a mutant thereof, or an equivalent thereof, or both a peptide linker and a Fc region of an immunoglobulin, a mutant thereof, or an equivalent thereof.

In some embodiments, the CAR of the polypeptide comprises, or consists essentially of, or yet further consists of (1) an antigen binding amino acid sequence that recognizes and binds a tumor associated antigen (TAA) (anti-TAA antigen binding sequence) or an equivalent thereof and wherein the equivalent recognizes and binds the TAA, (2) a hinge domain, (3) a transmembrane domain, and (4) an intracellular domain. In some embodiments, the TAA of the CAR is different or the same with the TAA of the bispecific antibody. In further embodiments, the anti-TAA antigen binding sequence of the CAR is different or the same with the anti-TAA antigen binding sequence of the bispecific antibody.

In further embodiments, the TAA of the CAR is B-cell maturation antigen (BCMA). Additionally or alternatively, the bispecific antibody of the polypeptide comprises, or consists essentially of, or yet further consists of (1) an antigen binding sequence that recognizes and binds NKG2D (anti-NKG2D antigen binding sequence) or an equivalent thereof with the proviso that the equivalent recognizes and binds NKG2D, and (2) an antigen binding sequence that recognizes and binds G Protein-Coupled Receptor Class C Group 5 Member D (GPRC5D) (anti-GPRC5D antigen binding sequence) or an equivalent thereof and wherein the equivalent recognizes and binds GPRC5D.

In some embodiments, the CAR is located at the N terminus side of the bispecific antibody. In other embodiments, the CAR is located at the C terminus side of the bispecific antibody. In some embodiments, the polypeptide further comprises a cleavable peptide (such as a self-cleaving peptide) located between the CAR amino acid sequence and the amino acid sequence of the bispecific antibody. In further embodiments, the polypeptide may be cleaved by itself or by an enzyme, into two or more peptides, for example, a peptide comprising, or consisting essentially of, or consisting of the CAR (which is referred to herein as the CAR peptide) and the bispecific antibody. In some embodiments, the cleavable peptide permits the polypeptide to be cleaved into two or more peptide fragments.

In some embodiments, the CAR of the polypeptide comprises, or consists essentially of, or yet further consists of (1) an antigen binding amino acid sequence that recognizes and binds G Protein-Coupled Receptor Class C Group 5 Member D (GPRC5D) (anti-GPRC5D antigen binding sequence) or an equivalent thereof and wherein the equivalent recognizes and binds GPRC5D, (2) a hinge domain, (3) a transmembrane domain, and (4) an intracellular domain. Additionally or alternatively, the bispecific antibody comprises, or consists essentially of, or yet further consists of (1) an antigen binding sequence that recognizes and binds NKG2D (anti-NKG2D antigen binding sequence) or an equivalent thereof and wherein the equivalent recognizes and binds NKG2D, and (2) an antigen binding sequence that recognizes and binds a tumor associated antigen (TAA) (anti-TAA antigen binding sequence) or an equivalent thereof and wherein the equivalent recognizes and binds the TAA. In further embodiments, the TAA is BCMA.

In another aspect, provided is an antibody, such as a bispecific antibody, that recognizes and binds to both NKG2D and G Protein-Coupled Receptor Class C Group 5 Member D (GPRC5D), or a fragment thereof with the proviso that the fragment recognizes and binds to both NKG2D and GPRC5D. In some embodiments, the bispecific antibody comprises, or consists essentially of, or yet further consists of (1) an antigen binding sequence that recognizes and binds NKG2D (anti-NKG2D antigen binding sequence) or an equivalent thereof and wherein the equivalent recognizes and binds NKG2D, and (2) an antigen binding sequence that recognizes and binds GPRC5D (anti-GPRC5D antigen binding sequence) or an equivalent thereof and wherein the equivalent recognizes and binds GPRCSD.

In yet another aspect, provided is a Chimeric Antigen Receptor (CAR) comprising, or consisting essentially of, or yet further consisting of (1) an antigen binding sequence that recognizes and binds G Protein-Coupled Receptor Class C Group 5 Member D (GPRCSD) (anti-GPRCSD antigen binding sequence) or an equivalent thereof with the proviso that the equivalent recognizes and binds GPRCSD, (2) a hinge domain, (3) a transmembrane domain, and (4) an intracellular domain. In some embodiments, the CAR further comprising an antigen binding sequence that recognizes and binds B-cell maturation antigen (BCMA) or an equivalent thereof and wherein the equivalent recognizes and binds BCMA.

In further embodiments, the CAR further comprises an antigen binding sequence that recognizes and binds a non-GPRCSD TAA or an equivalent thereof and wherein the equivalent recognizes and binds the non-GPRCSD TAA, i.e., the CAR is a bispecific CAR.

In yet further embodiments, a TAA as disclosed herein comprises, or consists essentially of, or yet further consists of an epitope from a protein or polypeptide selected from: B-cell maturation antigen (BCMA), SLAMF7 (CS1 or CD319), EGFR, wildtype epidermal growth factor receptor (EGFRwt), epidermal growth factor receptor variant III (EGFRVIII), FLT3, CD70, mesothelin, CD123, CD19, carcinoembryonic antigen (CEA), CD133, human epidermal growth factor receptor 2 (HER2), ERBB2 (Her2/neu), CD22, CD30, CD171, CLL-1 (CLECL1), GTPase-activating protein (GAP), CDS, interleukin 13 receptor alpha 2 (IL13Ra2), guanylyl cyclase C (GUCY2C), tumor-associated glycoprotein-72 (TAG-72), thymidine kinase 1 (TK1), hypoxanthine guanine phosphoribosyltransferase (HPRT1), cancer/testis (CT), CD33, ganglioside G2 (GD2), GD3, Tn Ag, prostate specific membrane antigen (PSMA), receptor tyrosine kinase-like orphan receptor 1 (ROR1), TAG72, CD38, CD44v6, epithelial cell adhesion molecule precursor (EpCam or EPCAM), B7H3, KIT, IL-13Ra2, IL-11Ra, prostate stem cell antigen (PSCA), PRSS21, vascular endothelial growth factor receptor 2 (VEGFR2), LewisY, CD24, PDGFR-beta, SSEA-4, CD20, Folate receptor alpha, mucin 1 (Muc1), NCAM, Prostase, PAP, ELF2M, Ephrin B2, fibroblast activation protein alpha (FAP), IGF-I receptor, CAIX, LMP2, gp100, bcr-abl, tyrosinase, ephrin type-A receptor 2 precursor (EphA2), Fucosyl GM1, sLe, GM3, TGS5, HMWMAA, o-acetyl-GD2, Folate receptor beta, TEM1/CD248, TEM7R, CLDN6, TSHR, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-1a, MAGE-A1, MAGE A1, ETV6-AML, sperm protein 17, XAGE1, Tie 2, MAD-CT-1, MAD-CT-2, Fos-related antigen 1, p53, p53 mutant, prostein, survivin and telomerase, PCTA-1/Galectin 8, MelanA/MART1, Ras mutant, hTERT, sarcoma translocation breakpoints, ML-IAP, ERG (TMPRSS2 ETS fusion gene), NA17, PAX3, Androgen receptor, Cyclin B1, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, legumain, HPV E6, E7, intestinal carboxyl esterase, mut hsp70-2, CD79a, CD79b, CD72, LAIR1, FCAR, LILRA2, CD300LF, CLEC12A, BST2, EMR2, LY75, glypican 3 (GPC3), FCRL5, or IGLL1. In further embodiments, a TAA as disclosed herein comprises, or consists essentially of, or yet further consists of an epitope from a protein or polypeptide as listed above. In yet further embodiments, the TAA comprises, or consists essentially of, or yet further consists of an epitope of BCMA. In some embodiments, the TAA comprises, or consists essentially of, or yet further consists of a protein or polypeptide of BCMA.

In some embodiments, the CAR is a tandem CAR, i.e., comprising an anti-GPRC5D antigen binding sequence (such as an anti-GPRC5D scFv) or an equivalent thereof and wherein the equivalent recognizes and binds GPRC5D linked to an anti-non-GPRC5D-TAA antigen binding sequence (such as an anti-non-GPRC5D-TAA scFv) or an equivalent thereof and wherein the equivalent recognizes and binds the non-GPRC5D TAA optionally via a linker. In one embodiment, the anti-GPRC5D antigen binding sequence (such as an anti-GPRC5D scFv) or an equivalent thereof is at the N terminus side of the anti-non-GPRC5D-TAA antigen binding sequence (such as an anti-non-GPRC5D-TAA scFv) or an equivalent thereof. In one embodiment, the anti-GPRC5D antigen binding sequence (such as an anti-GPRC5D scFv) or an equivalent thereof is at the C terminus side of the anti-non-GPRC5D-TAA antigen binding sequence (such as an anti-non-GPRC5D-TAA scFv) or an equivalent thereof.

In some embodiments, the CAR is a looped tandem CAR. A looped tandem CAR comprises an anti-GPRC5D antigen binding sequence (such as an anti-GPRC5D scFv) comprising, or consisting essentially of, or yet further consisting an anti-GPRC5D heavy chain variable region and an anti-GPRC5D light chain variable region, and an anti-non-GPRC5D-TAA antigen binding sequence (such as an anti-non-GPRC5D-TAA scFv) comprising, or consisting essentially of, or yet further consisting an anti-non-GPRC5D-TAA heavy chain variable region and an anti-non-GPRC5D-TAA light chain variable region. In further embodiments, the CAR comprises starting from the N terminus: an anti-non-GPRC5D-TAA light/heavy chain variable region, an anti-GPRC5D light/heavy chain variable region, an anti-GPRC5D heavy/light chain variable region, and an anti-non-GPRC5D-TAA heavy/light chain variable region. In other embodiments, the CAR comprises starting from the N terminus: an anti-GPRC5D light/heavy chain variable region, an anti-non-GPRC5D-TAA light/heavy chain variable region, an anti-non-GPRC5D-TAA heavy/light chain variable region, and an anti-GPRC5D heavy/light chain variable region. In further embodiments, the CAR further comprises a linker between any two of the variable regions.

Additionally or alternatively, the polypeptide, or the CAR, or the bispecific antibody as disclosed herein further comprises one or both of: (iii) an amino acid sequence of a cytokine or (iv) an amino acid sequence of a suicide gene product or a detectable marker or both. In some embodiments, the cytokine is selected from IL15 or IL-21. In some embodiments, the cytokine promotes development, differentiation, activation, or expansion of one or more of: T cells, NK cells, NKT cells, another immune cell as disclosed herein, or a precursor cell of each thereof. In further embodiments, the cytokine is IL15, for example, a soluble IL15 as disclosed herein, a membrane bound IL15 as disclosed herein, a soluble IL-15 and IL-15 receptor complex (SIL15C) as disclosed herein (i.e., a soluble complex comprising, or consisting essentially of, or consisting of a soluble IL-15 and a soluble IL-15 receptor), a membrane bound complex comprising, or consisting essentially of, or consisting of a soluble IL-15 and a membrane bound IL-15 receptor, a membrane bound complex comprising, or consisting essentially of, or consisting of a membrane bound IL-15 and a soluble IL-15 receptor, or a membrane bound complex comprising, or consisting essentially of, or consisting of a membrane bound IL-15 and a membrane bound IL-15 receptor. In some embodiments, the cytokine is IL21, for example, a soluble IL21 as disclosed herein or a membrane bound IL21 as disclosed herein. In some embodiment, the suicide gene product or a detectable marker or both is a truncated epidermal growth factor receptor (tEGFR). In some embodiment, the suicide gene product or a detectable marker or both comprises, or consists essentially of, or yet further consists of RQR8.

In yet another aspect, provided is a polypeptide comprising, or consisting essentially of, or yet further consisting of (i) an amino acid sequence of a Chimeric Antigen Receptor (CAR), (ii) an amino acid sequence of a bispecific antibody, and one or both of (iii) an amino acid sequence of a cytokine or (iv) an amino acid sequence of a suicide gene product or a detectable marker or both. In some embodiments, the CAR is located at the N terminus side of the bispecific antibody. In other embodiments, the CAR is located at the C terminus side of the bispecific antibody. In some embodiments, the components of (i) (ii) (iii) and (iv) can be located in the polypeptide in any order, such as, from the N terminus to the C terminus, (i)— (ii)— (iii) if present— (iv) if present, (i)— (ii)— (iv) if present— (iii) if present, (i)— (iii) if present— (ii)— (iv) if present, (i)— (iii) if present-(iv) if present-(ii), (i)— (iv) if present-(ii)— (iii) if present, (i)— (iv) if present-(iii) if present-(ii), (ii)— (i)— (iii) if present— (iv) if present, (ii)— (i)— (iv) if present— (iii) if present, (ii)— (iii) if present— (i)— (iv) if present, (ii)— (iii) if present-(iv) if present-(i), (ii)— (iv) if present-(i)— (iii) if present, (ii)— (iv) if present-(iii) if present-(i), (iii) if present— (i)— (ii)— (iv) if present, (iii) if present— (i)— (iv) if present— (ii), (iii) if present— (ii)— (i)— (iv) if present, (iii) if present— (ii)— (iv) if present— (i), (iii) if present— (iv) if present— (i)— (ii), (iii) if present— (iv) if present— (ii)— (i), (iv) if present— (i)— (ii)— (iii) if present, (iv) if present— (i)— (iii) if present— (ii), (iv) if present— (ii)— (i)— (iii) if present, (iv) if present— (ii)— (iii) if present— (i), (iv) if present— (iii) if present— (i)— (ii), or (iv) if present— (iii) if present— (ii)— (i). The “—” as used herein can consists of any number (such as 0, 1, 2, 3, 4, 5, 6, 7, 8, 9, 10, 15, 20, 25, 30 or more) of amino acid residues and each amino acid residue can be any amino acid (i.e., random amino acid). In some embodiment, the “—” as used herein comprises, or consists essentially of, or yet further consists of one or more of: a peptide linker as disclosed herein, a marker as disclosed herein, a cleavable peptide, or a signal peptide as disclosed herein. In yet further embodiments, the polypeptide further comprises a signal peptide at its N terminus.

In some embodiments, the CAR of the polypeptide comprises, or consists essentially of, or yet further consists of (1) an antigen binding amino acid sequence that recognizes and binds a tumor associated antigen (TAA) (anti-TAA antigen binding sequence) or an equivalent thereof with the proviso that the equivalent recognizes and binds the TAA, (2) a hinge domain, (3) a transmembrane domain, and (4) an intracellular domain. In further embodiments, the TAA is B-cell maturation antigen (BCMA). Additionally or alternatively, the bispecific antibody comprises, or consists essentially of, or yet further consists of (1) an antigen binding sequence that recognizes and binds NKG2D (anti-NKG2D antigen binding sequence) or an equivalent thereof with the proviso that the equivalent recognizes and binds NKG2D, and (2) an antigen binding sequence that recognizes and binds G Protein-Coupled Receptor Class C Group 5 Member D (GPRC5D) (anti-GPRC5D antigen binding sequence) or an equivalent thereof and wherein the equivalent recognizes and binds GPRC5D.

In some embodiments, the CAR comprises, or consists essentially of, or yet further consists of (1) an antigen binding amino acid sequence that recognizes and binds a tumor associated antigen (TAA) (anti-TAA antigen binding sequence) or an equivalent thereof and wherein the equivalent recognizes and binds the TAA, (2) a hinge domain, (3) a transmembrane domain, and (4) an intracellular domain. In further embodiments, the TAA is G Protein-Coupled Receptor Class C Group 5 Member D (GPRC5D). Additionally or alternatively, the bispecific antibody comprises, or consists essentially of, or yet further consists of (1) an antigen binding sequence that recognizes and binds NKG2D (anti-NKG2D antigen binding sequence) or an equivalent thereof and wherein the equivalent recognizes and binds NKG2D, and (2) an antigen binding sequence that recognizes and binds B-cell maturation antigen (BCMA) (anti-BCMA antigen binding sequence) or an equivalent thereof and wherein the equivalent recognizes and binds BCMA.

In some embodiments, the cytokine is selected from IL15 or IL-21. In some embodiments, the cytokine promotes development, differentiation, activation, or expansion of one or more of: T cells, NK cells, NKT cells, another immune cell as disclosed herein, or a precursor cell of each thereof. In further embodiments, the cytokine is IL15, for example, a soluble IL15 as disclosed herein, a membrane bound IL15 as disclosed herein, a soluble IL-15 and IL-15 receptor complex (SIL15C) as disclosed herein, a membrane bound complex comprising, or consisting essentially of, or consisting of a soluble IL-15 and a membrane bound IL-15 receptor, a membrane bound complex comprising, or consisting essentially of, or consisting of a membrane bound IL-15 and a soluble IL-15 receptor, or a membrane bound complex comprising, or consisting essentially of, or consisting of a membrane bound IL-15 and a membrane bound IL-15 receptor. In some embodiments, the cytokine is IL21, for example, a soluble IL21 as disclosed herein or a membrane bound IL21 as disclosed herein. In some embodiment, the suicide gene product or a detectable marker or both is a truncated epidermal growth factor receptor (tEGFR). In some embodiment, the suicide gene product or a detectable marker or both comprises, or consists essentially of, or yet further consists of RQR8.

In some embodiments of any aspect as disclosed herein, the anti-NKG2D antigen binding sequence is located at the N terminus side of the anti-TAA antigen binding sequence of the CAR or the bispecific antibody. In some embodiments of any aspect as disclosed herein, the anti-NKG2D antigen binding sequence is located at the C terminus side of the anti-TAA antigen binding sequence of the CAR or the bispecific antibody. In further embodiments, the anti-NKG2D antigen binding sequence is linked to the anti-TAA antigen binding sequence of the CAR or the bispecific antibody via one or more random amino acid residue(s), a peptide linker as disclosed herein, such as an HMA linker, a fragment crystallizable (Fc) region of an immunoglobulin, a mutant thereof, or an equivalent thereof, or any combination thereof.

In some embodiments of any aspect as disclosed herein, either or both of the antigen binding sequence of a bispecific antibody is a single-chain variable fragment (scFv). In some embodiments, the anti-NKG2D scFv is located at the N terminus side of the anti-TAA scFv. In some embodiments, the anti-NKG2D scFv is located at the C terminus side of the anti-TAA scFv. In further embodiments, the anti-NKG2D scFv is linked to the anti-TAA scFv via one or more random amino acid residue(s), a peptide linker, such as an HMA linker, or a fragment crystallizable (Fc) region of an immunoglobulin, a mutant thereof, or an equivalent thereof, or any combination thereof. In some embodiments, the anti-NKG2D scFv is replaced with an scFv that recognizes and binds to an antigen which is not NKG2D but also present on the cell surface of an immune cell as disclosed herein, such as a T cell, an NK cell, or an NKT cell. Suitable antigens are described herein.

In some embodiments of any aspect as disclosed herein, anti-NKG2D antigen binding sequence comprises, or consists essentially of, or yet further consists of one or two or three or four or five or all six of the following complementarity-determining regions (CDRs): a light chain complementarity-determining region 1 (CDRL1) comprising SGSSSNIGNNAVN (SEQ ID NO: 1) or an equivalent thereof; a light chain complementarity-determining region 2 (CDRL2) comprising YDDLLPS (SEQ ID NO: 2) or an equivalent thereof; a light chain complementarity-determining region 3 (CDRL3) comprising AAWDDSLNGPV (SEQ ID NO: 3) or an equivalent thereof; a heavy chain complementarity-determining region 1 (CDRH1) comprising GFTFSSY (SEQ ID NO: 4) or an equivalent thereof; a heavy chain complementarity-determining region 2 (CDRH2) comprising RYDGSN (SEQ ID NO: 5) or an equivalent thereof; and a heavy chain complementarity-determining region 3 (CDRH3) comprising DRGLGDGTYFDY (SEQ ID NO: 6) or an equivalent thereof. In further embodiments, the equivalents thereof recognizes and binds NKG2D.

In some embodiments of any aspect as disclosed herein, the anti-NKG2D antigen binding sequence comprises, or consists essentially of, or yet further consists of a light chain variable region comprising SEQ ID NO: 7, or an equivalent thereof; or a heavy chain variable region comprising SEQ ID NO: 8, or an equivalent thereof; or both the light chain variable region or an equivalent thereof and the heavy chain variable region or an equivalent thereof. In further embodiments, the equivalent thereof recognizes and binds NKG2D.

In some embodiments of any aspect as disclosed herein, the equivalent of SEQ ID NO: 7 or 8 is at least 80%, or at least 85%, or at least 90%, or at least 95%, or at least 96%, or at least 97%, or at least 98%, or at least 99% identical to SEQ ID NO: 7 or 8, respectively. In some embodiments, the equivalent of SEQ ID NOs: 1-8 comprises, or consists essentially of, or yet further consists of the amino acid sequence of C terminus to N terminus of SEQ ID NO: 1-8, respectively.

In some embodiments of any aspect as disclosed herein, the anti-NKG2D antigen binding sequence further comprises a peptide linker between the light chain variable region or the equivalent thereof and the heavy chain variable region or the equivalent thereof. In further embodiments, the anti-NKG2D antigen binding sequence further comprises a peptide linker comprising, or consisting essentially of, or yet further consisting of SEQ ID NO: 9 or an equivalent thereof between the light chain variable region or the equivalent thereof and the heavy chain variable region or the equivalent thereof.

In some embodiments of any aspect as disclosed herein, the anti-GPRCSD antigen binding sequence comprises, or consists essentially of, or yet further consists of one or two or three or four or five or all six of the following CDRs: a CDRL1 comprising KASQNVATHVG (SEQ ID NO: 10) or an equivalent thereof; a CDRL2 comprising SASYRYS (SEQ ID NO: 11) or an equivalent thereof; a CDRL3 comprising QQYNRYPYT (SEQ ID NO: 12) or an equivalent thereof; a CDRH1 comprising GYSFTGY (SEQ ID NO: 13) or an equivalent thereof; a CDRH2 comprising NPYNSD (SEQ ID NO: 14) or an equivalent thereof; and a CDRH3 comprising VALRVALDY (SEQ ID NO: 15) or an equivalent thereof. In further embodiments, the equivalents thereof recognizes and binds GPRC5D.

In some embodiments of any aspect as disclosed herein, the anti-GPRC5D antigen binding sequence comprises, or consists essentially of, or yet further consists of a light chain variable region comprising SEQ ID NO: 16, or an equivalent thereof; or a heavy chain variable region comprising SEQ ID NO: 17, or an equivalent thereof; or both the light chain variable region or an equivalent thereof and the heavy chain variable region or an equivalent of one or both thereof. In further embodiments, the equivalents thereof recognizes and binds GPRC5D.

In some embodiments of any aspect as disclosed herein, the equivalent of SEQ ID NO: 16 or 17 is at least 80%, or at least 85%, or at least 90%, or at least 95%, or at least 96%, or at least 97%, or at least 98%, or at least 99% identical to SEQ ID NO: 16 or 17, respectively.

In some embodiments of any aspect as disclosed herein, the equivalent of SEQ ID NOs: 10-17 comprises, or consists essentially of, or yet further consists of the amino acid sequence of C terminus to N terminus of SEQ ID NOs: 10-17, respectively.

In some embodiments of any aspect as disclosed herein, the anti-GPRC5D antigen binding sequence further comprises a peptide linker between the light chain variable region or the equivalent thereof and the heavy chain variable region or the equivalent thereof. In further embodiments, the anti-GPRC5D antigen binding sequence further comprises a peptide linker comprising, or consisting essentially of, or yet further consisting of SEQ ID NO: 9 or an equivalent thereof between the light chain variable region or the equivalent thereof and the heavy chain variable region or the equivalent thereof.

In some embodiments of any aspect as disclosed herein, the bispecific antibody further comprises a fragment crystallizable (Fc) region of an immunoglobulin, a mutant thereof, or an equivalent thereof. In one embodiment, the Fc region or a mutant thereof is a human Fc region or a mutant thereof. In some embodiments, the Fc region comprises, or consists essentially of, or yet further consists of SEQ ID NO: 18, or an equivalent thereof. In one embodiment, the equivalent also substantially stabilizes the bispecific antibody as SEQ ID NO: 18, for example for secretion. Additionally or alternatively, the equivalent binds an Fc receptor on an immune cell or on a platelet or that binds a complement protein, optionally in a level substantially similar to that of SEQ ID NO: 18. In some embodiments, the equivalent binds an Fc receptor on an immune cell or on a platelet or that binds a complement protein, optionally in a level substantially less compared to that of SEQ ID NO: 18. In some embodiments, strong binding to an Fc gamma receptor (FcγR) is not desirable in view of in vivo adverse events including the induction of pro-inflammatory cytokines (i.e., cytokine storm). In order to reduce this unintended effector function, an Fc region equivalent may comprises one or more of mutations, which reduced inflammatory cytokine release. In some embodiments, such as those relating to an antibody comprising an Fc fragment and expressed by an immune cell, an Fc fragment (for example an IgG4 Fc fragment) with mutation(s) is used in order to reduce or avoid antibody-dependent cellular cytotoxicity (ADCC) and complement-dependent cytotoxicity (CDC). In further embodiments, the immune cell is an NK cell. Without wishing to be bound by the theory, retaining the capability of an antibody or an Fc fragment thereof in inducing ADCC or CDC may lead to fratricide of the immune cells, such as NK self-killing or NK-depletion. Additionally or alternatively, in some embodiments, the ADCC or CDC is reduced or avoided by a method comprising mutating one or more amino acid residues in the Fc part of the antibody and reducing its binding to a complement or an Fc receptor. One example used herein is a three-site mutated human IgG4 Fc. IgG4-Fc had been mutated at three sites, F234A, L235A within hinge region and N297Q within CH2 region, substitutions in its Fc region were aimed to avoid the Fc receptors binding. See, for example, Wang et al. Protein Cell. 2018 January;9(1):63-73. Epub 2017 Oct 6. In some embodiments, the Fc region comprises SEQ ID NO: 19 or an Fc equivalent having mutations at a position corresponding to amino acid (aa) 16, aa 17 and aa 79 of SEQ ID NO: 19. In one embodiment, the equivalent also substantially stabilizes the bispecific antibody as SEQ ID NO: 19, for example for secretion. Additionally or alternatively, the equivalent binds an Fc receptor on an immune cell or on a platelet or that binds a complement protein, optionally in a level substantially similar to that of SEQ ID NO: 19. In some embodiments, the equivalent binds an Fc receptor on an immune cell or on a platelet or that binds a complement protein, optionally in a level substantially less compared to that of SEQ ID NO: 19. In some embodiments, an Fc or an equivalent thereof as used herein reduces or does not induce antibody-dependent cellular cytotoxicity (ADCC). Additionally or alternatively, an Fc or an equivalent thereof as used herein reduces or does not induce complement-dependent cytotoxicity (CDC). In some embodiments, the equivalent binds an Fc receptor or a complement protein in a level substantially (such as about 10%, or about 20%, or about 30%, or about 40%, or about 50%, or about 60%, or about 70%, or about 80%, or about 90%, or about 95%, or more) less compared to that of SEQ ID NO: 19 or at a level similar (such as about 90% to about 110% or any ranges or percentage in between) to that of SEQ ID NO:19. Non-limiting examples of such Fc or an equivalent thereof are disclosed herein, or for example, as shown in WO2018107058A1.

In some embodiments of any aspect as disclosed herein, a bispecific antibody as disclosed herein comprises, or consists essentially of, or yet further consists of, optionally from N terminus to C terminus, a first antibody binding sequence, an optional first linker, an optional first Fc region, an optional second linker, a second antibody binding sequence, an optional third linker, and an optional second Fc region. In some embodiments of any aspect as disclosed herein, a bispecific antibody as disclosed herein comprises, or consists essentially of, or yet further consists of, optionally from N terminus to C terminus, a first antibody binding sequence, an optional first linker, a second antibody binding sequence, an optional second linker, and an optional Fc region. In some embodiments of any aspect as disclosed herein, a bispecific antibody as disclosed herein comprises, or consists essentially of, or yet further consists of, optionally from N terminus to C terminus, a first antibody binding sequence, an optional first linker, an optional Fc region, an optional second linker, and a second antibody binding sequence. In further embodiments, the second antibody binding sequence may comprises, or consists essentially of, or yet further consists of an equivalent comprising, or consisting essentially of, or yet further consisting of an amino acid sequence of C terminus to N terminus of an antibody binding sequence as disclosed herein. In another word, the second antibody binding sequence may be flipped while constructing or engineering the bispecific antibody. In some embodiments, the first antibody binding sequence is anti-GPRCSD and optionally the second antibody binding sequence is anti-NKG2D. In some embodiments, the second antibody binding sequence is anti-GPRCSD and optionally the first antibody binding sequence is anti-NKG2D. Additionally or alternatively, any of the linkers may be selected from the ones as disclosed herein, such as a (GGGGS)n peptide linker, wherein n is any one of 1, 2, 3, 4, 5, 6, 7, 8, 9, 10, 11, 12, 13,14 of 15 (SEQ ID NO: 130), an HMA peptide linker, a cleavable peptide, or any combination thereof.

In some embodiments of any aspect as disclosed herein, an antigen binding sequence may be an antibody or a fragment thereof, such as an antigen binding domain, a variable region, an antigen binding fragment (Fab), an scFv, a di-scFv, a diabody, a triabody, a minibody, an Fab′, an F(ab′)₂, an F(ab)₂, an scFv-Fc, an scFv-CH, or an scFab.

In some embodiments of any aspect as disclosed herein, the bispecific antibody further comprises a peptide linker between the anti-NKG2D antigen binding sequence and the anti-GPRCSD antigen binding sequence. In further embodiments, the peptide linker comprises, or consists essentially of, or yet further consists of a human muscle aldolase (HMA) linker, such as PSGQAGAAASESLFVSNHAY (SEQ ID NO: 81), or an equivalent thereof.

In some embodiments of any aspect as disclosed herein, the CAR further comprises a signal peptide, optionally at its N terminus. In further embodiments, the signal peptide is suitable for directing the CAR to cell membrane. In yet further embodiments, the signal peptide comprises, or consists essentially of, or yet further consists of MGWSSIILFLVATATGVH (SEQ ID NO: 21), or an equivalent thereof that directs the CAR to cell membrane. In one embodiment, the signal peptide comprises, or consists essentially of, or yet further consists of MYRMQLLSCIALSLALVTNS (SEQ ID NO: 20), or MWLQSLLLLGTVACSIS (SEQ ID NO: 122), or MRISKPHLRSISIQCYLCLLLNSHFLTEA (SEQ ID NO: 123), or MRSSPGNMERIVICLMVIFLGTLV (SEQ ID NO: 124), or an equivalent of each thereof that directs the CAR to cell membrane. In some embodiments, the signal peptide comprises, or consists essentially of, or yet further consists of MYRMQLLSCIALSLALVTNS (SEQ ID NO: 20), or MWLQSLLLLGTVACSIS (SEQ ID NO: 122), or MRISKPHLRSISIQCYLCLLLNSHFLTEA (SEQ ID NO: 123), or MRSSPGNMERIVICLMVIFLGTLV (SEQ ID NO: 124), or MGWSSIILFLVATATGVH (SEQ ID NO: 21), or MLLLVTSLLLCELPHPAFLLIP (SEQ ID NO: 125) or an equivalent of each thereof that directs the CAR to cell membrane.

In some embodiments of any aspect as disclosed herein, the bispecific antibody further comprises a signal peptide, optionally at its N terminus. In further embodiments, the signal peptide is suitable for directing secretion of the antibody. In yet further embodiments, the signal peptide comprises, or consists essentially of, or yet further consists of MGWSSIILFLVATATGVH (SEQ ID NO: 21), or an equivalent thereof that facilitates secretion of the bispecific antibody, fragment or equivalent thereof. In one embodiment, the signal peptide comprises, or consists essentially of, or yet further consists of MYRMQLLSCIALSLALVTNS (SEQ ID NO: 20), or MWLQSLLLLGTVACSIS (SEQ ID NO: 122), or MRISKPHLRSISIQCYLCLLLNSHFLTEA (SEQ ID NO: 123), or MRSSPGNMERIVICLMVIFLGTLV (SEQ ID NO: 124), or an equivalent of each thereof that facilitates secretion of the bispecific antibody, fragment or equivalent thereof. In some embodiments, the signal peptide comprises, or consists essentially of, or yet further consists of MYRMQLLSCIALSLALVTNS (SEQ ID NO: 20), or MWLQSLLLLGTVACSIS (SEQ ID NO: 122), or MRISKPHLRSISIQCYLCLLLNSHFLTEA (SEQ ID NO: 123), or MRSSPGNMERIVICLMVIFLGTLV (SEQ ID NO: 124), or MGWSSIILFLVATATGVH (SEQ ID NO: 21), or MLLLVTSLLLCELPHPAFLLIP (SEQ ID NO: 125) or an equivalent of each thereof that facilitates secretion of the bispecific antibody, fragment or equivalent thereof.

In some embodiments, a signal peptide as disclosed herein comprises, or consists essentially of, or yet further consists of any one or more of: SEQ ID NO: 20, SEQ ID NO: 21, MTRLTVLALLAGLLASSRA (SEQ ID NO: 126), or an equivalent of each thereof.

In some embodiments of any aspect as disclosed herein, the bispecific antibody further comprises a detectable or purification marker. In further embodiments, the detectable marker comprises, or consists essentially of, or yet further consists of

(SEQ ID NO: 22)
YPYDVPDYA.

In some embodiments as disclosed herein, the TAA is not GPRCSD. In further embodiments, the TAA comprises, or consists essentially of, or yet further consists of an epitope from a protein or polypeptide selected from: B-cell maturation antigen (BCMA), SLAMF7 (CS1 or CD319), EGFR, wildtype epidermal growth factor receptor (EGFRwt), epidermal growth factor receptor variant III (EGFRVIII), FLT3, CD70, mesothelin, CD123, CD19, carcinoembryonic antigen (CEA), CD133, human epidermal growth factor receptor 2 (HER2), ERBB2 (Her2/neu), CD22, CD30, CD171, CLL-1 (CLECL1), GTPase-activating protein (GAP), CDS, interleukin 13 receptor alpha 2 (IL13Ra2), guanylyl cyclase C (GUCY2C), tumor-associated glycoprotein-72 (TAG-72), thymidine kinase 1 (TK1), hypoxanthine guanine phosphoribosyltransferase (HPRT1), cancer/testis (CT), CD33, ganglioside G2 (GD2), GD3, Tn Ag, prostate specific membrane antigen (PSMA), receptor tyrosine kinase-like orphan receptor 1 (ROR1), TAG72, CD38, CD44v6, epithelial cell adhesion molecule precursor (EpCam or EPCAM), B7H3, KIT, IL-13Ra2, IL-11Ra, prostate stem cell antigen (PSCA), PRSS21, vascular endothelial growth factor receptor 2 (VEGFR2), LewisY, CD24, PDGFR-beta, SSEA-4, CD20, Folate receptor alpha, mucin 1 (Muc1), NCAM, Prostase, PAP, ELF2M, Ephrin B2, fibroblast activation protein alpha (FAP), IGF-I receptor, CAIX, LMP2, gp100, bcr-abl, tyrosinase, ephrin type-A receptor 2 precursor (EphA2), Fucosyl GM1, sLe, GM3, TGS5, HMWMAA, o-acetyl-GD2, Folate receptor beta, TEM1/CD248, TEM7R, CLDN6, TSHR, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-1a, MAGE-A1, MAGE Al, ETV6-AML, sperm protein 17, XAGE1, Tie 2, MAD-CT-1, MAD-CT-2, Fos-related antigen 1, p53, p53 mutant, prostein, survivin and telomerase, PCTA-1/Galectin 8, MelanA/MART1, Ras mutant, hTERT, sarcoma translocation breakpoints, ML-IAP, ERG (TMPRSS2 ETS fusion gene), NA17, PAX3, Androgen receptor, Cyclin B1, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, legumain, HPV E6, E7, intestinal carboxyl esterase, mut hsp70-2, CD79a, CD79b, CD72, LAIR1, FCAR, LILRA2, CD300LF, CLEC12A, BST2, EMR2, LY75, glypican 3 (GPC3), FCRL5, or IGLL1. In further embodiments, the TAA comprises, or consists essentially of, or yet further consists of an epitope of BCMA.

In some embodiments of any aspect as disclosed herein, the antigen binding sequence that recognizes and binds BCMA (anti-BCMA antigen binding sequence) comprises, or consists essentially of, or yet further consists of one or two or three or four or five or six of the following CDRs: a CDRL1 comprising a sequence selected from RASESVTILGSHLIH (SEQ ID NO: 23), SASQDISNYLN (SEQ ID NO: 24), RASESVTILGSHLIY (SEQ ID NO: 25), or an equivalent of each thereof; a CDRL2 comprising a sequence selected from LASNVQT (SEQ ID NO: 26), YTSNLHS (SEQ ID NO: 27), LASNVQT (SEQ ID NO: 28), or an equivalent of each thereof; a CDRL3 comprising a sequence selected from LQSRTIPRT (SEQ ID NO: 29), QQYRKLPWT (SEQ ID NO: 30), LQSRTIPRT (SEQ ID NO: 31), an equivalent of each thereof; a CDRH1 comprising a sequence selected from GYTFTDY (SEQ ID NO: 32), GGTFSNY (SEQ ID NO: 33), GYTFRHY (SEQ ID NO: 34), or an equivalent of each thereof; a CDRH2 comprising a sequence selected from INTETRE (SEQ ID NO: 35), YRGHSD (SEQ ID NO: 36), NTESGV (SEQ ID NO: 37), or an equivalent of each thereof; and a CDRH3 comprising a sequence selected from DYSYAMDY (SEQ ID NO: 38), GAIYNGYDVLDN (SEQ ID NO: 39), DYLYSLDF (SEQ ID NO: 40), or an equivalent of each thereof.

In some embodiments, the equivalents thereof recognizes and binds BCMA. In some embodiments, the anti-BCMA antigen binding sequence comprises, or consists essentially of, or yet further consists of a light chain variable region comprising a sequence selected from SEQ ID NO: 41, SEQ ID NO: 42, SEQ ID NO: 43, or an equivalent of each thereof or a heavy chain variable region comprising a sequence selected from SEQ ID NO: 44, SEQ ID NO: 45, SEQ ID NO: 46, or an equivalent of each thereof; or both the light chain variable region or an equivalent thereof and the heavy chain variable region or an equivalent thereof or equivalents of both. In some embodiments, the equivalents thereof recognizes and binds BCMA.

In some embodiments of any aspect as disclosed herein, the equivalent of SEQ ID NOs: 41 to 46 is at least 80%, or at least 85%, or at least 90%, or at least 95%, or at least 96%, or at least 97%, or at least 98%, or at least 99% identical to SEQ ID NOs: 41 to 46, respectively. In some embodiments, the equivalent of SEQ ID NOs: 23-46 comprises, or consists essentially of, or yet further consists of the amino acid sequence of C terminus to N terminus of SEQ ID NOs: 23-46, respectively.

In some embodiments of any aspect as disclosed herein, the anti-BCMA antigen binding sequence or an equivalent thereof further comprises a peptide linker between the light chain variable region or the equivalent thereof and the heavy chain variable region or the equivalent thereof. In further embodiments, the anti-BCMA antigen binding sequence further comprises a peptide linker comprising, or consisting essentially of, or yet further consisting of SEQ ID NO: 9 or an equivalent thereof between the light chain variable region or the equivalent thereof and the heavy chain variable region or the equivalent thereof.

In some embodiments of any aspect as disclosed herein, the amino acid sequence of the cytokine comprises, or consists essentially of, or yet further consists of at least one of: SEQ ID NO: 106, SEQ ID NO: 104, SEQ ID NO: 116, SEQ ID NO: 117, SEQ ID NO: 118, or an equivalent of each thereof.

In some embodiments of any aspect as disclosed herein, the amino acid sequence of the cytokine comprises, or consists essentially of, or yet further consists of SEQ ID NO: 106 or an equivalent thereof.

In some embodiments of any aspect as disclosed herein, the amino acid sequence of the cytokine comprises, or consists essentially of, or yet further consists of SEQ ID NO: 106 and SEQ ID NO: 107 or an equivalent of each thereof. In further embodiments, the two sequences are linked by a linker, such as a peptide linker as disclosed herein. In some embodiments, the peptide linker comprises, or consists essentially of, or consists of

(SEQ ID NO: 108)
SGGGSGGGGSGGGGSGGGGSGGGSLQ.

In some embodiments, the cytokine further comprises a signal peptide. In some embodiments, the signal peptide is suitable for secreting the soluble cytokine out of the cell expressing the cytokine. In other embodiments, the signal peptide is suitable for directing the membrane bound cytokine to locate in the target membrane, such as cell membrane. In some embodiments, the signal peptide comprises, or consists essentially of, or yet further consists of MYRMQLLSCIALSLALVTNS (SEQ ID NO: 20), MWLQSLLLLGTVACSIS (SEQ ID NO: 131), MRISKPHLRSISIQCYLCLLLNSHFLTEA (SEQ ID NO: 123), MRSSPGNMERIVICLMVIFLGTLV (SEQ ID NO: 124), or an equivalent of each thereof. In some embodiments, the signal peptide comprises, or consists essentially of, or yet further consists of MGWSSIILFLVATATGVH (SEQ ID NO: 21), MLLLVTSLLLCELPHPAFLLIP (SEQ ID NO: 125), or an equivalent of each thereof. In some embodiments, the signal peptide comprises, or consists essentially of, or yet further consists of MYRMQLLSCIALSLALVTNS (SEQ ID NO: 20), or MWLQSLLLLGTVACSIS (SEQ ID NO: 122), or MRISKPHLRSISIQCYLCLLLNSHFLTEA (SEQ ID NO: 123), or MRSSPGNMERIVICLMVIFLGTLV (SEQ ID NO: 124), or MGWSSIILFLVATATGVH (SEQ ID NO: 21), or MLLLVTSLLLCELPHPAFLLIP (SEQ ID NO: 125) or an equivalent of each thereof.

In some embodiments, the tEGFR comprises, or consists essentially of, or yet further consists of a sequence of SEQ ID NO: 120 or an equivalent thereof. In some embodiments, the tEGFR comprises, or consists essentially of, or yet further consists of aa 67 to aa 401 of the human epidermal growth factor receptor (EGFR) isoform i precursor or another fragment of the EGFR isoform I precursor, optionally comprising the extracellular domain(s) of the protein. In some embodiments, the sequence of an EGFR isoform I precursor is as disclosed in NP_001333870.1.

In some embodiments, the tEGFR further comprises a signal peptide. In some embodiments, the signal peptide is suitable for directing the tEGFR to locate in the cell membrane. In some embodiments, the signal peptide comprises, or consists essentially of, or yet further consists of a peptide of the group: MYRMQLLSCIALSLALVTNS (SEQ ID NO: 20), MWLQSLLLLGTVACSIS (SEQ ID NO: 122), MRISKPHLRSISIQCYLCLLLNSHFLTEA (SEQ ID NO: 123), MRSSPGNMERIVICLMVIFLGTLV (SEQ ID NO: 124), or an equivalent of each thereof that directs the tEGFR to locate in the cell membrane. In some embodiments, the signal peptide comprises, or consists essentially of, or yet further consists of MGWSSIILFLVATATGVH (SEQ ID NO: 21) or MLLLVTSLLLCELPHPAFLLIP (SEQ ID NO: 125), or an equivalent of each thereof that directs the tEGFR to locate in the cell membrane. In some embodiments, the signal peptide comprises, or consists essentially of, or yet further consists of MYRMQLLSCIALSLALVTNS (SEQ ID NO: 20), or MWLQSLLLLGTVACSIS (SEQ ID NO: 122), or MRISKPHLRSISIQCYLCLLLNSHFLTEA (SEQ ID NO: 123), or MRSSPGNMERIVICLMVIFLGTLV (SEQ ID NO: 124), or MGWSSIILFLVATATGVH (SEQ ID NO: 21), or MLLLVTSLLLCELPHPAFLLIP (SEQ ID NO: 125) or an equivalent of each thereof that directs the tEGFR to locate in the cell membrane.

In some embodiment, the suicide gene product or a detectable marker or both comprises, or consists essentially of, or yet further consists of RQR8.

In some embodiments, the RQR8 comprises, or consists essentially of, or yet further consists of SEQ ID NO: 132 or an equivalent thereof.

In some embodiments, the amino acid sequence of any one or more of (i) to (iv) further comprises a detectable marker.

In some embodiments, the polypeptide further comprises a cleavable peptide located between any two of (i), (ii), (iii) and (iv), if present. In one embodiment, the cleavable peptide is a self-cleaving peptide. In a further embodiment, the self-cleaving peptide is selected from a T2A peptide or a P2A. In yet a further embodiment, cleavable peptide or self-cleaving peptide comprises, or consists essentially of, or yet further consists of a peptide selected from HVGSGEGRGSLLTCGDVEENPGP (SEQ ID NO: 50), GSGEGRGSLLTCGDVEENPGP (aa 3 to aa 23 of SEQ ID NO: 50), GSGATNFSLLKQAGDVEENPGP (SEQ ID NO: 133), EGRGSLLTCGDVEENPGP (SEQ ID NO: 134), AEGRGSLLTCGDVEENPGP (SEQ ID NO: 135), or an equivalent of each thereof.

In some embodiments, the polypeptide, or the CAR, or the bispecific antibody comprises, or consists essentially of, or yet further consists of one or more of amino acid sequences provided in any one or more of Tables 1a-1h, Tables 2a-2x, and Tables 3a-3d. In some embodiments, the polypeptide, or the CAR, or the bispecific antibody comprises, or consists essentially of, or yet further consists of one or more of amino acid sequences encoded by a nucleotide sequence provided in any one or more of Tables 1a-1h, Tables 2a-2x, and Tables 3a-3d. In further embodiments, the polypeptide as disclosed herein comprises, or consists essentially of, or yet further consists of an amino acid sequence provided in any one of Tables 1b-1h, Tables 2b-2x, and Tables 3a-3d. In yet further embodiments, the polypeptide as disclosed herein comprises, or consists essentially of, or yet further consists of an amino acid sequence encoded by a nucleotide sequence provided in any one of Tables 1b-1h, Tables 2b-2x, and Tables 3a-3d. In some embodiments, the polypeptide as disclosed herein comprises, or consists essentially of, or yet further consists of one or more of the following sequences or a fragment thereof: amino acid sequences of P4 to P9, P11E, P12E, P19, P20, P61, P62, or P72 as disclosed herein.

In some embodiments, the polypeptide comprises, or consists essentially of, or consists of (1) a signal peptide as disclosed herein, such as a signal peptide comprising, or consisting essentially of, or consisting of an amino acid sequence of MGWSSIILFLVATATGVH (SEQ ID NO: 21) or an equivalent thereof; (2) an anti-BCMA antigen binding sequence as disclosed herein, such as the one comprising, or consisting essentially of, or consisting of (2-i) a light chain variable region that comprises, or consists essentially of, or consists of SEQ ID NO: 41 or SEQ ID NO: 42 or an equivalent of each thereof, (2-ii) an optional peptide linker as disclosed herein for example that comprises, or consists essentially of, or further consists of SEQ ID NO: 9 or an equivalent thereof, and (2-iii) a heavy chain variable region that comprises, or consists essentially of, or consists of SEQ ID NO: 44 or SEQ ID NO: 45 or an equivalent of each thereof, and wherein the equivalents of the variable regions recognize and bind BCMA); (3) a hinge domain as disclosed herein, such as the one comprising, or consisting essentially of, or consisting of LEPKSCDKTHTCPPCPDPKGT (SEQ ID NO: 47) or an equivalent thereof; (4) a transmembrane domain as disclosed herein, such as a CD28 transmembrane domain comprising, or consisting essentially of, or consisting of FWVLVVVGGVLACYSLLVTVAFIIFWV (SEQ ID NO: 91) or an equivalent thereof; (5) a costimulatory signaling region as disclosed herein, such as a CD28 costimulatory signaling region comprising, or consisting essentially of, or consisting of RSKRSRLLHSDYMNMTPRRPGPTRKHYQPYAPPRDFAAYRS (SEQ ID NO: 95) or an equivalent thereof; (6) an CD3 zeta signaling domain as disclosed herein, such as the one comprising, or consisting essentially of, or consisting of RVKFSRSADAPAYQQGQNQLYNELNLGRREEYDVLDKRRGRDPEMGGKPRRKNP QEGLYNELQKDKMAEAYSEIGMKGERRRGKGHDGLYQGLSTATKDTYDALHMQ ALPPR (SEQ ID NO: 49) or an equivalent thereof; (7) a cleaving peptide as disclosed herein, such as a T2A self-cleaving peptide comprising, or consisting essentially of, or consisting of HVGSGEGRGSLLTCGDVEENPGP (SEQ ID NO: 50) or an equivalent thereof; (8) a signal peptide as disclosed herein, such as a signal peptide comprising, or consisting essentially of, or consisting of MYRMQLLSCIALSLALVTNS (SEQ ID NO: 20) or an equivalent thereof; (9) an anti-GPRCSD antigen binding sequence as disclosed herein, such as the one comprising, or consisting essentially of, or consisting of (9-i) a light chain variable region that comprises, or consists essentially of, or consists of SEQ ID NO: 16, or an equivalent thereof, (9-ii) an optional peptide linker as disclosed herein for example that comprises, or consists essentially of, or consists of SEQ ID NO: 9 or an equivalent thereof, and (9-iii) a heavy chain variable region that comprises, or consists essentially of, or consists of SEQ ID NO: 17, or an equivalent thereof, wherein the equivalents of the variable regions recognize and bind GPRCSD; (10) an optional linker as disclosed herein, such as a human muscle aldolase (HMA) linker comprising, or consisting essentially of, or consisting of PSGQAGAAASESLFVSNHAY (SEQ ID NO: 81) or an equivalent thereof; (11) an anti-NKG2D antigen binding sequence as disclosed herein, such as the one comprising, or consisting essentially of, or consisting of (11-i) a light chain variable region that comprises, or consists essentially of, or consists of SEQ ID NO: 7 or an equivalent thereof, (11-ii) an optional peptide linker as disclosed herein for example that comprises, or consists essentially of, or consists of SEQ ID NO: 9 or an equivalent thereof, and (11-iii) a heavy chain variable region that comprises, or consists essentially of, or consists of SEQ ID NO: 8 or an equivalent thereof, wherein the equivalents of the variable regions recognize and bind NKG2D; (12) an Fc region as disclosed herein, such as the one comprising, or consisting essentially of, or consisting of SEQ ID NO: 19 or an equivalent thereof; (13) a cleaving peptide as disclosed herein, such as a T2A self-cleaving peptide comprising, or consisting essentially of, or consisting of EGRGSLLTCGDVEENPGP (SEQ ID NO: 134) or an equivalent thereof; (14) a signal peptide as disclosed herein, such as the one comprising, or consisting essentially of, or consisting of MYRMQLLSCIALSLALVTNS (SEQ ID NO:20) or MRISKPHLRSISIQCYLCLLLNSHFLTEA (SEQ ID NO: 123) or MWLQSLLLLGTVACSIS (SEQ ID NO: 122) or an equivalent of each thereof; (15) an amino acid sequence of the cytokine as disclosed herein, such as the one comprising, or consisting essentially of, or consisting of at least one of SEQ ID NO: 106, SEQ ID NO: 104, SEQ ID NO: 117, SEQ ID NO: 118, SEQ ID NO: 116, or an equivalent of each thereof; (16) a cleaving peptide as disclosed herein, such as a P2A self-cleaving peptide comprising, or consisting essentially of, or consisting of GSGATNFSLLKQAGDVEENPGP (SEQ ID NO: 133) or an equivalent thereof; (17) a signal peptide as disclosed herein, such as the one comprising, or consisting essentially of, or consisting of MLLLVTSLLLCELPHPAFLLIP (SEQ ID NO: 125) or an equivalent thereof and (18) a suicide gene product or a detectable marker or both as disclosed herein, such as the one comprising, or consisting essentially of, or consisting of SEQ ID NO: 120 or an equivalent thereof. In further embodiments, the amino acid sequence of the cytokine further comprises a transmembrane domain, for example the one comprising, or consisting essentially of, or consisting of AVGQDTQEVIVVPHSLPFKVVVISAILALVVLTIISLIILIMLWQKKPR (SEQ ID NO: 119) or an equivalent thereof. In yet further embodiments, the amino acid sequence of the cytokine further comprises a cytokine receptor, such as an IL15 receptor, or a fragment thereof. In some embodiments, the amino acid sequence of the cytokine comprises, or consists essentially of, or consists of SEQ ID NO: 106 or an equivalent thereof, an optional peptide linker as disclosed herein, such as the one comprising, or consisting essentially of, or consisting of SGGGSGGGGSGGGGSGGGGSGGGSLQ (SEQ ID NO: 108) or SEQ ID NO: 9 or an equivalent of each thereof, and ITCPPPMSVEHADIWVKSYSLYSRERYICNSGFKRKAGTSSLTECVLNKATNVAHW TTPSLKCIR (SEQ ID NO: 107) or an equivalent thereof. In some embodiments, the parts or components of the polypeptide are provided or numbered in this paragraph without indicating any arrangement or order in the polypeptide. In some embodiments, the parts or components of the polypeptide are provided or numbered in this paragraph in an order from the N terminus to the C terminus of the polypeptide (i.e., arranged in the polypeptide from the N terminus to the C terminus with or without other additional sequences). In other embodiments, the parts or components of the polypeptide are provided or numbered in this paragraph in an order from the C terminus to the N terminus of the polypeptide (i.e., arranged in the polypeptide from the C terminus to the N terminus with or without other additional sequences). In some embodiments, the parts or components of the polypeptide are provided or numbered in this paragraph in an order from the N terminus to the C terminus or from the C terminus to the N terminus of the polypeptide except that one or more of the light chain variable region is switched with the heavy chain variable region of the same antigen binding sequence. In some embodiments, the parts or components of the polypeptide are provided or numbered in this paragraph in an order from the N terminus to the C terminus or from the C terminus to the N terminus of the polypeptide except that at least two of the antigen binding sequences are switched. In some embodiments, the parts or components of the polypeptide are provided or numbered in an order from the N terminus to the C terminus or from the C terminus to the N terminus of the polypeptide except that at least two of the antigen binding sequences are switched and one or more of the light chain variable region is switched with the heavy chain variable region of the same antigen binding sequence.

In some embodiments, the polypeptide comprises, or consists essentially of, or consists of one or more of the amino acid sequence as disclosed in any one of Tables 1a, 1b, 1c, 1f, 1g, 2a, 2d, 2e, 2h, 2i, 21, 2m, 2p, 2q, 2t, 2u, 3a, 3b, 3c, or 3d, or any fragment thereof. In some embodiments, the polypeptide comprises an amino acid sequence or a fragment thereof of any one of the following construct as disclosed herein: P4 to P9, P11E, P12E, P19, P20, P61, P62, or P72.

A therapeutic antibody does not guarantee its successful use in a chimeric antigen receptor (CAR) in view of their different mechanisms of action and safety profilings. They cannot predict each other even for targeting a same antigen. Antibodies take advantage of antigen binding to induce tumor apoptosis or Fc receptor binding to macrophages, natural killer (NK) cells and neutrophils to induce multifaceted immune responses, or both. However, CAR immune cells (e.g., T cells and NK cells) utilize specific binding to a tumor antigen to subsequently induce immune cell activation via an artificial chimeric receptor. Although both CAR immune cells and antibodies can have off-target toxicities, specifically, antibodies can induce Fc-receptor-mediated toxicity (Schlothauer et al. Protein Eng. Des. Sel. 29, 457-466 (2016)), while CAR immune cells usually lead to lethal cytokine release syndrome (CRS) and neurological toxicities (Gupta et al. J Emerg Med. 2020 May 27;50736-4679(20)30352-8). Thus, one of skill in the art cannot predict CAR efficacy and safety solely based on common antigen binding domain. For this reason, the dominant therapeutic response with the disclosed CAR constructs was unexpected and surprising. In some embodiments, and in one aspect, IgG4 Fc fragments with three sites mutation can be used to avoid Fc-receptor-mediated toxicity.

Although in some aspects, an scFv in a CAR can be derived from an antibody; to be functional, both are required to bind to a tumor antigen (a TAA). However, the binding of an antibody to a tumor antigen is usually in a soluble form, while the binding of a scFv on CAR to a tumor antigen is from the surface of immune cells. There are at least three differences that determine these two types of interactions: 1) protein or peptide conformation requirement is different in the two settings; 2) in the antibody setting, a full-length antibody consisting of two Fab regions (each consisting of a part of heavy chain and a light chain) and a Fc on a heavy chain governs binding affinity and the binding is bivalent due to the two Fab regions, while for CAR, scFv, in which the one Fab region consisting of a heavy chain variable region (VH) and a light chain viable region (VL) that are linked to each other by a linker, is univalent binding; 3) due to univalent binding and a shorter length of scFv compared to its corresponding antibody, a CAR may have less binding affinity while less immunogenic in patients when a murine scFv is used for a CAR, which is the most cases of CARs on the market (Dotti et al. Immunol Rev 257, 107-126 (2014)). Thus, functionality of an antibody cannot predict that of a CAR. In fact, Haso et al. (Blood 121, 1165-1174 (2013)) recently demonstrated that increasing the binding affinity of a CAR using a high-affinity antibody-derived scFv failed to increase CAR activity in vitro and in vivo (Haso et al. Blood 121, 1165-1174 (2013) and Handgretinger et al. Blood 121, 1065-1066 (2013)).

In some embodiments of any aspect as disclosed herein, the hinge domain comprises, or consists essentially of, or yet further consists of a CD8 a hinge domain or an IgG1 hinge domain. In further embodiments, the IgG1 hinge domain comprises, or consists essentially of, or yet further consists of LEPKSCDKTHTCPPCPDPKGT (SEQ ID NO: 47) or an equivalent thereof.

In some embodiments of any aspect as disclosed herein, the transmembrane domain comprises, or consists essentially of, or yet further consists of a CD8 a transmembrane domain or a CD28 transmembrane domain.

In some embodiments of any aspect as disclosed herein, the intracellular domain comprises, or consists essentially of, or yet further consists of one or more or two or more costimulatory regions selected from a CD28 costimulatory signaling region, a 4-1BB costimulatory signaling region, an ICOS costimulatory signaling region, an OX40 costimulatory region, a DAP 10 costimulatory region, a DAP12 costimulatory region, a PD-1 costimulatory domain, a CTLA-4 costimulatory domain, a LAG-3 costimulatory domain, a 2B4 costimulatory domain, a BTLA costimulatory domain, or any combination thereof.

In some embodiments of any aspect as disclosed herein, the transmembrane domain is a CD28 transmembrane domain. In further embodiments, the CD28 transmembrane domain comprises, or consists essentially of, or yet further consists of FWVLVVVGGVLACYSLLVTVAFIIFWV (SEQ ID NO: 91) or an equivalent thereof.

In some embodiments of any aspect as disclosed herein, the intracellular domain comprises a costimulatory region which is also referred to herein as a costimulatory signaling region. In further embodiments, the costimulatory region is a CD28 costimulatory signaling region. In yet further embodiments, the CD28 costimulatory signaling region comprises, or consists essentially of, or yet further consists of RSKRSRLLHSDYMNMTPRRPGPTRKHYQPYAPPRDFAAYRS (SEQ ID NO: 95) or equivalent thereof.

In some embodiments of any aspect as disclosed herein, the CAR comprises a CD28 transmembrane domain and a CD 28 cytoplasmic domain (e.g., a CD28 costimulatory signaling region) comprising, or consisting essentially of, or yet further consisting of SEQ ID NO: 48 or an equivalent thereof.

In some embodiments of any aspect as disclosed herein, the intracellular domain comprises, or consists essentially of, or yet further consists of a CD3 zeta signaling domain. In further embodiments, the CD3 zeta signaling domain comprises, or consists essentially of, or yet further consists of SEQ ID NO: 49 or an equivalent thereof.

In some embodiments of any aspect as disclosed herein, the intracellular domain further comprises an IL2Rβ or a fragment thereof comprising an JAK-STAT activation domain. In some embodiments, a CAR or a cytoplasmic/intracellular domain thereof as disclosed herein further comprise an IL2Rβ or a fragment thereof. In further embodiments, the fragments of IL2Rβ comprises, or alternatively consists essentially of, or yet consists of an JAK-STAT activation domain of the IL2Rβ, facilitating activation of the immune cell. In some embodiments, the CAR or the intracellular domain of the CAR further comprises an IL2Rβ or a fragment thereof comprising an JAK-STAT activation domain. In some embodiments, the JAK-STAT activation domain comprises, or alternatively consists essentially of, or yet consists of a JAK binding domain (also known as a box-1 motif which allows for tyrosine kinase JAK association, for example JAK1) or a Signal Transducer and Activator of Transcription (STAT, such as STAT3 or STATS) association motif or both. An example of the JAK binding domain can be found amino acid numbers 278 to 286 of NCBI RefSeq:NP_000869.1. In further embodiments, the intracellular domain further comprises an endogenous or exogenous JAK-binding motif, or an endogenous or exogenous STAT association motif, or both. In some embodiments, the exogenous STAT3 association motif is YXXQ (SEQ ID NO: 136), optionally YRHQ (SEQ ID NO: 137). Cells expressing such CAR cells show antigen-dependent JAK-STAT3/5 pathway activation, which promotes their proliferation and prevented terminal differentiation in vitro or ex vivo. Alternatively, JAK-STAT activation domain from a protein other than IL2Rβ may be used here as a substitution. Exemplified of such protein may include Erythropoietin receptor (EpoR), thrombopoietin receptor (TpoR), granulocyte macrophage colony stimulating factor receptor (GM-CSFR), or growth hormone receptor (GHR). See, for example, US20190359685A1 and Kagoya et al. Nat Med. 2018 March;24(3):352-359. doi: 10.1038/nm.4478. Epub 2018 Feb. 5.

In some embodiments of any aspect as disclosed herein, the polypeptide or the CAR further comprises a suicide gene product or a detectable marker or both. In further embodiments, the suicide gene product is selected from one or more of: HSV-TK (Herpes simplex virus thymidine kinase), cytosine deaminase, nitroreductase, carboxylesterase, cytochrome P450 or PNP (Purine nucleoside phosphorylase), truncated EGFR (tEGFR), RQR8, or inducible caspase (“iCasp”).

In some embodiments of any aspect as disclosed herein, the CAR further comprises a signal peptide at its N terminus, optionally different from the signal peptide at the N terminus of the bispecific antibody. In further embodiments, the signal peptide facilitates locating the CAR to be on the cell surface. In yet further embodiments, the signal peptide comprises, or consists essentially of, or yet further consists of MYRMQLLSCIALSLALVTNS (SEQ ID NO: 20) or an equivalent of each thereof. In one embodiment, the signal peptide comprises, or consists essentially of, or yet further consists of MGWSSIILFLVATATGVH (SEQ ID NO: 21), or MLLLVTSLLLCELPHPAFLLIP (SEQ ID NO: 125) or an equivalent of each thereof. In some embodiments, the signal peptide comprises, or consists essentially of, or yet further consists of MYRMQLLSCIALSLALVTNS (SEQ ID NO: 20), or MWLQSLLLLGTVACSIS (SEQ ID NO: 122), or MRISKPHLRSISIQCYLCLLLNSHFLTEA (SEQ ID NO: 123), or MRSSPGNMERIVICLMVIFLGTLV (SEQ ID NO: 124), or MGWSSIILFLVATATGVH (SEQ ID NO: 21), or MLLLVTSLLLCELPHPAFLLIP (SEQ ID NO: 125) or an equivalent of each thereof that facilitates locating the CAR to be on the cell surface.

In some embodiments of any aspect as disclosed herein, the CAR or the polypeptide further comprises a detectable marker. In some embodiments, the CAR can be detected by the TAA which the CAR recognizes and binds. In further embodiments, the TAA is conjugated to a detectable marker. In some embodiments, the CAR can be detected by an antibody recognizing and binding its antigen binding sequence. For example, when the antigen binding sequence of the CAR is an mouse Fab or scFv, such CAR can be detected by an antibody recognizing and binding a mouse Fab or scFv, for example a goat anti mouse antibody.

In some embodiments of any aspect as disclosed herein, the polypeptide further comprises a cleavable peptide located between any two of the following: the CAR; the bispecific antibody (also referred to herein as BiTE, bispecific T cell engager or BiKE, bispecific NK cell engager); the optional cytokine; or the optional suicide gene product or a detectable marker or both. In some embodiments, the cleavable peptide is a self-cleaving peptide. In further embodiments, the self-cleaving peptide is a T2A peptide. In yet further embodiments, cleavable peptide or self-cleaving peptide or the T2A peptide comprises, or consists essentially of, or yet further consists of HVGSGEGRGSLLTCGDVEENPGP (SEQ ID NO: 50) or an equivalent thereof.

In some embodiments, the bispecific antibody comprises, or consists essentially of, or yet further consists of, from the N terminus to the C terminus, the anti-GPRCSD antigen binding sequence and the anti-NKG2D antigen binding sequence. In some embodiments, the bispecific antibody comprises, or consists essentially of, or yet further consists of, from the N terminus to the C terminus, the anti-NKG2D antigen binding sequence and the anti-GPRCSD antigen binding sequence. The binding sequences are described herein and incorporated herein and include for example, scFv, the HCVR and the LCVR as well as the minimal CDRs.

In some embodiments, an equivalent of a variable region retains the CDRs of the variable region. In some embodiments, the equivalent of SEQ ID NO: 7 retains the CDRs of SEQ ID NO: 7. In some embodiments, the equivalent of SEQ ID NO: 8 retains the CDRs of SEQ ID NO: 8. In some embodiments, the equivalent of SEQ ID NO: 16 retains the CDRs of SEQ ID NO: 16. In some embodiments, the equivalent of SEQ ID NO: 17 retains the CDRs of SEQ ID NO: 17. In some embodiments, the equivalent of SEQ ID NOs: 41-46 retains the CDRs of SEQ ID NOs: 41-46, respectively.

In some embodiments, the polypeptide as disclosed herein comprises, or consists essentially of, or yet further consists of one or more of the following or a fragment thereof: SEQ ID NO: 51; SEQ ID NO: 52; SEQ ID NO: 53; SEQ ID NO: 54; amino acid sequences listed in Tables 1b-1h, Tables 2b-2x, and Tables 3a-3d; or an amino acid sequence encoded by a nucleotide sequence listed in Tables 1b-1h, Tables 2b-2x, and Tables 3a-3d.

In some embodiments, the polypeptide as disclosed herein comprises, or consists essentially of, or yet further consists of one or more of: SEQ ID NO: 138 or SEQ ID NO: 139, or an equivalent of each thereof.

### Polynucleotides

Additionally provided is a polynucleotide encoding one or more of the following: a polypeptide as disclosed herein or any fragment thereof, a bispecific antibody as disclosed herein, or a CAR as disclosed herein, along with a reverse or complement or reverse-complement polynucleotide thereto. In some embodiments, the polynucleotide further comprises any one or any two or all three of the following: a first regulatory sequence directing the expression of the polypeptide; a second regulatory sequence directing the expression of the bispecific antibody; and a third regulatory sequence directing the expression of the CAR.

In some embodiments, the polynucleotide comprises, or consists essentially of, or consists of (1) a signal peptide coding sequence as disclosed herein, such as the one comprising, or consisting essentially of, or consisting of ATGGGGTGGTCAAGCATTATTCTGTTTCTGGTCGCTACCGCTACAGGCGTCCAT (SEQ ID NO: 55) or an equivalent thereof; (2) an anti-BCMA antigen binding sequence coding sequence as disclosed herein, such as the one comprising, or consisting essentially of, or consisting of (2-i) a light chain variable region coding sequence that comprises, or consists essentially of, or consists of SEQ ID NO: 58 or SEQ ID NO: 59 or an equivalent of each thereof, (2-ii) an optional peptide linker coding sequence for example that comprises, or consists essentially of, or consists of GGTGGGGGCGGCTCTGGTGGCGGTGGCAGCGGCGGAGGTGGCAGT (SEQ ID NO: 57) or an equivalent thereof, and (2-iii) a heavy chain variable region coding sequence that comprises, or consists essentially of, or consists of SEQ ID NO: 62 or SEQ ID NO: 63 or an equivalent of each thereof, and wherein the equivalents encode variable regions recognizing and binding BCMA; (3) a hinge domain coding sequence as disclosed herein, such as the one comprising, or consisting essentially of, or consisting of CTCGAGCCCAAATCTTGTGACAAAACTCACACATGCCCACCGTGCCCGGATCCC AAAGGTACC (SEQ ID NO: 66) or an equivalent thereof; (4) an transmembrane domain coding sequence as disclosed herein, such as an CD28 transmembrane domain coding sequence comprising, or consisting essentially of, or consisting of TTTTGGGTGCTGGTGGTGGTTGGTGGAGTCCTGGCTTGCTATAGCTTGCTAGTAA CAGTGGCCTTTATTATTTTCTGGGTG (SEQ ID NO: 90) or an equivalent thereof; (5) a costimulatory signaling region coding sequence as disclosed herein, such as a CD28 costimulatory signaling region coding sequence comprising, or consisting essentially of, or consisting of SEQ ID NO: 96 or an equivalent thereof; (6) an CD3 zeta signaling domain coding sequence, such as the one comprising, or consisting essentially of, or consisting of SEQ ID NO: 68 or an equivalent thereof; (7) a cleaving peptide coding sequence as disclosed herein, such as a T2A self-cleaving peptide coding sequence comprising, or consisting essentially of, or consisting of SEQ ID NO: 69 or SEQ ID NO: 140 or an equivalent thereof; (8) a signal peptide coding sequence as disclosed herein, such as the one comprising, or consisting essentially of, or consisting of SEQ ID NO: 56 or an equivalent thereof; (9) an anti-GPRCSD antigen binding sequence coding sequence as disclosed herein, such as the one comprising, or consisting essentially of, or consisting of (9-i) a light chain variable region coding sequence that comprises, or consists essentially of, or consists of SEQ ID NO: 71 or SEQ ID NO: 141 or an equivalent of each thereof, (9-ii) a peptide linker coding sequence that comprises, or consists essentially of, or consists of SEQ ID NO: 57 or SEQ ID NO: 142 or an equivalent of each thereof, and (9-iii) a heavy chain variable region coding sequence that comprises, or consists essentially of, or consists of SEQ ID NO: 72, or SEQ ID NO: 143, or an equivalent of each thereof, wherein the equivalents encode variable regions recognizing and binding GPRCSD; (10) a linker coding sequence as disclosed herein, such as a human muscle aldolase (HMA) linker coding sequence comprising, or consisting essentially of, or consisting of SEQ ID NO: 70 or SEQ ID NO: 144 or an equivalent of each thereof; (11) an anti-NKG2D antigen binding sequence coding sequence as disclosed herein, such as the one comprising, or consisting essentially of, or consisting of (11-i) a light chain variable region coding sequence that comprises, or consists essentially of, or consists of SEQ ID NO: 73 or SEQ ID NO: 145 or an equivalent of each thereof, (11-ii) a peptide linker coding sequence that comprises, or consists essentially of, or consists of SEQ ID NO: 57 or SEQ ID NO: 146 or an equivalent of each thereof, and (11-iii) a heavy chain variable region coding sequence that comprises, or consists essentially of, or consists of SEQ ID NO: 74 or an equivalent thereof, wherein the equivalents encode variable regions recognizing and binding NKG2D; (12) an Fc region coding sequence as disclosed herein, such as the one comprising, or consisting essentially of, or consisting of SEQ ID NO: 75 or SEQ ID NO: 147 or an equivalent of each thereof; (13) a cleaving peptide coding sequence as disclosed herein, such as a T2A self-cleaving peptide coding sequence comprising, or consisting essentially of, or consisting of SEQ ID NO: 148 or SEQ ID NO: 149 or an equivalent of each thereof; (14) a signal peptide coding sequence as disclosed herein, such as the one comprising, or consisting essentially of, or consisting of SEQ ID NO: 56 or SEQ ID NO: 150 or SEQ ID NO: 200, SEQ ID NO: 127, or SEQ ID NO: 151 or an equivalent of each thereof; (15) a cytokine coding sequence as disclosed herein, such as the one comprising, or consisting essentially of, or consisting of at least one of SEQ ID NO: 152, SEQ ID NO: 153, SEQ ID NO: 154, SEQ ID NO: 155, SEQ ID NO: 156, SEQ ID NO: 157, SEQ ID NO: 158 or an equivalent of each thereof; (16) a cleaving peptide coding sequence as disclosed herein, such as a P2A self-cleaving peptide coding sequence comprising, or consisting essentially of, or consisting of SEQ ID NO: 159 or SEQ ID NO: 160 or an equivalent thereof; (17) an optional signal peptide coding sequence as disclosed herein, such as the one comprising, or consisting essentially of, or consisting of SEQ ID NO: 161 or an equivalent thereof; and (18) a detectable marker and/or suicide gene product coding sequence or a suicide gene as disclosed herein, such as the one comprising, or consisting essentially of, or consisting of SEQ ID NO: 162 or SEQ ID NO: 163 or an equivalent of each thereof. In some embodiments, the cytokine coding sequence further comprises a transmembrane domain coding sequence comprising, or consisting essentially of, or consisting of SEQ ID NO: 164 or an equivalent thereof. In some embodiments, the cytokine coding sequence comprises, or consists essentially of, or consists of SEQ ID NO: 152, or SEQ ID NO: 153 or an equivalent of each thereof, an optional peptide linker coding sequence comprising, or consisting essentially of, or consisting of SEQ ID NO: 165 or SEQ ID NO: 166 or SEQ ID NO: 57 or an equivalent of each thereof, and SEQ ID NO: 167 or an equivalent thereof. In some embodiments, the parts or components of the polynucleotide are provided or numbered without any limiting or indication of their location or arrangement in the polynucleotide. In some embodiments, the parts or components of the polynucleotide are provided or numbered in an order from the 5′ to the 3′ of the polynucleotide (i.e., arranged in the polynucleotide as from the 5′ to the 3′ with or without other sequences). In some embodiments, the parts or components of the polynucleotide are provided or numbered in an order from the 3′ to the 5′ of the polynucleotide (i.e., arranged in the polynucleotide as from the 3′ to the 5′ with or without other sequences). In some embodiments, the parts or components of the polynucleotide are provided or numbered in an order from the 5′ to the 3′ or from the 3′ to the 5′ in the polynucleotide except that one or more of the light chain variable region coding sequence is switched with the heavy chain variable region coding sequence of the same antigen binding sequence. In some embodiments, the parts or components of the polynucleotide are provided or numberred in an order from the 5′ to the 3′ or from the 3′ to the 5′ in the polynucleotide except that at least two of the antigen binding sequence coding sequences are switched. In some embodiments, the parts or components of the polynucleotide are listed in an order from the 5′ to the 3′ or from the 3′ to the 5′ in the polynucleotide except that at least two of the antigen binding sequence coding sequences are switched and one or more of the light chain variable region coding sequence is switched with the heavy chain variable region coding sequence of the same antigen binding sequence.

In some embodiments, the polynucleotide comprises, or consists essentially of, or further consists of one or more of the nucleotide sequence as disclosed in any one of Tables 1a, 1b, 1c, 1f, 1g, 2a, 2d, 2e, 2h, 2i, 21, 2m, 2p, 2q, 2t, 2u, 3a, 3b, 3c, or 3d, or any fragment thereof. In some embodiments, the polynucleotide comprises, or consists essentially of, or further consists of one or more of the following sequences or a fragment thereof: nucleotide sequences of P4 to P9, P11E, P12E, P19, P20, P61, P62, or P72, as disclosed herein.

In some embodiments, the polynucleotide further comprises one or more of the following: a first regulatory sequence directing the expression of the CAR, a second regulatory sequence directing the expression of the bispecific antibody, or an internal ribosome entry site (IRES) located between the sequence encoding the CAR and the sequence encoding the bispecific antibody. In some embodiments, the first regulatory sequence is different from the second regulatory sequence in sequence or location in the polynucleotide or both. In some embodiments, the first regulatory sequence and the second regulatory sequence constitutes a bidirectional regulatory sequence. Accordingly, provided is a composition comprising, or consisting essentially of, or yet further consisting of a CAR peptide and a bispecific antibody, both of which are encoded by the polynucleotide.

In some embodiments, the polynucleotide further comprises one or more of the following: a first regulatory sequence directing the expression of the CAR, a second regulatory sequence directing the expression of the bispecific antibody, a third regulatory sequence directing the expression of the cytokine, a fourth regulatory sequence directing the expression of the suicide gene product or detectable marker or both. In some embodiments, any two of the regulatory sequences are different from each other in sequence or location in the polynucleotide or both. In some embodiments, any two of the regulatory sequences constitute a bidirectional regulatory sequence. Additionally or alternatively, the polynucleotide further comprises one or more internal ribosome entry site(s) (IRES) located between any two of the following: a nucleotide sequence encoding the CAR, a nucleotide sequence encoding the bispecific antibody, a nucleotide sequence encoding the cytokine if present, or a nucleotide sequence encoding the suicide gene product (for example, a suicide gene) or a detectable marker or both if present. Accordingly, provided is a composition comprising, or consisting essentially of, or yet further consisting of one or more of: a CAR, a bispecific antibody, a cytokine, or a suicide gene product or a detectable marker or both, all of which are encoded by the polynucleotide.

In yet further embodiments, each of the regulatory sequences comprise one or more of the following: a promoter, an intron, an enhancer, or a polyadenylation signal. In some embodiments, the regulatory sequences direct the expression of the polypeptide, CAR, bispecific antibody, cytokine, suicide gene product, detectable marker, or any combination thereof. In some embodiments, the regulatory sequences is suitable for replicating the polynucleotide. In some embodiments, the polynucleotide further comprises a detectable label or a polynucleotide conferring antibiotic resistance or both. In further embodiments, the label or polynucleotide are useful to select cells successfully transduced with the polynucleotide. In some embodiments, the polynucleotide is isolated or engineered or both isolated and engineered.

In some embodiments, the polynucleotide comprises, or consists essentially of, or yet further consists of one or more of the nucleotide sequences as disclosed in Tables 1a-1h, Tables 2a-2x, and Tables 3a-3d or an equivalent of each thereof encoding the same amino acid sequence.

In some embodiments, the polynucleotide further comprises a detectable marker. Additionally or alternatively, the polynucleotide further comprises a suicide gene, such as an inducible caspase (iCasp) suicide gene.

In some embodiments, a polynucleotide comprises, or consists essentially of, or yet further consists of one or more of the following: (I) a nucleotide sequence encoding a signal peptide, and wherein the nucleotide sequence comprises, or consists essentially of, or yet further consists of SEQ ID NO: 55 or an equivalent thereof; (II) a nucleotide sequence encoding a signal peptide, and wherein the nucleotide sequences comprises, or consists essentially of, or yet further consists of SEQ ID NO: 56 or an equivalent thereof; (III) a nucleotide sequence encoding a linker peptide, and wherein the nucleotide sequence comprises, or consists essentially of, or yet further consists of SEQ ID NO: 57 or an equivalent thereof; (IV) a nucleotide sequence encoding an anti-BCMA light chain variable region, and wherein the nucleotide sequence is selected from: SEQ ID NO: 58, SEQ ID NO: 59, SEQ ID NO: 60, or SEQ ID NO: 61 or an equivalent thereof; (V) a nucleotide sequence encoding an anti-BCMA heavy chain variable region, and wherein the nucleotide sequence is selected from: SEQ ID NO: 62, SEQ ID NO: 63, SEQ ID NO: 64, or SEQ ID NO: 65 or an equivalent thereof; (VI) a nucleotide sequence encoding a hinge domain, and wherein the nucleotide sequence comprises, or consists essentially of, or yet further consists of SEQ ID NO: 66 or an equivalent thereof; (VII) a nucleotide sequence encoding a transmembrane and cytoplasmic/intracellular domain, and wherein the nucleotide sequence comprises, or consists essentially of, or yet further consists of SEQ ID NO: 67 or an equivalent thereof; (VIII) a nucleotide sequence encoding a signaling domain, and wherein the nucleotide sequence comprises, or consists essentially of, or yet further consists of SEQ ID NO: 68 or an equivalent thereof; (IX) a nucleotide sequence encoding a self-cleaving peptide, and wherein the nucleotide sequence comprises, or consists essentially of, or yet further consists of SEQ ID NO: 69 or an equivalent thereof; (X) a nucleotide sequence encoding a linker, and wherein the nucleotide sequence comprises, or consists essentially of, or yet further consists of SEQ ID NO: 70 or an equivalent thereof; a nucleotide sequence encoding an anti-GPRCSD light chain variable region, and wherein the nucleotide sequence comprises, or consists essentially of, or yet further consists of SEQ ID NO: 71 or an equivalent thereof; (XII) a nucleotide sequence encoding an anti-GPRCSD heavy chain variable region, and wherein the nucleotide sequence comprises, or consists essentially of, or yet further consists of SEQ ID NO: 72 or an equivalent thereof; (XIII) a nucleotide sequence encoding an anti-NKG2D light chain variable region, and wherein the nucleotide sequence comprises, or consists essentially of, or yet further consists of SEQ ID NO: 73 or an equivalent thereof; (XIV) a nucleotide sequence encoding an anti-NKG2D heavy chain variable region, and wherein the nucleotide sequence comprises, or consists essentially of, or yet further consists of SEQ ID NO: 74 or an equivalent thereof; (XV) a nucleotide sequence encoding a mutant Fc region, and wherein the nucleotide sequence comprises, or consists essentially of, or yet further consists of SEQ ID NO: 75 or an equivalent thereof; (XVI) a nucleotide sequence encoding a tag, wherein the nucleotide sequence comprises, or consists essentially of, or yet further consists of SEQ ID NO: 76 or an equivalent thereof; (XVII) a nucleotide sequence of SEQ ID NO: 77 or an equivalent thereof; (XVIII) a nucleotide sequence of SEQ ID NO: 78 or an equivalent thereof; (XIX) a nucleotide sequence of SEQ ID NO: 79 or an equivalent thereof; and (XX) a nucleotide sequence of SEQ ID NO: 80 or an equivalent thereof. In some embodiments, the polynucleotide comprises, or consists essentially of, or yet further consists of any one or more of the nucleotide sequences listed in Tables 1a-1h.

In some embodiments, a polynucleotide as disclosed herein further comprises a suicide gene product or a detectable marker or both. In further embodiments, the suicide gene encodes a product selected from one or more of: HSV-TK (Herpes simplex virus thymidine kinase), cytosine deaminase, nitroreductase, carboxylesterase, cytochrome P450 or PNP (Purine nucleoside phosphorylase), truncated EGFR, or inducible caspase (“iCasp”).

In some embodiments, the polynucleotide further comprises a detectable marker.

In some embodiments, the polynucleotide comprises, or consists essentially of, or yet further consists of any one or more of SEQ ID NO: 168; or SEQ ID NO: 169, or an equivalent of each thereof.

In some embodiments, an equivalent of a reference polynucleotide encodes the same polypeptide that the reference polynucleotide encodes. In some embodiments, an equivalent of a reference polynucleotide encodes an equivalent of the polypeptide that the reference polynucleotide encodes.

In some embodiments, a polynucleotide as disclosed herein further comprises one or more regulatory sequence directing the replication of the polynucleotide.

### Vectors

Additionally, provided is a vector comprising, or consisting essentially of, or yet further consisting of one or more of: a polynucleotide as disclosed herein, a complement thereof, a reverse sequence thereof, or a reverse-complement thereof. In some embodiments, the vector is a non-viral vector or a viral vector. In one embodiment, the non-viral vector is a plasmid. In one embodiment, the viral vector is selected from the group of a retroviral vector, a lentiviral vector, an adenoviral vector, an adeno-associated viral vector, or Herpes viral vector. Additionally, the vector may further comprises a regulatory sequence directing the replication or the expression of the polynucleotide or both. In some embodiment, a plasmid is constructed and prepared, for example as shown in the Examples 1 and 2, to produce a viral vector, such as a lentiviral vector.

In some embodiments, a vector is used to replicate or amplify a polynucleotide as disclosed herein. In further embodiments, the vector comprises one or more of: the polynucleotide, a complement thereof, a reverse sequence thereof, or a reverse-complement thereof. In yet further embodiments, the vector further comprises a regulatory sequence directing the replication of the polynucleotide.

In some embodiments, a vector is used to transcript or translate or both transcript and translate a polynucleotide as disclosed herein, for example, to a polypeptide as disclosed herein or a fragment thereof. In further embodiments, the vector comprises one or more of: the polynucleotide, a complement thereof, a reverse sequence thereof, or a reverse-complement thereof. In yet further embodiments, the vector further comprises a regulatory sequence directing the expression of the polynucleotide. Additionally or alternatively, the vector further comprises one or more internal ribosome entry site(s) (IRES) located between any two of the following: a nucleotide sequence encoding the CAR, a nucleotide sequence encoding the bispecific antibody, a nucleotide sequence encoding the cytokine if present, or a nucleotide sequence encoding the suicide gene product or a detectable marker or both (for example, a suicide gene) if present.

In one aspect, provided is a vector system comprising, or consisting essentially of, or yet further consisting of more than one vectors. In some embodiments, the vectors encodes (i) an amino acid sequence of a Chimeric Antigen Receptor (CAR) comprising, or consisting essentially of, or yet further consisting of: (1) an antigen binding amino acid sequence that recognizes and binds B-cell maturation antigen (BCMA) (anti-BCMA antigen binding sequence), (2) a hinge domain, (3) a transmembrane domain, and (4) an intracellular domain; (ii) an amino acid sequence of a bispecific antibody comprising, or consisting essentially of, or yet further consisting of (1) an antigen binding sequence that recognizes and binds NKG2D (anti-NKG2D antigen binding sequence), and (2) an antigen binding sequence that recognizes and binds G Protein-Coupled Receptor Class C Group 5 Member D (GPRC5D) (anti-GPRC5D antigen binding sequence); (iii) an optional amino acid sequence of a cytokine selected from IL15 or IL-21; and (iv) an optional amino acid sequence of a suicide gene product or a detectable marker or both, optionally a truncated epidermal growth factor receptor (tEGFR) or RQR8.

In some embodiments, the vector system comprises, or consists essentially of, or yet further consists of at least two vectors. In some embodiments, the first vector encodes (i) an amino acid sequence of a Chimeric Antigen Receptor (CAR) comprising, or consisting essentially of, or yet further consisting of: (1) an antigen binding amino acid sequence that recognizes and binds B-cell maturation antigen (BCMA) (anti-BCMA antigen binding sequence), (2) a hinge domain, (3) a transmembrane domain, and (4) an intracellular domain; (iii) an optional amino acid sequence of a cytokine selected from IL15 or IL-21; and (iv) an optional amino acid sequence of a suicide gene product or a detectable marker or both, optionally a truncated epidermal growth factor receptor (tEGFR) or RQR8. In some embodiments, the second vector encodes (ii) an amino acid sequence of a bispecific antibody comprising, or consisting essentially of, or yet further consisting of (1) an antigen binding sequence that recognizes and binds NKG2D (anti-NKG2D antigen binding sequence), and (2) an antigen binding sequence that recognizes and binds G Protein-Coupled Receptor Class C Group 5 Member D (GPRC5D) (anti-GPRC5D antigen binding sequence); (iii) an optional amino acid sequence of a cytokine selected from IL15 or IL-21; and (iv) an optional amino acid sequence of a suicide gene product or a detectable marker or both, optionally a truncated epidermal growth factor receptor (tEGFR) or RQR8. In some embodiments, the suicide gene product or detectable marker or both of the first vector is different from the suicide gene product or detectable marker or both of the second vector. In some embodiments, the suicide gene product or detectable marker or both of the first vector is the same with the suicide gene product or detectable marker or both of the second vector.

In some embodiments, the suicide gene products or detectable markers in different vectors of the vector system are different. In some embodiments, the suicide gene products or detectable markers in different vectors of the vector system are the same.

### Cells

Further provided is an isolated or engineered or both isolated and engineered cell comprising one or more of the following: a polypeptide as disclosed herein, a bispecific antibody or a fragment thereof of as disclosed herein, a CAR as disclosed herein, a cytokine as disclosed herein, a suicide gene product or a detectable marker or both as disclosed herein, a suicide gene as disclosed herein, a polynucleotide as disclosed herein, a vector as disclosed herein, or a vector system as disclosed herein.

In some embodiments, the cell is a prokaryotic cell or a eukaryotic cell. In some embodiments, the cell is a eukaryotic cell, optionally selected from an animal cell, a mammalian cell, a bovine cell, a feline cell, a canine cell, a murine cell, an equine cell, or a human cell. In some embodiments, the eukaryotic cell is an immune cell, optionally a T-cell, a B cell, a NK cell, a NKT cell, a dendritic cell, a myeloid cell, a monocyte, or a macrophage, optionally derived from Hematopoietic stem cells (HSCs) or induced pluripotent stem cells (iPSCs). In some embodiments, HSCs and iPSCs are referred to herein as a precursor cell. In some embodiments, the cell is a stem cell, such as an HSC or an iPSC. In some embodiments, the isolated cell expresses a CAR as disclosed herein or secretes a bispecific antibody as disclosed herein or both. Additionally, the isolated cell further expresses or expresses and secretes a cytokine as disclosed herein. Additionally or alternatively, the isolated cell further expresses a suicide gene product or a detectable marker or both as disclosed herein.

In some embodiments, the cell is a T-cell, a B cell, an NK cell, an NKT cell, a dendritic cell, a myeloid cell, a monocyte, a macrophage, any subsets thereof, or any other immune cell. In some embodiments, the cell is an immune cell optionally selected from a T-cell, a B cell, an NK cell, an NKT cell, a dendritic cell, a myeloid cell, a monocyte, a macrophage. In further embodiments, the immune cell is derived from hematopoietic stem cells (HSCs) or induced pluripotent stem cells (iPSCs). The eukaryotic cell can be from any preferred species, e.g., an animal cell, a mammalian cell such as a human, a bovine cell, a murine cell, an equine cell, a feline cell, or a canine cell. The cells may be derived from patients, donors, or cell lines, such as those available off-the-shelf. The cells can be autologous or allogeneic to the subject being treated. In some embodiments, the cell further comprise a detectable or purification marker. In some embodiments, the cell expresses a CAR as disclosed herein. Additionally or alternatively, the cell expresses a bispecific antibody as disclosed herein. In further embodiments, the cell secretes the bispecific antibody out of the cell.

Also provided is a cell population comprising, or alternatively consisting essentially of, or yet consisting of a cell as disclosed herein. In some embodiments, the cell population is homogenous or heterogeneous. In further embodiments, the cell population is substantially homogenous. In some embodiments, the cell is a T cell that has been modified to remove CD52 expression using gene editing technology, e.g., CRISPR or TALEN.

In one aspect, provided is a cell comprising a polypeptide comprising, or consisting essentially of, or yet further consisting of one or more of (i) an amino acid sequence of a Chimeric Antigen Receptor (CAR) as disclosed herein, (ii) an amino acid sequence of a bispecific antibody as disclosed herein, (iii) an amino acid sequence of a cytokine as disclosed herein, or (iv) an amino acid sequence of a suicide gene product or a detectable marker or both as disclosed herein; or a polynucleotide as disclosed herein; or both of the polypeptide and the polynucleotide. In some embodiments, the polypeptide is further processed, for example, cleaved by itself or by the cell, to yield one or more of the CAR peptide, the bispecific antibody, the cytokine, or the suicide gene product or detectable marker or both. In further embodiments, the cell further comprises one or more of the CAR peptide, the bispecific antibody, the cytokine, or the suicide gene product a detectable marker or both. In yet further embodiments, the cell expresses the CAR on the cell membrane. In further embodiments, the cell secrets the bispecific antibody outside of the cell. In some embodiments, the cell expresses the cytokine. In further embodiments, the cell secrets the cytokine. In some embodiments, the cell expresses the cytokine on the cell membrane. In some embodiments, the cell expresses the suicide gene product or detectable marker or both. In further embodiments, the cell expresses the suicide gene product or detectable marker or both on the cell membrane. In some embodiments, the cell comprises or further comprises the polynucleotide as disclosed herein and a regulatory sequence suitable for replicating or expressing the polynucleotide in the cell. In some embodiments, the cell is isolated. Additionally or alternatively, the cell is engineered.

In some embodiments, the cell is for replicating the polynucleotide or the vector or a vector system. In further embodiments, the cell is a cell line. In some embodiments, the cell may be a prokaryotic or a eukaryotic cell. In further embodiments, the cell is an E. coli cell. In yet further embodiments, the cell is a MAX Efficiency™ Stb12™ Competent Cell (Invitrogen, 10268019) which is for propagating a polynucleotide with a size larger than 10 Kb (such as P4, P5, P6, P7, P8, P9, P11E, P12E, P61, P62, or P72) with optimized efficiency and quality. In some embodiments, the cell is the NEB® Stable Competent E. coli (High Efficiency) (NEB, C3030H), which is suitable for other plasmids with a size equal to or less than 10 Kb (such as, P1, P2, P3, P10E, P36E, P37E, P38E, P39E, P17, P18, P19, P20).

In a further aspect, provided is a cell comprising one or more (such as two, or three, or all four) of a CAR peptide as disclosed herein, a bispecific antibody as disclosed herein, a cytokine as disclosed herein, or a suicide gene product or a detectable marker or both as disclosed herein. In some embodiments, the cell comprises the CAR on the cell membrane and secretes the bispecific antibody out of the cell. In some embodiments, the cell expresses the cytokine. In further embodiments, the cell secrets the cytokine. In some embodiments, the cell expresses the cytokine on the cell membrane. In some embodiments, the cell expresses the suicide gene product or detectable marker or both. In further embodiments, the cell expresses the suicide gene product or detectable marker or both on the cell membrane. Additionally, or alternatively, any one or more of the CAR peptide, the bispecific antibody, the cytokine, or the suicide gene produce is or are produced, for example in the cell, via cleaving a polypeptide comprising, or consisting essentially of, or yet further consisting of (i) an amino acid sequence of the CAR, (ii) an amino acid sequence of the bispecific antibody, (iii) an optional amino acid sequence of a cytokine as disclosed herein, (iv) an optional amino acid sequence of a suicide gene product or a detectable marker or both as disclosed herein, and one or more cleavable peptide(s) located between any two of (i)-(iv). In some embodiments, the cell is isolated. Additionally or alternatively, the cell is engineered.

Accordingly, provided is a cell comprising a polypeptide as described herein, or a polynucleotide encoding the polypeptide, or both. In some embodiments, the polypeptide is further processed, for example, cleaved by itself or by the cell, to yield any one or two or three or all four of the following: the CAR peptide, the bispecific antibody, the cytokine, and the suicide gene product or detectable marker or both. In some embodiments, the cell further comprises any one or two or three or all four of the following: the CAR peptide, the bispecific antibody, the cytokine, and the suicide gene product or detectable marker or both. In some embodiments, the cell expresses the CAR on the cell membrane and secretes the bispecific antibody out of the cell. In further embodiments, the cell secretes the cytokine out of the cell or expresses the cytokine on the cell membrane. Additionally or alternatively, the cell expresses the suicide gene product or detectable marker or both, for example on the cell membrane. In some embodiments, the cell comprises the polynucleotide as disclosed herein and a regulatory sequence suitable for replicating or expressing the polynucleotide in the cell. In some embodiments, the cell is isolated. Additionally or alternatively, the cell is engineered.

Further provided is an isolated or engineered cell comprising one or more of the following: a polypeptide as disclosed herein, a bispecific antibody or a fragment thereof as disclosed herein, a CAR as disclosed herein, a polynucleotide as disclosed herein, or a vector as disclosed herein, or a vector system as disclosed herein. In some embodiments, the cell comprises the CAR in the cell membrane and secretes the bispecific antibody out of the cell. In further embodiments, the cell secretes a cytokine as disclosed herein. Additionally or alternatively, the cell expresses a suicide gene product or a detectable marker or both as disclosed herein, for example, on the cell surface.

Prior to expansion and genetic modification of the cells disclosed herein, cells may be obtained from a subject—for instance, in embodiments involving autologous therapy—or a commercially available cell line or culture, or a stem cell such as an induced pluripotent stem cell (iPSC).

Cells can be obtained from a number of sources in a subject, including peripheral blood mononuclear cells, bone marrow, lymph node tissue, cord blood, thymus tissue, tissue from a site of infection, ascites, pleural effusion, spleen tissue, and tumors.

### Production Methods and Compositions

In one aspect, provided is a method of producing a cell expressing a CAR or secreting a bispecific antibody or both expressing a CAR and secreting a bispecific antibody. In some embodiments, the cell further secretes a cytokine or expresses a suicide gene product (or a detectable marker or both) or both secretes a cytokine and expresses a suicide gene product or a detectable marker or both. In some embodiments, the method comprises, or consists essentially of, or yet further consists of transducing a cell or a population thereof with a polynucleotide as disclosed herein or a vector as disclosed herein or a vector system as disclosed herein. In some embodiments, the method comprises, or consists essentially or, or yet further consists of transducing a cell or a population thereof with the vectors of the vector system concurrently or subsequently. Additionally or alternatively, the method comprises, or consists essentially of, or yet further consists of culturing a cell or a cell population as disclosed herein.

Methods of isolating relevant cells are well known in the art and can be readily adapted to the present application; an exemplary method is described in the examples below. Isolation methods for use in relation to this disclosure include, but are not limited to Life Technologies Dynabeads® System; STEMcell Technologies EasySep™, RoboSep™, RosetteSep™, SepMate™; Miltenyi Biotec MACS™ cell separation kits, and other commercially available cell separation and isolation kits. Particular subpopulations of immune cells may be isolated through the use of beads or other binding agents available in such kits specific to unique cell surface markers. For example, MACS™ CD4+ and CD8+MicroBeads may be used to isolate CD4+ and CD8+T-cells. Alternate non-limiting examples of cells that may be isolated according to known techniques include bulked T-cells, NK T-cells, and gamma delta T-cells.

Alternatively, cells may be obtained through commercially available cell cultures, including but not limited to, for T-cells, lines BCL2 (AAA) Jurkat (ATCC® CRL-2902™), BCL2 (570A) Jurkat (ATCC® CRL-2900™), BCL2 (S87A) Jurkat (ATCC® CRL-2901™), BCL2 Jurkat (ATCC® CRL-2899™), Neo Jurkat (ATCC® CRL-2898™); for B cells, lines AHH-1 (ATCC® CRL-8146™), BC-1 (ATCC® CRL-2230™), BC-2 (ATCC® CRL-2231™), BC-3 (ATCC® CRL-2277™), CA46 (ATCC® CRL-1648™), DG-75 [D.G.-75] (ATCC® CRL-2625™), DS-1 (ATCC® CRL-11102™), EB-3 [EB3] (ATCC® CCL-85™), Z-138 (ATCC #CRL-3001), DB (ATCC CRL-2289), Toledo (ATCC CRL-2631), Pfiffer (ATCC CRL-2632), SR (ATCC CRL-2262), JM-1 (ATCC CRL-10421), NFS-5 C-1 (ATCC CRL-1693); NFS-70 C10 (ATCC CRL-1694), NFS-25 C-3 (ATCC CRL-1695), and SUP-B15 (ATCC CRL-1929); and, for NK cells, lines NK-92 (ATCC® CRL-2407™), NK-92MI (ATCC® CRL-2408™). Further examples include but are not limited to mature T-cell lines, e.g., Deglis, EBT-8, HPB-MLp-W, HUT 78, HUT 102, Karpas 384, Ki 225, My-La, Se-Ax, SKW-3, SMZ-1 and T34; immature T-cell lines, e.g., ALL-SIL, Be13, CCRF-CEM, CML-T1, DND-41, DU.528, EU-9, HD-Mar, HPB-ALL, H-SB2, HT-1, JK-T1, Jurkat, Karpas 45, KE-37, KOPT-K1, K-T1, L-KAW, Loucy, MAT, MOLT-1, MOLT 3, MOLT-4, MOLT 13, MOLT-16, MT-1, MT-ALL, P12/Ichikawa, Peer, PER0117, PER-255, PF-382, PFI-285, RPMI-8402, ST-4, SUP-T1 to T14, TALL-1, TALL-101, TALL-103/2, TALL-104, TALL-105, TALL-106, TALL-107, TALL-197, TK-6, TLBR-1, -2, -3, and-4, CCRF-HSB-2 (CCL-120.1), J.RT3-T3.5 (ATCC TIB-153), J45.01 (ATCC CRL-1990), J.CaM1.6 (ATCC CRL-2063), RS4;11 (ATCC CRL-1873), CCRF-CEM (ATCC CRM-CCL-119); cutaneous T-cell lymphoma lines, e.g., HuT78 (ATCC CRM-TIB-161), MJ[G11] (ATCC CRL-8294), HuT102 (ATCC TIB-162); B-cell lines derived from anaplastic and large cell lymphomas, e.g., DEL, DL-40, FE-PD, JB6, Karpas 299, Ki-JK, Mac-2A Plyl, SR-786, SU-DHL-1, -2, -4,-5,-6,-7,-8,-9,-10, and-16, DOHH-2, NU-DHL-1, U-937, Granda 519, USC-DHL-1, RL; Hodgkin's lymphomas, e.g., DEV, HD-70, HDLM-2, HD-MyZ, HKB-1, KM-H2, L 428, L 540, L1236, SBH-1, SUP-HD1, and SU/RH-HD-1; and NK lines such as HANK1, KHYG-1, NKL, NK—YS, NOI-90, and YT. Null leukemia cell lines, including but not limited to REH, NALL-1, KM-3, L92-221, are a another commercially available source of immune cells, as are cell lines derived from other leukemias and lymphomas, such as K562 erythroleukemia, THP-1 monocytic leukemia, U937 lymphoma, HEL erythroleukemia, HL60 leukemia, HMC-1 leukemia, KG-1 leukemia, U266 myeloma. Non-limiting exemplary sources for such commercially available cell lines include the American Type Culture Collection, or ATCC, (atcc.org/) and the German Collection of Microorganisms and Cell Cultures (dsmz.de/).

In some embodiments, T-cells expressing the disclosed CARs may be further modified to reduce or eliminate expression of endogenous TCRs. Reduction or elimination of endogenous TCRs can reduce off-target effects and increase the effectiveness of the T cells. T cells stably lacking expression of a functional TCR may be produced using a variety of approaches. T cells internalize, sort, and degrade the entire T cell receptor as a complex, with a half-life of about 10 hours in resting T cells and 3 hours in stimulated T cells (von Essen, M. et al. 2004. J. Immunol. 173:384-393). Proper functioning of the TCR complex requires the proper stoichiometric ratio of the proteins that compose the TCR complex. TCR function also requires two functioning TCR zeta proteins with ITAM motifs. The activation of the TCR upon engagement of its MHC-peptide ligand requires the engagement of several TCRs on the same T cell, which all must signal properly. Thus, if a TCR complex is destabilized with proteins that do not associate properly or cannot signal optimally, the T cell will not become activated sufficiently to begin a cellular response.

Accordingly, in some embodiments, TCR expression may eliminated using RNA interference (e.g., shRNA, siRNA, miRNA, etc.), CRISPR, or other methods that target the nucleic acids encoding specific TCRs (e.g., TCR-α and TCR-β) and/or CD3 chains in primary T cells. By blocking expression of one or more of these proteins, the T cell will no longer produce one or more of the key components of the TCR complex, thereby destabilizing the TCR complex and preventing cell surface expression of a functional TCR. Even though some TCR complexes can be recycled to the cell surface when RNA interference is used, the RNA (e.g., shRNA, siRNA, miRNA, etc.) will prevent new production of TCR proteins resulting in degradation and removal of the entire TCR complex, resulting in the production of a T cell having a stable deficiency in functional TCR expression.

Expression of inhibitory RNAs (e.g., shRNA, siRNA, miRNA, etc.) in primary T cells can be achieved using any conventional expression system, e.g., a lentiviral expression system. Although lentiviruses are useful for targeting resting primary T cells, not all T cells will express the shRNAs. Some of these T cells may not express sufficient amounts of the RNAs to allow enough inhibition of TCR expression to alter the functional activity of the T cell. Thus, T cells that retain moderate to high TCR expression after viral transduction can be removed, e.g., by cell sorting or separation techniques, so that the remaining T cells are deficient in cell surface TCR or CD3, enabling the expansion of an isolated population of T cells deficient in expression of functional TCR or CD3.

Expression of CRISPR in primary T cells can be achieved using conventional CRISPR/Cas systems and guide RNAs specific to the target TCRs. Suitable expression systems, e.g. lentiviral or adenoviral expression systems are known in the art. Similar to the delivery of inhibitor RNAs, the CRISPR system can be used to specifically target resting primary T cells or other suitable immune cells for CAR cell therapy. Further, to the extent that CRISPR editing is unsuccessful, cells can be selected for success according to the methods disclosed above. For example, as noted above, T cells that retain moderate to high TCR expression after viral transduction can be removed, e.g., by cell sorting or separation techniques, so that the remaining T cells are deficient in cell surface TCR or CD3, enabling the expansion of an isolated population of T cells deficient in expression of functional TCR or CD3. It is further appreciated that a CRISPR editing construct may be useful in both knocking out the endogenous TCR and knocking in the CAR constructs disclosed herein. Accordingly, it is appreciated that a CRISPR system can be designed for to accomplish one or both of these purposes.

The preparation of exemplary vectors and the generation of CAR expressing cells using said vectors is discussed herein. In summary, the expression of natural or synthetic nucleic acids encoding a polypeptide as disclosed herein, a CAR as disclosed herein a bispecific antibody as disclosed herein, or any combination thereof is typically achieved by operably linking a nucleic acid encoding the polypeptide, CAR or bispecific antibody to a promoter, and incorporating the construct into an expression vector. The vectors can be suitable for replication and integration eukaryotes. Methods for producing cells comprising vectors or exogenous nucleic acids are well-known in the art. See, for example, Sambrook et al. (2001, Molecular Cloning: A Laboratory Manual, Cold Spring Harbor Laboratory, New York).

The polynucleotide can be packaged into a retroviral packaging system by using a packaging vector and cell lines. The packaging vector includes, but is not limited to retroviral vector, lentiviral vector, adenoviral vector, and adeno-associated viral vector. The packaging vector contains elements and sequences that facilitate the delivery of genetic materials into cells. For example, the retroviral constructs are packaging vectors comprising at least one retroviral helper DNA sequence derived from a replication-incompetent retroviral genome encoding in trans all virion proteins required to package a replication incompetent retroviral vector, and for producing virion proteins capable of packaging the replication-incompetent retroviral vector at high titer, without the production of replication-competent helper virus. The retroviral DNA sequence lacks the region encoding the native enhancer or promoter of the viral 5′ LTR of the virus, and lacks both the psi function sequence responsible for packaging helper genome and the 3′ LTR, but encodes a foreign polyadenylation site, for example the SV40 polyadenylation site, and a foreign enhancer or promoter or both which directs or direct efficient transcription in a cell type where virus production is desired. The retrovirus is a leukemia virus such as a Moloney Murine Leukemia Virus (MMLV), the Human Immunodeficiency Virus (HIV), or the Gibbon Ape Leukemia virus (GALV). The foreign enhancer and promoter may be the human cytomegalovirus (HCMV) immediate early (IE) enhancer and promoter, the enhancer and promoter (U3 region) of the Moloney Murine Sarcoma Virus (MMSV), the U3 region of Rous Sarcoma Virus (RSV), the U3 region of Spleen Focus Forming Virus (SFFV), or the HCMV IE enhancer joined to the native Moloney Murine Leukemia Virus (MMLV) promoter. The retroviral packaging vector may consist of two retroviral helper DNA sequences encoded by plasmid based expression vectors, for example where a first helper sequence contains a cDNA encoding the gag and pol proteins of ecotropic MMLV or GALV and a second helper sequence contains a cDNA encoding the env protein. The Env gene, which determines the host range, may be derived from the genes encoding xenotropic, amphotropic, ecotropic, polytropic (mink focus forming) or 10A1 murine leukemia virus env proteins, or the Gibbon Ape Leukemia Virus (GALV env protein, the Human Immunodeficiency Virus env (gp160) protein, the Vesicular Stomatitus Virus (VSV) G protein, the Human T cell leukemia (HTLV) type I and II env gene products, chimeric envelope gene derived from combinations of one or more of the aforementioned env genes or chimeric envelope genes encoding the cytoplasmic and transmembrane of the aforementioned env gene products and a monoclonal antibody directed against a specific surface molecule on a desired target cell.

In the packaging process, the packaging vectors and retroviral vectors are transiently co-transfected into a first population of mammalian cells that are capable of producing virus, such as human embryonic kidney cells, for example 293 cells (ATCC No. CRL1573, ATCC, Rockville, Md.) to produce high titer recombinant retrovirus-containing supernatants. In another method of the disclosure this transiently transfected first population of cells is then co-cultivated with mammalian target cells, for example human lymphocytes, to transduce the target cells with the foreign gene at high efficiencies. In yet another method of the disclosure the supernatants from the above described transiently transfected first population of cells are incubated with mammalian target cells, for example human lymphocytes or hematopoietic stem cells, to transduce the target cells with the foreign gene at high efficiencies.

In another aspect, the packaging vectors are stably expressed in a first population of mammalian cells that are capable of producing virus, such as human embryonic kidney cells, for example 293 cells. Retroviral or lentiviral vectors are introduced into cells by either co-transfection with a selectable marker or infection with pseudotyped virus. In both cases, the vectors integrate. Alternatively, vectors can be introduced in an episomally maintained plasmid. High titer recombinant retrovirus-containing supernatants are produced.

In some embodiments, the cells expressing a CAR or a suicide gene product (or a detectable marker or both) or both are enriched, for example, via selection based on the binding to the antigen that the CAR recognizes and binds (such as a BCMA or a fragment thereof) or the binding to a ligand of the suicide gene product (such as an antibody) or both. Such selection may be achieved by immobilizing the antigen or the ligand or both on a plate, a bead, a column, a membrane, a matrix, or any other suitable solid support, and selecting the cells recognizing and binding the immobilized antigen or ligand or both. One exemplified method is detailed in Example 2.

Activation and Expansion of CAR Cells. Whether prior to or after genetic modification of the cells to express a desirable CAR, the cells can be activated and expanded using generally known methods such as those described in U.S. Pat. Nos. 6,352,694; 6,534,055; 6,905,680; 6,692,964; 5,858,358; 6,887,466; 6,905,681; 7, 144,575; 7,067,318; 7, 172,869; 7,232,566; 7, 175,843; 5,883,223; 6,905,874; 6,797,514; 6,867,041 and references such as Lapateva et al. (2014) Crit Rev Oncog 19(1-2):121-32; Tam et al. (2003) Cytotherapy 5(3):259-72; Garcia-Marquez et al. (2014) Cytotherapy 16(11):1537-44. Stimulation with the tumor relevant antigen ex vivo can activate and expand the selected CAR expressing cell subpopulation. Alternatively, the cells may be activated in vivo by interaction with a tumor relevant antigen. Without wishing to be bound by the theory, a cytokine as disclosed herein, such as IL15, expressed by the cell, improves activation and expansion of the cells.

In the case of certain immune cells, additional cell populations, soluble ligands or cytokines or both, or other stimulating agents may be required to activate and expand cells. The relevant reagents are well known in the art and are selected according to known immunological principles. For instance, soluble CD-40 ligand may be helpful in activating and expanding certain B-cell populations; similarly, irradiated feeder cells may be used in the procedure for activation and expansion of NK cells.

Methods of activating relevant cells are well known in the art and can be readily adapted to the present application; an exemplary method is described in the examples below. Isolation methods for use in relation to this disclosure include, but are not limited to Life Technologies Dynabeads® System activation and expansion kits; BD Biosciences Phosflow™ activation kits, Miltenyi Biotec MACS™ activation/expansion kits, and other commercially available cell kits specific to activation moieties of the relevant cell. Particular subpopulations of immune cells may be activated or expanded through the use of beads or other agents available in such kits. For example, α-CD3/α-CD28 Dynabeads® may be used to activate and expand a population of isolated T-cells.

In one aspect, provided is a composition comprising, or consisting essentially of, or yet further consisting of a carrier (optionally a pharmaceutically acceptable carrier) and one or more of the following: a polypeptide as disclosed herein or a fragment thereof, a bispecific antibody or a fragment thereof as disclosed herein, a CAR as disclosed herein, a polynucleotide as disclosed herein, a vector as disclosed herein, a vector system as disclosed herein an isolated complex, an isolated cell as disclosed herein, or a cell population as disclosed herein. In a further embodiment, the carrier is a pharmaceutically acceptable carrier.

In another aspect, provided is an isolated complex comprising, or consisting essentially of, or yet further consisting of one or more of the following: a polypeptide as disclosed herein bound to a cancer cell, a bispecific antibody or a fragment thereof as disclosed herein bound to a cancer cell, a CAR as disclosed herein bound to a cancer cell, an isolated cell as disclosed herein bound to a cancer cell, a cancer cell bound with an isolated or engineered cell and a bispecific antibody, an isolated or engineered cell as disclosed herein bound to a cytokine and a cancer cell; or a cancer cell bound to a bispecific antibody and an isolated or engineered cell as disclosed herein which is further bound to a cytokine.

In one aspect, provided is a method of producing a CAR expressing cell. The method comprises, or consists essentially of, or yet further consists of transducing a cell or a population thereof with a polynucleotide as disclosed herein or a vector as disclosed herein or a vector system as disclosed herein. In some embodiments, the polynucleotide or the vector or a vector system encodes the CAR.

In another aspect, provided is a method of producing a cell secreting a bispecific antibody. The method comprises, or consists essentially of, or yet further consists of transducing a cell or a population thereof with a polynucleotide as disclosed herein or a vector as disclosed herein or a vector system as disclosed herein. In some embodiments, the polynucleotide or the vector or the vector system encodes the bispecific antibody.

In yet another aspect, provided is a method of producing a CAR expressing cell secreting a bispecific antibody. The method comprises, or consists essentially of, or yet further consists of transducing a cell or a population thereof with a polynucleotide as disclosed herein or a vector as disclosed herein or a vector system as disclosed herein. In some embodiments, the polynucleotide or the vector or the vector system encodes the CAR and the bispecific antibody

In one aspect, provided is a method of producing a CAR expressing cell secreting a bispecific antibody and expressing a cytokine (for example, expressing a cytokine on the cell membrane, or secreting a cytokine). The method comprises, or consists essentially of, or yet further consists of transducing a cell or a population thereof with a polynucleotide as disclosed herein or a vector as disclosed herein or a vector system as disclosed herein. In some embodiments, the polynucleotide or the vector or the vector system encodes the CAR, the bispecific antibody and the cytokine.

In another aspect, provided is a method of producing a CAR expressing cell secreting a bispecific antibody and expressing a suicide gene product or a detectable marker or both. The method comprises, or consists essentially of, or yet further consists of transducing a cell or a population thereof with a polynucleotide as disclosed herein or a vector as disclosed herein or a vector system as disclosed herein. In some embodiments, the polynucleotide or the vector or the vector system encodes the CAR, the bispecific antibody and the suicide gene.

In one aspect, provided is a method of producing a CAR expressing cell secreting a bispecific antibody, expressing a cytokine (for example, expressing a cytokine on the cell membrane, or secreting a cytokine), and expressing a suicide gene product or a detectable marker or both. The method comprises, or consists essentially of, or yet further consists of transducing a cell or a population thereof with a polynucleotide as disclosed herein or a vector as disclosed herein or a vector system as disclosed herein. In some embodiments, the polynucleotide or the vector or the vector system encodes the CAR, the bispecific antibody, the cytokine and the suicide gene.

In some embodiments of any aspect as disclosed herein, the cell is selected from a Hematopoietic stem cell (HSC), an induced pluripotent stem cell (iPSCs), or an immune cell.

In some embodiments of any aspect as disclosed herein, the cell population comprises, or consists essentially of, or yet further consists of one or more of the following: an HSC, an iPSC, or an immune cell.

In some embodiments of any aspect as disclosed herein, the immune cell is selected from T-cells, B cells, NK cells, dendritic cells, myeloid cells, monocytes, or macrophages. In some embodiments of any aspect as disclosed herein, the immune cell is derived from an HSC or an iPSC.

In some embodiments of any aspect as disclosed herein, the method further comprises selecting or enriching the cell comprising the polynucleotide or the vector or a vector system as disclosed herein. Additionally or alternatively, the method further comprises selecting or enriching the cell expressing the CAR. In some embodiments, the method further comprises culturing the cell under a suitable condition, such as in a suitable culture medium, under a suitable culture temperature, with a suitable oxygen supply, or any combination thereof. In some embodiments, the method further comprises preserving the cell.

### Treatment Methods

In one aspect, provided is a method of inhibiting the growth of a cancer cell expressing a tumor associated antigen (TAA, such as GPRC5D or BCMA or both) or a tissue comprising the cancer cell. The method comprises, or consists essentially of, or yet further consists of contacting the cancer cell or the tissue with a cell as disclosed herein, such as an engineered or isolated cell as disclosed herein, that in some embodiments, is selected to target the TAA, or a cell population as disclosed herein. Some embodiments of the method further comprise contacting the cancer cell or the tissue with a bispecific antibody or a fragment thereof as disclosed herein. In some embodiments, the contacting is in vitro or ex vivo or in vivo. In one embodiment, the contacting is in vivo and the isolated cells are autologous or allogeneic to a subject being treated. In another embodiment, the contacting is in vivo and the isolated cells are allogenic to a subject being treated. In some embodiments, the cancer cell expresses a TAA of BCMA. Additionally or alternatively, the cancer cell expresses a TAA of GPRC5D. In some embodiments, the cancer cell expresses both BCMA and GPRC5D. In some embodiments, an effective amount of the cell is used.

In another aspect, provided is a method of inhibiting the growth of a cancer cell expressing GPRC5D or a tissue comprising the cancer cell. The method comprises, or consists essentially of, or yet further consists of contacting the cancer or tissue with a bispecific antibody or a fragment thereof as disclosed herein, or a cell or a cell population as disclosed herein, or both a bispecific antibody or a fragment thereof and a cell or a cell population as disclosed herein. In some embodiments, the contacting is in vitro or ex vivo or in vivo. In some embodiments, an effective amount of the cell or the antibody or both is used.

Some embodiments of the method further comprises contacting the cancer cell or the tissue with an effective amount of an anti-cancer therapy, a therapy depleting an immune cell, a cytoreductive therapy, a therapy that upregulates the expression of the TAA on the cancer cell, or any combination thereof. In some embodiments, the contacting is in vitro or ex vivo or in vivo.

In yet another aspect, provided is a method for treating a cancer in a subject in need thereof. The method comprises, or consists essentially of, or yet further consists of administering a cell or a cell population as disclosed herein, such as an isolated or engineered cell as disclosed herein, to the subject. In some embodiments, the subject is selected for the therapy by determining expression of one or more tumor associated antigens (TAAs) in a sample isolated from the subject and administered with the cell that expresses a CAR that recognizes and bind the one or more TAAs. In some embodiments, the method further comprises selecting a subject by determining expression of one or more tumor associated antigens (TAAs) in a sample isolated from the subject. In some embodiments, the TAA expression is determined by contacting the sample with an anti-TAA antibody or an antigen binding fragment thereof in vitro or ex vivo or in vivo and detecting binding between the sample and the anti-TAA antibody or antigen binding fragment thereof. In further embodiments, the anti-TAA antibody further comprises a detectable marker. In one embodiment, at least one of the TAAs comprises, or consists essentially of, or yet further consists of an epitope of BCMA. Additionally or alternatively, at least one of the TAAs comprises, or consists essentially of, or yet further consists of an epitope of GPRC5D. In some embodiments, an effective amount of the cell is used.

Some embodiments of a method as disclosed herein further comprises administering a bispecific antibody or a fragment thereof of as disclosed herein to the subject.

In one aspect, provided is a method for treating a cancer in a subject selected for the treatment. The method comprises, or consists essentially of, or yet further consists of administering a cell or a cell population as disclosed herein, such as the isolated or engineered cell as disclosed herein, to the subject. In some embodiments, the subject is selected via if a cancer cell of the subject expresses one or more of tumor associated antigen(s) (TAAs). In further embodiments, the TAA expression is determined by contacting a cancer cell of the subject with an anti-TAA antibody or an antigen binding fragment thereof in vitro or ex vivo or in vivo. In some embodiments, the anti-TAA antibody comprises a detectable marker. In some embodiments, the TAA is BCMA. Additionally or alternatively, the TAA is GPRC5D. In some embodiments, an effective amount of the cell is used.

In some embodiments of any aspect as disclosed herein, the cell (such as the isolated or engineered cell) and the bispecific antibody or a fragment thereof are administered together. In some embodiments, the cell (such as the isolated or engineered cell) and the bispecific antibody or a fragment thereof are administered separately. In further embodiments, the cell (such as the isolated or engineered cell) and the bispecific antibody or a fragment thereof are administered concurrently. In yet further embodiments, the cell (such as the isolated or engineered cell) and the bispecific antibody or a fragment thereof are administered 1 hours apart, 4 hours apart, 24 hours apart, 2 days apart, 3 days apart, or 1 week apart.

In some embodiments, the cell (such as the isolated or engineered cell) is autologous or allogeneic to the subject in need. In some embodiments, the cell (such as the isolated or engineered cell) is allogenic to the subject in need.

In one aspect, provided is a method for treating a GPRC5D-expressing cancer in a subject. The method comprises, or consists essentially of, or yet further consists of administering the bispecific antibody or a fragment thereof as disclosed herein to the subject.

Some embodiments of a method as disclosed herein further comprises administering to the subject another anti-cancer therapy, such as a cytoreductive therapy, or a therapy that upregulates the expression of the tumor associated antigen (TAA), such as BCMA or GPRC5D or both. In further embodiments, a cytoreductive therapy comprises, or consists essentially of, or yet further consists of one or more of the following: a chemotherapy, a cryotherapy, a hyperthermia, a targeted therapy, or a radiation therapy.

In some embodiments of a method as disclosed herein, the administration is applied to the subject as a first line therapy, or a second line therapy, or third line therapy, or a fourth line therapy.

In some embodiments of any aspect as disclosed herein, the subject is a mammal, a canine, a feline, an equine, a murine, or a human patient.

In some embodiments of any aspect as disclosed herein, the cancer is multiple myeloma (MM). In some embodiments of any aspect as disclosed herein, the cancer is an acute myeloid leukemia (AML).

In some embodiments where tEGFR is used as the suicide gene product or the detectable marker or both, the method further comprises administering an antibody that recognizes and binds tEGFR (an anti-tEGFR antibody) after the administrating the cell, thereby eliminating tEGFR expressing cells. In some embodiments, the administration of the anti-tEGFR antibody is about 4 weeks, or about 1.5 months, or about 2 months, or about 3 months, or about 4 months, or about 5 months, or about 6 months, or about 7 months, or about 8 months, or about 9 months, or about 10 months, or about 11 months, or about 12 months, or about 1.5 years after the administration of the cells. Suitable anti-tEGFR antibody may be selected from one or more of the following: cetuximab, necitumumab, or panitumumab.

In some embodiments where the suicide gene product or detectable marker or both comprises, or consists essentially of, or yet further consists of RQR8, the method further comprises administering an antibody that recognizes and binds RQR8 (i.e., an anti-RQR8 antibody) after the administrating the cell, thereby eliminating RQR8 expressing cells. In some embodiments, the anti-RQR8 antibody recognizes and binds a CD34 epitope in the RQR8. Additionally or alternatively, the anti-RQR8 antibody recognizes and binds a CD20 epitope in the RQR8. In some embodiments, the administration of the anti-RQR8 antibody is about 4 weeks, or about 1.5 months, or about 2 months, or about 3 months, or about 4 months, or about 5 months, or about 6 months, or about 7 months, or about 8 months, or about 9 months, or about 10 months, or about 11 months, or about 12 months, or about 1.5 years after the administration of the cells. Suitable anti-RQR8 antibody may be selected from one or more of the following: rituximab, ofatumumab, ocrelizumab, obinutuzumab, ibritumomab, or tositumomab.

In some embodiments where the suicide gene product or detectable marker or both comprises, or consists essentially of, or yet further consists of RQR8, the method further comprises purifying the cells by selecting RQR8 expressing cells. In some embodiments, an anti-RQR8 antibody is used to select the RQR8 expressing cells. In further embodiments, a system available to one of skill in the art, such as CliniMACS CD34 Reagent System is used to select the RQR expressing cells.

In some embodiments, the cancer cell is selected from cancer cells of the: circulatory system, for example, heart (sarcoma [angiosarcoma, fibrosarcoma, rhabdomyosarcoma, liposarcoma], myxoma, rhabdomyoma, fibroma, and lipoma), mediastinum and pleura, and other intrathoracic organs, vascular tumors and tumor-associated vascular tissue; respiratory tract, for example, nasal cavity and middle ear, accessory sinuses, larynx, trachea, bronchus and lung such as small cell lung cancer (SCLC), non-small cell lung cancer (NSCLC), bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; gastrointestinal system, for example, esophagus (squamous cell carcinoma, adenocarcinoma, leiomyosarcoma, lymphoma), stomach (carcinoma, lymphoma, leiomyosarcoma), gastric, pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma); gastrointestinal stromal tumors and neuroendocrine tumors arising at any site; genitourinary tract, for example, kidney (adenocarcinoma, Wilm's tumor [nephroblastoma], lymphoma, leukemia), bladder and/or urethra (squamous cell carcinoma, transitional cell carcinoma, adenocarcinoma), prostate (adenocarcinoma, sarcoma), testis (seminoma, embryonal carcinoma, teratocarcinoma, choriocarcinoma, sarcoma, interstitial cell carcinoma, fibroma, fibroadenoma, adenomatoid tumors, lipoma); liver, for example, hepatoma (hepatocellular carcinoma), cholangiocarcinoma, hepatoblastoma, angiosarcoma, hepatocellular adenoma, hemangioma, pancreatic endocrine tumors (such as pheochromocytoma, insulinoma, vasoactive intestinal peptide tumor, islet cell tumor and glucagonoma); bone, for example, osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; nervous system, for example, neoplasms of the central nervous system (CNS), primary CNS lymphoma, skull cancer (osteoma, hemangioma, granuloma, xanthoma, osteitis deformans), meninges (meningioma, meningiosarcoma, gliomatosis), brain cancer (astrocytoma, medulloblastoma, glioma, ependymoma, germinoma [pinealoma], glioblastoma multiform, oligodendroglioma, schwannoma, retinoblastoma, congenital tumors), spinal cord neurofibroma, meningioma, glioma, sarcoma); reproductive system, for example, gynecological, uterus (endometrial carcinoma), cervix (cervical carcinoma, pre-tumor cervical dysplasia), ovaries (ovarian carcinoma [serous cystadenocarcinoma, mucinous cystadenocarcinoma, unclassified carcinoma], granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), placenta, vagina (clear cell carcinoma, squamous cell carcinoma, botryoid sarcoma (embryonal rhabdomyosarcoma), fallopian tubes (carcinoma) and other sites associated with female genital organs;, penis, prostate, testis, and other sites associated with male genital organs; hematologic system, for example, blood (myeloid leukemia [acute and chronic], acute lymphoblastic leukemia, chronic lymphocytic leukemia, myeloproliferative diseases, multiple myeloma, myelodysplastic syndrome), Hodgkin's disease, non-Hodgkin's lymphoma [malignant lymphoma]; oral cavity, for example, lip, tongue, gum, floor of mouth, palate, and other parts of mouth, parotid gland, and other parts of the salivary glands, tonsil, oropharynx, nasopharynx, pyriform sinus, hypopharynx, and other sites in the lip, oral cavity and pharynx; skin, for example, malignant melanoma, cutaneous melanoma, basal cell carcinoma, squamous cell carcinoma, Kaposi's sarcoma, moles dysplastic nevi, lipoma, angioma, dermatofibroma, and keloids; adrenal glands: neuroblastoma; and other tissues comprising connective and soft tissue, retroperitoneum and peritoneum, eye, intraocular melanoma, and adnexa, breast, head or neck, anal region, thyroid, parathyroid, adrenal gland and other endocrine glands and related structures, secondary and unspecified malignant neoplasm of lymph nodes, secondary malignant neoplasm of respiratory and digestive systems and secondary malignant neoplasm of other sites.

In some embodiments, the cancer cell is a solid tumor cell. In other embodiments, the cancer cell is not a cell of a solid tumor. In further embodiments, the cancer cell is a leukemia cancer cell. In some embodiments, the cancer cell is a primary cancer cell or a metastatic cancer cell. In some embodiments, the cancer cell is from a carcinoma, a sarcoma, a myeloma, a leukemia, or a lymphoma.

Method aspects of the present disclosure relate to methods for inhibiting the growth of a tumor or cancer cells, (e.g., Multiple Myeloma (MIN), Acute Myeloid Leukemia (AML) or glioblastoma multiforme (GB) cells) in vitro or ex vivo or in vivo; or for treating a cancer patient in need thereof; or both. In some embodiments, the tumor is a solid tumor. In some embodiments, the cancer is a cancer affecting blood or bone marrow or both, e.g., MM. In some embodiments, the cancer or tumor cell expresses or overexpresses a cancer or tumor antigen BCMA or GPRC5D or both. When practiced in vitro or ex vivo, the methods provide in vitro or ex vivo assays for precision medicine application and useful assays for testing new combination and therapies.

In some embodiments, the administering step may be repeated for once, twice, three times, four times, five times, six times, seven times, eight times, nine times, ten times or more. In further embodiments, two administrations are about 1 day apart, about 2 days apart, about 3 days apart, about 4 days apart, about 5 days apart, about 6 days apart, about 1 week apart, about 10 days apart, about 2 weeks apart, about 3 weeks apart, about 4 weeks apart, about 1 month apart, about 2 months apart, about 3 months apart, about 4 months apart, about 5 months apart, about 6 months apart, about 7 months apart, about 8 months apart, about 9 months apart, about 10 months a part, about 11 months apart, about 1 year apart, about 1.5 years apart, about 2 years apart, about 3 years apart, about 5 years apart, about 10 years apart or longer.

Additional aspects of the disclosure relate to compositions comprising, or alternatively consisting essentially of, or yet further consisting of, a carrier and one or more of the products—e.g., a cell population as disclosed herein, a CAR, an isolated cell comprising a CAR, a polypeptide, a polynucleotide, an isolated nucleic acid, a vector, a vector system, a cell, a cell population, or an isolated cell containing the CAR or the bispecific antibody or both as disclosed herein or nucleic acids encoding such—described in the embodiments disclosed herein. In some embodiments, the carrier is a pharmaceutically acceptable carrier. In further aspects, the composition may additionally comprise an immunoregulatory molecule.

Briefly, pharmaceutical compositions of the present disclosure including but not limited to any one of the claimed compositions as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present disclosure may be formulated for local or systemic administration, e.g., oral, intravenous, intracranial, topical, enteral, or parenteral administration. In certain embodiments, the compositions of the present disclosure are formulated for intravenous administration.

Administration of the cells or compositions can be performed in one dose, continuously or intermittently throughout the course of treatment and an effective amount to achieve the desired therapeutic benefit is provided. Methods of determining the most effective means and dosage of administration are known to those of skill in the art and will vary with the composition used for therapy, the purpose of the therapy and the subject being treated. Single or multiple administrations can be carried out with the dose level and pattern being selected by the treating physician. Suitable dosage formulations and methods of administering the agents are known in the art. In a further aspect, the cells and composition of the disclosure can be administered in combination with other treatments.

The cells and populations of cell are administered to the host or subject using methods known in the art and described, for example, in WO2012079000A1. This administration of the cells or compositions of the disclosure can be done to generate an animal model of the desired disease, disorder, or condition for experimental and screening assays.

### Combination Therapies

The compositions as described herein can be administered as first line, second line, third line, fourth line, or other therapy and can be combined with another anti-cancer therapy, such as cytoreductive interventions. The can be administered sequentially or concurrently as determined by the treating physician. In some embodiments, they can be combined with therapies that may upregulate the expression of a tumor or other antigen to which the CAR or BsAb binds. In some embodiments, some clinical drugs can increase targeted antigens. In some embodiments, they can be combined with surgical removal of the cancer or tumor. In some embodiments, the cytoreductive therapy comprises, or alternatively consists essentially of, or yet consists of one or more of chemotherapy, cryotherapy, hyperthermia, targeted therapy, or radiation therapy. The compositions and therapies can be combined with other therapies, e.g., lymphodepletion chemotherapy followed by infusions (e.g., four weekly infusions) of the therapy, defining one cycle, followed by additional cycles until a partial or complete response is seen or alternatively utilized as a “bridging” therapy to another modality, such as hematopoietic stem cell transplantation or CAR T cell therapy.

### Kits

As set forth herein, the present disclosure provides methods for producing and administering CAR or BsAb CAR cells. In one particular aspect, the present disclosure provides kits for performing these methods as well as instructions for carrying out the methods of the present disclosure such as collecting cells or tissues, performing the screen/transduction/etc., analyzing the results, or any combination thereof.

In one aspect, the kit comprises, or alternatively consists essentially of, or yet further consists of, any one or more of: a polypeptide as disclosed herein, a CAR as disclosed herein, a bispecific antibody as disclosed herein, a polynucleotide as disclosed herein, a vector as disclosed herein, a vector system as disclosed herein a cell as disclosed herein, such as isolated allogenic cells, preferably T cells or NK cells, a cell population as disclosed herein, a composition as disclosed herein, an isolated complex as disclosed herein, or an optional instruction for use in a method as disclosed herein, for example, on the procuring of autologous cells from a patient. Such a kit may also comprise, or alternatively consist essentially of, or yet further comprise media and other reagents appropriate for the transduction, selection, activation, expansion or any combination thereof of CAR or BsAb CAR expressing cells, such as those disclosed herein.

In one aspect the kit comprises, or alternatively consists essentially of, or yet further consists of, an isolated CAR or BsAb CAR expressing cell or population thereof. In some embodiments, the cells of this kit may require activation or expansion or both prior to administration to a subject in need thereof. In further embodiments, the kit may further comprise, or consist essentially of, media and reagents, such as those covered in the disclosure above, to activate or expand or both activate and expand the isolated CAR or BsAb CAR expressing cell. In some embodiments, the cell is to be used for a CAR therapy. In further embodiments, the kit comprises instructions on the administration of the isolated cell to a patient in need of a CAR therapy.

The kits of this disclosure can also comprise, e.g., a buffering agent, a preservative or a protein-stabilizing agent. The kits can further comprise components necessary for detecting the detectable-label, e.g., an enzyme or a substrate. The kits can also contain a control sample or a series of control samples, which can be assayed and compared to the test sample. Each component of a kit can be enclosed within an individual container and all of the various containers can be within a single package, along with instructions for interpreting the results of the assays performed using the kit. The kits of the present disclosure may contain a written product on or in the kit container. The written product describes how to use the reagents contained in the kit.

As amenable, these suggested kit components may be packaged in a manner customary for use by those of skill in the art. For example, these suggested kit components may be provided in solution or as a liquid dispersion or the like.

### Experimental Methods

The following examples are illustrative of procedures which can be used in various instances in carrying the disclosure into effect.

### EXAMPLE 1—Anti BCMA CAR-T with Secretion of Anti-GPRC5D and NKG2D-Bispecific Engager (BiTE) Enhances Anti-Multiple Myeloma (MM) Efficacy (In Vitro)

Immune Therapy for Multiple Myeloma (MM) and Target Selection

The CAR-T therapy and Bispecific T cell engager (BiTE) are two popular immune therapy strategies used for Multiple Myeloma recently. Targets selection is critical for immune therapy. The two criterions for making the choice are the roles in the development of Multiple Myeloma and the specifically expressing on MM. B cell maturation antigen (BCMA) and the orphan G protein—coupled receptor, class C group 5 member D (GPRC5D) are the most popular targets for Multiple Myeloma.

In Applicant's blind study, it was found that both mouse anti human BCMA and humanized anti BCMA CAR-T, as well as GPRC5D CAR-T dramatically kill BCMA and GPRC5D double positive MM cell line MM1 (Data not shown). More importantly, GPRC5D expression on multiple myeloma cells was independent of BCMA expression, suggesting that GPRC5D-targeted can overcome the BCMA-antigen loss mediated relapse.

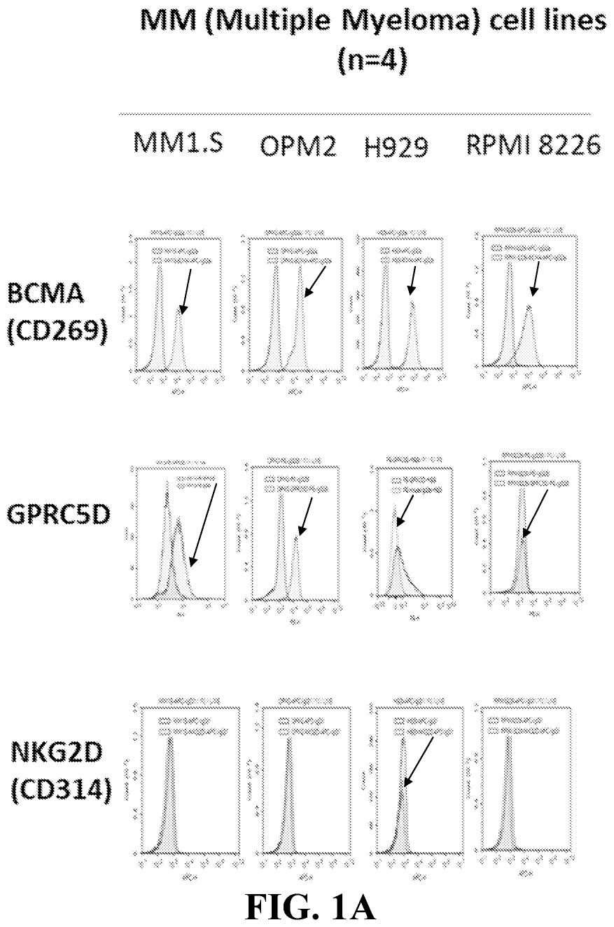

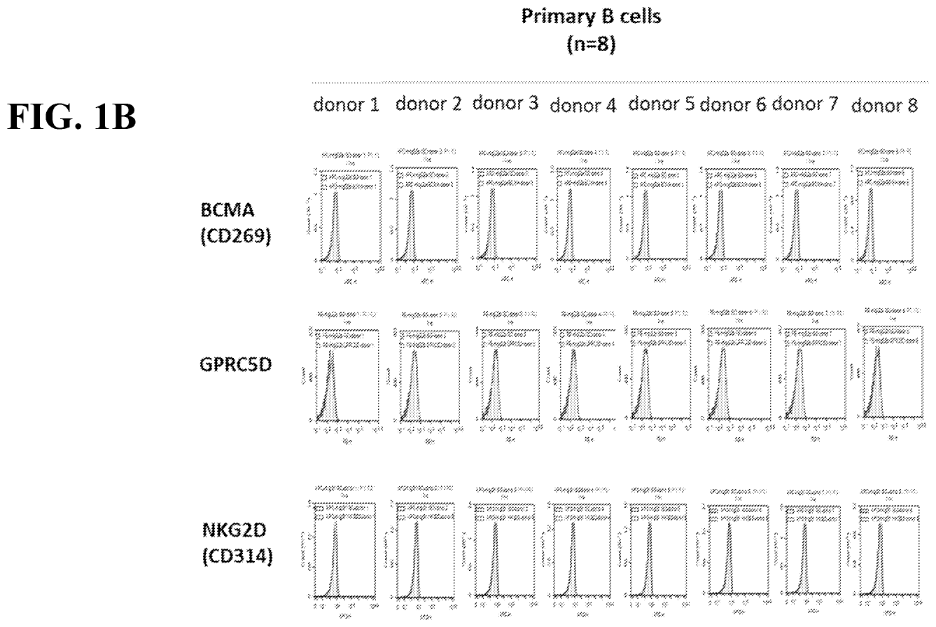

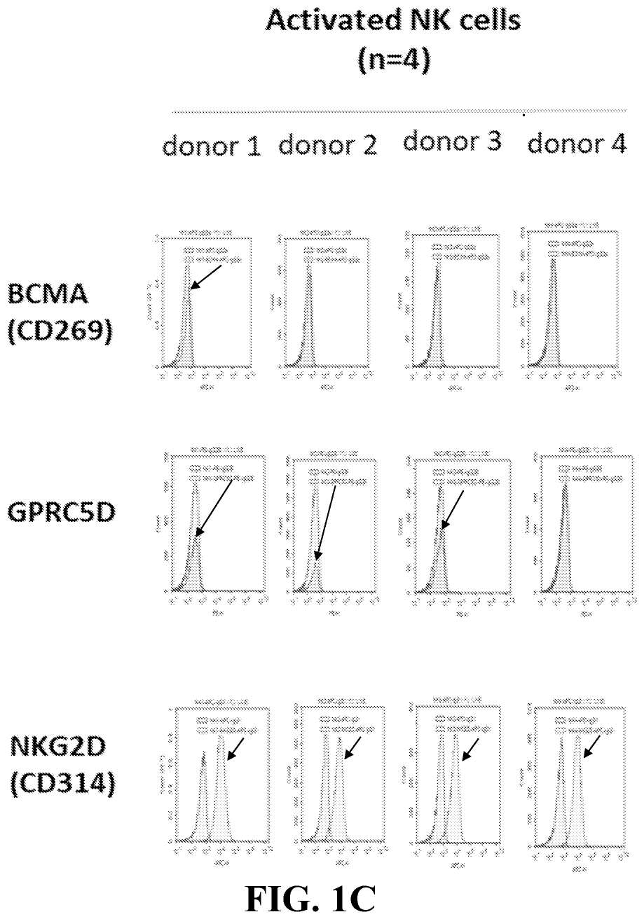

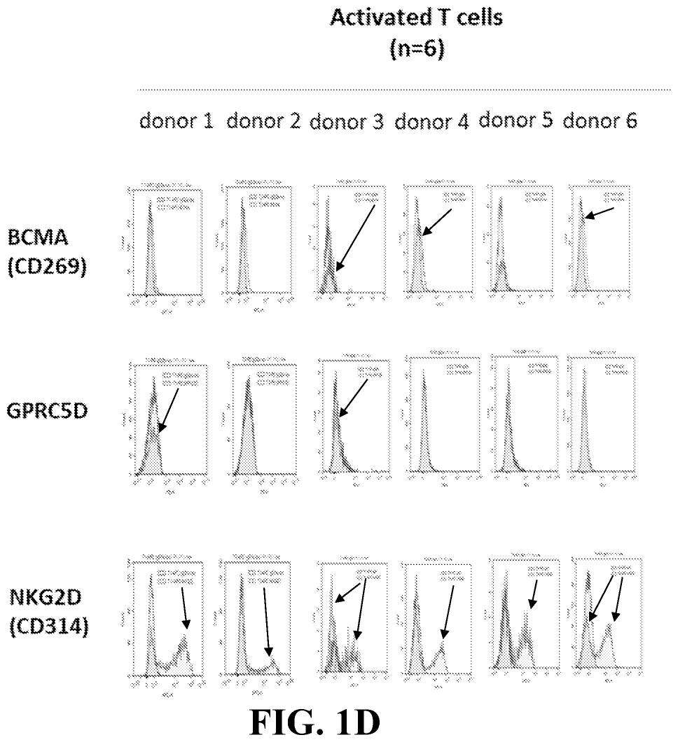

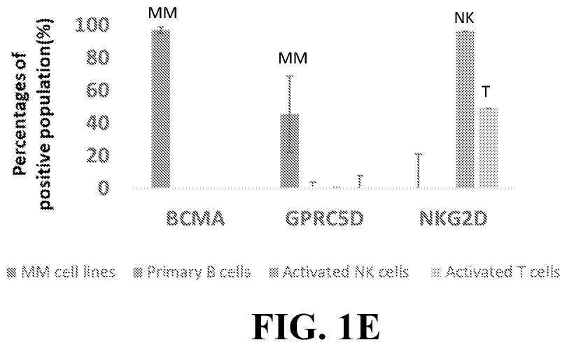

For specific targets identification, Applicant performed flow analysis of BCMA and GPRC5D surface expression on four Multiple Myeloma (MM) cell lines, primary B cell, NK cells and T cells. MM1.S, OPM2, H929, and RPMI8226 are Multiple Myeloma (MM) cell lines and were cultures in complete RPMI 1640 media. The freshly isolated B cells, and activated NK cells and T cells were used in this study. Total 2×10⁵ cells were blocked using Human TruStain FcX™ (Fc Receptor Blocking Solution) at 1:50 dilution, then stain BCMA, GPRC5D and NKG2D with their isotype controls. NovoCyte Flow Cytometer 3005 (ACEA Biosciences. Inc), and NovoExpress software 1.4.1 were used to detect and analyze data. The data demonstrated that, the BCMA is expressed on the surface of all the four MM cell lines, but not primary B cells and activated NK and T cells. GPRC5D were detected only on the surface of three MM cell lines MM1.S, OPM2 and H929, but excepted for all Primary B cells, NK, and T cells (FIGS. 1A-1B).

“Two in One” Combination of BCMA-CAR-T and BiTE Targeting GPRC5D and NKG2D

Applicant hypothesized that the “Two in One” combination of anti-BCMA CAR-T and a BiTE targeting both NKG2D expressed on T cells and a specific tumor antigen GPRC5D on MM would be an advanced therapy strategy to outperform the anti BCMA CAR-T and BiTE alone.

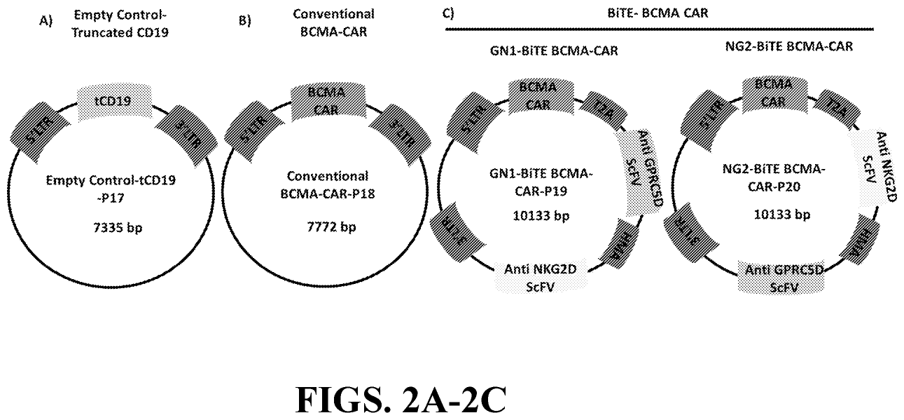

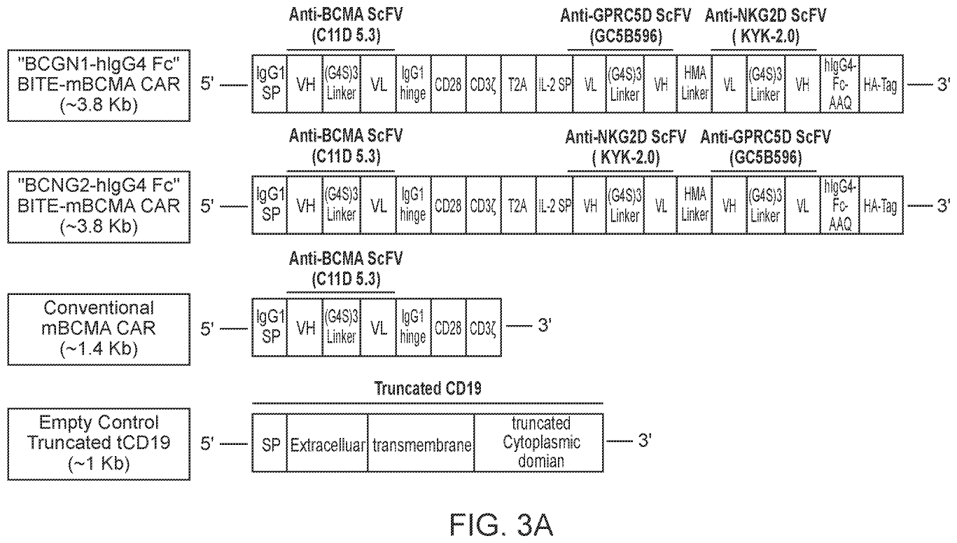

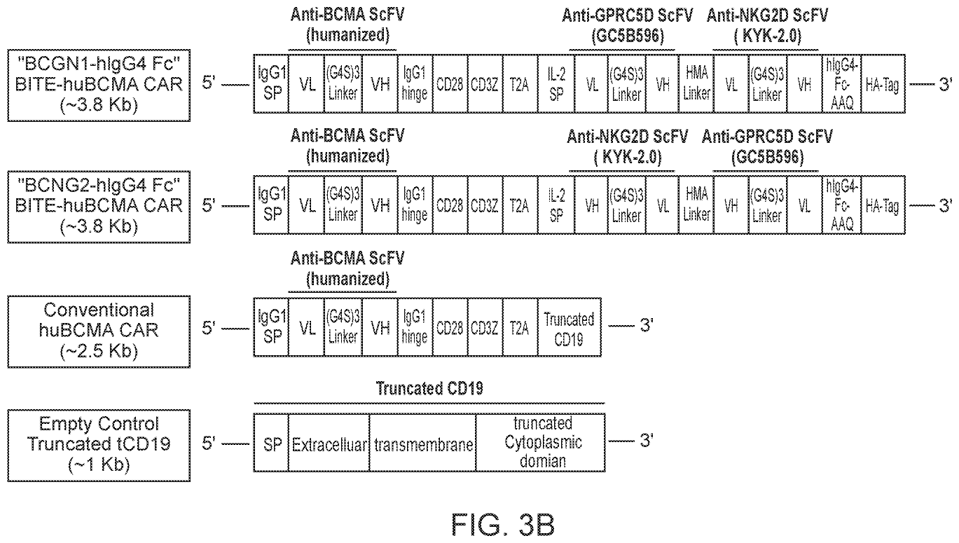

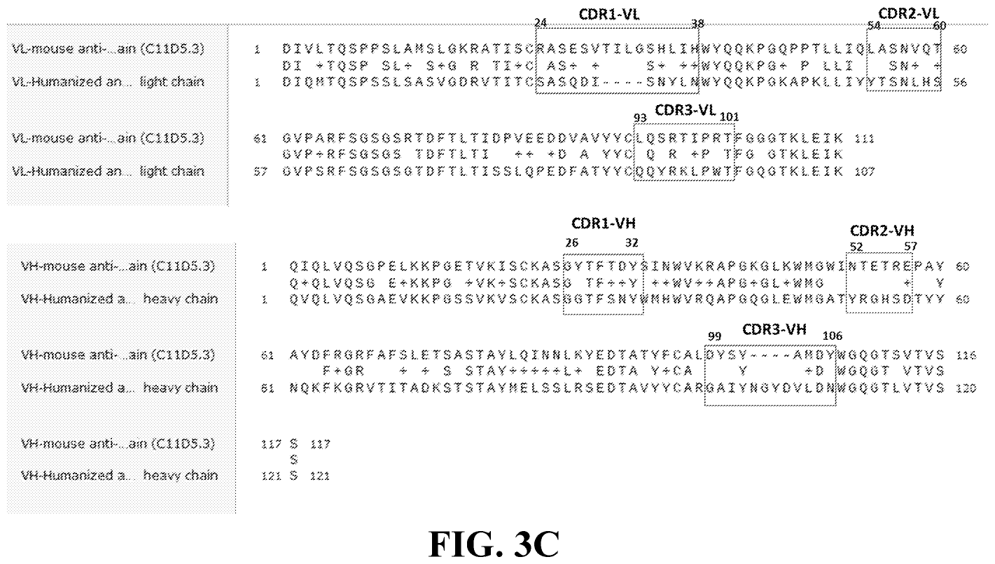

Schematic representations and diagraphs of anti-BCMA conventional CAR and two anti GPRC5D and NKG2D BiTE CAR retroviral vectors were illustrated in FIGS. 2A-3C.

The anti BCMA scFv was extracted from both mouse anti human BCMA antibody C11D 5.3 (WO 2015/158671 A1) (FIG. 3A) and humanized anti human BCMA (SEQ ID NO: 271 in U.S. Ser. No. 10/174,095 B2) (FIG. 3B). For the BiTE, full human source human anti human NKG2D antibody KYK-2.0 (US20110150870A1) and human anti human GPRC5D antibody GC5B596 (US Patent Application 20180037651) were used.

There were two BiTE-BCMA CAR constructs used in this study with the differences being the first scFv and second scFv in BiTE components. The human IgG4 Fc used in BiTE was aimed to stabilize the BiTE secreted. To avoid the Fc receptors binding, Applicant initially mutated IgG4-Fc at three sites, F234A and L235A, within hinge region, and N297Q within CH2 region substitutions in its Fc region. All above four inserts were cloned into retrovirus vectors PCIR between 5′ and 3′ LTR.

The amino acids and nucleotide sequences for each components and whole sequences of above constructs were provided in Tables 1a—1h.

TABLE 1a
CAR-Components used in this study.

| | Amino acid | Nucleotide |
|---|---|---|
| | sequence | sequence |
| | (SEQ ID NO:) | (SEQ ID NO:) |
| IgG1 SP | 21 | 55 |
| VL-mouse anti-human BCMA light | 41 | 58 |
| chain (C11D5.3) | | |
| CDR1-VL-C11D5.3 | 23 | 171 |
| CDR2-VL-C11D5.3 | 26 | 172 |
| CDR3-VL-C11D5.3 | 29 | 173 |
| VH-mouse anti-human BCMA heavy | 44 | 62 |
| chain (C11D5.3) | | |
| CDR1-VH-C11D5.3 | 32 | 174 |
| CDR2-VH-C11D5.3 | 35 | 175 |
| CDR3-VH-C11D5.3 | 38 | 176 |
| VL-Humanized anti-BCMA light | 42 | 59 |
| chain | | |
| CDR1-VL Humanized anti-BCMA | 24 | 177 |
| light chain | | |
| CDR2-VL-Humanized anti-BCMA | 27 | 178 |
| light chain | | |
| CDR3-VL-Humanized anti-BCMA | 30 | 179 |
| light chain | | |
| VH-Humanized anti-BCMA heavy | 45 | 63 |
| chain | | |
| CDR1-VH Humanized anti-BCMA | 33 | 180 |
| light chain | | |
| CDR2-VH-Humanized anti-BCMA | 36 | 181 |
| light chain | | |
| CDR3-VH-Humanized anti-BCMA | 39 | 182 |
| light chain | | |
| (G4S)3 linker | 9 | 57 |
| IgG1 hinge | 47 | 66 |
| CD28 transmembrane and | 48 | 67 |
| cytoplasmic/intracellular domain | | |
| CD3Z isoform 3 | 49 | 68 |
| T2A | 50 | 69 |
| IL-2 signal sequence | 20 | 56 |
| VL-human anti-human GPRC5D | 16 | 71 |
| light chain (GC5B596) | | |
| CDR1-VL-GC5B596 | 10 | 183 |
| CDR2-VL-GC5B596 | 11 | 184 |
| CDR3-VL-GC5B596 | 12 | 185 |
| VH-human anti-human GPRC5D | 17 | 72 |
| heavy chain (GC5B596) | | |
| CDR1-VH-GC5B596 | 13 | 186 |
| CDR2-VH-GC5B596 | 14 | 187 |
| CDR3-VH-GC5B596 | 15 | 188 |
| VL-human anti-human NKG2D light | 7 | 73 |
| chain (KYK-2.0) | | |
| CDR1-VL-KYK-2.0 | 1 | 189 |
| CDR2-VL-KYK-2.0 | 2 | 190 |
| CDR3-VL-KYK-2.0 | 3 | 191 |
| VH-human anti-human NKG2D | 8 | 74 |
| heavy chain (KYK-2.0) | | |
| CDR1-VH-KYK-2.0 | 4 | 192 |
| CDR2-VH-KYK-2.0 | 5 | 193 |
| CDR3-VH-KYK-2.0 | 6 | 194 |
| HMA linker | 81 | 70 |
| three-site mutated human IgG4 Fc - | 19 | 75 |
| AAQ | | |
| HA-Tag | 22 | 76 |
| Truncated CD19 | 170 | 195 |

TABLE 1b
BCGN1-hIgG4 Fc BITE-mBCMA CAR (m: Mouse anti human)

| | Amino acid | Nucleotide |
|---|---|---|
| | sequence | sequence |
| | (SEQ ID NO:) | (SEQ ID NO:) |
| BCGN1-hIgG4 Fc″ BITE- | 51 | 77 |
| mBCMA CAR | | |

TABLE 1c
BCNG2-hIgG4 Fc BITE-mBCMA CAR (m: Mouse anti human)

| | Amino acid | Nucleotide |
|---|---|---|
| | sequence | sequence |
| | (SEQ ID NO:) | (SEQ ID NO:) |
| BCNG2-hIgG4 Fc″ BITE- | 52 | 78 |
| mBCMA CAR | | |

TABLE 1d
Conventional mBCMA-CAR (m: Mouse anti human)

| | Amino acid | Nucleotide |
|---|---|---|
| | sequence | sequence |
| | (SEQ ID NO:) | (SEQ ID NO:) |
| Conventional mBCMA-CAR | 196 | 197 |
| (mouse anti human BCMA | | |
| antibody C11D5.3) | | |

TABLE 1e
Truncated CD19 empty vector

| | Amino acid | Nucleotide |
|---|---|---|
| | sequence | sequence |
| | (SEQ ID NO:) | (SEQ ID NO:) |
| Truncated CD19 empty vector | 170 | 195 |

*TABLE 1f
BCGN1-hIgG4 Fc BITE-huBCMA (hu: humanized)

| | Amino acid | Nucleotide |
|---|---|---|
| | sequence | sequence |
| | (SEQ ID NO:) | (SEQ ID NO:) |
| BCGN1-hIgG4 Fc″ BITE- | 53 | 79 |
| huBCMA CAR | | |

*TABLE 1g
BCNG2-hIgG4 Fc BITE-huBCMA (hu: humanized)

| | Amino acid | Nucleotide |
|---|---|---|
| | sequence | sequence |
| | (SEQ ID NO:) | (SEQ ID NO:) |
| BCNG2-hIgG4 Fc″ BITE- | 54 | 80 |
| huBCMA CAR | | |

*TABLE 1h
Conventional huBCMA-CAR (hu: humanized)

| | Amino acid | Nucleotide |
|---|---|---|
| | sequence | sequence |
| | (SEQ ID NO:) | (SEQ ID NO:) |
| Conventional huBCMA-CAR | 198 | 199 |
| (humanized) | | |

Unless specified, the data in this disclosure was based on mouse anti-human BCMA CAR-T.

In summary, as shown in FIGS. 1A-1E, expression of BCMA, GPRC5D and NKG2D surface expression was demonstrated on Multiple Myeloma (MM) cell lines, primary B cell, NK cells and T cells. FIGS. 2A-2B provide schematic representations of anti-BCMA conventional CAR and two anti GPRC5D and NKG2D BiTE CAR retroviral vectors. The mouse anti-human BCMA scFv is replaced with humanized anti-Human BCMA scFv. FIGS. 3A-3B provide diagraphs of BiTE-BCMA CAR and controls with amino acid and nucleotide sequences (Tables 1a-1h).

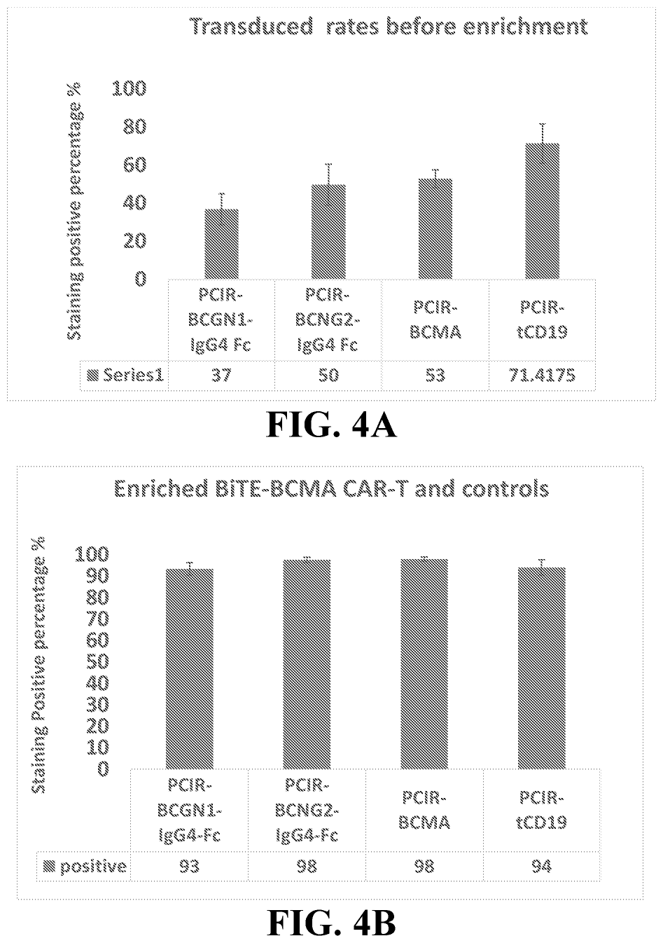

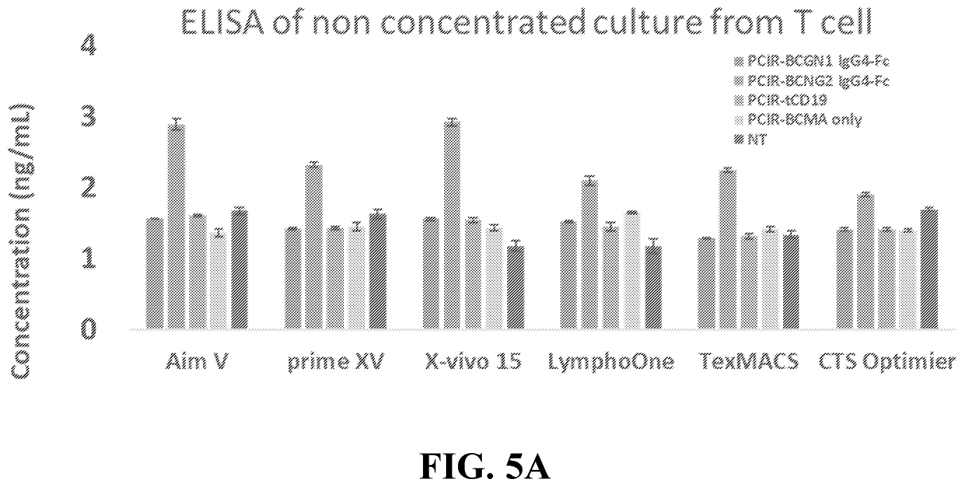

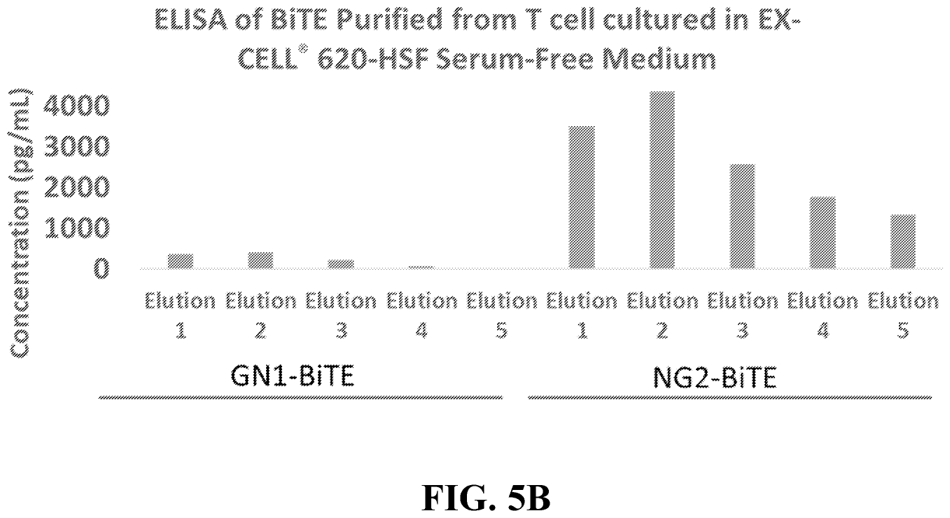

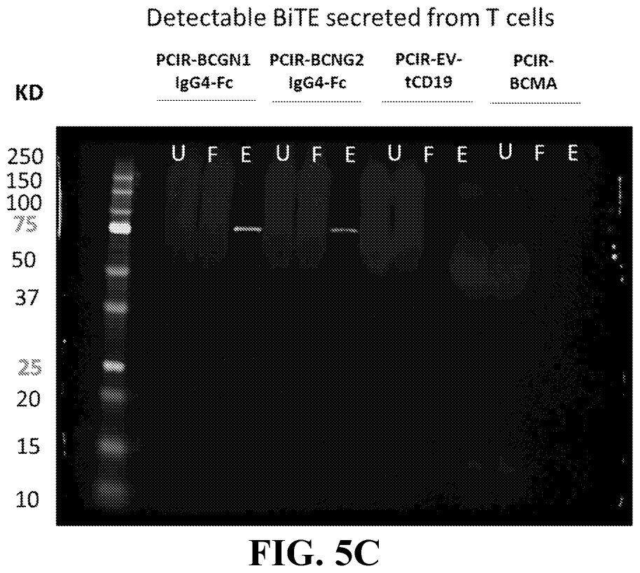

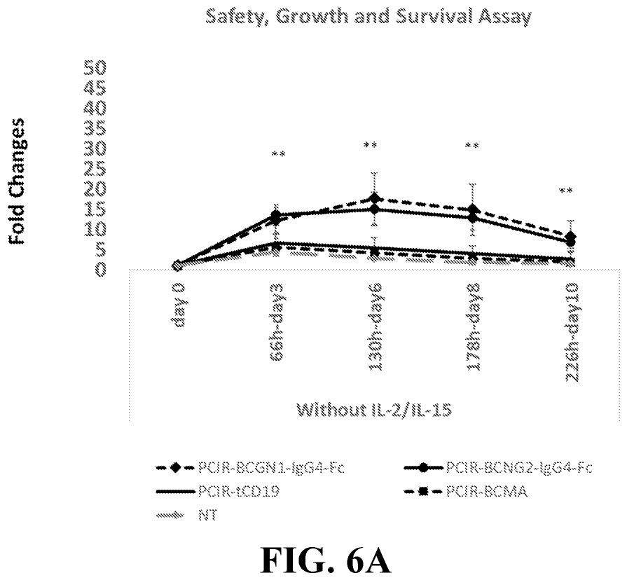

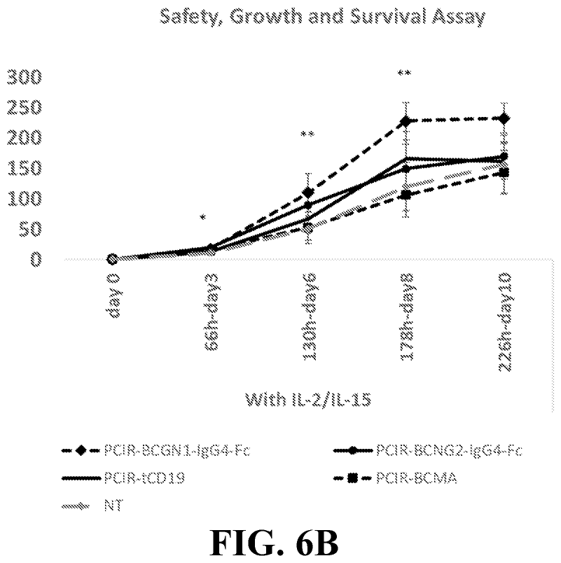

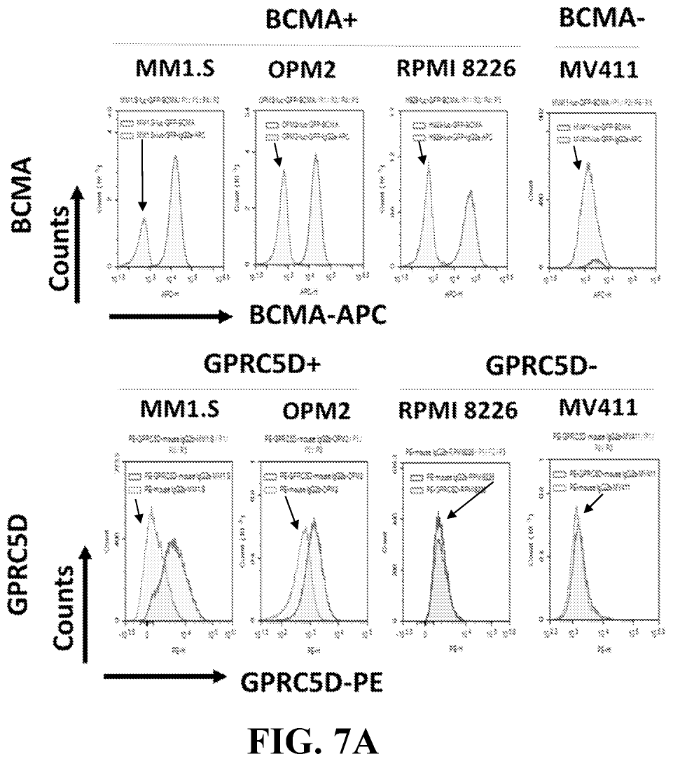

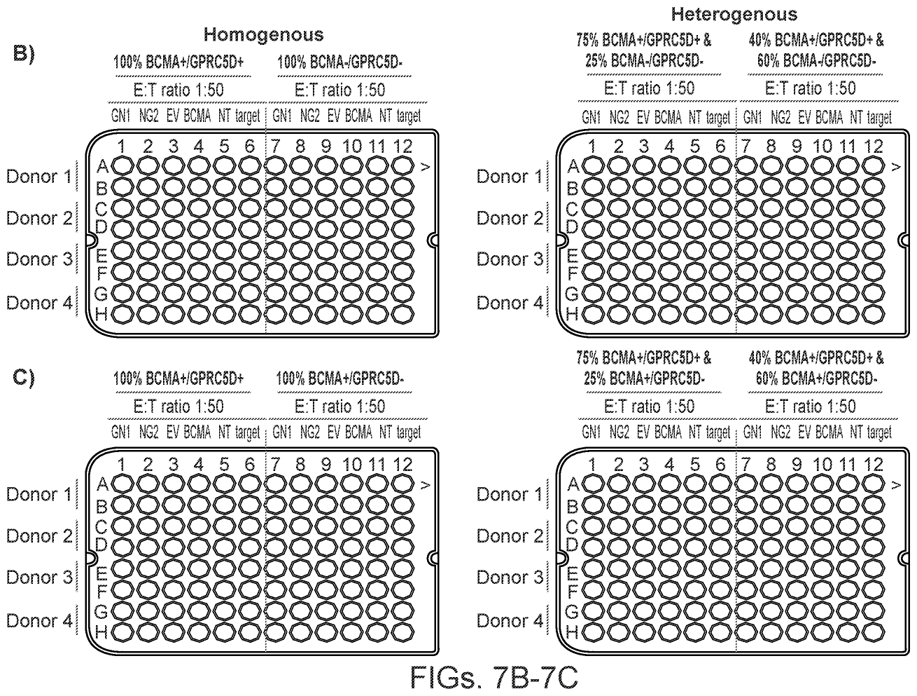

Transduction was evaluated and purity was detected by flow assay before and after enrichment. The results are provided in FIGS. 4A-4B. The expression and concentration of bispecific antibody secreted from T cells were determined by Western Blotting and ELISA. See, FIGS. 5A-5C. Safety, growth and survival study was performed via cytokine independent growth experiment to ensure CAR T cells are not transformed. See, FIGS. 6A-6B. Specific killing models were generated for two tumor targets in both homogenous and heterogeneous tumor target environment with a mixture containing BCMA+/GPRC5D+cell lines MM1.S or OPM2, and BCMA+/GPRC5D-RPMI8226, as well as BCMA-/GPRC5D-MV411. See, FIGS. 7A-7C. BCMA negative, GPRC5D positive MINI cell lines is under investigation to generate BCMA target homogenous and heterogeneous MINI tumor environment. The long-term killing activity in both homogenous and heterogeneous models in 96 well plates with flat bottom was assessed using NovoCyte flow cytometry. See, FIGS. 8A-11D. Detailed discussions are provided below.

CAR-T Transduction, Enrichment, and Expansion

All PCIR plasmids were packaged with PEQ-PEM 3(e), enveloped with BaevTR at 1.5:1.5:1 ratio and transfected 293 T cell via PEI MAX 40K (Polysciences, Inc, Cat #24765). The virus supernatant at 48 h and 72 h were collected and test titration using HT1080 cell line. Four donors' T cells were isolated using MACSxpress Whole Blood Pan T Cell Isolation Kit (Miltenyi Biotec, human,130-098-193), and cultured with T Cell TransAct™ (Miltenyi Biotec, human,130-111-160), 50 IU/ml of IL-2 (Fisher Scientific, human, CTP0023), 0.5 ng/ml of IL-15 (Miltenyi Biotec, human, 130-095-765), and 5% AB human serum (Fisher BioReagents, BP2525100).

Using retronectin reagent (TakaRa, T100A/B) coated non-tissue culture plate, Applicant transduced above constructs (FIG. 3A) into activated T cells (2-5 days) with MOI 3. Flow analysis determined the transduction rates on the day 6 of post transduction using Alexa Fluor® 647 AffiniPure Goat Anti-Mouse IgG, F(ab′)₂ fragment specific (Jackson ImmunoResearch_115-605-006) and PE anti-human CD19 Antibody (BioLegend, #302208) were used to detection the BCMA-CAR and truncated CD19 expression (FIG. 4A).

For enrichment of all the three BCMA CAR-T with two BiTE-BCMA CAR-T and one conventional BCMA-CAR-T), the cells were stained with the primary antibody Biotin-SP (long spacer) AffiniPure Goat Anti-Mouse IgG, F(ab′)₂ Fragment Specific (115-065-072 Jackson Immunoresearch) and followed by secondary staining with Invitrogen™ Dynabeads™ M-280 Streptavidin (Thermo Fisher, 11205D). Positive selection was further performed on DynaMag™-15 Magnet (Thermo Fisher, 12301D).

For enrichment of transduced T cell with truncated CD19 empty control, the REAlease CD19 MicroBead Kit (Miltenyi Biotec, human, 130-117-034) was used and positive selection was further performed with LS column before proceeding to magnetic separation.

Flow analysis demonstrated there were highly enriched CAR-T cells and empty vector control (FIG. 4B).

Determine the Expression and Concentration of Bispecific Antibody Secreted from T Cells by ELISA and Western Blotting

Applicant developed an unique ELISA assay for detection the concentration of full human source scFv-anti NKG2D and GPRC5D. Firstly, Applicant coated the Nunc MaxiSorp™ flat-bottom plate (Invitrogen™, 44-2404-21) with 100 ng/well of Recombinant Human NKG2D, amino acid Phe78-Va1216 (Accession #P26718) (Biolegends, #781908) dissolved in 1000 of DPBS (Gibco, 14190) at 4 degree overnight. After washing with 1x DPBS buffer with final 0.05% Tween 20 and blocking with 1% BSA in 1x DPBS buffer with final 0.05% Tween 20, 1000 of samples and standard in blocking buffer were added to each well. The standard used in this assay is a human anti human NKG2D antibody, CD314 (NKG2D)-PE, human, Cat: 130-111-645, recombinant human IgG1, Miltenyi Biotec GmbH (0.25 mg/ml). For detection, a Biotinylated Goat anti-Human IgG (H+L) Cross-Adsorbed Secondary Antibody and HRP-Conjugated Streptavidin (Thermo Scientific™, N100), as well as 1-Step™ Ultra TMB-ELISA Substrate Solution, (Bio-Rad #34029). Stop buffer used is 1 M H₃PO₄.

ELISA analysis showed there was detectable BiTE from non-concentrated supernatant of transduced BITE-BCMA CAR-T cells cultures in 6 different commercial T cell expansion Media, AIM V™ (Gibco™, 12055083), PRIME-XV T Cell CDM (FUJIFILM, 91154), X-VIVO 15 (Lonza,BE02-054Q), LymphoONE (Takara,WK552S), TexMACS™ (Miltinenyi, 130-097-196), and CTS™ OpTrnizer™ T Cell Expansion SFM (Gibco™ A1048501). Data is presented as an average from 4 independent donors as fold changes±SEM. See, FIG. 5A. The pool of T cell supernatant from four donors cultured in EX-CELL® 620-HSF Serum-Free Medium for Hybridoma Cells (Sigma:14621C) was purified using Pierce™ Anti-HA Agarose (Thermo 26182), five fractions of elution were performed ELISA assay (FIG. 5B).

For Western Blotting assay, total 10 μl of Unpurified Supernatant (U), Flow through (F), and Elution (E) from transduced T cells with “PCIR-BCGN1-IgG4 Fc”, “PCIR-BCNG2-IgG4 Fc”, “PCIR-Tcd19-EV”, and “PCIR-BCMA CAR only” were loaded onto the 4-20% Mini-PROTEAN® TGX™ Precast Protein Gels (Bio-Rag, #4561096). Primary antibody used was Rabbit-Anti-HA (CST, #3724) and the secondary antibody, LI-COR IRDye® 800CW Goat anti-Rabbit IgG (H+L) (Li-COR, #926-32211). There were expected˜75 KD HA-Tagged BITE protein bands in only Elution from “PCIR-BCGN1-IgG4 Fc” and “PCIR-BCNG2-IgG4 Fc”, but not from supernatant of samples “PCIR-Tcd19-EV” and “PCIR-BCMA CAR only.” See. FIG. 5C.

Safety, Growth, and Survival Study-Cytokine Independent Growth Experiment to Ensure CAR T Cells are not Transformed

Equal number of cells (1.5×10⁴ cells were seeded in the 96 well plates with media X-VIVO 15 containing 5% human AB type serum at the condition “no cytokine” and “cytokine plus” (IL-2 50 IU/ml; IL-15 0.5 ng/ml). NovoCyte 3005 Flow Cytometry used to count the cell number. The fold changes of total DAPI negative live cells verses the starting 15000 cell number were used to evaluate the cell survival and growth. All the wells were feed fresh media with and without cytokines every two days to provide enough nutrition condition to grow.

Firstly, the data disclosed herein clearly showed that, without cytokines, all the transduced CAR-T and control groups showed growth decreasing pattern on day 3 and almost died on day 10 (FIG. 6A). However, with cytokines kept adding, all groups grew up to 100-250 folds on day 10 (FIG. 6B), suggesting all transduced T cell growth was dependent on cytokines. This assay provided safety evidences for these transduced CAR-T cells and showing they are not transformed.

More importantly, Applicant found that, there were significantly beneficial effects on survival and growth of “PCIR-BCGN1-IgG4 Fc” and “PCIR-BCNG2-IgG4 Fc” compared to both “PCIR-BCMA CAR only”, and “non transduced” groups in condition of absent cytokines, which may greatly benefit for these non-transformed BiTE-BCMA CAR-T cells in vivo survival compared to conventional BCMA-CAR-T (FIGS. 6A-6B).

Generation of Specific Killing Models for Two Tumor Targets in Both Homogenous and Mimic Heterogeneous Tumor Targets Environment

For the in vitro cytotoxicity with two tumor targets, BCMA and GPRCSD on Multiple Myeloma, Applicant initially generated specific killing Models for two tumor targets in both homogenous and heterogeneous tumor target environment with a mixture containing BCMA+/GPRC5D+cell lines MM1.S or OPM2, and BCMA+/GPRC5D-RPMI8226, as well as BCMA-/GPRC5D-MV411 cell lines. See, FIGS. 7A-7C.

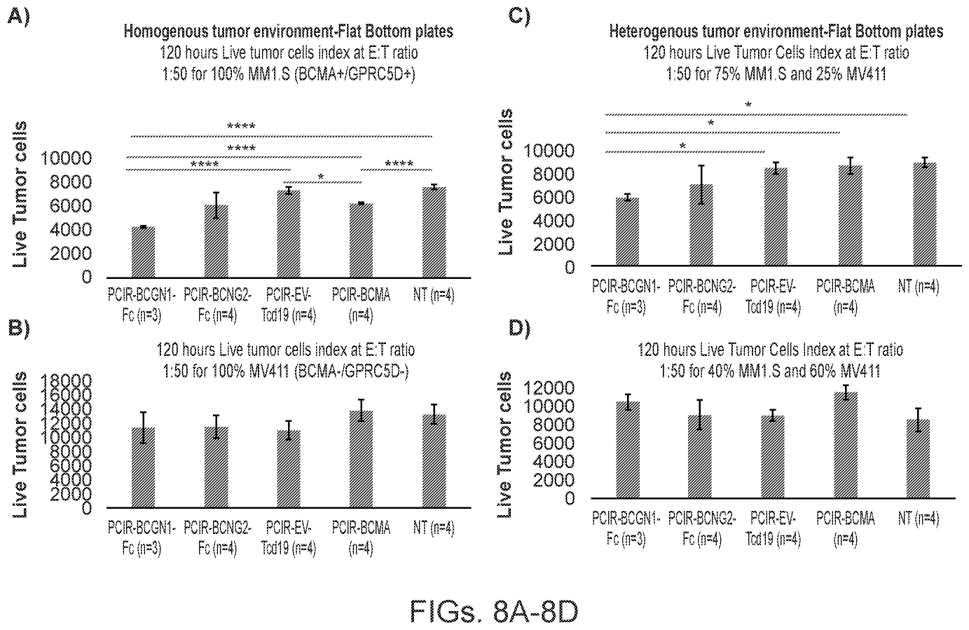

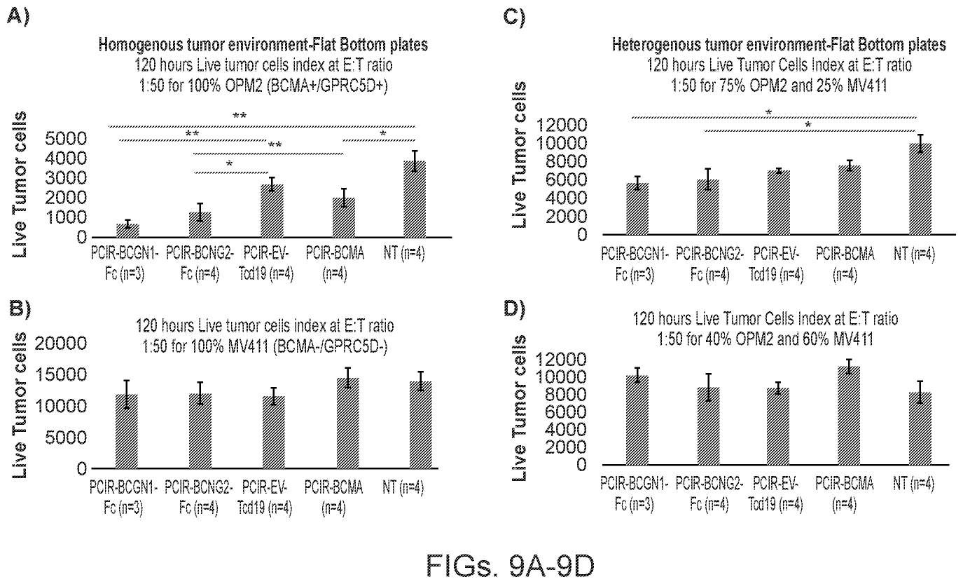

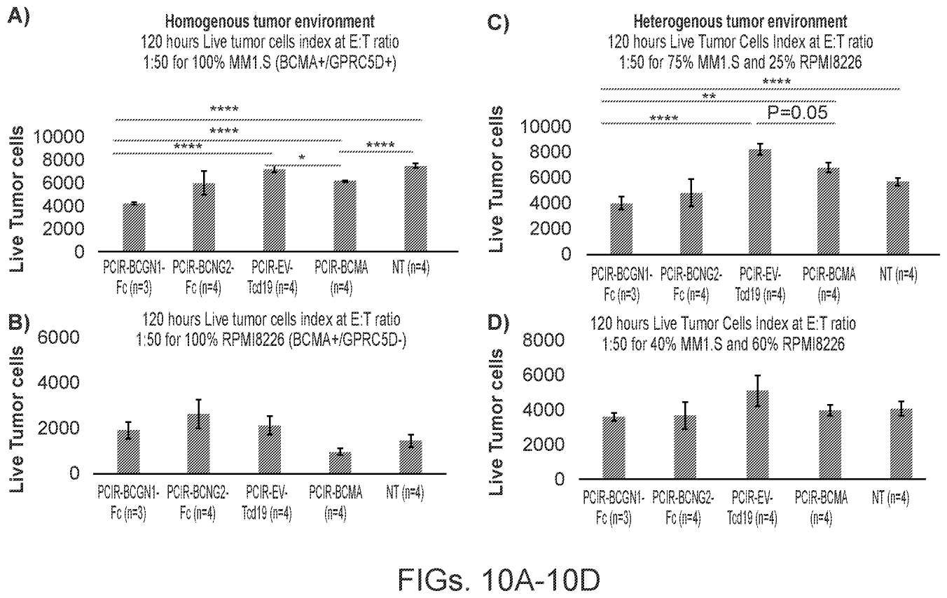

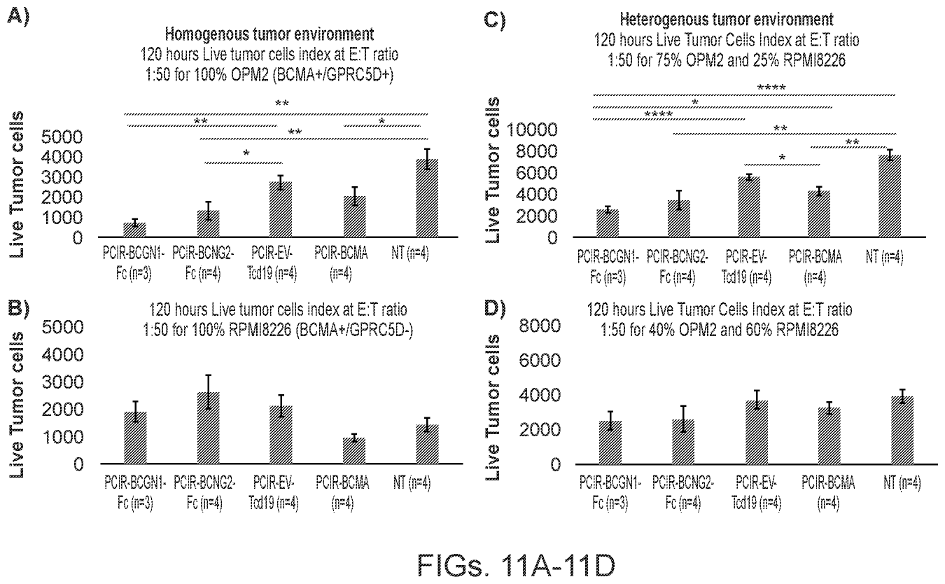

Flow Analysis determined that in the MM1.S and OPM2 are BCMA and GPRC5D positive MINI cell lines, the RPMI8226 is a BCMA positive but GPRC5D negative MINI cell line. The MV411 used in this study is a BCMA and GPRC5D double negative AML cell line. See, FIG. 7A. For generation of both BCMA and GPRC5D targets homogenous and heterogeneous MM tumor environment, Applicant mixed 75% of MM1.S or OPM2 with 25% of MV411, and 40% of MM1.S or OPM2 with 60% of MV411 (FIG. 7B) and the long-term 120 hours killing assay was performed (FIGS. 8A-9D). For generation of GPRC5D target homogenous and heterogeneous MINI tumor environment, 75% of MM1.S or OPM2 was mixed with 25% of RPMI 8226, and 40% of MM1.S or OPM2 was mixed with 60% of RPMI 8226 (FIG. 7C). The long-term 120 hours killing assay results are provided in FIGS. 10A-11D. BCMA negative, GPRC5D positive MM cell lines are under construction with Proteins Expression Blockers (PEBLs). anti-BCMA-PEBLs are used to generate BCMA target homogenous and heterogeneous MINI tumor environment.

Access the long-term killing activity in both homogenous and Heterogeneous models in 96 well plates with flat bottom using NovoCyte Flow cytometry.

For long term killing assay, Applicant set up the plate with effectors and tumor targets as FIG. 7B, and the live tumor cell numbers in the mixtures of effectors and targets were used to evaluate the killing activity. All tumors used in killing assay were introduced with Luc.ZSGreen plasmids using Lenti or Retrovirus systems, and NovoCyte 3005 Flow Cytometry was used to collect 40 μl of sample and count the ZsGreen positive and DAPI negative live tumor numbers in samples.

There were significantly tumor killing effects for GN1-BiTE BCMA-CAR (PCIR-BCGN1 IgG4-Fc), NG2-BiTE BCMA-CAR (PCIR-BCGN1 IgG4-Fc), and conventional BCMA-CAR-T compared to empty control non transduced T cells in homogenous and heterogeneous models (FIGS. 8A-11D). However, BiTE BCMA-CAR demonstrated dramatically tumor killing effects compared to conventional BCMA-CAR (P<0.0001). Data is presented as an average from 4 independent donors as Tumor lysis SEM. Two tails Student's T-Test was used to evaluate the significances between two groups, and the index for P value were: * p<0.05; ** p<0.01; *** p<0.001; **** p<0.0001.

### EXAMPLE 2— Anti-BCMA CAR-NK with Secretion of Anti-GPRC5D and NKG2D-Bispecific NK Engager (BiKE), Onboard Cytokine and Suicide Gene Enhances Anti-Multiple Myeloma (MM) Efficacy (In Vitro)

BCMA CAR-T with Secretion of Anti-GPRC5D and NKG2D-Bispecific T Cell Engager (BITE) Enhances Anti Multiple Myeloma (MM) Efficacy.

B cell maturation antigen (BCMA) and the orphan G protein—coupled receptor, class C group 5 member D (GPRC5D) are the most popular targets for Multiple Myeloma, and GPRC5D expression on multiple myeloma cells was independent of BCMA expression, suggesting that GPRC5D-targeted can overcoming the BCMA-antigen loss mediated relapse (FIG. 1).

Applicant's studies, such as Example 1, demonstrated anti BCMA CAR-T with secretion of Anti-GPRC5D and NKG2D-Bispecific T cell Engager (BiTE) can dramatically kill BCMA and GPRC5D double positive MINI cell line MM1. S and OPM2 compared to conventional anti BCMA CAR-T cells.

Development of BCMA CAR-NK with Secretion of Anti-GPRC5D and NKG2D-Bispecific NK Cell Engager (BIKE).

Introducing IL-15 and suicide gene into CAR-NK enhances its efficacy by sustained CAR-NK's survival time in vivo and safety.

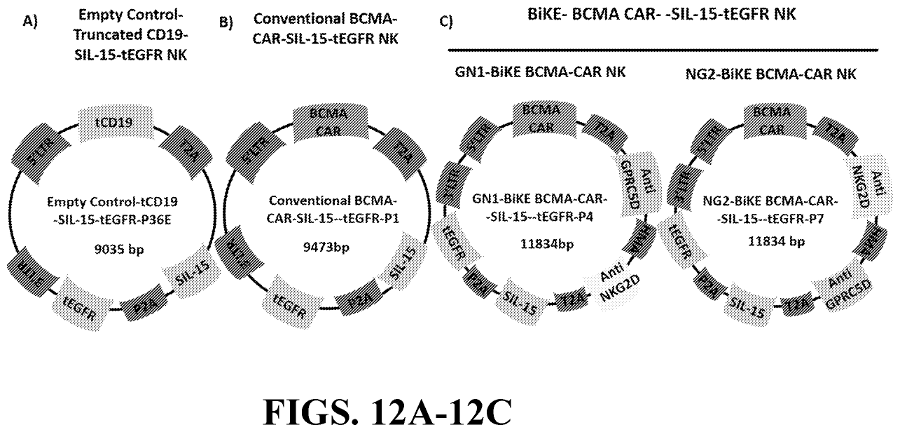

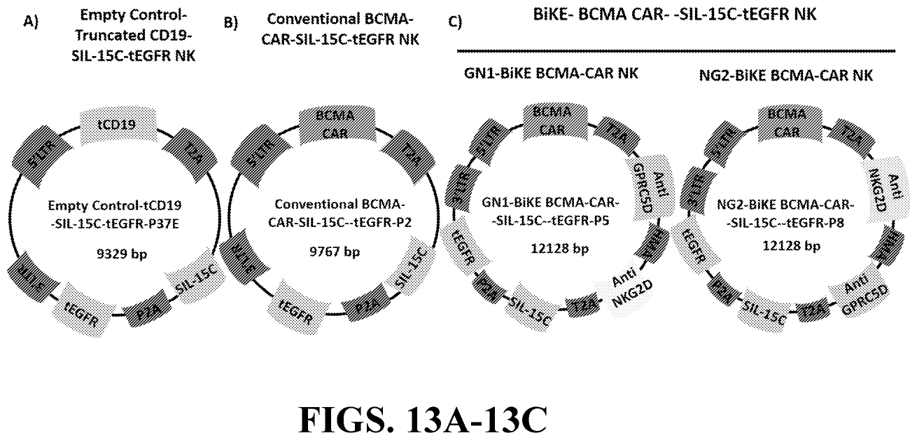

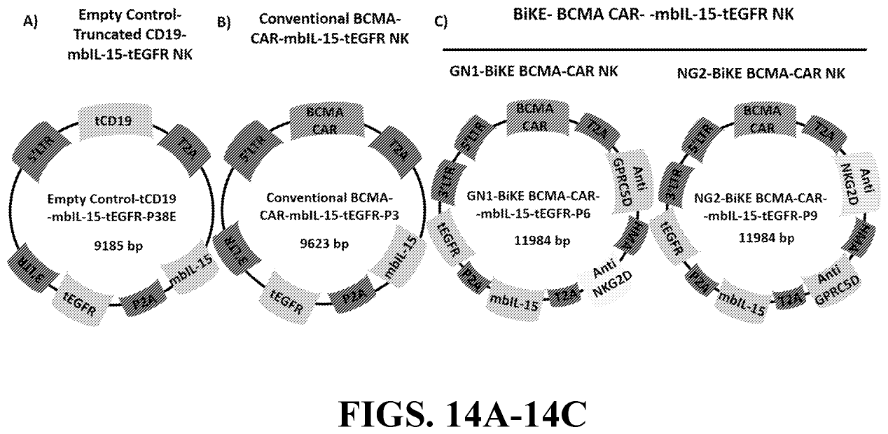

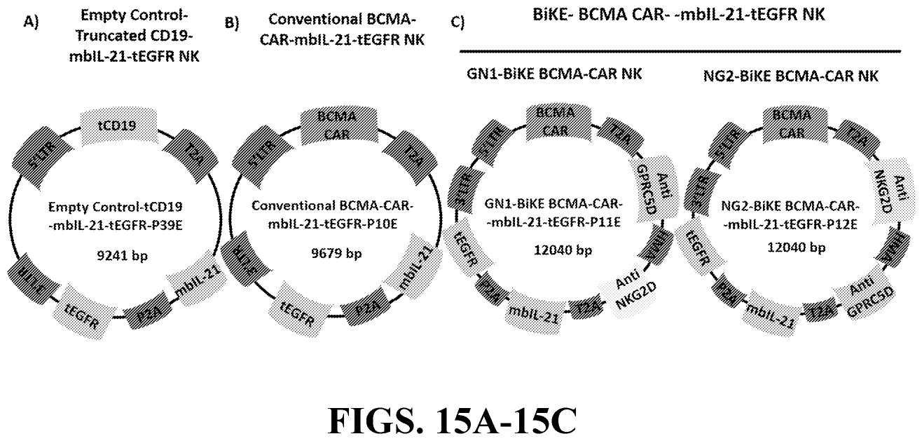

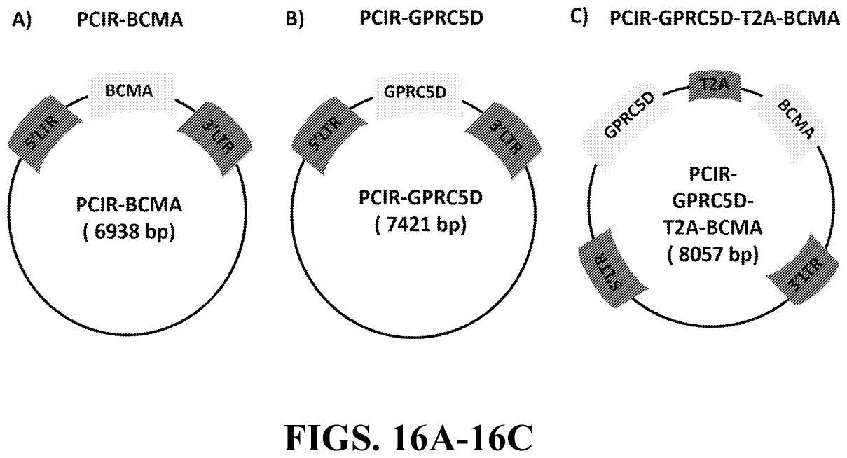

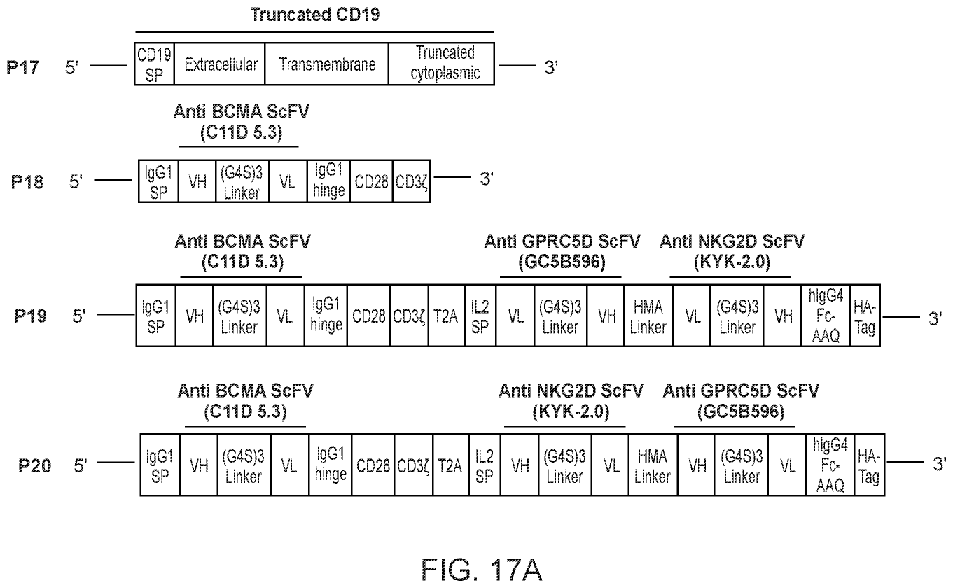

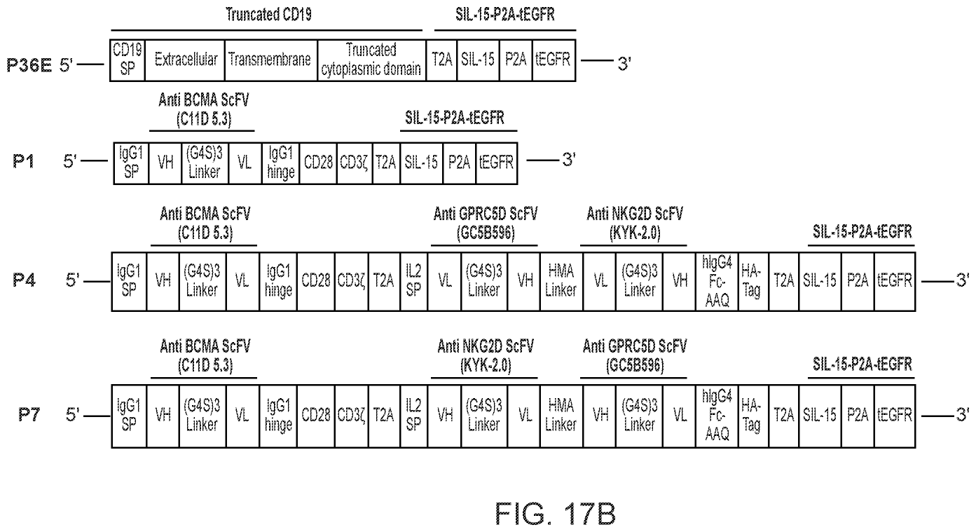

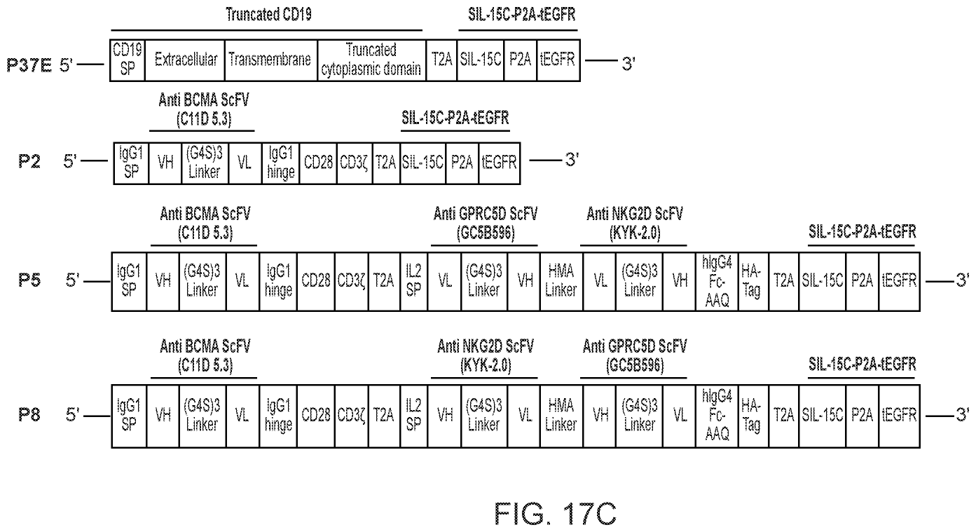

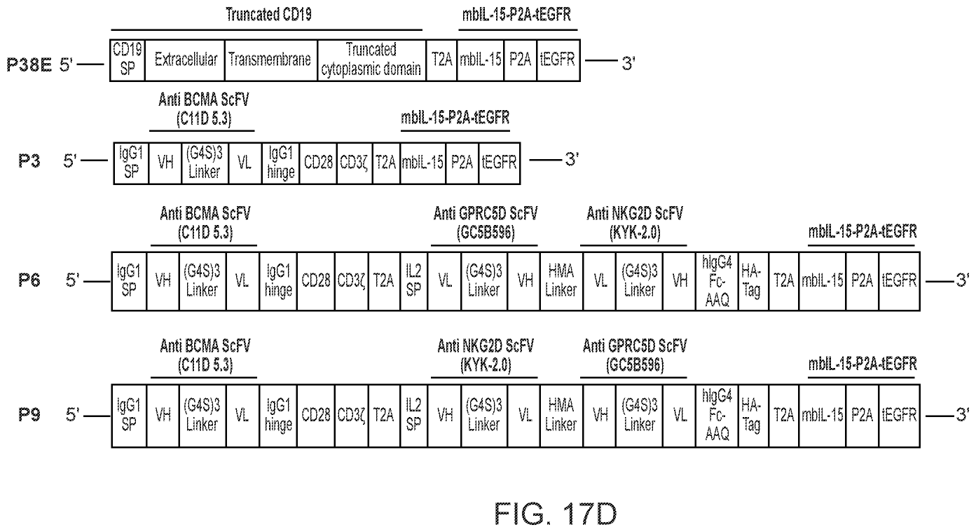

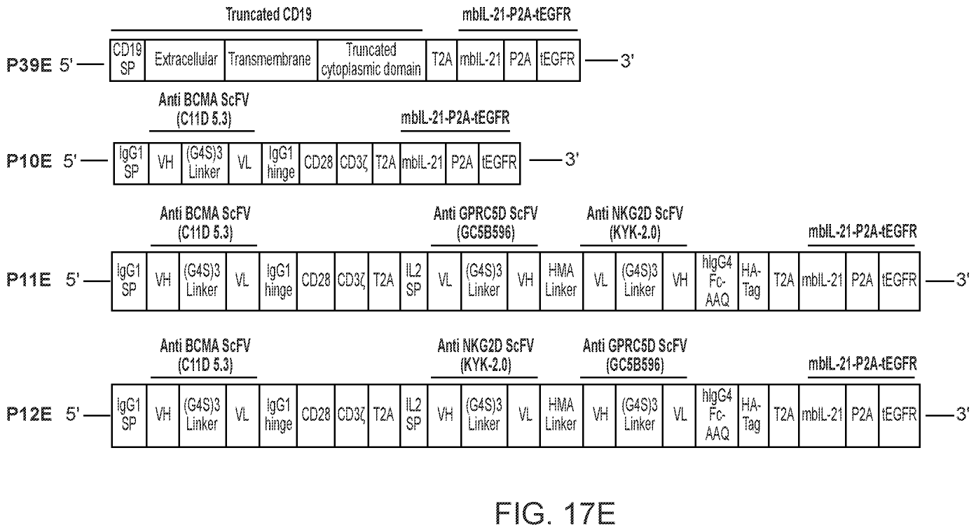

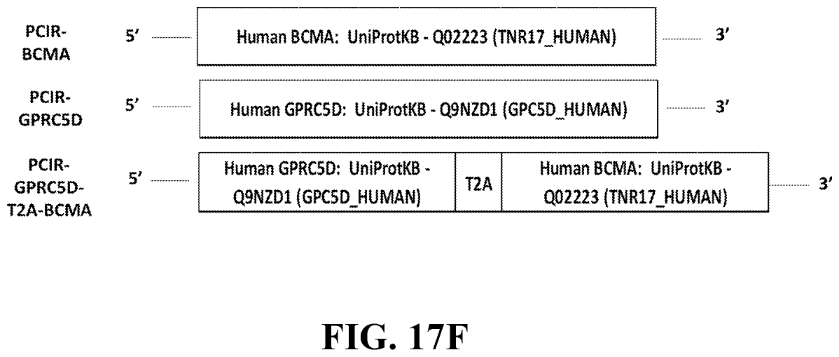

With modification of introducing onboard cytokine IL-15 and IL-21 and suicide gene “truncated EGFR” (tEGFR) to the BiTE-CAR-T construct described above (FIG. 2) Applicant created several constructs aimed to generate an anti BCMA CAR-NK with secretion of Anti-GPRC5D and NKG2D-Bispecific NK cell Engager (BiKE). The schematic representations and diagraphs of CAR-NK are illustrated in FIGS. 2 and 12-17F.

The amino acids and nucleotide sequences for each components and whole sequences of above constructs are provided in Table 2.

TABLE 2a
CAR-Components used in this study.

| | Amino acid | Nucleotide |
|---|---|---|
| | sequence | sequence |
| | (SEQ ID NO:) | (SEQ ID NO:) |
| IgG1 SP | 21 | 55 |
| VL-mouse anti-human BCMA light | 41 | 58 |
| chain (C11D5.3) | | |
| CDR1-VL-C11D5.3 | 23 | 171 |
| CDR2-VL-C11D5.3 | 26 | 172 |
| CDR3-VL-C11D5.3 | 29 | 173 |
| VH-mouse anti-human BCMA heavy | 44 | 62 |
| chain (C11D5.3) | | |
| CDR1-VH-C11D5.3 | 32 | 174 |
| CDR2-VH-C11D5.3 | 35 | 175 |
| CDR3-VH-C11D5.3 | 38 | 176 |
| VL-Humanized anti-BCMA light chain | 42 | 59 |
| CDR1-VL Humanized anti-BCMA light | 24 | 177 |
| chain | | |
| CDR2-VL-Humanized anti-BCMA light | 27 | 178 |
| chain | | |
| CDR3-VL-Humanized anti-BCMA light | 30 | 179 |
| chain | | |
| VH-Humanized anti-BCMA heavy | 45 | 63 |
| chain | | |
| CDR1-VH Humanized anti-BCMA | 33 | 180 |
| light chain | | |
| CDR2-VH-Humanized anti-BCMA | 36 | 181 |
| light chain | | |
| CDR3-VH-Humanized anti-BCMA | 39 | 182 |
| light chain | | |
| (G4S)3 linker | 9 | 57 |
| IgG1 hinge | 47 | 66 |
| CD28 transmembrane and | 48 | 67 |
| cytoplasmic/intracellular domain | | |
| CD3Z isoform 3 | 49 | 68 |
| T2A | 50 | 69 |
| IL-2 signal sequence | 20 | 56 |
| VL-human anti-human GPRC5D light | 16 | 71 |
| chain (GC5B596) | | |
| CDR1-VL-GC5B596 | 10 | 183 |
| CDR2-VL-GC5B596 | 11 | 184 |
| CDR3-VL-GC5B596 | 12 | 185 |
| VH-human anti-human GPRC5D heavy | 17 | 72 |
| chain (GC5B596) | | |
| CDR1-VH-GC5B596 | 13 | 186 |
| CDR2-VH-GC5B596 | 14 | 187 |
| CDR3-VH-GC5B596 | 15 | 188 |
| VL-human anti-human NKG2D light | 7 | 73 |
| chain (KYK-2.0) | | |
| CDR1-VL-KYK-2.0 | 1 | 189 |
| CDR2-VL-KYK-2.0 | 2 | 190 |
| CDR3-VL-KYK-2.0 | 3 | 191 |
| VH-human anti-human NKG2D heavy | 8 | 74 |
| chain (KYK-2.0) | | |
| CDR1-VH-KYK-2.0 | 4 | 192 |
| CDR2-VH-KYK-2.0 | 5 | 193 |
| CDR3-VH-KYK-2.0 | 6 | 194 |
| HMA linker | 81 | 70 |
| three-site mutated human IgG4 Fc - | 19 | 75 |
| AAQ | | |
| HA-Tag | 22 | 76 |
| Truncated CD19 | 170 | 195 |
| Truncated EGFR | 120 | 162 |
| human IL-15 without endogenous signal | 106 | 152 |
| peptide | | |
| human IL-15 with endogenous signal | 104 | 154 |
| peptide | | |
| Chain A, Interleukin-15 receptor alpha | 107 | 167 |
| chain [Homo sapiens] Sequence ID: | | |
| 2ERS_A | | |
| CSF2R SP | 125 | 161 |
| P2A | 133 | 159 |
| PDGFRβ TM domain | 119 | 164 |
| GM-CSF SP | 122 | 200 |
| Human IL-21 (identifier: Q9HBE4-2) | 116 | 157 |

TABLE 2b
Truncated CD19 empty vector-P17

| | Amino acid | Nucleotide |
|---|---|---|
| | sequence | sequence |
| | (SEQ ID NO:) | (SEQ ID NO:) |
| Truncated CD19 empty vector-P17 | 170 | 195 |

TABLE 2c
Conventional anti BCMA-CAR -P18

| | Amino acid | Nucleotide |
|---|---|---|
| | sequence | sequence |
| | (SEQ ID NO:) | (SEQ ID NO:) |
| Conventional BCMA-CAR-P18 | 196 | 197 |

TABLE 2d
GN1-BiTE BCMA-CAR-P19

| | Amino acid | Nucleotide |
|---|---|---|
| | sequence | sequence |
| | (SEQ ID NO:) | (SEQ ID NO:) |
| GN1-BiTE BCMA-CAR-P19 | 51 | 77 |

TABLE 2e
NG2-BiTE BCMA-CAR-P20

| | Amino acid | Nucleotide |
|---|---|---|
| | sequence | sequence |
| | (SEQ ID NO:) | (SEQ ID NO:) |
| NG2-BiTE BCMA-CAR-P20 | 52 | 78 |

TABLE 2f
Empty Control-tCD19-SIL-15-tEGFR-P36E

| | Amino acid | Nucleotide |
|---|---|---|
| | sequence | sequence |
| | (SEQ ID NO:) | (SEQ ID NO:) |
| Empty Control-tCD19- | 201 | 202 |
| SIL-15-tEGFR-P36E | | |

TABLE 2g
Conventional BCMA-CAR-SIL-15--tEGFR-P1

| | Amino acid | Nucleotide |
|---|---|---|
| | sequence | sequence |
| | (SEQ ID NO:) | (SEQ ID NO:) |
| Conventional BCMA-CAR-SIL-15-- | 203 | 204 |
| tEGFR-P1 | | |

TABLE 2h
GN1-BiTE BCMA-CAR--SIL-15--tEGFR-P4

| | Amino acid | Nucleotide |
|---|---|---|
| | sequence | sequence |
| | (SEQ ID NO:) | (SEQ ID NO:) |
| GN1-BiTE BCMA-CAR--SIL-15-- | 205 | 206 |
| tEGFR-P4 | | |

TABLE 2i
NG2-BiTE BCMA-CAR--SIL-15--tEGFR-P7

| | Amino acid | Nucleotide |
|---|---|---|
| | sequence | sequence |
| | (SEQ ID NO:) | (SEQ ID NO:) |
| NG2-BiTE BCMA-CAR--SIL-15-- | 207 | 208 |
| tEGFR-P7 | | |

TABLE 2j
Empty Control-tCD19-SIL-15C-tEGFR-P37E

| | Amino acid | Nucleotide |
|---|---|---|
| | sequence | sequence |
| | (SEQ ID NO:) | (SEQ ID NO:) |
| Empty Control-tCD19- | 209 | 210 |
| SIL-15C-tEGFR-P37E | | |

TABLE 2k
Conventional BCMA-CAR-SIL-15C--tEGFR-P2

| | Amino acid | Nucleotide |
|---|---|---|
| | sequence | sequence |
| | (SEQ ID NO:) | (SEQ ID NO:) |
| Conventional BCMA-CAR-SIL-15C-- | 211 | 212 |
| tEGFR-P2 | | |

TABLE 2l
GN1-BIKE BCMA-CAR--SIL-15C--tEGFR-P5

| | Amino acid | Nucleotide |
|---|---|---|
| | sequence | sequence |
| | (SEQ ID NO:) | (SEQ ID NO:) |
| GN1-BIKE BCMA-CAR--SIL-15C-- | 213 | 214 |
| tEGFR-P5 | | |

TABLE 2m
NG2-BIKE BCMA-CAR--SIL-15C--tEGFR-P8

| | Amino acid | Nucleotide |
|---|---|---|
| | sequence | sequence |
| | (SEQ ID NO:) | (SEQ ID NO:) |
| NG2-BIKE BCMA-CAR--SIL-15C-- | 215 | 216 |
| tEGFR-P8 | | |

TABLE 2n
Empty Control-tCD19-mbIL-15-tEGFR-P38E

| | Amino acid | Nucleotide |
|---|---|---|
| | sequence | sequence |
| | (SEQ ID NO:) | (SEQ ID NO:) |
| Empty Control-tCD19- | 217 | 218 |
| mbIL-15-tEGFR-P38E | | |

TABLE 2o
Conventional BCMA-CAR-mbIL-15-tEGFR-P3

| | Amino acid | Nucleotide |
|---|---|---|
| | sequence | sequence |
| | (SEQ ID NO:) | (SEQ ID NO:) |
| Conventional BCMA-CAR-mbIL-15- | 219 | 220 |
| tEGFR-P3 | | |

TABLE 2p
GN1-BiKE BCMA-CAR--mbIL-15-tEGFR-P6

| | Amino acid | Nucleotide |
|---|---|---|
| | sequence | sequence |
| | (SEQ ID NO:) | (SEQ ID NO:) |
| GN1-BiKE BCMA-CAR--mbIL-15- | 221 | 222 |
| tEGFR-P6 | | |

TABLE 2q
NG2-BiKE BCMA-CAR--mbIL-15-tEGFR-P9

| | Amino acid | Nucleotide |
|---|---|---|
| | sequence | sequence |
| | (SEQ ID NO:) | (SEQ ID NO:) |
| NG2-BiKE BCMA-CAR--mbIL-15- | 223 | 224 |
| tEGFR-P9 | | |

TABLE 2r
Empty Control-tCD19-mbIL-21-tEGFR-P39E

| | Amino acid | Nucleotide |
|---|---|---|
| | sequence | sequence |
| | (SEQ ID NO:) | (SEQ ID NO:) |
| Empty Control-tCD19- | 225 | 226 |
| mbIL-21-tEGFR-P39E | | |

TABLE 2s
Conventional BCMA-CAR-mbIL-21-tEGFR-P10E

| | Amino acid | Nucleotide |
|---|---|---|
| | sequence | sequence |
| | (SEQ ID NO:) | (SEQ ID NO:) |
| Conventional BCMA-CAR-mbIL-21- | 227 | 228 |
| tEGFR-P10E | | |

TABLE 2t
GN1-BiKE BCMA-CAR--mbIL-21-tEGFR-P11E

| | Amino acid | Nucleotide |
|---|---|---|
| | sequence | sequence |
| | (SEQ ID NO:) | (SEQ ID NO:) |
| GN1-BiKE BCMA-CAR--mbIL-21- | 229 | 230 |
| tEGFR-P11E | | |

TABLE 2u
NG2-BiKE BCMA-CAR--mbIL-21-tEGFR-P12E

| | Amino acid | Nucleotide |
|---|---|---|
| | sequence | sequence |
| | (SEQ ID NO:) | (SEQ ID NO:) |
| NG2-BiKE BCMA-CAR--mbIL-21- | 231 | 232 |
| tEGFR-P12E | | |

TABLE 2v
PCIR-BCMA

| | Amino acid | Nucleotide |
|---|---|---|
| | sequence | sequence |
| | (SEQ ID NO:) | (SEQ ID NO:) |
| PCIR-BCMA | 233 | 234 |

TABLE 2w
PCIR-GPRC5D

| | Amino acid | Nucleotide |
|---|---|---|
| | sequence | sequence |
| | (SEQ ID NO:) | (SEQ ID NO:) |
| PCIR-GPRC5D | 235 | 236 |

TABLE 2x
PCIR-GPRC5D-T2A-BCMA

| | Amino acid | Nucleotide |
|---|---|---|
| | sequence | sequence |
| | (SEQ ID NO:) | (SEQ ID NO:) |
| PCIR-GPRC5D-T2A-BCMA | 237 | 238 |

Optimization of the Large Size (>11 kb) LTR Retrovirus Plasmid Preparation

In this study, Applicant used MAX Efficiency™ Stb12™ Competent Cells (Invitrogen, 10268019) to propagate several constructs with size>10 Kb (P4, P5, P6, P7, P8, P9, P11E, P12E, P61, P62, or P72) with optimized efficiency and quality.

For other plasmids with size<=10 Kb (P1, P2, P3, P10E, P36E, P37E, P38E, P39E, P17, P18, P19, P20), the NEB® Stable Competent E. coli (High Efficiency) (NEB, C3030H) were used.

All the plasmid colonies were selected on the LB Agar plates and liquid culture with 100 μg/ml of Carbenicillin (Teknova C2136) at 30 degree. For liquid culture grown, 180 rpm in a shaker was used.

The candidate plasmids with estimated bands identified with proper enzymes digestion were sent to perform gene sequencing (Retrogene Inc) to confirm the sequences were correct.

Preparation of CAR-NK Cells.

Briefly, all PCIR plasmids were packaged with PEQ-PEM 3(e), enveloped with BaevTR at 1.5:1.5:1 ratio and transfected 293 T cell via PEI MAX 40K (Polysciences, Inc, Cat #24765). Four donor's NK cells were isolated using MACSxpress Whole Blood NK Cell Isolation Kit, human (Miltenyi Biotec,130-098-185), and cultured with NK MACS media (130-114-429) in the presence of 50 IU/ml of human IL-2, 5% AB human serum (Fisher BioReagents, BP2525100) and Irradiated K562-mb21-41BBL feeders with the ratio of 1:1.

Using retronectin reagent (TakaRa, T100A/B) coated non-tissue culture plate, Applicant transduced the above constructs (FIGS. 2 and 12-16) into activated NK cells (7 days). In this study, for enrichment of constructs containing BCMA-CAR (P1, P4, P7, P2, P5, P8, P3, P6, P9, P10E, P11E, P12E, P18, P19, P20, P61, P62, or P72), Applicant stained these transduced cells with the BCMA protein-coupled magnetic beads (ACROBiosystems, MBS-K004) and further performed positive selection on DynaMag™-15 Magnet (Thermo Fisher,12301D).

For enrichment of transduced NK cell with truncated CD19 empty control, the REAlease CD19 MicroBead Kit (Miltenyi Biotec, human, 130-117-034) was used and further performed positive selection with LS column and proceeded to magnetic separation.

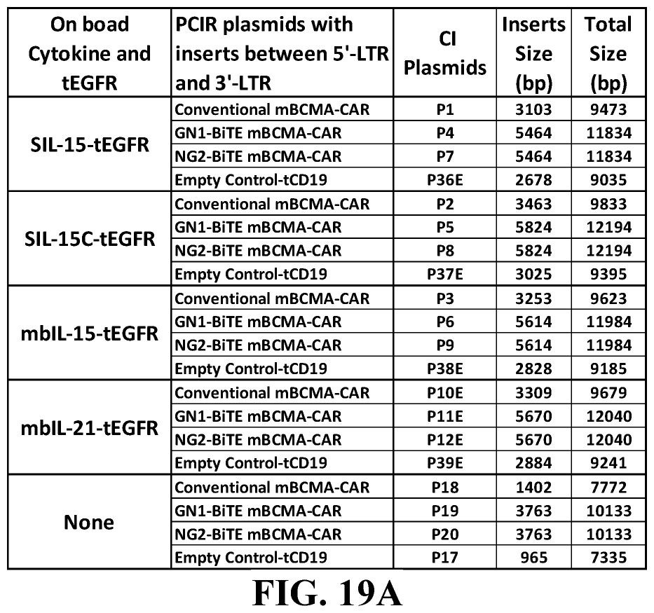

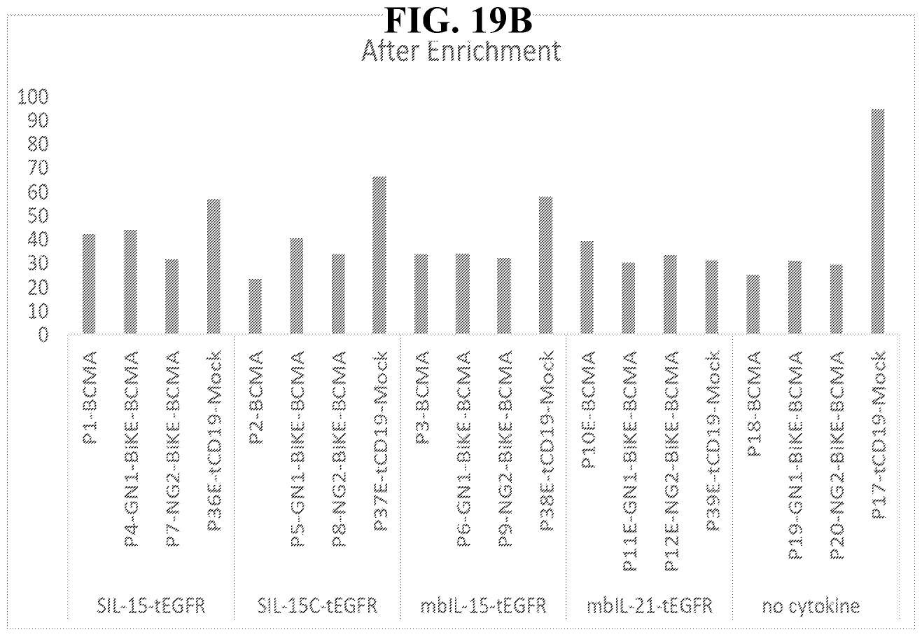

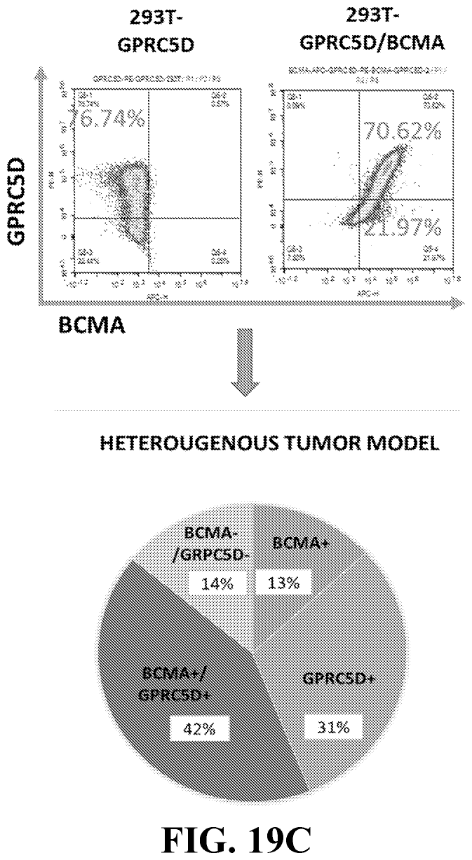

Flow analysis determined the transduction rates using Alexa Fluor® 647 AffiniPure Goat Anti-Mouse IgG, F(ab′)2 fragment specific (Jackson ImmunoResearch_115-605-006) and PE anti-human CD19 Antibody (BioLegend, #302208) to detect the BCMA-CAR and truncated CD19 expression (FIGS. 19A and 19B).

Determination of the Expression and Concentration of Bispecific Antibody Secreted from Transduced NK Cells by ELISA.

Applicant developed an unique ELISA assay for detection the concentration of full human Source ScFV-anti NKG2D and GPRCSD. Firstly, Applicant coated the Nunc MaxiSorp™ flat-bottom plate (Invitrogen™, 44-2404-21) with 100 ng/well of Recombinant Human NKG2D, amino acid Phe78-Va1216 (Accession #P26718) (Biolegends, #781908) dissolved in 100 μl of DPBS (Gibco, 14190) at 4 degree overnight. After washing with 1x DPBS buffer with final 0.05% Tween 20 and blocking with 1% BSA in 1x DPBS buffer with final 0.05% Tween 20, 100 μl of samples and standard in blocking buffer were added to each well. The standard used in this assay was a human anti human NKG2D antibody, CD314 (NKG2D)-PE, human, Cat: 130-111-645, recombinant human IgG1, Miltenyi Biotec GmbH (0.25 mg/ml). For detection, a Biotinylated Goat anti-Human IgG (H+L) Cross-Adsorbed Secondary Antibody and HRP-Conjugated Streptavidin (Thermo Scientific™, N100), as well as 1-Step™ Ultra TMB-ELISA Substrate Solution, (Bio-Rad #34029). Stop buffer used is 1 M H₃PO₄.

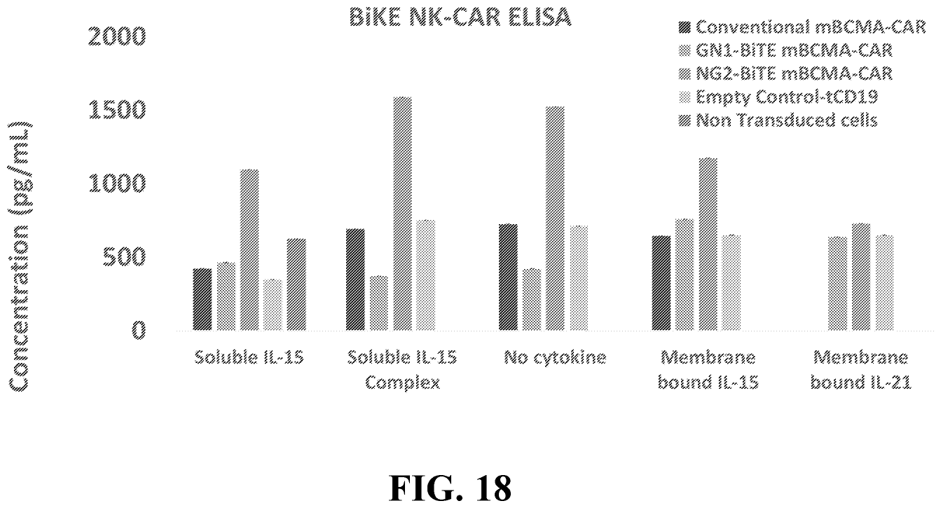

ELISA analysis showed there was detectable BiKE from non-concentrated supernatant of transduced BITE-BCMA CAR-NK cells cultures in all 5 groups with different onboard cytokine modification. Data presented as an average from 4 independent donors as average±SEM. (FIG. 18).

Generation of Specific Killing Models for Heterogeneous Tumor Model Using RTCA (Real Time Cytotoxicity Assay)

For the in vitro long-term cytotoxicity assay with RTCA system, Applicant initially generated specific killing models for two tumor targets in mimic heterogeneous tumor targets environment with a mixture of artificial over expression GPRC5D and double positive BCMA/GPRC5D antigens.

BCMA, GPRC5D and double BCMA/GPRC5D gene were transduced into 293T using modified Retrovirus gene delivery system. With mixtures of two parts of 293T-GPRC5D (76.74%) and three parts of 293T-GPRC5D/BCMA (70.62% double positive, and 21.97% BCMA+), Applicant generated heterogeneous tumor model to mimic the Multiple Myeloma tumor environment (FIG. 19C).

Accessing the Long-Term Real Time Killing Assay Using xCELLigence RTCA.

For long term killing assay, total 5000 targets were seeded on E-Plate 96 PET (Agilent REF 300600910) and incubated at 37 degree with 5% CO₂ for ˜20 h, then added total 500 effectors into the wells with targets. The ratio of Effectors and Targets used in this study was 1:10.

Data presented as % Cytolysis from 2 independent donors. The cells were real-time monitored at 37° C. in humidified 5% CO₂ atm. Cell Index (CI) data was recorded every 15 min for 120 h. The selected normalization time point was 20 h after seeding. The time to reach maximum Cell Index for targets alone group get was ˜80h, so the % Cytolysis was calculated and compared at ˜60h with addition of effectors

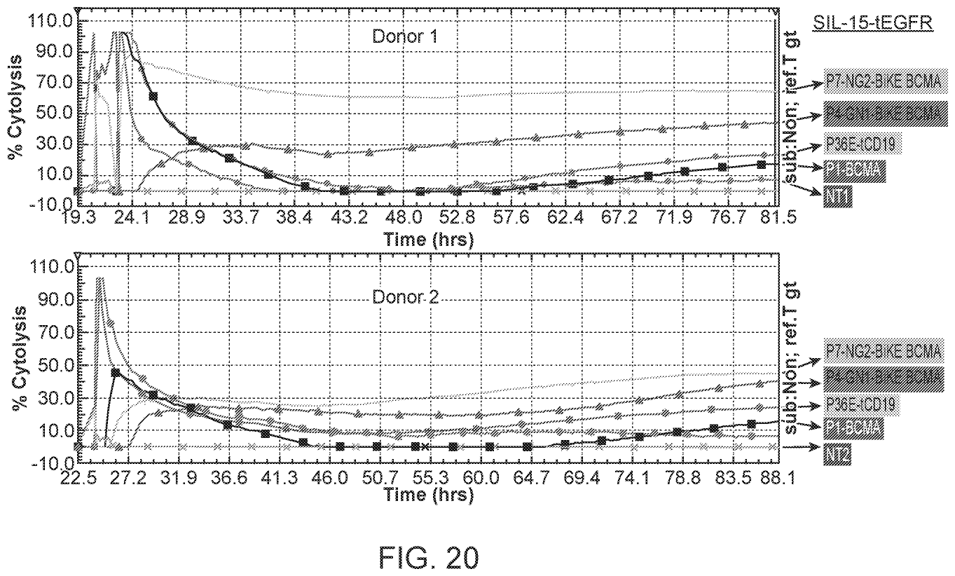

FIG. 20 shows results from the RTCA assay for BiKE-CAR-NK with soluble cytokines (IL-15) and suicide gene tEGFR modification.

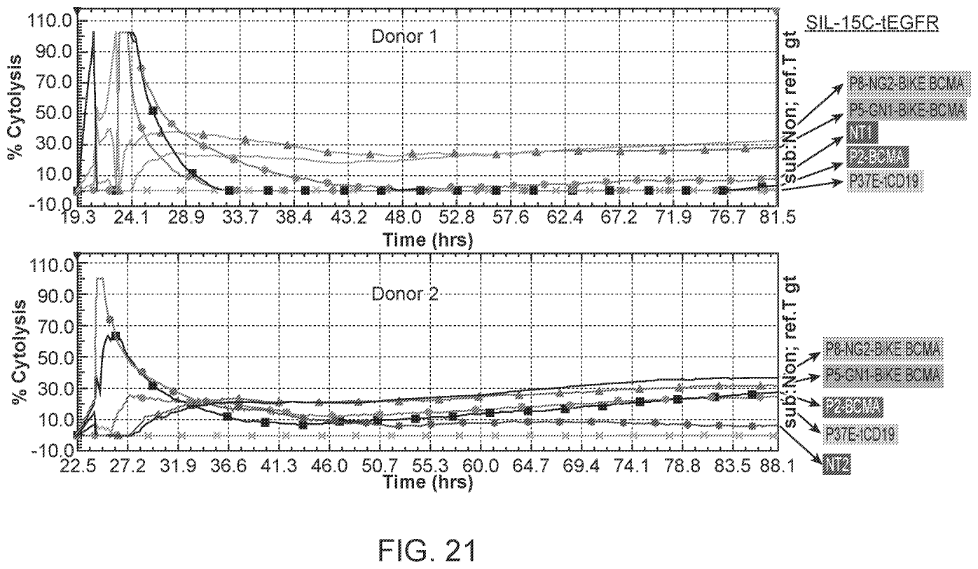

FIG. 21 shows results from the RTCA assay for BiKE-CAR-NK with soluble IL-15 and IL-15 receptor complex (SIL15C) and suicide gene tEGFR modification.

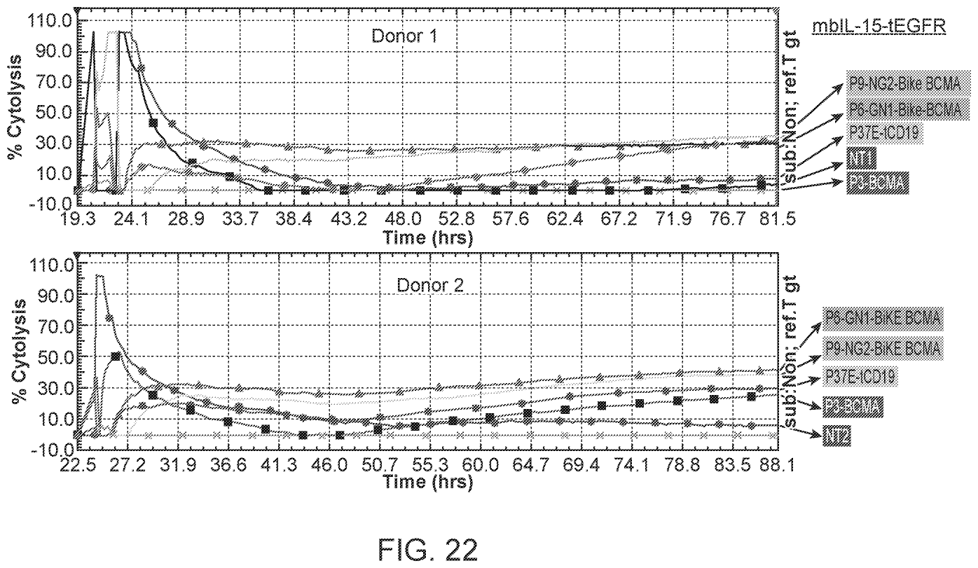

FIG. 22 shows results from the RTCA assay for BiKE-CAR-NK with membrane bound IL-15 (mbIL15) and suicide gene tEGFR modification.

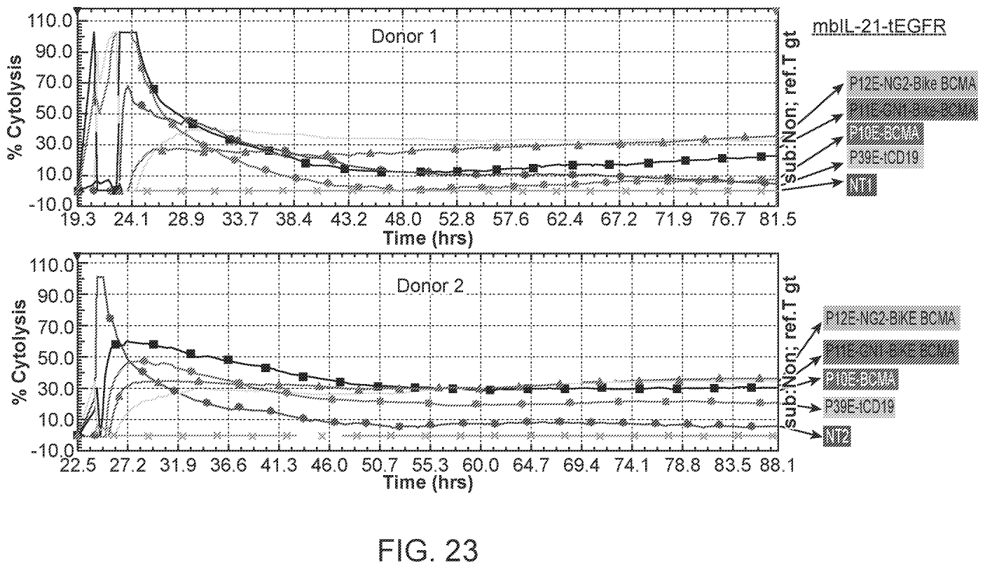

FIG. 23 shows results from the RTCA assay for BiKE-CAR-NK with membrane bound IL-21(mbIL21) and suicide gene tEGFR modification.

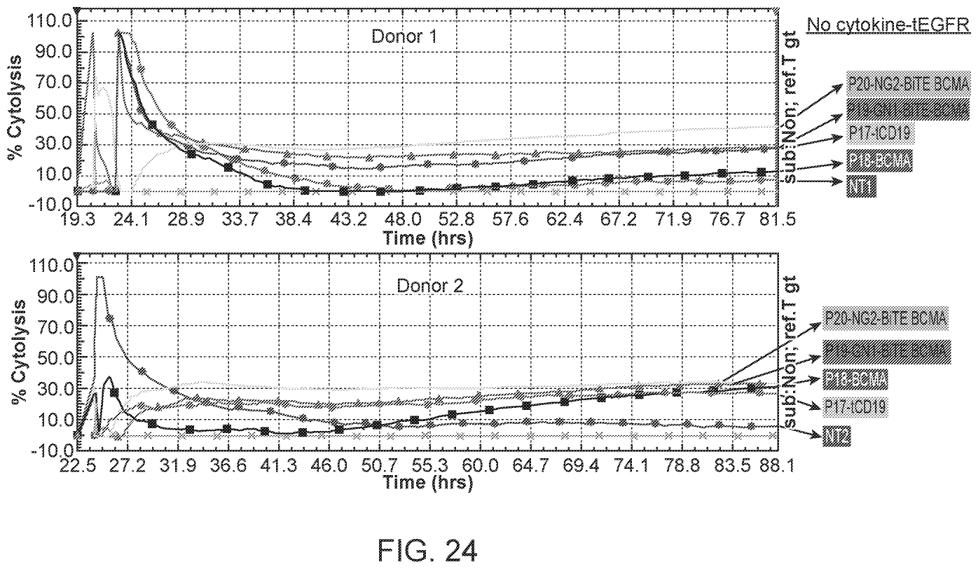

FIG. 24 shows results from the RTCA assay for BiTE-CAR-NK without modification.

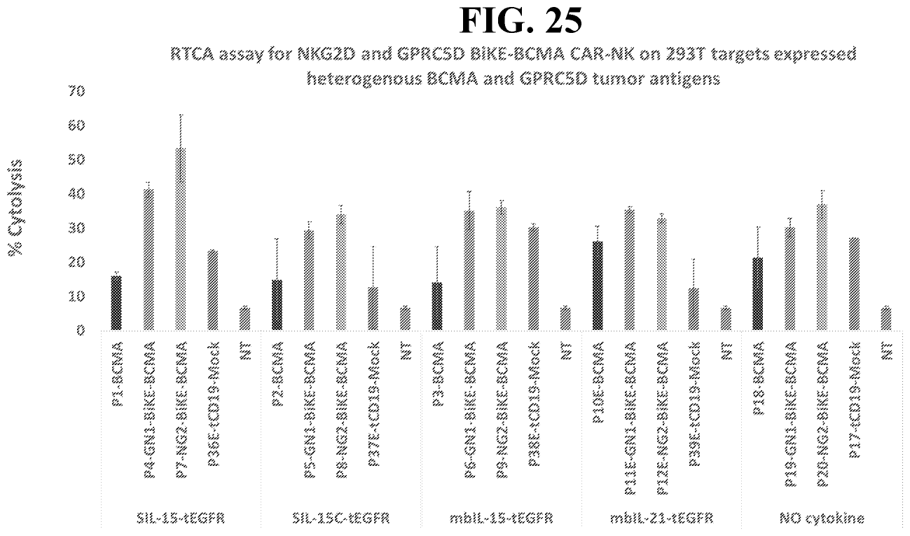

FIG. 25 shows the increased cytolysis of NKG2D and GPRC5D BiKE-BCMA CAR-NK compared to conventional BCMA-CAR-NK determine by RTCA. Data presented as an average from 2 independent donors as Tumor lysis±SD at reaction of 60h.

### EXAMPLE 3-Anti BCMA CAR-NK with Secretion of Anti-GPRC5D and NKG2D Bispecific NK Engager (BiKE), Onboard Cytokine and Suicide Gene Enhances Anti-Multiple Myeloma (MM) Efficacy (In Vitro)

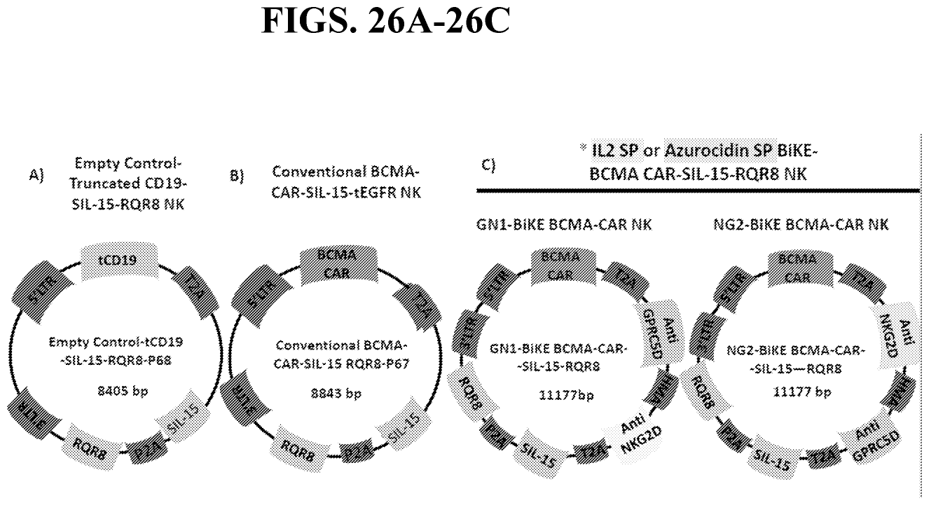

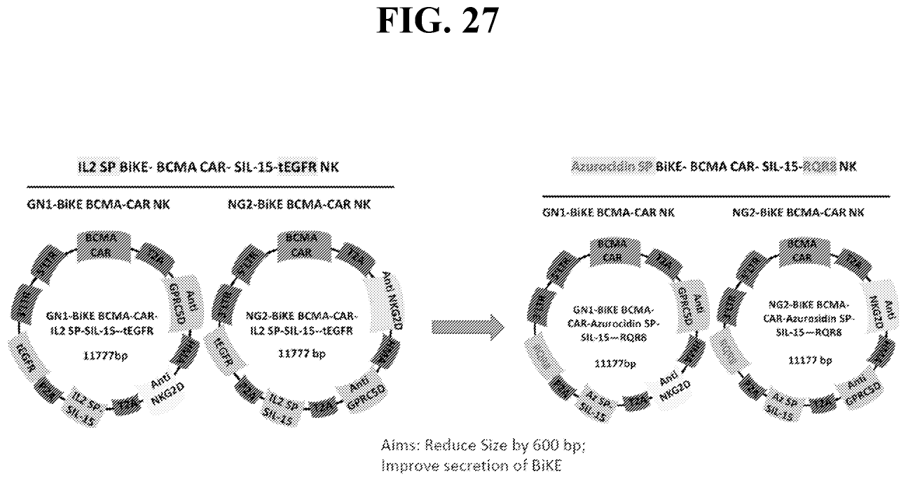

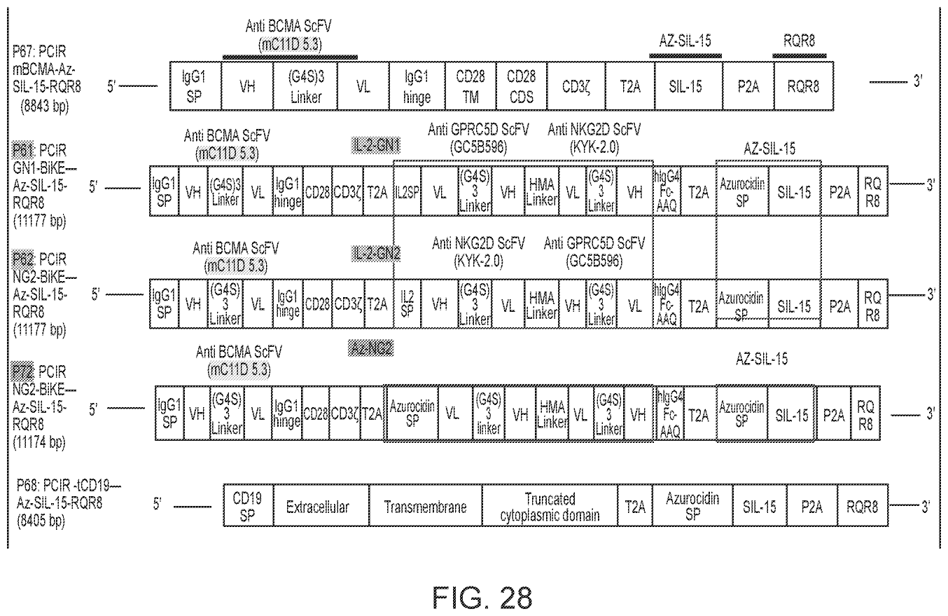

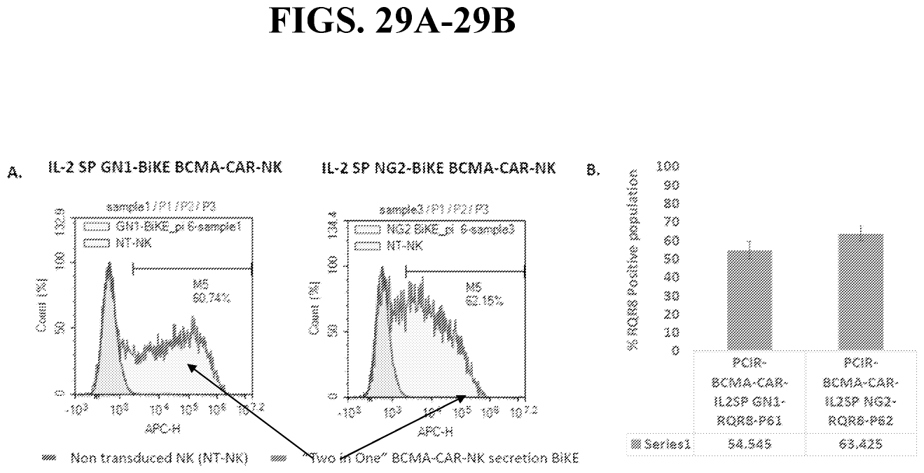

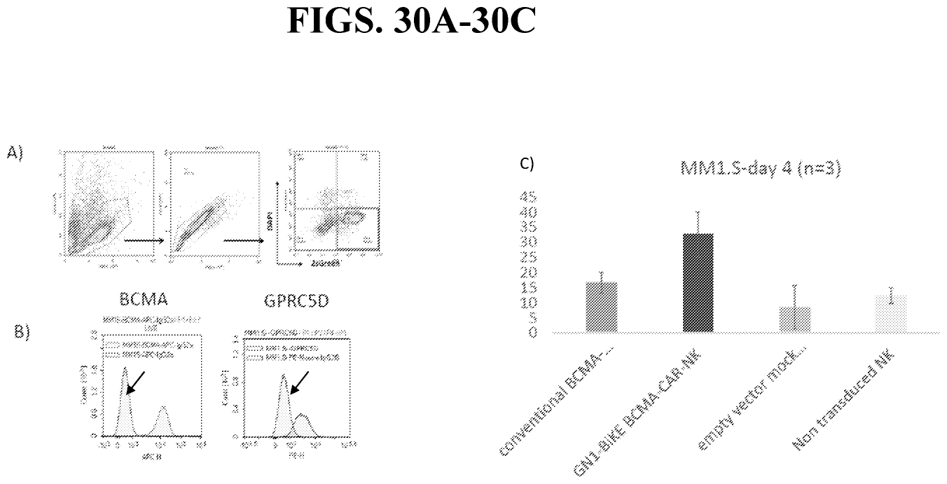

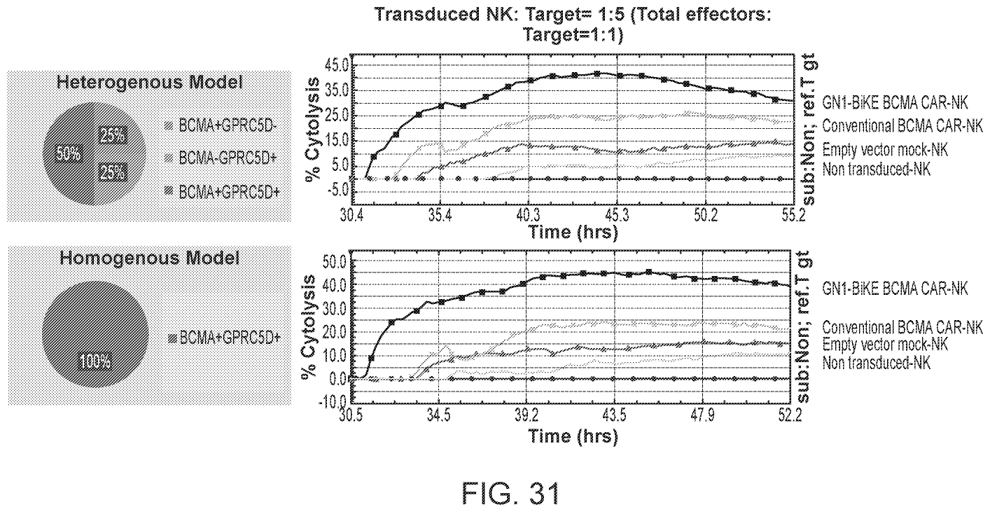

Vectors as illustrated in FIGS. 26-27 were constructed. The polypeptide expressed by the vectors are illustrated in FIG. 28. Expression of the polypeptide or a fragment thereof was investigated and the result is shown in FIG. 29. Efficacy of cells expressing such polypeptide in killing tumor cells was further evaluated and the result is shown in FIGS. 30-31.

### EXAMPLE 4-Efficacy of Killing Activity of “CAR-NK” Combination with “Bispecific Antibody”

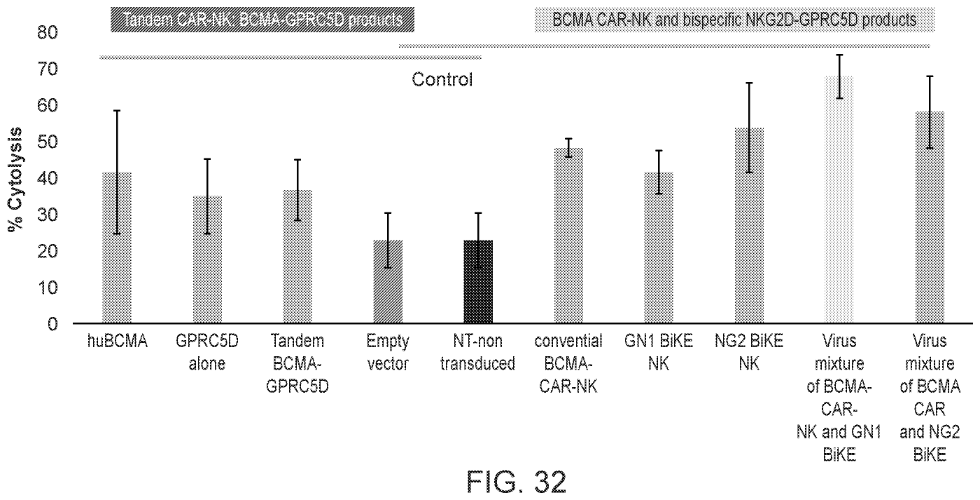

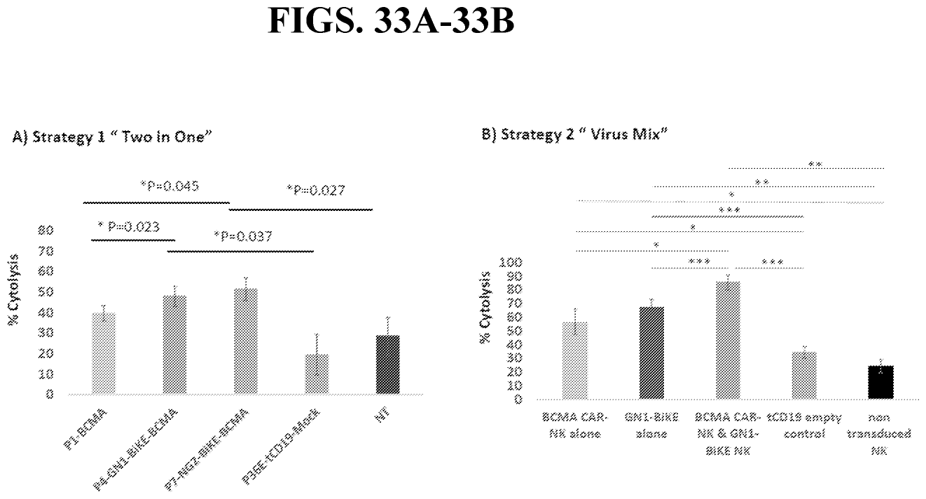

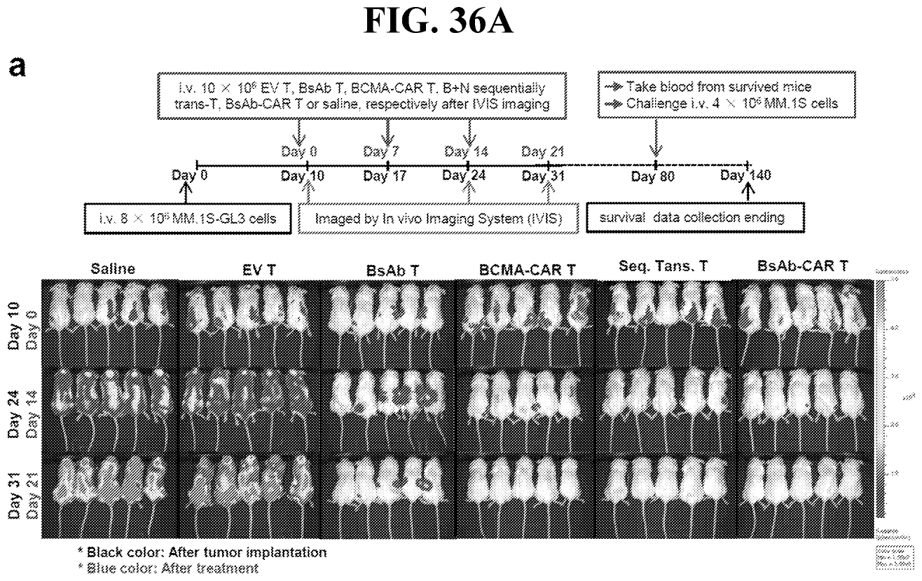

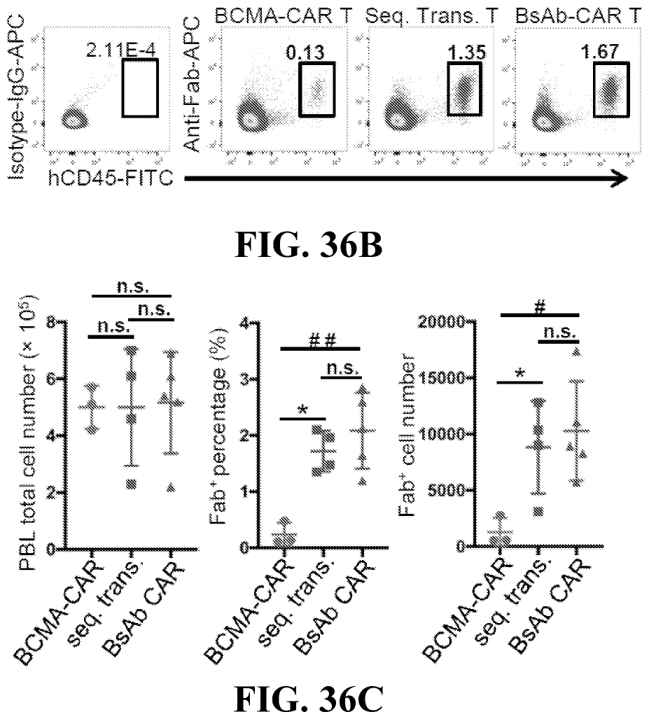

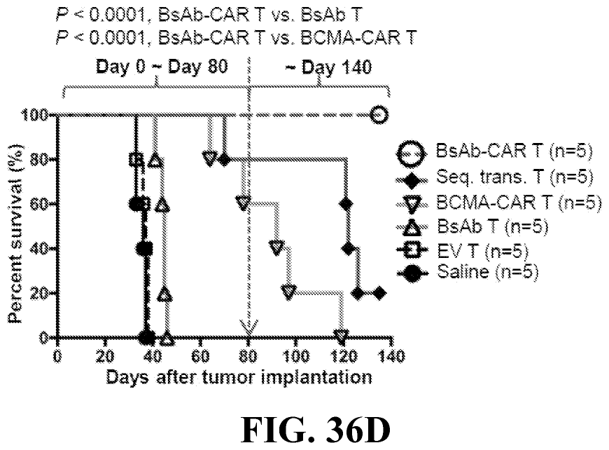

The constructs under investigation are illustrated in FIG. 28. Efficacy of cells expressing such construct in killing tumor cells was further evaluated and the result is provided in FIGS. 32-33 and 36. FIG. 32 clearly shows “CAR-NK” combination with “Bispecific Antibody” outperform “CAR-NK” based products.

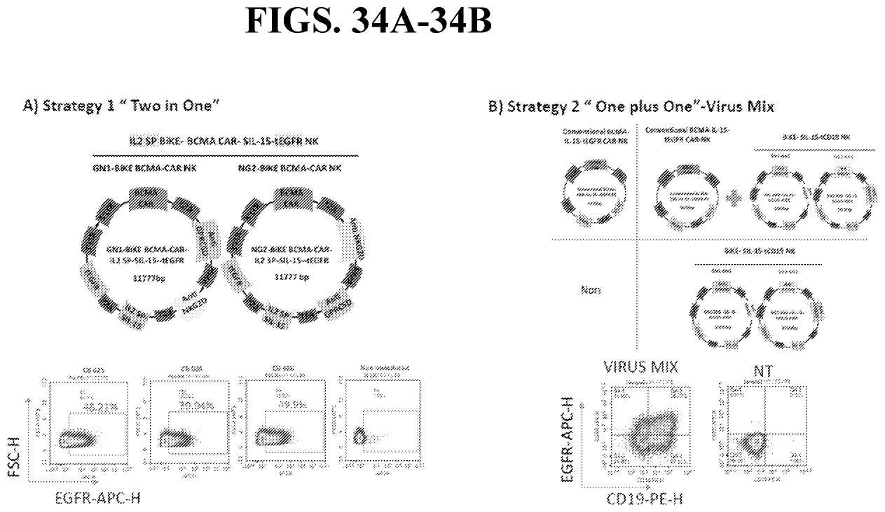

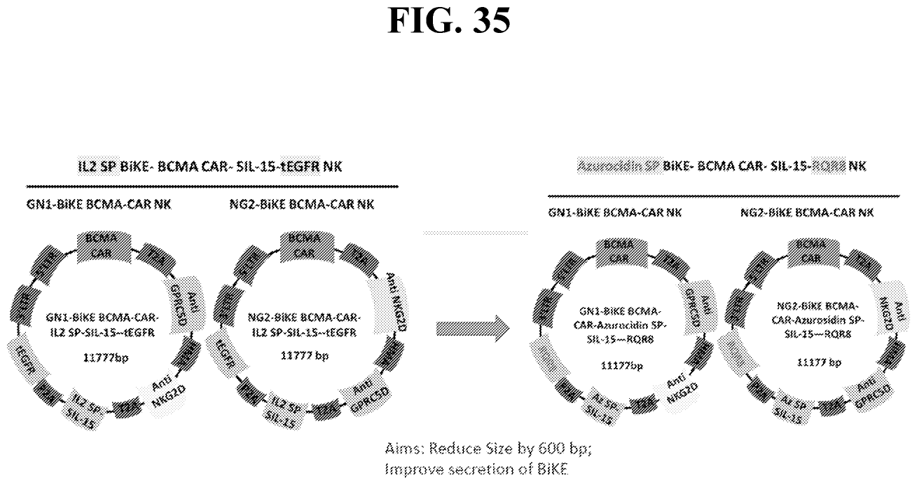

Two strategies of “CAR-NK” combination with “Bispecific Antibody” were tested: FIG. 34A shows strategy 1 “Two in One”, while FIG. 34B shows virus mixture strategy 2 “One plus One”. FIG. 35 shows optimizing of constructs “Two in One” with highly compact sort-suicide reporter RQR8 and strong azurocidin signal peptide for BiKE secretion. Transgenes expression and efficacy of antitumor after the improvement are tested.

### EXAMPLE 5-Anti BCMA CAR-NK with Secretion of Anti-GPRC5D and NKG2D-Bispecific NK Engager (BiKE) Enhances Anti-Multiple Myeloma (MM) Efficacy (In Vitro)

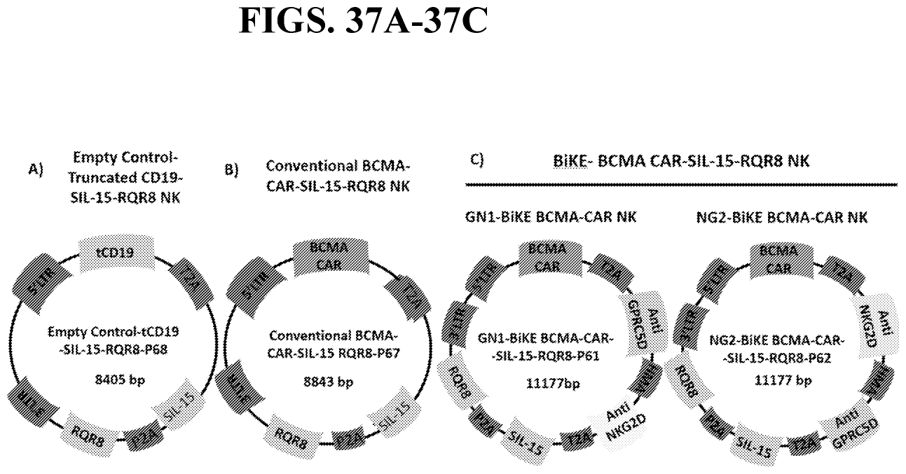

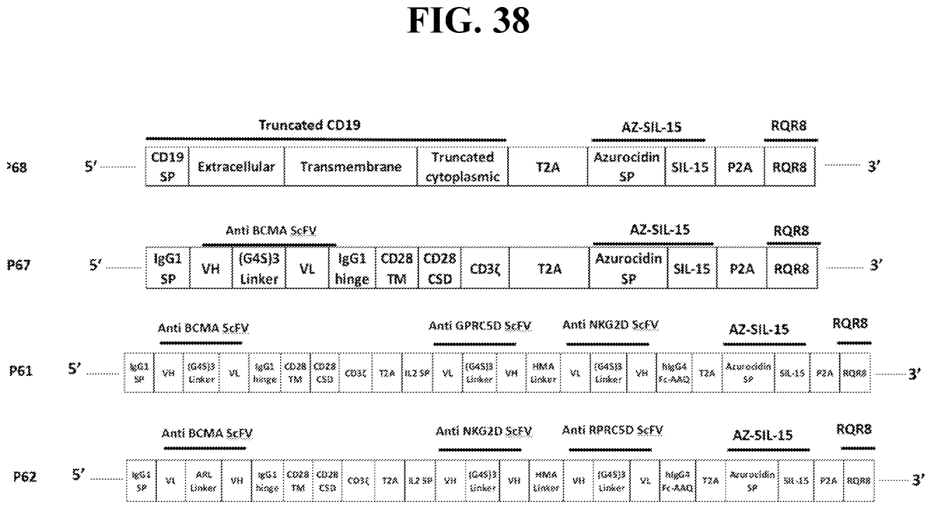

Schematic representation of anti-BCMA conventional CAR and anti NKG2D and GPRC5D BiKE CAR retroviral vectors is provided in FIG. 37. The corresponding polypeptide expressed are illustrated in FIG. 38. Examples 1˜4 demonstrated the super anti-Multiple Myeloma efficacy using the “Two in One” strategy for BCMA CAR with secretion of anti NKG2D and GPRC5D bispecific antibody for both CAR-T cells and CAR-NK cells.

In this study, with modification of introducing highly compact dual roles selection/suicide gene “RQR8”(471 bp) to replace the truncated EGFR (1071 bp), two new formats of “Two in One” constructs were created having an anti BCMA CAR-NK with secretion of anti NKG2D and GPRC5D bispecific NK cell engager (BiKE), which can greatly improve the ability for virus producing and NK cell transduction.

The amino acids and nucleotide sequences for each component and whole sequences of above constructs are provided in Table 3.

Optimization the Large Size (>11 kb) LTR Retrovirus Plasmids Preparation

In this study, MAX Efficiency™ Stb12™ Competent Cells (Invitrogen, 10268019) were used to propagate construct GPRC5D-NKG2D BiKE-BCMA CAR-SIL-15-RQR8-P61 and NKG2D-GPRC5D BiKE-BCMA CAR-SIL-15-RQR8-P62 (insert size>11 Kb) with optimized efficiency and quality.

For other plasmids with size<10 Kb (P68, P67), the NEB® Stable Competent E. coli (High Efficiency) (NEB, C3030H) was used.

All the plasmid colonies were selected on the LB Agar plates and liquid culture with 100 μg/ml of Carbenicillin (Teknova C2136) at 30 degree. For liquid culture grown, 180 rpm in a shaker was used.

The candidate plasmids with estimated bands identified with proper enzymes digestion were sent to perform gene sequencing (Retrogene Inc) to confirm the sequences were correct.

Preparation Cord Blood Derived CAR-NK Cells

Cord Blood NK cells were isolated using MACSxpress Whole Blood NK Cell Isolation Kit, human (Miltenyi Biotec,130-098-185), and cultured with NK MACS media (130-114-429) in the presence of 50 IU/ml of human IL-2, 5% AB human serum (Fisher BioReagents,BP2525100) and irradiated feeder cells with the ratio of 1:1. Viruses expressing a polypeptide as disclosed herein were produced. Using retronectin reagent (TakaRa, T100A/B) coated non-tissue culture plate, above constructs (FIG. 38) were transduced into activated NK cells at MOI 1 (7 days).

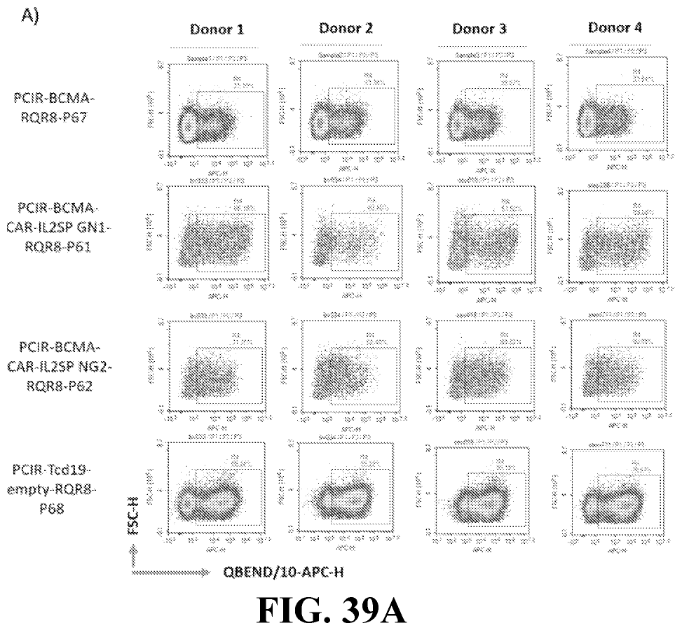

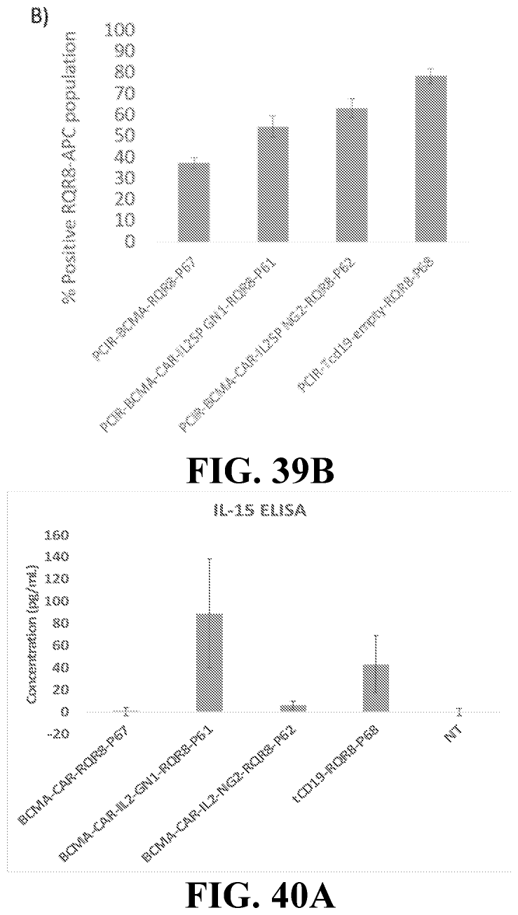

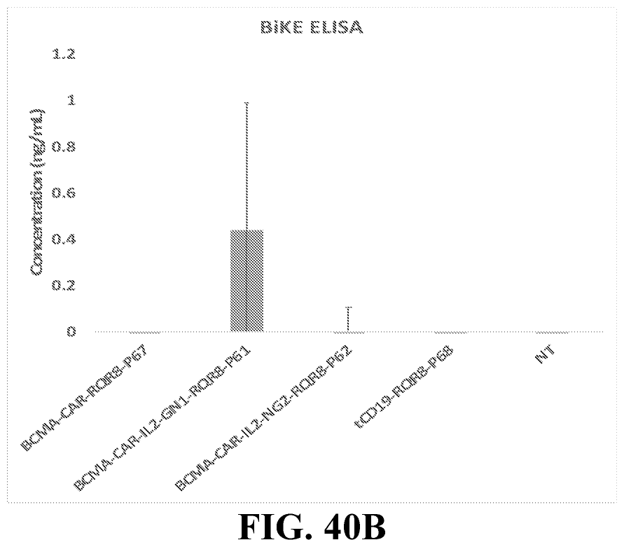

Flow analysis determined the transduction rates using APC anti-human CD34 Antibody (QBEnd10) (Allophycocyanin) (Novus Biologicals, LLC, #FAB7227A) for reporter RQR8 used in this study (FIG. 39).

IL-15 Secretion from all Constructs and Bi-Specific Antibody (BIKE) from Only Cd 19-Bike-Car.

For assessment of soluble IL-15 secretion, R&D System Human IL-15 Quantikine ELISA plate (R&D Systems catalog #S1500) and the manufacture's protocol was followed.

An unique ELISA assay was developed for detecting the concentration of full human Source Scfv-anti NKG2D and GPRCSD. Firstly, the Nunc MaxiSorp™ flat-bottom plate (Invitrogen™, 44-2404-21) was coated with 100 ng/well of Recombinant Human NKG2D, amino acid Phe78-Va1216 (Accession #P26718) (Biolegends, #781908) dissolved in 100 μl of DPBS (Gibco, 14190) at 4 degree overnight. After washing with 1x DPBS buffer with final 0.05% Tween 20 and blocking with 1% BSA in 1x DPBS buffer with final 0.05% Tween 20, 100 μl of samples and standard in blocking buffer were added to each well. The standard used in this assay was a human anti human NKG2D antibody, CD314 (NKG2D)-PE, human, Cat: 130-111-645, recombinant human IgG1, Miltenyi Biotec GmbH (0.25 mg/ml). For detection, a Biotinylated Goat anti-Human IgG (H+L) Cross-Adsorbed Secondary Antibody and HRP-Conjugated Streptavidin (Thermo Scientific™, N100), as well as 1-Step™ Ultra TMB-ELISA Substrate Solution, (Bio-Rad #34029). Stop buffer used was 1 M H₃PO₄.

0.5-1×10⁶ transduced NK cells (FIG. 39) were seeded and cultured in serum free media for 72 hours. Supernatant was subsequently collected, centrifuged, and immediately added to IL-15 pre-coated ELISA plate (R&D Systems S1500) and home developed BiKE detection ELISA with NKG2D-fusion protein coated plate. ELISA analysis showed there were detectable and Soluble IL 15 in each group and IL2 SP BiKE from non-concentrated supernatant of transduced BIKE-BCMA CAR-NK cells cultures in all 5 groups at culture 72 hours. Data present here was as average concentration from four independent donors as mean±SD.

Access the Long-Term Real Time Killing Assay Using xCELLigence RTCA.

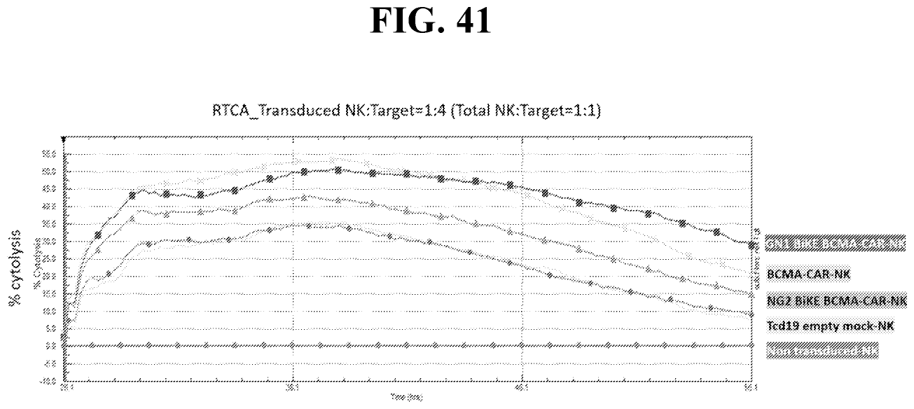

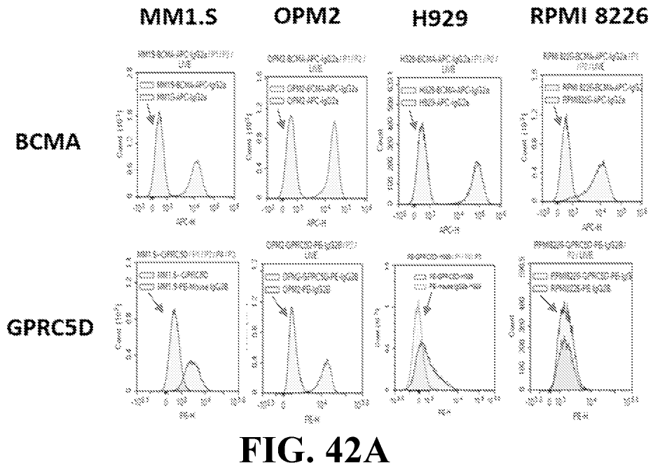

xCELLigence MP system instruments and RTCA Software Pro 2.3 (ACEA Biosciences, Agilent Technologies, Inc.) were used to perform and analyze the RTCA. Effector cells groups and Transduction evaluation used in RTCA were illustrated in FIG. 39, and the primary NK cells from cord blood cell were used in this study. Forced expression GPRCSD and BCMA gene on HT1080 used as tumor antigen targets in RTCA assay. Total 5000 targets were seeded on E-Plate 96 PET (Agilent REF 300600910) and incubated at 37 degree with 5% CO₂ for ˜23 h, then added total 5000 effectors into the wells with targets. Equal 1250 transduced NK used in each group by adjusting with non-transduced NK form same donor according to transgene expression. The ratio of transduced NK and Targets used in this study was 1:4. Cell Index (CI) data were recorded every 15 min for 120 h. The time to reach maximum Cell Index for targets alone group get was ˜56h, so the % Cytolysis was calculated and compared at ˜26h with addition of effectors. The data demonstrated there were enhanced tumor killing efficacy of GN1 BiKE BCMA CAR NK compared to conventional BCMA-CAR-NK, and others (FIG. 41).

Access the Long-Term Real Time Killing Assay by Flow Cytometry.

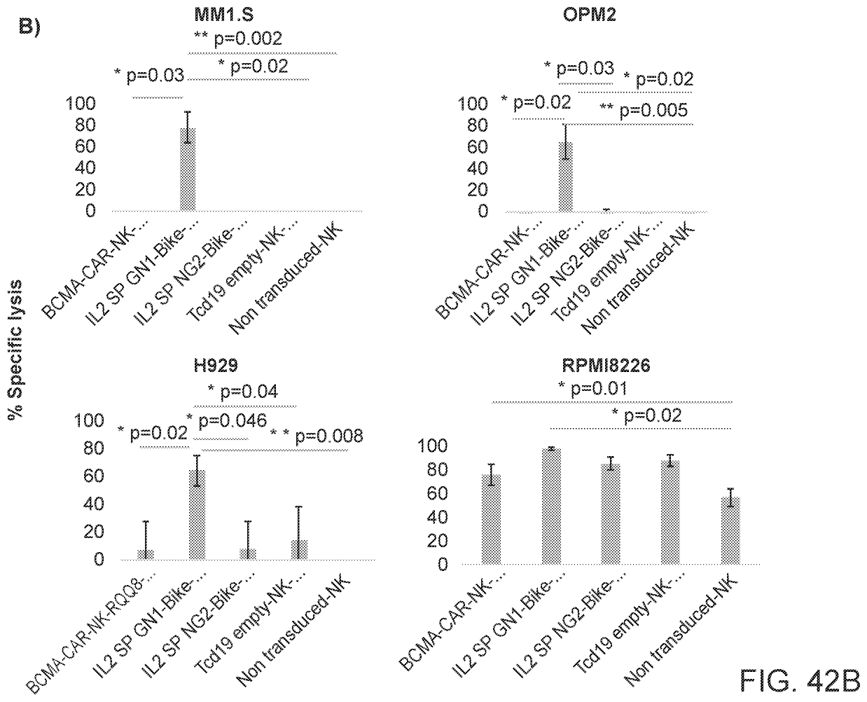

BCMA and GPRCSD surface expression on Multiple Myeloma (MM) cell lines, MM1.S, OPM2, H929 and RPMI8226 were assessed by using the flow antibody anti human BCMA (BioLegend, Cat #357506) and GPRCSD (R&D Systems, FAB6300P-100) with their isotype controls. The data demonstrated that, there are broad BCMA and GPRCSD on the surface of MM cell. Effectors CAR-NK cells and their mock empty vectors (Tcd19) control as well as non-transduced NK were co-culture with four Multiple Myeloma cell lines, MM1.S, OPM2, H929, and RPMI 8226 at the transduced CAR-NK: tumor ratio 1:8 (Total NK: Tumor 1:2) for 96h. Data presented as an average from 4 independent donors as Tumor lysis±SEM. Two tails Student's T-Test were used to evaluate the significances between two groups, and the index for P value were: * p<0.05; ** p<0.01. There are dramatically tumor killing activity of BCMA-CAR-NK with secretion bispecific antibody GPRCSD-NKG2D (GN1-BiKE) compared to conventional BCMA-CAR-NK, and others (FIG. 42).

TABLE 3a
P68: Empty Control-tCD19-SIL-15-RQR8-P68

| | | Nucleotide |
|---|---|---|
| | | sequence |
| | | (SEQ ID NO:) |
| P68: Empty Control- | 239 | 242 |
| Truncated CD19-SIL-15- | | |
| RQR8 | | |
| trancated CD19 | 240 | 243 |
| T2A | 135 | 244 |
| Azurocidin signal peptide | 126 | 151 |
| human IL-15 | 106 | 158 |
| P2A | 241 | 245 |
| RQR8 SEQUENCE | 132 | 163 |

TABLE 3b
P67: Conventional BCMA-CAR-SIL-15-RQR8-P67

| | Amino acid | Nucleotide |
|---|---|---|
| | sequence | sequence |
| | (SEQ ID NO:) | (SEQ ID NO:) |
| P67: Conventional BCMA- | 246 | 247 |
| CAR-SIL-15 RQR8 | | |
| IgG1 SP | 21 | 55 |
| VH of Anti-BCMA ScFV | 44 | 62 |
| (mouse C11D5.3) | | |
| (G4S)3 linker | 9 | 57 |
| VL of Anti-BCMA ScFV | 41 | 58 |
| (mouse C11D5.3) | | |
| IgG1 heavy chain hinge | 83 | 82 |
| CD28 transmembrane and | 48 | 67 |
| cytoplasmic domain | | |
| CD3Z isoform 3 | 49 | 68 |
| T2A | 135 | 244 |
| Azurocidin signal peptide | 126 | 151 |
| human IL-15 | 106 | 158 |
| P2A | 133 | 160 |
| RQR8 SEQUENCE | 132 | 163 |

TABLE 3c
P61: GPRC5D-NKG2D BiKE-BCMA CAR-SIL-15-RQR8-P61

| | Amino acid | Nucleotide |
|---|---|---|
| | sequence | sequence |
| | (SEQ ID NO:) | (SEQ ID NO:) |
| P61: GN1-BiKE BCMA-CAR-- | 138 | 168 |
| SIL-15-RQR8 | | |
| IgG1 SP | 21 | 55 |
| VH of Anti-BCMA ScFV | 44 | 62 |
| (mouse C11D5.3) | | |
| (G4S)3 linker | 9 | 57 |
| VL of Anti-BCMA ScFV | 41 | 58 |
| (mouse C11D5.3) | | |
| IgG1 heavy chain hinge | 47 | 66 |
| CD28 transmembrane and | 48 | 67 |
| cytoplasmic domain | | |
| CD3Z isoform 3 | 49 | 68 |
| T2A | 248 | 140 |
| IL2 signal peptide | 20 | 56 |
| VL of anti GPRC5D SCFV | 16 | 71 |
| (G4S)3 linker | 9 | 57 |
| VH of anti GPRC5D SCFV | 17 | 72 |
| HMA Linker | 81 | 70 |
| VL of anti NKG2D SCFV | 7 | 73 |
| (G4S)3 linker | 9 | 57 |
| VH of anti NKG2D SCFV | 8 | 249 |
| hIgG4-Fc | 19 | 250 |
| T2A | 135 | 251 |
| Azurocidin signal peptide | 126 | 151 |
| human IL-15 | 106 | 158 |
| P2A | 133 | 160 |
| RQR8 SEQUENCE | 132 | 163 |

TABLE 3d
P62: NKG2D-GPRC5D BiKE-BCMA CAR-SIL-15-RQR8-P62

| | Amino acid | Nucleotide |
|---|---|---|
| | sequence | sequence |
| | (SEQ ID NO:) | (SEQ ID NO:) |
| P62: NG2-BiKE BCMA- | 139 | 169 |
| CAR--SIL-15-RQR8 | | |
| IgG1 SP | 21 | 55 |
| VH of Anti-BCMA ScFV | 44 | 62 |
| (mouse C11D5.3) | | |
| (G4S)3 linker | 9 | 57 |
| VL of Anti-BCMA ScFV | 41 | 58 |
| (mouse C11D5.3) | | |
| IgG1 heavy chain hinge | 47 | 66 |
| CD28 transmembrane | 48 | 67 |
| and cytoplasmic domain | | |
| CD3Z isoform 3 | 49 | 68 |
| T2A | 248 | 140 |
| IL2 signal peptide | 20 | 56 |
| VH of anti NKG2D | 8 | 74 |
| SCFV | | |
| (G4S)3 linker | 9 | 146 |
| VL of anti NKG2D SCFV | 7 | 145 |
| HMA Linker | 81 | 144 |
| VH of anti GPRC5D | 17 | 143 |
| SCFV | | |
| (G4S)3 linker | 9 | 142 |
| VL of anti GPRC5D | 16 | 141 |
| SCFV | | |
| hIgG4-Fc | 19 | 147 |
| T2A | 135 | 149 |
| Azurocidin signal peptide | 126 | 151 |
| human IL-15 | 106 | 158 |
| P2A | 133 | 160 |
| RQR8 SEQUENCE | 132 | 163 |

### Embodiments of the Disclosure

Embodiment 1. A Polypeptide Comprising:

(i) an amino acid sequence of a Chimeric Antigen Receptor (CAR) comprising:
(1) an antigen binding amino acid sequence that recognizes and binds a tumor associated antigen (TAA) (anti-TAA antigen binding sequence),
(2) a hinge domain,
(3) a transmembrane domain, and
(4) an intracellular domain; and

(ii) an amino acid sequence of a bispecific antibody comprising
(1) an antigen binding sequence that recognizes and binds NKG2D (anti-NKG2D antigen binding sequence), and
(2) an antigen binding sequence that recognizes and binds G Protein-Coupled Receptor Class C Group 5 Member D (GPRC5D) (anti-GPRC5D antigen binding sequence).

Embodiment 2. The polypeptide of Embodiment 1, wherein the anti-NKG2D antigen binding sequence comprises one or two or three or four or five or all six of the following complementarity-determining regions (CDRs):

a light chain complementarity-determining
region 1 (CDRL1) comprising
(SEQ ID NO: 1)
SGSSSNIGNNAVN
or an equivalent thereof;
a light chain complementarity-determining
region 2 (CDRL2) comprising
(SEQ ID NO: 2)
YDDLLPS
or an equivalent thereof;
a light chain complementarity-determining
region 3 (CDRL3) comprising
(SEQ ID NO: 3)
AAWDDSLNGPV
or an equivalent thereof;
a heavy chain complementarity-determining
region 1 (CDRH1) comprising
(SEQ ID NO: 4)
GFTFSSY
or an equivalent thereof,
a heavy chain complementarity-determining
region 2 (CDRH2) comprising
(SEQ ID NO: 5)
RYDGSN
or an equivalent thereof;
and
a heavy chain complementarity-determining
region 3 (CDRH3) comprising
(SEQ ID NO: 6)
DRGLGDGTYFDY
or an equivalent thereof,

wherein the equivalents thereof recognizes and binds NKG2D.

Embodiment 3. The polypeptide of Embodiment 1 or 2, wherein the anti-NKG2D antigen binding sequence comprises:
a light chain variable region comprising SEQ ID NO: 7, or an equivalent thereof; and/or
a heavy chain variable region comprising SEQ ID NO: 8, or an equivalent thereof,
wherein the equivalent thereof recognizes and binds NKG2D.

Embodiment 4. The polypeptide of Embodiment 3, wherein the equivalent of SEQ ID NO: 7 or 8 is at least 80%, or at least 85%, or at least 90%, or at least 95%, or at least 96%, or at least 97%, or at least 98%, or at least 99% identical to SEQ ID NO: 7 or 8, respectively.

Embodiment 5. The polypeptide of Embodiment 2 or 3, wherein the equivalent of SEQ ID NOs: 1-8 comprises the amino acid sequence of C terminus to N terminus of SEQ ID NO: 1-8, respectively.

Embodiment 6. The polypeptide of any one of Embodiments 3 to 5, wherein the anti-NKG2D antigen binding sequence further comprises a peptide linker between the light chain variable region or the equivalent thereof and the heavy chain variable region or the equivalent thereof.

Embodiment 7. The polypeptide of any one of Embodiments 3 to 6, wherein the anti-NKG2D antigen binding sequence further comprises a peptide linker comprising SEQ ID NO: 9 or an equivalent thereof between the light chain variable region or the equivalent thereof and the heavy chain variable region or the equivalent thereof.

Embodiment 8. The polypeptide of any one of Embodiments 1 to 7, wherein the anti-GPRCSD antigen binding sequence comprises one or two or three or four or five or all six of the following CDRs:

a CDRL1 comprising
(SEQ ID NO: 10)
KASQNVATHVG
or an equivalent thereof;
a CDRL2 comprising
(SEQ ID NO: 11)
SASYRYS
or an equivalent thereof;
a CDRL3 comprising
(SEQ ID NO: 12)
QQYNRYPYT
or an equivalent thereof,
a CDRH1 comprising
(SEQ ID NO: 13)
GYSFTGY
or an equivalent thereof;
a CDRH2 comprising
(SEQ ID NO: 14)
NPYNSD
or an equivalent thereof;
and
a CDRH3 comprising
(SEQ ID NO: 15)
VALRVALDY
or an equivalent thereof,

wherein the equivalents thereof recognizes and binds GPRC5D.

Embodiment 9. The polypeptide of any one of Embodiments 1 to 8, wherein the anti-GPRC5D antigen binding sequence comprises
a light chain variable region comprising SEQ ID NO: 16, or an equivalent thereof; and/or
a heavy chain variable region comprising SEQ ID NO: 17, or an equivalent thereof,
wherein the equivalents thereof recognizes and binds GPRC5D.

Embodiment 10. The polypeptide of Embodiment 9, wherein the equivalent of SEQ ID NO: 16 or 17 is at least 80%, or at least 85%, or at least 90%, or at least 95%, or at least 96%, or at least 97%, or at least 98%, or at least 99% identical to SEQ ID NO: 16 or 17, respectively.

Embodiment 11. The polypeptide of Embodiment 8 or 9, wherein the equivalent of SEQ ID NOs: 10-17 comprises the amino acid sequence of C terminus to N terminus of SEQ ID NOs: 10-17, respectively.

Embodiment 12. The polypeptide of any one of Embodiments 9 to 11, wherein the anti-GPRC5D antigen binding sequence further comprises a peptide linker between the light chain variable region or the equivalent thereof and the heavy chain variable region or the equivalent thereof.

Embodiment 13. The polypeptide of any one of Embodiments 9 to 12, wherein the anti-GPRC5D antigen binding sequence further comprises a peptide linker comprising SEQ ID NO: 9 or an equivalent thereof between the light chain variable region or the equivalent thereof and the heavy chain variable region or the equivalent thereof.

Embodiment 14. The polypeptide of any one of Embodiments 1 to 13, wherein the bispecific antibody further comprises a fragment crystallizable (Fc) region of an immunoglobulin, a mutant thereof, or an equivalent thereof.

Embodiment 15. The polypeptide of Embodiment 14, wherein the Fc region or a mutant thereof is a human Fc region or a mutant thereof.

Embodiment 16. The polypeptide of Embodiment 14 or 15, wherein the Fc region comprises SEQ ID NO: 18, or an equivalent thereof that stabilizes the bispecific antibody.

Embodiment 17. The polypeptide of any one of Embodiments 14 to 16, wherein the Fc region comprises SEQ ID NO: 19 or an Fc equivalent having mutations at a position corresponding to amino acid (aa) 16, aa 17 and aa 79 of SEQ ID NO: 19, and stabilizing the bispecific antibody.

Embodiment 18. The polypeptide of any one of Embodiments 1 to 17, wherein the bispecific antibody further comprises a peptide linker between the anti-NKG2D antigen binding sequence and the anti-GPRCSD antigen binding sequence.

Embodiment 19. The polypeptide of Embodiment 18, wherein the peptide linker comprises PSGQAGAAASESLFVSNHAY (SEQ ID NO: 81) or an equivalent thereof.

Embodiment 20. The polypeptide of any one of Embodiments 1 to 19, wherein the bispecific antibody further comprises a signal peptide at its N terminus.

Embodiment 21. The polypeptide of Embodiment 20, wherein the signal peptide comprises MGWSSIILFLVATATGVH (SEQ ID NO: 21), MYRMQLLSCIALSLALVTNS (SEQ ID NO: 20), or MWLQSLLLLGTVACSIS (SEQ ID NO: 122), or MRISKPHLRSISIQCYLCLLLNSHFLTEA (SEQ ID NO: 123), or MRSSPGNMERIVICLMVIFLGTLV (SEQ ID NO: 124), or an equivalent of each thereof that facilitates secretion of the bispecific antibody, fragment or equivalent thereof.

Embodiment 22. The polypeptide of any one of Embodiments 1 to 21, wherein the bispecific antibody further comprises a detectable or purification marker.

Embodiment 23. The polypeptide of Embodiment 22, wherein the detectable marker comprises YPYDVPDYA (SEQ ID NO: 22).

Embodiment 24. The polypeptide of any one of Embodiments 1 to 23, wherein the TAA is not GPRCSD.

Embodiment 25. The polypeptide of any one of Embodiments 1 to 24, wherein the TAA comprises an epitope from a protein or polypeptide selected from: B-cell maturation antigen (BCMA), FLT3, CD19, mesothelin, human epidermal growth factor receptor 2 (HER2), prostate stem cell antigen (PSCA), carcinoembryonic antigen (CEA), CD33, GTPase-activating protein (GAP), ganglioside G2 (GD2), CD5, prostate specific membrane antigen (PSMA), receptor tyrosine kinase-like orphan receptor 1 (ROR1), CD123, CD70, CD38, mucin 1, (Muc1), ephrin type-A receptor 2 precursor (EphA2), epidermal growth factor receptor variant III (EGFRVIII), interleukin 13 receptor alpha 2 (IL13Ra2), CD133, glypican 3 (GPC3), epithelial cell adhesion molecule precursor (EpCam), fibroblast activation protein alpha (FAP), vascular endothelial growth factor receptor 2 (VEGFR2), cancer/testis (CT), guanylyl cyclase C (GUCY2C), tumor-associated glycoprotein-72 (TAG-72), thymidine kinase 1 (TK1), and hypoxanthine guanine phosphoribosyltransferase (HPRT1).

Embodiment 26. The polypeptide of any one of Embodiments 1 to 25, wherein the TAA comprises an epitope of BCMA.

Embodiment 27. The polypeptide of Embodiment 25 or 26, wherein the antigen binding sequence that recognizes and binds BCMA (anti-BCMA antigen binding sequence) comprises one or two or three or four or five or six of the following CDRs:

a CDRL1 comprising a sequence selected from
(SEQ ID NO: 23)
RASESVTILGSHLIH,
(SEQ ID NO: 24)
SASQDISNYLN,
(SEQ ID NO: 25)
RASESVTILGSHLIY,
or an equivalent of each thereof;
a CDRL2 comprising a sequence selected from
(SEQ ID NO: 26)
LASNVQT,
(SEQ ID NO: 27)
YTSNLHS,
(SEQ ID NO: 28)
LASNVQT,
or an equivalent of each thereof;
a CDRL3 comprising a sequence selected from
(SEQ ID NO: 29)
LQSRTIPRT,
(SEQ ID NO: 30)
QQYRKLPWT,
(SEQ ID NO: 31)
LQSRTIPRT,
an equivalent of each thereof;
a CDRH1 comprising a sequence selected from
(SEQ ID NO: 32)
GYTFTDY,
(SEQ ID NO: 33)
GGTFSNY,
(SEQ ID NO: 34)
GYTFRHY,
or an equivalent of each thereof;
a CDRH2 comprising a sequence selected from
(SEQ ID NO: 35)
INTETRE,
(SEQ ID NO: 36)
YRGHSD,
(SEQ ID NO: 37)
NTESGV,
or an equivalent of each thereof;
and
a CDRH3 comprising a sequence selected from
(SEQ ID NO: 38)
DYSYAMDY,
(SEQ ID NO: 39)
GAIYNGYDVLDN,
(SEQ ID NO: 40)
DYLYSLDF,
or an equivalent of each thereof,

wherein the equivalents thereof recognizes and binds BCMA.

Embodiment 28. The polypeptide of Embodiment 27, wherein the anti-BCMA antigen binding sequence comprises a light chain variable region comprising a sequence selected from SEQ ID NO: 41, SEQ ID NO: 42, SEQ ID NO: 43, or an equivalent of each thereof and/or a heavy chain variable region comprising a sequence selected from SEQ ID NO: 44, SEQ ID NO: 45, SEQ ID NO: 46, or an equivalent of each thereof, wherein the equivalents thereof recognizes and binds BCMA.

Embodiment 29. The polypeptide of Embodiment 28, wherein the equivalent of SEQ ID NOs: 41 to 46 is at least 80%, or at least 85%, or at least 90%, or at least 95%, or at least 96%, or at least 97%, or at least 98%, or at least 99% identical to SEQ ID NOs: 41 to 46, respectively.

Embodiment 30. The polypeptide of Embodiment 27 or 28, wherein the equivalent of SEQ ID NOs: 23-46 comprises the amino acid sequence of C terminus to N terminus of SEQ ID NOs: 23-46, respectively.

Embodiment 31. The polypeptide of any one of Embodiments 28 to 30, wherein the anti-BCMA antigen binding sequence or an equivalent thereof further comprises a peptide linker between the light chain variable region or the equivalent thereof and the heavy chain variable region or the equivalent thereof.

Embodiment 32. The polypeptide of any one of Embodiments 28 to 31, wherein the anti-BCMA antigen binding sequence further comprises a peptide linker comprising SEQ ID NO: 9 or an equivalent thereof between the light chain variable region or the equivalent thereof and the heavy chain variable region or the equivalent thereof.

Embodiment 33. The polypeptide of any one of Embodiments 1 to 32, wherein the hinge domain comprises a CD8 a hinge domain or an IgG1 hinge domain.

Embodiment 34. The polypeptide of Embodiment 33, wherein the IgG1 hinge domain comprises LEPKSCDKTHTCPPCPDPKGT (SEQ ID NO: 47) or an equivalent thereof.

Embodiment 35. The polypeptide of any one of Embodiments 1 to 34, wherein the transmembrane domain comprises a CD8 a transmembrane domain or a CD28 transmembrane domain.

Embodiment 36. The polypeptide of any one of Embodiments 1 to 35, wherein the intracellular domain comprises one or more or two or more costimulatory regions selected from a CD28 costimulatory signaling region, a 4-1BB costimulatory signaling region, an ICOS costimulatory signaling region, or an OX40 costimulatory region.

Embodiment 37. The polypeptide of Embodiment 35 or 36, wherein the CAR comprises a CD28 transmembrane and cytoplasmic/intracellular domain (such as a CD28 transmembrane domain and a CD28 costimulatory signaling region) comprising SEQ ID NO: 48 or an equivalent thereof.

Embodiment 38. The polypeptide of any one of Embodiments 1 to 37, wherein the intracellular domain further comprises a CD3 zeta signaling domain.

Embodiment 39. The polypeptide of Embodiment 38, wherein the CD3 zeta signaling domain comprises SEQ ID NO: 49 or an equivalent thereof.

Embodiment 40. The polypeptide of any one of Embodiments 1 to 39, wherein the intracellular domain further comprises an IL2Rβ or a fragment thereof comprising an JAK-STAT activation domain.

Embodiment 41. The polypeptide of any one of Embodiments 1 to 40, further comprising a suicide gene product or a detectable marker or both.

Embodiment 42. The polypeptide of Embodiment 41, wherein the suicide gene product is selected from one or more of: HSV-TK (Herpes simplex virus thymidine kinase), cytosine deaminase, nitroreductase, carboxylesterase, cytochrome P450 or PNP (Purine nucleoside phosphorylase), truncated EGFR, or inducible caspase (“iCasp”).

Embodiment 43. The polypeptide of any one of Embodiments 1 to 42, wherein the CAR further comprises a signal peptide at its N terminus, optionally different from the signal peptide at the N terminus of the bispecific antibody.

Embodiment 44. The polypeptide of Embodiment 43, wherein the signal peptide comprises MYRMQLLSCIALSLALVTNS (SEQ ID NO: 20), MGWSSIILFLVATATGVH (SEQ ID NO: 21), MLLLVTSLLLCELPHPAFLLIP (SEQ ID NO: 125), or MWLQSLLLLGTVACSIS (SEQ ID NO: 122), or MRISKPHLRSISIQCYLCLLLNSHFLTEA (SEQ ID NO: 123), or MRSSPGNMERIVICLMVIFLGTLV (SEQ ID NO: 124), or an equivalent of each thereof.

Embodiment 45. The polypeptide of any one of Embodiments 1 to 44, wherein the CAR further comprises a detectable marker.

Embodiment 46. The polypeptide of any one of Embodiments 1 to 45, further comprising a cleavable peptide located between any two of the following:
the CAR;
the bispecific antibody (also referred to herein as BiTE, bispecific T cell engager or BiKE, bispecific NK cell engager); and
an optional suicide gene product or detectable marker or both.

Embodiment 47. The polypeptide of Embodiment 46, wherein the cleavable peptide is a self-cleaving peptide.

Embodiment 48. The polypeptide of Embodiment 47, wherein the self-cleaving peptide is a T2A peptide.

Embodiment 49. The polypeptide of any one of Embodiments 46 to 48, wherein cleavable peptide or self-cleaving peptide or the T2A peptide comprises HVGSGEGRGSLLTCGDVEENPGP (SEQ ID NO: 50) GSGATNFSLLKQAGDVEENPGP (SEQ ID NO: 133), EGRGSLLTCGDVEENPGP (SEQ ID NO: 134), or an equivalent of one or each thereof.

Embodiment 50. The polypeptide of any one of Embodiments 1 to 49, comprising one or more of the following sequences or a fragment thereof: SEQ ID NO: 51; SEQ ID NO: 52; SEQ ID NO: 53; and SEQ ID NO: 54.

Embodiment 51. A bispecific antibody or a fragment thereof with the proviso that the fragment recognizes and binds to both NKG2D and G Protein-Coupled Receptor Class C Group 5 Member D (GPRCSD), comprising
(1) an antigen binding sequence that recognizes and binds NKG2D (anti-NKG2D antigen binding sequence), and
(2) an antigen binding sequence that recognizes and binds GPRC5D (anti-GPRC5D antigen binding sequence).

Embodiment 52. The antibody or fragment of Embodiment 51, wherein the anti-NKG2D antigen binding sequence comprises one or two or three or four or five or all six of the following complementarity-determining regions (CDRs):

a light chain complementarity-
determining region 1 (CDRL1) comprising
(SEQ ID NO: 1)
SGSSSNIGNNAVN
or an equivalent thereof;
a light chain complementarity-
determining region 2 (CDRL2) comprising
(SEQ ID NO: 2)
YDDLLPS
or an equivalent thereof;
a light chain complementarity-
determining region 3 (CDRL3) comprising
(SEQ ID NO: 3)
AAWDDSLNGPV
or an equivalent thereof;
a heavy chain complementarity-
determining region 1 (CDRH1) comprising
(SEQ ID NO: 4)
GFTFSSY
or an equivalent thereof;
a heavy chain complementarity-
determining region 2 (CDRH2) comprising
(SEQ ID NO: 5)
RYDGSN
or an equivalent thereof;
and
a heavy chain complementarity-
determining region 3 (CDRH3) comprising
(SEQ ID NO: 6)
DRGLGDGTYFDY
or an equivalent thereof,

wherein the equivalents thereof recognizes and binds NKG2D.

Embodiment 53. The antibody or fragment of Embodiment 51 or 52, wherein the anti-NKG2D antigen binding sequence comprises:

a light chain variable region comprising SEQ ID NO: 7, or an equivalent thereof;
and/or a heavy chain variable region comprising SEQ ID NO: 8, or an equivalent thereof, wherein the equivalent thereof recognizes and binds NKG2D.

Embodiment 54. The antibody or fragment of Embodiment 53, wherein the equivalent of SEQ ID NO: 7 or 8 is at least 80%, or at least 85%, or at least 90%, or at least 95%, or at least 96%, or at least 97%, or at least 98%, or at least 99% identical to SEQ ID NO: 7 or 8, respectively.

Embodiment 55. The antibody or fragment of Embodiment 52 or 53, wherein the equivalent of SEQ ID NOs: 1-8 comprises the amino acid sequence of C terminus to N terminus of SEQ ID NO: 1-8, respectively.

Embodiment 56. The antibody or fragment of any one of Embodiments 53 to 55, wherein the anti-NKG2D antigen binding sequence further comprises a peptide linker between the light chain variable region or the equivalent thereof and the heavy chain variable region or the equivalent thereof.

Embodiment 57. The antibody or fragment of any one of Embodiments 53 to 56, wherein the anti-NKG2D antigen binding sequence further comprises a peptide linker comprising SEQ ID NO: 9 or an equivalent thereof between the light chain variable region or the equivalent thereof and the heavy chain variable region or the equivalent thereof.

Embodiment 58. The antibody or fragment of any one of Embodiments 51 to 57, wherein the anti-GPRCSD antigen binding sequence comprises one or two or three or four or five or all six of the following CDRs:

a CDRL1 comprising
(SEQ ID NO: 10)
KASQNVATHVG
or an equivalent thereof;
a CDRL2 comprising
(SEQ ID NO: 11)
SASYRYS
or an equivalent thereof;
a CDRL3 comprising
(SEQ ID NO: 12)
QQYNRYPYT
or an equivalent thereof,
a CDRH1 comprising
(SEQ ID NO: 13)
GYSFTGY
or an equivalent thereof;
a CDRH2 comprising
(SEQ ID NO: 14)
NPYNSD
or an equivalent thereof, and
a CDRH3 comprising
(SEQ ID NO: 15)
VALRVALDY
or an equivalent thereof,

wherein the equivalents thereof recognizes and binds GPRCSD.

Embodiment 59. The antibody or fragment of any one of Embodiments 51 to 58, wherein the anti-GPRCSD antigen binding sequence comprises and/or a light chain variable region comprising SEQ ID NO: 16, or an equivalent thereof;
a heavy chain variable region comprising SEQ ID NO: 17, or an equivalent thereof, wherein the equivalents thereof recognizes and binds GPRCSD.

Embodiment 60. The antibody or fragment of Embodiment 59, wherein the equivalent of SEQ ID NO: 16 or 17 is at least 80%, or at least 85%, or at least 90%, or at least 95%, or at least 96%, or at least 97%, or at least 98%, or at least 99% identical to SEQ ID NO: 16 or 17, respectively.

Embodiment 61. The antibody or fragment of Embodiment 58 or 59, wherein the equivalent of SEQ ID NOs: 10-17 comprises the amino acid sequence of C terminus to N terminus of SEQ ID NOs: 10-17, respectively.

Embodiment 62. The antibody or fragment of any one of Embodiments 59 to 61, wherein the anti-GPRCSD antigen binding sequence further comprises a peptide linker between the light chain variable region or the equivalent thereof and the heavy chain variable region or the equivalent thereof.

Embodiment 63. The antibody or fragment of any one of Embodiments 59 to 62, wherein the anti-GPRCSD antigen binding sequence further comprises a peptide linker comprising SEQ ID NO: 9 or an equivalent thereof between the light chain variable region or the equivalent thereof and the heavy chain variable region or the equivalent thereof.

Embodiment 64. The antibody or fragment of any one of Embodiments 51 to 63, wherein the bispecific antibody further comprises a fragment crystallizable (Fc) region of an immunoglobulin, a mutant thereof, or an equivalent thereof.

Embodiment 65. The antibody or fragment of Embodiment 64, wherein the Fc region or a mutant thereof is a human Fc region or a mutant thereof.

Embodiment 66. The antibody or fragment of Embodiment 64 or 65, wherein the Fc region comprises SEQ ID NO: 18, or an equivalent thereof that stabilizes the bispecific antibody.

Embodiment 67. The antibody or fragment of any one of Embodiments 64 to 66, wherein the Fc region comprises SEQ ID NO: 19 or an Fc equivalent having mutations at a position corresponding to amino acid (aa) 16, aa 17 and aa 79 of SEQ ID NO: 19, and stabilizing the bispecific antibody.

Embodiment 68. The antibody or fragment of any one of Embodiments 51 to 67, wherein the bispecific antibody further comprises a peptide linker between the anti-NKG2D antigen binding sequence and the anti-GPRCSD antigen binding sequence.

Embodiment 69. The antibody or fragment of Embodiment 68, wherein the peptide linker comprises PSGQAGAAASESLFVSNHAY (SEQ ID NO: 81) or an equivalent thereof.

Embodiment 70. The antibody or fragment of any one of Embodiments 51 to 69, wherein the bispecific antibody further comprises a signal peptide at its N terminus.

Embodiment 71. The antibody or fragment of Embodiment 70, wherein the signal peptide comprises MGWSSIILFLVATATGVH (SEQ ID NO: 21), MYRMQLLSCIALSLALVTNS (SEQ ID NO: 20), or MWLQSLLLLGTVACSIS (SEQ ID NO: 122), or MRISKPHLRSISIQCYLCLLLNSHFLTEA (SEQ ID NO: 123), or MRSSPGNMERIVICLMVIFLGTLV (SEQ ID NO: 124), or an equivalent of each thereof that facilitates secretion of the bispecific antibody, fragment or equivalent thereof.

Embodiment 72. The antibody or fragment of any one of Embodiments 51 to 71, wherein the bispecific antibody further comprises a detectable or purification marker.

Embodiment 73. The antibody or fragment of Embodiment 72, wherein the detectable marker comprises YPYDVPDYA (SEQ ID NO: 22).

Embodiment 74. A Chimeric Antigen Receptor (CAR) comprising:
(1) an antigen binding sequence that recognizes and binds G Protein-Coupled Receptor Class C Group 5 Member D (GPRCSD) (anti-GPRCSD antigen binding sequence) or an equivalent thereof with the proviso that the equivalent recognizes and binds GPRCSD,
(2) a hinge domain,
(3) a transmembrane domain, and
(4) an intracellular domain.

Embodiment 75. The CAR of Embodiment 74, further comprising an antigen binding sequence that recognizes and binds a non-GPRCSD TAA.

Embodiment 76. The CAR of Embodiment 75, wherein the TAA comprises an epitope from a protein or polypeptide selected from: B-cell maturation antigen (BCMA), FLT3, CD19, mesothelin, human epidermal growth factor receptor 2 (HER2), prostate stem cell antigen (PSCA), carcinoembryonic antigen (CEA), CD33, GTPase-activating protein (GAP), ganglioside G2 (GD2), CDS, prostate specific membrane antigen (PSMA), receptor tyrosine kinase-like orphan receptor 1 (ROR1), CD123, CD70, CD38, mucin 1, (Muc1), ephrin type-A receptor 2 precursor (EphA2), epidermal growth factor receptor variant III (EGFRVIII), interleukin 13 receptor alpha 2 (IL13Ra2), CD133, glypican 3 (GPC3), epithelial cell adhesion molecule precursor (EpCam), fibroblast activation protein alpha (FAP), vascular endothelial growth factor receptor 2 (VEGFR2), cancer/testis (CT), guanylyl cyclase C (GUCY2C), tumor-associated glycoprotein-72 (TAG-72), thymidine kinase 1 (TK1), and hypoxanthine guanine phosphoribosyltransferase (HPRT1).

Embodiment 77. The CAR of Embodiment 75 or 76, wherein the TAA comprises an epitope of BCMA.

Embodiment 78. The CAR of any one of Embodiments 74 to 77, wherein the antigen binding sequence that recognizes and binds GPRC5D comprises one or two or three or four or five or six of the following CDRs:

a CDRL1 comprising
(SEQ ID NO: 10)
KASQNVATHVG
or an equivalent thereof;
a CDRL2 comprising
(SEQ ID NO: 11)
SASYRYS
or an equivalent thereof;
a CDRL3 comprising
(SEQ ID NO: 12)
QQYNRYPYT
or an equivalent thereof;
a CDRH1 comprising
(SEQ ID NO: 13)
GYSFTGY
or an equivalent thereof;
a CDRH2 comprising
(SEQ ID NO: 14)
NPYNSD
or an equivalent thereof;
and
a CDRH3 comprising
(SEQ ID NO: 15)
VALRVALDY
or an equivalent thereof,

wherein the equivalents thereof recognizes and binds GPRC5D.

Embodiment 79. The CAR of Embodiment 78, wherein the anti-GPRC5D antigen binding sequence comprises
a light chain variable region comprising SEQ ID NO: 16, or an equivalent thereof; and/or
a heavy chain variable region comprising SEQ ID NO: 17, or an equivalent thereof,
wherein the equivalents thereof recognizes and binds GPRC5D.

Embodiment 80. The CAR of Embodiment 79, wherein the equivalent of SEQ ID NO: 16 or 17 is at least 80%, or at least 85%, or at least 90%, or at least 95%, or at least 96%, or at least 97%, or at least 98%, or at least 99% identical to SEQ ID NO: 16 or 17, respectively.

Embodiment 81. The CAR of Embodiment 78 or 79, wherein the equivalent of SEQ ID NOs: 10-17 comprises the amino acid sequence of C terminus to N terminus of SEQ ID NOs: 10-17, respectively.

Embodiment 82. The CAR of any one of Embodiments 79 to 81, wherein the anti-GPRC5D antigen binding sequence further comprises a peptide linker between the light chain variable region or the equivalent thereof and the heavy chain variable region or the equivalent thereof.

Embodiment 83. The CAR of any one of Embodiments 79 to 81, wherein the anti-GPRC5D antigen binding sequence further comprises a peptide linker comprising SEQ ID NO: 9 or an equivalent thereof between the light chain variable region or the equivalent thereof and the heavy chain variable region or the equivalent thereof.

Embodiment 84. The CAR of any one of Embodiments 74 to 83, wherein the hinge domain comprises a CD8 a hinge domain or an IgG1 hinge domain.

Embodiment 85. The CAR of Embodiment 84, wherein the IgG1 hinge domain comprises LEPKSCDKTHTCPPCPDPKGT (SEQ ID NO: 47) or an equivalent thereof.

Embodiment 86. The CAR of any one of Embodiments 74 to 85, wherein the transmembrane domain comprises a CD8 a transmembrane domain or a CD28 transmembrane domain.

Embodiment 87. The CAR of any one of Embodiments 74 to 86, wherein the intracellular domain comprises one or more or two or more costimulatory regions selected from a CD28 costimulatory signaling region, a 4-1BB costimulatory signaling region, an ICOS costimulatory signaling region, or an OX40 costimulatory region.

Embodiment 88. The CAR of Embodiment 86 or 87, wherein the CAR comprises a CD28 transmembrane and cytoplasmic/intracellular domain (such as a CD28 transmembrane domain and a CD28 costimulatory signaling region) comprising SEQ ID NO: 48 or an equivalent thereof.

Embodiment 89. The CAR of any one of Embodiments 74 to 88, wherein the intracellular domain further comprises a CD3 zeta signaling domain.

Embodiment 90. The CAR of Embodiment 89, wherein the CD3 zeta signaling domain comprises SEQ ID NO: 49 or an equivalent thereof.

Embodiment 91. The CAR of any one of Embodiments 74 to 90, wherein the intracellular domain further comprises an IL2Rβ or a fragment thereof comprising an JAK-STAT activation domain.

Embodiment 92. The CAR of any one of Embodiments 74 to 91, further comprising a suicide gene product or a detectable marker or both.

Embodiment 93. The CAR of Embodiment 92, wherein the suicide gene product is selected from one or more of: HSV-TK (Herpes simplex virus thymidine kinase), cytosine deaminase, nitroreductase, carboxylesterase, cytochrome P450 or PNP (Purine nucleoside phosphorylase), truncated EGFR, or inducible caspase (“iCasp”).

Embodiment 94. The CAR of any one of Embodiments 74 to 93, wherein the CAR further comprises a signal peptide at its N terminus, optionally different from the signal peptide at the N terminus of the bispecific antibody.

Embodiment 95. The CAR of Embodiment 94, wherein the signal peptide comprises MYRMQLLSCIALSLALVTNS (SEQ ID NO: 20), MGWSSIILFLVATATGVH (SEQ ID NO: 21), MLLLVTSLLLCELPHPAFLLIP (SEQ ID NO: 125), or MWLQSLLLLGTVACSIS (SEQ ID NO: 122), or MRISKPHLRSISIQCYLCLLLNSHFLTEA (SEQ ID NO: 123), or MRSSPGNIVIERIVICLMVIFLGTLV (SEQ ID NO: 124), or an equivalent of each thereof.

Embodiment 96. The CAR of any one of Embodiments 74 to 95, wherein the CAR further comprises a detectable marker.

Embodiment 97. A polynucleotide encoding one or more of the following: a polypeptide of any one of Embodiments 1 to 50, a bispecific antibody of any one of Embodiments 51 to 73, or a CAR of any one of Embodiments 74 to 96.

Embodiment 98. The polynucleotide of Embodiment 97, further comprising any one or any two or all three of the following:
a first regulatory sequence directing the expression of the polypeptide;
a second regulatory sequence directing the expression of the bispecific antibody; and
a third regulatory sequence directing the expression of the CAR.

Embodiment 99. The polynucleotide of Embodiment 98, wherein each of the regulatory sequences comprise one or more of the following: a promoter, an intron, an enhancer, or a polyadenylation signal.

Embodiment 100. The polynucleotide of any one of Embodiments 97 to 99, comprising one or more of the following:
(I) a nucleotide sequence encoding a signal peptide, and wherein the nucleotide sequence comprises SEQ ID NO: 55 or an equivalent thereof;
(II) a nucleotide sequence encoding a signal peptide, and wherein the nucleotide sequences comprises SEQ ID NO: 56 or an equivalent thereof;
(III) a nucleotide sequence encoding a linker peptide, and wherein the nucleotide sequence comprises SEQ ID NO: 57 or an equivalent thereof;
(IV) a nucleotide sequence encoding an anti-BCMA light chain variable region, and wherein the nucleotide sequence is selected from: SEQ ID NO: 58, SEQ ID NO: 59, SEQ ID NO: 60, or SEQ ID NO: 61 or an equivalent thereof;
(V) a nucleotide sequence encoding an anti-BCMA heavy chain variable region, and wherein the nucleotide sequence is selected from: SEQ ID NO: 62, SEQ ID NO: 63, SEQ ID NO: 64, or SEQ ID NO: 65 or an equivalent thereof;
(VI) a nucleotide sequence encoding a hinge domain, and wherein the nucleotide sequence comprises SEQ ID NO: 66 or an equivalent thereof;
(VII) a nucleotide sequence encoding a transmembrane and cytoplasmic/intracellular domain, and wherein the nucleotide sequence comprises SEQ ID NO: 67 or an equivalent thereof;
(VIII) a nucleotide sequence encoding a signaling domain, and wherein the nucleotide sequence comprises SEQ ID NO: 68 or an equivalent thereof;
(IX) a nucleotide sequence encoding a self-cleaving peptide, and wherein the nucleotide sequence comprises SEQ ID NO: 69 or an equivalent thereof;
(X) a nucleotide sequence encoding a linker, and wherein the nucleotide sequence comprises SEQ ID NO: 70 or an equivalent thereof;
(XI) a nucleotide sequence encoding an anti-GPRCSD light chain variable region, and wherein the nucleotide sequence comprises SEQ ID NO: 71 or an equivalent thereof;
(XII) a nucleotide sequence encoding an anti-GPRC5D heavy chain variable region, and wherein the nucleotide sequence comprises SEQ ID NO: 72 or an equivalent thereof;
(XIII) a nucleotide sequence encoding an anti-NKG2D light chain variable region, and wherein the nucleotide sequence comprises SEQ ID NO: 73 or an equivalent thereof;
(XIV) a nucleotide sequence encoding an anti-NKG2D heavy chain variable region, and wherein the nucleotide sequence comprises SEQ ID NO: 74 or an equivalent thereof;
(XV) a nucleotide sequence encoding a mutant Fc region, and wherein the nucleotide sequence comprises SEQ ID NO: 75 or an equivalent thereof;
(XVI) a nucleotide sequence encoding a tag, wherein the nucleotide sequence comprises SEQ ID NO: 76 or an equivalent thereof;
(XVII) a nucleotide sequence of SEQ ID NO: 77 or an equivalent thereof;
(XVIII) a nucleotide sequence of SEQ ID NO: 78 or an equivalent thereof;
(XIX) a nucleotide sequence of SEQ ID NO: 79 or an equivalent thereof; and
(XX) a nucleotide sequence of SEQ ID NO: 80 or an equivalent thereof.

Embodiment 101. The polynucleotide of any one of Embodiments 97 to 100, further comprising a suicide gene product or a detectable marker or both.

Embodiment 102. The polynucleotide of Embodiment 101, wherein the suicide gene encodes a product selected from one or more of: HSV-TK (Herpes simplex virus thymidine kinase), cytosine deaminase, nitroreductase, carboxylesterase, cytochrome P450 or PNP (Purine nucleoside phosphorylase), truncated EGFR, or inducible caspase (“iCasp”).

Embodiment 103. The polynucleotide of any one of Embodiments 97 to 102, further comprising a detectable marker.

Embodiment 104. A vector comprising a polynucleotide of any of Embodiments 97 to 103.

Embodiment 105. The vector of Embodiment 104, wherein the vector is a non-viral vector or a viral vector.

Embodiment 106. The vector of Embodiment 105, wherein the non-viral vector is a plasmid.

Embodiment 107. The vector of Embodiment 105, wherein the viral vector is selected from the group of a retroviral vector, a lentiviral vector, an adenoviral vector, an adeno-associated viral vector, or Herpes viral vector.

Embodiment 108. The vector of any one of Embodiments 104 to 107, further comprising a regulatory sequence directing the replication of the polynucleotide.

Embodiment 109. An isolated cell comprising one or more of the following: a polypeptide of any one of Embodiments 1 to 50, a bispecific antibody or a fragment thereof of any one of Embodiments 51 to 73, a CAR of any one of Embodiments 74 to 96, a polynucleotide of any one of Embodiments 97 to 103, or a vector of any one of Embodiments 104 to 108.

Embodiment 110. The isolated cell of Embodiment 109, wherein the cell is a prokaryotic cell or a eukaryotic cell.

Embodiment 111. The isolated cell of Embodiment 109 or 110, wherein the cell is a eukaryotic cell.

Embodiment 112. The isolated cell of Embodiment 111, wherein the eukaryotic cell is selected from an animal cell, a mammalian cell, a bovine cell, a feline cell, a canine cell, a murine cell, an equine cell, or a human cell.

Embodiment 113. The isolated cell of Embodiment 111 or 112, wherein the eukaryotic cell is an immune cell, optionally a T-cell, a B cell, a NK cell, a NKT cell, a dendritic cell, a myeloid cell, a monocyte, or a macrophage, optionally derived from Hematopoietic stem cells (HSCs) and/or induced pluripotent stem cells (iPSCs).

Embodiment 114. The isolated cell of any one of Embodiments 109 to 113, wherein the isolated cell secretes the bispecific antibody.

Embodiment 115. A composition comprising a carrier and one or more of the following: a polypeptide of any one of Embodiments 1 to 50, a bispecific antibody or a fragment thereof of any one of Embodiments 51 to 73, a CAR of any one of Embodiments 74 to 96, a polynucleotide of any one of Embodiments 97 to 103, a vector of any one of Embodiments 104 to 108, or an isolated cell of any one of Embodiments 109 to 114.

Embodiment 116. The composition of Embodiment 115, wherein the carrier is a pharmaceutically acceptable carrier.

Embodiment 117. An isolated complex comprising one or more of the following:
a polypeptide of any one of Embodiments 1 to 50 bound to a cancer cell,
a bispecific antibody or a fragment thereof of any one of Embodiments 51 to 73 bound to a cancer cell,
a CAR of any one of Embodiments 74 to 96 bound to a cancer cell, and
an isolated cell of any one of Embodiments 109 to 114 bound to a cancer cell.

Embodiment 118. A method of producing a CAR expressing cell comprising transducing a cell or a population thereof with a polynucleotide of any one of Embodiments 97 to 103 and/or a vector of any one of Embodiments 104 to 108.

Embodiment 119. A method of producing a cell secreting a bispecific antibody comprising transducing a cell or a population thereof with a polynucleotide of any one of Embodiments 97 to 103 and/or a vector of any one of Embodiments 104 to 108.

Embodiment 120. A method of producing a CAR expressing cell secreting a bispecific antibody comprising transducing a cell or a population thereof with a polynucleotide of any one of Embodiments 97 to 103 and/or a vector of any one of Embodiments 104 to 108.

Embodiment 121. The method of any one of Embodiments 118 to 120, wherein the cell is selected from a Hematopoietic stem cell (HSC), an induced pluripotent stem cell (iPSCs), or an immune cell.

Embodiment 122. The method of any one of Embodiments 118 to 120, wherein the cell population comprises one or more of the following: an HSC, an iPSC, or an immune cell.

Embodiment 123. The method of Embodiment 121 or 122, wherein the immune cell is selected from T-cells, B cells, NK cells, dendritic cells, myeloid cells, monocytes, or macrophages.

Embodiment 124. The method of any one of Embodiments 121 to 123, wherein the immune cell is derived from an HSC or an iPSC.

Embodiment 125. A method of inhibiting the growth of a cancer cell expressing a tumor associated antigen (TAA) or a tissue comprising the cancer cell, comprising contacting the cancer cell or the tissue with an isolated cell of any one of Embodiments 109 to 114.

Embodiment 126. The method of Embodiment 125, further comprising contacting the cancer cell or the tissue with a bispecific antibody or a fragment thereof of any one of Embodiments 51 to 73.

Embodiment 127. The method of Embodiment 125 or 126, wherein the contacting is in vitro, or ex vivo, or in vivo.

Embodiment 128. The method of any one of Embodiments 125 to 127, wherein the contacting is in vivo and the isolated cells are autologous or allogeneic to a subject being treated.

Embodiment 129. The method of any one of Embodiments 125 to 127, wherein the contacting is in vivo and the isolated cells are allogenic to a subject being treated.

Embodiment 130. The method of any one of Embodiments 125 to 129, wherein the cancer cell expresses a TAA of BCMA.

Embodiment 131. The method of any one of Embodiments 125 to 130, wherein the cancer cell expresses a TAA of GPRCSD.

Embodiment 132. The method of any one of Embodiments 125 to 131, wherein the cancer cell expresses both BCMA and GPRCSD.

Embodiment 133. A method of inhibiting the growth of a cancer cell expressing GPRCSD or a tissue comprising the cancer cell, comprising contacting the cancer or tissue with a bispecific antibody or a fragment thereof of any one of Embodiments 51 to 73.

Embodiment 134. The method of Embodiment 133, wherein the contacting is in vitro, or ex vivo, or in vivo.

Embodiment 135. The method of any one of Embodiments 125 to 134, further comprising contacting the cancer cell or the tissue with an effective amount of a cytoreductive therapy or a therapy that upregulates the expression of the TAA on the cancer cell.

Embodiment 136. The method of Embodiment 135, wherein the contacting is in vitro, or ex vivo, or in vivo.

Embodiment 137. A method for treating a cancer in a subject in need thereof, comprising administering the isolated cell of Embodiment 113 to the subject.

Embodiment 138. The method of Embodiment 137, wherein the subject is selected for the therapy by determining expression of one or more tumor associated antigens (TAAs) in a sample isolated from the subject and administering the cell that expresses a CAR that recognizes and bind the one or more TAAs.

Embodiment 139. The method of Embodiment 138, wherein the TAA expression is determined by contacting the sample with an anti-TAA antibody or an antigen binding fragment thereof in vitro, or ex vivo, or in vivo and detecting binding between the sample and the anti-TAA antibody or antigen binding fragment thereof.

Embodiment 140. The method of Embodiment 139, wherein the anti-TAA antibody comprises a detectable marker.

Embodiment 141. The method of any one of Embodiments 138 to 140, wherein at least one of the TAAs comprises an epitope of BCMA.

Embodiment 142. The method of any one of Embodiments 138 to 141, wherein at least one of the TAAs comprises an epitope of GPRC5D.

Embodiment 143. The method of any one of Embodiments 137 to 141, further comprising administering a bispecific antibody or a fragment thereof of any one of Embodiments 51 to 73 to the subject.

Embodiment 144. The method of Embodiment 143, wherein the isolated cell and the bispecific antibody or a fragment thereof are administered together.

Embodiment 145. The method of Embodiment 143, wherein the isolated cell and the bispecific antibody or a fragment thereof are administered separately.

Embodiment 146. The method of Embodiment 145, wherein the isolated cell and the bispecific antibody or a fragment thereof are administered concurrently.

Embodiment 147. The method of Embodiment 145, wherein the isolated cell and the bispecific antibody or a fragment thereof are administered 1 hours apart, 4 hours apart, 24 hours apart, 2 days apart, 3 days apart, or 1 week apart.

Embodiment 148. A method for treating a cancer in a subject selected for the treatment, comprising administering the isolated cell of any one of Embodiments 109 to 114 to the subject.

Embodiment 149. The method of Embodiment 148, wherein the subject is selected via if a cancer cell of the subject expresses one or more of tumor associated antigen(s) (TAAs).

Embodiment 150. The method of Embodiment 149, wherein the TAA expression is determined by contacting a cancer cell of the subject with an anti-TAA antibody or an antigen binding fragment thereof in vitro, or ex vivo, or in vivo.

Embodiment 151. The method of Embodiment 150, wherein the anti-TAA antibody comprises a detectable marker.

Embodiment 152. The method of any one of Embodiments 149 to 151, wherein the TAA is BCMA.

Embodiment 153. The method of any one of Embodiments 149 to 152, wherein the TAA is GPRCSD.

Embodiment 154. The method of any one of Embodiments 148 to 153, further comprising administering a bispecific antibody or a fragment thereof of any one of Embodiments 51 to 73 to the subject.

Embodiment 155. The method of Embodiment 154, wherein the isolated cell and the bispecific antibody or a fragment thereof are administered together.

Embodiment 156. The method of Embodiment 154, wherein the isolated cell and the bispecific antibody or a fragment thereof are administered separately.

Embodiment 157. The method of Embodiment 156, wherein the isolated cell and the bispecific antibody or a fragment thereof are administered concurrently.

Embodiment 158. The method of Embodiment 156, wherein the isolated cell and the bispecific antibody or a fragment thereof are administered 1 hours apart, 4 hours apart, 24 hours apart, 2 days apart, 3 days apart, or 1 week apart.

Embodiment 159. The method of any one of Embodiments 137 to 158, wherein the isolated cell is autologous or allogeneic to the subject in need.

Embodiment 160. The method of any one of Embodiments 137 to 158, wherein the isolated cell is allogenic to the subject in need.

Embodiment 161. A method for treating a GPRC5D-expressing cancer in a subject, comprising administering the bispecific antibody or a fragment thereof of any one of Embodiments 51 to 73 to the subject.

Embodiment 162. The method of any one of Embodiments 137 to 161, further comprising administering to the subject a cytoreductive therapy or a therapy that upregulates the expression of the tumor associated antigen (TAA).

Embodiment 163. The method of any one of Embodiments 137 to 162, wherein the administration is applied to the subject as a first line therapy, or a second line therapy, or third line therapy, or a fourth line therapy.

Embodiment 164. The method of Embodiment 135 or 162, wherein the cytoreductive therapy comprises one or more of the following: a chemotherapy, a cryotherapy, a hyperthermia, a targeted therapy, or a radiation therapy.

Embodiment 165. The method of any one of Embodiments 128 to 164, wherein the subject is a mammal, a canine, a feline, an equine, a murine, or a human patient.

Embodiment 166. The method of any one of Embodiments 125 to 165, wherein the cancer is multiple myeloma (MM).

Embodiment 167. A kit comprising optional instructions for use and one or more of the following: a polypeptide of any one of Embodiments 1 to 50, a bispecific antibody or a fragment thereof of any one of Embodiments 51 to 73, a CAR of any one of Embodiments 74 to 96, a polynucleotide of any one of Embodiments 97 to 103, a vector of any one of Embodiments 104 to 108, or an isolated cell of any one of Embodiments 109 to 114, a composition of Embodiment 115 or 116, or an isolated complex of Embodiment 117.

### Equivalents

Unless otherwise defined, all technical and scientific terms used herein have the same meaning as commonly understood by one of ordinary skill in the art to which this technology belongs.

The present technology illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the present technology claimed.

Thus, it should be understood that the materials, methods, and examples provided here are representative of preferred aspects, are exemplary, and are not intended as limitations on the scope of the present technology.

The present technology has been described broadly and generically herein. Each of the narrower species and sub-generic groupings falling within the generic disclosure also form part of the present technology. This includes the generic description of the present technology with a proviso or negative limitation removing any subject matter from the genus, regardless of whether or not the excised material is specifically recited herein.

In addition, where features or aspects of the present technology are described in terms of Markush groups, those skilled in the art will recognize that the present technology is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

Other aspects are set forth within the following claims.

What is claimed is:

1. A polypeptide comprising:
(i) an amino acid sequence of a Chimeric Antigen Receptor (CAR) comprising:
(1) an antigen binding amino acid sequence that recognizes and binds B-cell maturation antigen (BCMA) (anti-BCMA antigen binding sequence),
(2) a hinge domain,
(3) a transmembrane domain, and
(4) an intracellular domain;
(ii) an amino acid sequence of a bispecific antibody comprising
(1) an antigen binding sequence that recognizes and binds NKG2D (anti-NKG2D antigen binding sequence), and
(2) an antigen binding sequence that recognizes and binds G Protein-Coupled Receptor Class C Group 5 Member D (GPRC5D) (anti-GPRC5D antigen binding sequence);
(iii) an optional amino acid sequence of a cytokine selected from IL15 or IL-21; and
(iv) an optional amino acid sequence of a detectable marker and a suicide gene product, optionally a truncated epidermal growth factor receptor (tEGFR) or RQR8.

2. The polypeptide of claim 1, wherein the bispecific antibody comprises, from the N terminus to the C terminus, the anti-GPRC5D antigen binding sequence and the anti-NKG2D antigen binding sequence.

3. The polypeptide of claim 1, wherein the bispecific antibody comprises, from the N terminus to the C terminus, the anti-NKG2D antigen binding sequence and the anti-GPRC5D antigen binding sequence.

4. The polypeptide of any one of claims 1-3, wherein the anti-NKG2D antigen binding sequence comprises one or two or three or four or five or all six of the following complementarity-determining regions (CDRs):
a light chain complementarity-
determining region 1 (CDRL1) comprising
(SEQ ID NO: 1)
SGSSSNIGNNAVN
or an equivalent thereof;
a light chain complementarity-
determining region 2 (CDRL2) comprising
(SEQ ID NO: 2)
YDDLLPS
or an equivalent thereof;
a light chain complementarity-
determining region 3 (CDRL3) comprising
(SEQ ID NO: 3)
AAWDDSLNGPV
or an equivalent thereof;
a heavy chain complementarity-
determining region 1 (CDRH1) comprising
(SEQ ID NO: 4)
GFTFSSY
or an equivalent thereof;
a heavy chain complementarity-
determining region 2 (CDRH2) comprising
(SEQ ID NO: 5)
RYDGSN
or an equivalent thereof, and
a heavy chain complementarity-
determining region 3 (CDRH3) comprising
(SEQ ID NO: 6)
DRGLGDGTYFDY
or an equivalent thereof,
wherein the equivalents thereof recognizes and binds NKG2D.

5. The polypeptide of any one of claims 1-4, wherein the anti-NKG2D antigen binding sequence comprises one or both of:
a light chain variable region comprising SEQ ID NO: 7, or an equivalent thereof; or
a heavy chain variable region comprising SEQ ID NO: 8, or an equivalent thereof,
wherein the equivalent thereof recognizes and binds NKG2D.

6. The polypeptide of claim 5, wherein the equivalent of SEQ ID NO: 7 or 8 is at least 80%, or at least 85%, or at least 90%, or at least 95%, or at least 96%, or at least 97%, or at least 98%, or at least 99% identical to SEQ ID NO: 7 or 8, respectively.

7. The polypeptide of claim 5 or 6, wherein the equivalent of SEQ ID NO: 7 retains the CDRs of SEQ ID NO: 7 and the equivalent of SEQ ID NO: 8 retains the CDRs of SEQ ID NO: 8.

8. The polypeptide of any one of claims 5 to 7, wherein the anti-NKG2D antigen binding sequence further comprises a peptide linker between the light chain variable region or the equivalent thereof and the heavy chain variable region or the equivalent thereof.

9. The polypeptide of any one of claims 5 to 8, wherein the anti-NKG2D antigen binding sequence further comprises a peptide linker comprising SEQ ID NO: 9 or an equivalent thereof between the light chain variable region or the equivalent thereof and the heavy chain variable region or the equivalent thereof.

10. The polypeptide of any one of claims 1 to 9, wherein the anti-GPRC5D antigen binding sequence comprises one or two or three or four or five or all six of the following CDRs:
a CDRL1 comprising
(SEQ ID NO: 10)
KASQNVATHVG
or an equivalent thereof;
a CDRL2 comprising
(SEQ ID NO: 11)
SASYRYS
or an equivalent thereof;
a CDRL3 comprising
(SEQ ID NO: 12)
QQYNRYPYT
or an equivalent thereof;
a CDRH1 comprising
(SEQ ID NO: 13)
GYSFTGY
or an equivalent thereof;
a CDRH2 comprising
(SEQ ID NO: 14)
NPYNSD
or an equivalent thereof;
and
a CDRH3 comprising
(SEQ ID NO: 15)
VALRVALDY
or an equivalent thereof,
wherein the equivalents thereof recognizes and binds GPRC5D.

11. The polypeptide of any one of claims 1 to 10, wherein the anti-GPRC5D antigen binding sequence comprises one or both of:
a light chain variable region comprising SEQ ID NO: 16, or an equivalent thereof; or
a heavy chain variable region comprising SEQ ID NO: 17, or an equivalent thereof,
wherein the equivalents thereof recognizes and binds GPRC5D.

12. The polypeptide of claim 11, wherein the equivalent of SEQ ID NO: 16 or 17 is at least 80%, or at least 85%, or at least 90%, or at least 95%, or at least 96%, or at least 97%, or at least 98%, or at least 99% identical to SEQ ID NO: 16 or 17, respectively.

13. The polypeptide of claim 11 or 12, wherein the equivalent of SEQ ID NO: 16 retains the CDRs of SEQ ID NO: 16 and the equivalent of SEQ ID NO: 17 retains the CDRs of SEQ ID NO: 17.

14. The polypeptide of any one of claims 11 to 13, wherein the anti-GPRC5D antigen binding sequence further comprises a peptide linker between the light chain variable region or the equivalent thereof and the heavy chain variable region or the equivalent thereof.

15. The polypeptide of any one of claims 11 to 14, wherein the anti-GPRC5D antigen binding sequence further comprises a peptide linker comprising SEQ ID NO: 9 or an equivalent thereof between the light chain variable region or the equivalent thereof and the heavy chain variable region or the equivalent thereof.

16. The polypeptide of any one of claims 1 to 15, wherein the bispecific antibody further comprises a fragment crystallizable (Fc) region of an immunoglobulin, a mutant thereof, or an equivalent thereof.

17. The polypeptide of claim 16, wherein the Fc region or a mutant thereof is a human Fc region or a mutant thereof.

18. The polypeptide of claim 16 or 17, wherein the Fc region comprises SEQ ID NO: 18, or an equivalent thereof that stabilizes the bispecific antibody.

19. The polypeptide of any one of claims 16 to 18, wherein the Fc region comprises SEQ ID NO: 19 or an Fc equivalent having mutations at a position corresponding to amino acid (aa) 16, aa 17 and aa 79 of SEQ ID NO: 19, and stabilizing the bispecific antibody.

20. The polypeptide of any one of claims 1 to 19, wherein the bispecific antibody further comprises a peptide linker between the anti-NKG2D antigen binding sequence and the anti-GPRCSD antigen binding sequence.

21. The polypeptide of claim 20, wherein the peptide linker comprises
(SEQ ID NO: 81)
PSGQAGAAASESLFVSNHAY
or an equivalent thereof.

22. The polypeptide of any one of claims 1 to 21, wherein the bispecific antibody further comprises a signal peptide at its N terminus.

23. The polypeptide of claim 22, wherein the signal peptide is selected from a polypeptide comprises MYRMQLLSCIALSLALVTNS (SEQ ID NO: 20), MWLQSLLLLGTVACSIS (SEQ ID NO: 122), MRISKPHLRSISIQCYLCLLLNSHFLTEA (SEQ ID NO: 123), MRSSPGNMERIVICLMVIFLGTLV (SEQ ID NO: 124), MTRLTVLALLAGLLASSRA (SEQ ID NO: 126) or an equivalent of each thereof.

24. The polypeptide of claim 22 or 23, wherein the signal peptide consists of SEQ ID NO: 20.

25. The polypeptide of any one of claims 1 to 24, wherein the bispecific antibody further comprises an additional detectable or purification marker.

26. The polypeptide of claim 25, wherein the detectable marker comprises
(SEQ ID NO: 22)
YPYDVPDYA.

27. The polypeptide of any one of claims 1 to 26, wherein the anti-BCMA antigen binding sequence comprises one or two or three or four or five or six of the following CDRs:
a CDRL1 comprising a sequence selected from
(SEQ ID NO: 23)
RASESVTILGSHLIH,
(SEQ ID NO: 24)
SASQDISNYLN,
(SEQ ID NO: 25)
RASESVTILGSHLIY,
or an equivalent of each thereof;
a CDRL2 comprising a sequence selected from
(SEQ ID NO: 26)
LASNVQT,
(SEQ ID NO: 27)
YTSNLHS,
(SEQ ID NO: 28)
LASNVQT,
or an equivalent of each thereof;
a CDRL3 comprising a sequence selected from
(SEQ ID NO: 29)
LQSRTIPRT,
(SEQ ID NO: 30)
QQYRKLPWT,
(SEQ ID NO: 31)
LQSRTIPRT,
an equivalent of each thereof;
a CDRH1 comprising a sequence selected from
(SEQ ID NO: 32)
GYTFTDY,
(SEQ ID NO: 33)
GGTFSNY,
(SEQ ID NO: 34)
GYTFRHY,
or an equivalent of each thereof;
a CDRH2 comprising a sequence selected from
(SEQ ID NO: 35)
INTETRE,
(SEQ ID NO: 36)
YRGHSD,
(SEQ ID NO: 37)
NTESGV,
or an equivalent of each thereof;
and
a CDRH3 comprising a sequence selected from
(SEQ ID NO: 38)
DYSYAMDY,
(SEQ ID NO: 39)
GAIYNGYDVLDN,
(SEQ ID NO: 40)
DYLYSLDF,
or an equivalent of each thereof,
wherein the equivalents thereof recognizes and binds BCMA.

28. The polypeptide of claim 27, wherein the anti-BCMA antigen binding sequence comprises
a light chain variable region comprising a sequence selected from SEQ ID NO: 41, SEQ ID NO: 42, SEQ ID NO: 43, or an equivalent of each thereof and/or
a heavy chain variable region comprising a sequence selected from SEQ ID NO: 44, SEQ ID NO: 45, SEQ ID NO: 46, or an equivalent of each thereof,
wherein the equivalents thereof recognizes and binds BCMA.

29. The polypeptide of claim 27 or 28, wherein the equivalent of SEQ ID NOs: 41 to 46 is at least 80%, or at least 85%, or at least 90%, or at least 95%, or at least 96%, or at least 97%, or at least 98%, or at least 99% identical to SEQ ID NOs: 41 to 46, respectively.

30. The polypeptide of claim 28 or 29, wherein the equivalent of SEQ ID NOs: 41-46 retains the CDRs of SEQ ID NOs: 41-46, respectively.

31. The polypeptide of any one of claims 28 to 30, wherein the anti-BCMA antigen binding sequence or an equivalent thereof further comprises a peptide linker between the light chain variable region or the equivalent thereof and the heavy chain variable region or the equivalent thereof.

32. The polypeptide of any one of claims 28 to 31, wherein the anti-BCMA antigen binding sequence further comprises a peptide linker comprising SEQ ID NO: 9 or an equivalent thereof between the light chain variable region or the equivalent thereof and the heavy chain variable region or the equivalent thereof.

33. The polypeptide of any one of claims 1 to 32, wherein the hinge domain comprises a CD8α hinge domain or an IgG1 hinge domain.

34. The polypeptide of claim 33, wherein the IgG1 hinge domain comprises SEQ ID NO: 47 or an equivalent thereof.

35. The polypeptide of any one of claims 1 to 34, wherein the transmembrane domain comprises a CD8 a transmembrane domain or a CD28 transmembrane domain.

36. The polypeptide of any one of claims 1 to 35, wherein the intracellular domain comprises one or more or two or more costimulatory regions selected from a CD28 costimulatory signaling region, a 4-1BB costimulatory signaling region, an ICOS costimulatory signaling region, an OX40 costimulatory region, a DAP 10 costimulatory region, or a DAP12 costimulatory region.

37. The polypeptide of claim 35 or 36, wherein the CAR comprises a CD28 transmembrane domain and costimulatory signaling region comprising SEQ ID NO: 48 or an equivalent or each thereof.

38. The polypeptide of any one of claims 1 to 37, wherein the intracellular domain further comprises a CD3 zeta signaling domain.

39. The polypeptide of claim 38, wherein the CD3 zeta signaling domain comprises SEQ ID NO: 49 or an equivalent thereof.

40. The polypeptide of any one of claims 1 to 39, wherein the CAR further comprises a signal peptide at its N terminus, optionally different from the signal peptide at the N terminus of the bispecific antibody.

41. The polypeptide of claim 40, wherein the signal peptide comprises MGWSSIILFLVATATGVH (SEQ ID NO: 21), MLLLVTSLLLCELPHPAFLLIP (SEQ ID NO: 125), MTRLTVLALLAGLLASSRA (SEQ ID NO: 126) or an equivalent of each thereof that facilitates secretion of the bispecific antibody, fragment or equivalent thereof.

42. The polypeptide of claim 40 or 41, wherein the signal peptide consists of SEQ ID NO: 21.

43. The polypeptide of any one of claims 1 to 42, wherein the amino acid sequence of the cytokine comprise at least one of SEQ ID NO: 106, SEQ ID NO: 104, SEQ ID NO: 117, SEQ ID NO: 118, or SEQ ID NO: 116 or an equivalent of each thereof.

44. The polypeptide of any one of claims 1 to 43, wherein the amino acid sequence of the cytokine comprise SEQ ID NO: 106 or an equivalent thereof.

45. The polypeptide of any one of claims 1 to 44, wherein the amino acid sequence of the cytokine comprise SEQ ID NO: 106 or an equivalent thereof and SEQ ID NO: 107 or an equivalent thereof.

46. The polypeptide of claim 45, wherein the two sequences are linked by a linker.

47. The polypeptide of claim 46, wherein the linker comprises SEQ ID NO: 108 or SEQ ID NO: 9, or an equivalent of each thereof.

48. The polypeptide of any one of claims 1 to 47, wherein the cytokine is a membrane bound cytokine.

49. The polypeptide of any one of claims 1 to 48, wherein the amino acid sequence of the cytokine further comprises a transmembrane domain.

50. The polypeptide of claim 49, wherein the transmembrane domain comprises a platelet-derived growth factor receptor beta (PDGFRβ) transmembrane domain comprising SEQ ID NO: 119 or an equivalent thereof.

51. The polypeptide of any one of claims 1 to 50, wherein the cytokine further comprises a signal peptide.

52. The polypeptide of claim 46, wherein the signal peptide comprises
(SEQ ID NO: 20)
MYRMQLLSCIALSLALVTNS,
(SEQ ID NO: 122)
MWLQSLLLLGTVACSIS,
(SEQ ID NO: 123)
MRISKPHLRSISIQCYLCLLLNSHFLTEA,
(SEQ ID NO: 124)
MRSSPGNMERIVICLMVIFLGTLV,
(SEQ ID NO: 126)
MTRLTVLALLAGLLASSRA
or an equivalent of each thereof.

53. The polypeptide of claim 51 or 52, wherein the signal peptide consists of
(SEQ ID NO: 126)
MTRLTVLALLAGLLASSRA
or an equivalent of each thereof.

54. The polypeptide of any one of claims 1 to 53, the tEGFR comprises a sequence of SEQ ID NO: 120 or an equivalent thereof.

55. The polypeptide of any one of claims 1 to 54, wherein the tEGFR further comprises a signal peptide.

56. The polypeptide of claim 55, wherein the signal peptide comprises MGWSSIILFLVATATGVH (SEQ ID NO: 21), MLLLVTSLLLCELPHPAFLLIP (SEQ ID NO: 125), MTRLTVLALLAGLLASSRA (SEQ ID NO: 126) or an equivalent of each thereof that facilitates secretion of the bispecific antibody, fragment or equivalent thereof.

57. The polypeptide of any one of claims 1 to 53, wherein the RQR8 comprises SEQ ID NO: 132 or an equivalent thereof.

58. The polypeptide of any one of claims 1 to 57, wherein the amino acid sequence of any one or more of (i) to (iv) further comprises a detectable marker.

59. The polypeptide of any one of claims 1 to 58, further comprising a cleavable peptide located between any two of (i), (ii), (iii) and (iv).

60. The polypeptide of claim 59, wherein the cleavable peptide is a self-cleaving peptide.

61. The polypeptide of claim 60, wherein the self-cleaving peptide is selected from a T2A peptide or a P2A.

62. The polypeptide of any one of claims 59 to 61, wherein cleavable peptide or self-cleaving peptide comprises a peptide selected from HVGSGEGRGSLLTCGDVEENPGP (SEQ ID NO: 50), GSGEGRGSLLTCGDVEENPGP (aa 3 to aa 23 of SEQ ID NO: 50), GSGATNFSLLKQAGDVEENPGP (SEQ ID NO: 133), EGRGSLLTCGDVEENPGP (SEQ ID NO: 134), AEGRGSLLTCGDVEENPGP (SEQ ID NO: 135) or an equivalent of each thereof.

63. The polypeptide of any one of claims 1 to 62, comprising
a signal peptide comprising an
amino acid sequence of
(SEQ ID NO: 21)
MGWSSIILFLVATATGVH
or
(SEQ ID NO: 126)
MTRLTVLALLAGLLASSRA
or an equivalent of each thereof;
an anti-BCMA antigen binding sequence comprising a light chain variable region that comprises SEQ ID NO: 41 or SEQ ID NO: 42 or an equivalent of each thereof, a peptide linker that comprises SEQ ID NO: 9 or an equivalent thereof, and a heavy chain variable region that comprises SEQ ID NO: 44 or SEQ ID NO: 45 or an equivalent of each thereof, and wherein the equivalents of the variable regions recognize and bind BCMA;
a hinge domain comprising
(SEQ ID NO: 47)
LEPKSCDKTHTCPPCPDPKGT
or
an equivalent thereof;
an CD28 transmembrane domain comprising
SEQ ID NO: 91 or an equivalent thereof;
an CD28 costimulatory signaling region
comprising SEQ ID NO: 95 or an
equivalent thereof;
an CD3 zeta signaling domain comprising
SEQ ID NO: 49 or an equivalent
thereof;
a T2A self-cleaving peptide comprising
SEQ ID NO: 50 or aa 3 to aa 23 of
SEQ ID NO: 50 or an equivalent thereof;
a signal peptide comprising
(SEQ ID NO: 20)
MYRMQLLSCIALSLALVTNS,
(SEQ ID NO: 126)
MTRLTVLALLAGLLASSRA
or an equivalent of each thereof;
an anti-GPRC5D antigen binding sequence comprising a light chain variable region that comprises SEQ ID NO: 16, or an equivalent thereof, a peptide linker that comprises SEQ ID NO: 9 or an equivalent thereof, and a heavy chain variable region that comprises SEQ ID NO: 17, or an equivalent thereof, wherein the equivalents of the variable regions recognize and bind GPRCSD;
a human muscle aldolase (HMA) linker comprising SEQ ID NO: 81 or an equivalent thereof;
an anti-NKG2D antigen binding sequence comprising a light chain variable region that comprises SEQ ID NO: 7 or an equivalent thereof, a peptide linker that comprises SEQ ID NO: 9 or an equivalent thereof, and a heavy chain variable region that comprises SEQ ID NO: 8 or an equivalent thereof, wherein the equivalents of the variable regions recognize and bind NKG2D;
an Fc region comprising SEQ ID NO: 19
or an equivalent thereof;
a T2A self-cleaving peptide comprising
(SEQ ID NO: 134)
EGRGSLLTCGDVEENPGP
or
(SEQ ID NO: 135)
AEGRGSLLTCGDVEENPGP
or an equivalent thereof;
a signal peptide comprising
(SEQ ID NO: 20)
MYRMQLLSCIALSLALVTNS
or
(SEQ ID NO: 123)
MRISKPHLRSISIQCYLCLLLNSHFLTEA
or
(SEQ ID NO: 122)
MWLQSLLLLGTVACSIS
or
(SEQ ID NO: 126)
MTRLTVLALLAGLLASSRA
or an equivalent of each thereof;
an amino acid sequence of the cytokine comprising at least one of SEQ ID NO: 106, SEQ ID NO: 104, SEQ ID NO: 117, SEQ ID NO: 118, SEQ ID NO: 116, or an equivalent of each thereof, optionally wherein the amino acid sequence of the cytokine further comprises a transmembrane domain comprising SEQ ID NO: 119 or an equivalent thereof, and optionally wherein the amino acid sequence of the cytokine comprise SEQ ID NO: 106 or an equivalent thereof, an optional peptide linker comprising SEQ ID NO: 108 or SEQ ID NO: 9, or an equivalent of each thereof, and SEQ ID NO: 107 or an equivalent thereof;
a P2A self-cleaving peptide comprising SEQ ID NO: 133 or an equivalent thereof;
an optional signal peptide comprising MLLLVTSLLLCELPHPAFLLIP (SEQ ID NO: 125), MTRLTVLALLAGLLASSRA (SEQ ID NO: 126) or an equivalent of each thereof; and
a detectable marker and a suicide gene product comprising SEQ ID NO: 120 or SEQ ID NO: 132 or an equivalent of each thereof.

64. The polypeptide of any one of claims 1 to 63, comprising
a signal peptide comprising an amino acid sequence of
(SEQ ID NO: 21)
MGWSSIILFLVATATGVH
or
(SEQ ID NO: 126)
MTRLTVLALLAGLLASSRA;
an anti-BCMA antigen binding sequence comprising a light chain variable region that comprises SEQ ID NO: 41 or SEQ ID NO: 42, a peptide linker that comprises SEQ ID NO: 9, and a heavy chain variable region that comprises SEQ ID NO: 44 or SEQ ID NO: 45;
a hinge domain comprising SEQ ID NO: 47;
an CD28 transmembrane domain comprising SEQ ID NO: 91;
an CD28 costimulatory signaling region comprising SEQ ID NO: 95;
an CD3 zeta signaling domain comprising SEQ ID NO: 49;
a T2A self-cleaving peptide comprising SEQ ID NO: 50;
a signal peptide comprising SEQ ID NO: 20 or SEQ ID NO: 126;
an anti-GPRCSD antigen binding sequence comprising a light chain variable region that comprises SEQ ID NO: 16, a peptide linker that comprises SEQ ID NO: 9, and a heavy chain variable region that comprises SEQ ID NO: 17;
a human muscle aldolase (HMA) linker comprising SEQ ID NO: 81;
an anti-NKG2D antigen binding sequence comprising a light chain variable region that comprises SEQ ID NO: 7, a peptide linker that comprises SEQ ID NO: 9, and a heavy chain variable region that comprises SEQ ID NO: 8;
an Fc region comprising SEQ ID NO: 19;
a T2A self-cleaving peptide comprising SEQ ID NO: 134;
a signal peptide comprising MYRMQLLSCIALSLALVTNS (SEQ ID NO:20) or MRISKPHLRSISIQCYLCLLLNSHFLTEA (SEQ ID NO: 123) or MWLQSLLLLGTVACSIS (SEQ ID NO: 122) or MTRLTVLALLAGLLASSRA (SEQ ID NO: 126);
an amino acid sequence of the cytokine comprising at least one of SEQ ID NO: 106, SEQ ID NO: 104, SEQ ID NO: 117, SEQ ID NO: 118, or SEQ ID NO: 116, optionally wherein the amino acid sequence of the cytokine further comprises a transmembrane domain comprising SEQ ID NO: 119, and optionally wherein the amino acid sequence of the cytokine comprise SEQ ID NO: 106, an optional peptide linker comprising SEQ ID NO: 108, and SEQ ID NO: 107;
a P2A self-cleaving peptide comprising SEQ ID NO: 133;
a signal peptide comprising MLLLVTSLLLCELPHPAFLLIP (SEQ ID NO: 125) or MTRLTVLALLAGLLASSRA (SEQ ID NO: 126); and
a suicide gene product comprising SEQ ID NO: 120.

65. The polypeptide of claim 63 or 64, components of which are listed in an order from the N terminus to the C terminus in the polypeptide.

66. The polypeptide of claim 63 or 64, components of which are listed in an order from the C terminus to the N terminus in the polypeptide.

67. The polypeptide of claim 63 or 64, components of which are listed in an order from the N terminus to the C terminus or from the C terminus to the N terminus in the polypeptide except that one or more of the light chain variable region is switched with the heavy chain variable region of the same antigen binding sequence.

68. The polypeptide of claim 63 or 64, components of which are listed in an order from the N terminus to the C terminus or from the C terminus to the N terminus in the polypeptide except that at least two of the antigen binding sequences are switched.

69. The polypeptide of claim 63 or 64, components of which are listed in an order from the N terminus to the C terminus or from the C terminus to the N terminus in the polypeptide except that at least two of the antigen binding sequences are switched and one or more of the light chain variable region is switched with the heavy chain variable region of the same antigen binding sequence.

70. The polypeptide of any one of claims 1 to 69, comprising one or more of the amino acid sequence as disclosed in any one of Tables 1a, 1b, 1c, 1f, 1g, 2a, 2d, 2e, 2h, 2i, 21, 2m, 2p, 2q, 2t, 2u, 3a, 3b, 3c, or 3d optionally free a detectable marker.

71. The polypeptide of any one of claims 1 to 70, comprising one or more of the following sequences or a fragment thereof: amino acid sequences of P4 to P9, P11E, P12E, P19, P20, P61, P62, or P72, as disclosed herein, optionally free a detectable marker.

72. The polypeptide of claim 1, comprising any one of: SEQ ID NO: 138 or SEQ ID NO: 139.

73. A polynucleotide encoding a polypeptide of any one of claims 1 to 72, or a polynucleotide encoding
(i) an amino acid sequence of a Chimeric Antigen Receptor (CAR) comprising:
(1) an antigen binding amino acid sequence that recognizes and binds B-cell maturation antigen (BCMA) (anti-BCMA antigen binding sequence),
(2) a hinge domain,
(3) a transmembrane domain, and
(4) an intracellular domain;
(ii) an amino acid sequence of a bispecific antibody comprising
(1) an antigen binding sequence that recognizes and binds NKG2D (anti-NKG2D antigen binding sequence), and
(2) an antigen binding sequence that recognizes and binds G Protein-Coupled Receptor Class C Group 5 Member D (GPRC5D) (anti-GPRC5D antigen binding sequence);
(iii) an optional amino acid sequence of a cytokine selected from IL15 or IL-21; and
(iv) an optional amino acid sequence of a suicide gene product, optionally a truncated epidermal growth factor receptor (tEGFR) or RQR8.

74. The polynucleotide of claim 73, comprising
a signal peptide coding sequence comprising SEQ ID NO: 55 or an equivalent thereof;
an anti-BCMA antigen binding sequence coding sequence comprising a light chain variable region coding sequence that comprises SEQ ID NO: 58 or SEQ ID NO: 59 or an equivalent of each thereof, a peptide linker coding sequence that comprises SEQ ID NO: 57 or an equivalent thereof, and a heavy chain variable region coding sequence that comprises SEQ ID NO: 62 or SEQ ID NO: 63 or an equivalent of each thereof, and wherein the equivalents encode variable regions recognizing and binding BCMA;
a hinge domain coding sequence comprising SEQ ID NO: 66 or an equivalent thereof;
an CD28 transmembrane domain coding sequence comprising SEQ ID NO: 90 or an equivalent thereof;
an CD28 costimulatory signaling region coding sequence comprising SEQ ID NO: 96 or an equivalent thereof;
an CD3 zeta signaling domain coding sequence comprising SEQ ID NO: 68 or an equivalent thereof;
a T2A self-cleaving peptide coding sequence comprising SEQ ID NO: 69 or SEQ ID NO: 140 or an equivalent thereof;
a signal peptide coding sequence comprising SEQ ID NO: 56 or SEQ ID NO: 127 or an equivalent thereof;
an anti-GPRC5D antigen binding sequence coding sequence comprising a light chain variable region coding sequence that comprises SEQ ID NO: 71 or SEQ ID NO: 141 or an equivalent of each thereof, a peptide linker coding sequence that comprises SEQ ID NO: 57 or SEQ ID NO: 142 or an equivalent of each thereof, and a heavy chain variable region coding sequence that comprises SEQ ID NO: 72, or SEQ ID NO: 143, or an equivalent of each thereof, wherein the equivalents encode variable regions recognizing and binding GPRCSD;
a human muscle aldolase (HMA) linker coding sequence comprising SEQ ID NO: 70 or SEQ ID NO: 144 or an equivalent of each thereof;
an anti-NKG2D antigen binding sequence coding sequence comprising a light chain variable region coding sequence that comprises SEQ ID NO: 73 or SEQ ID NO: 145 or an equivalent of each thereof, a peptide linker coding sequence that comprises SEQ ID NO: 57 or SEQ ID NO: 146 or an equivalent of each thereof, and a heavy chain variable region coding sequence that comprises SEQ ID NO: 74 or an equivalent thereof, wherein the equivalents encode variable regions recognizing and binding NKG2D;
an Fc region coding sequence comprising SEQ ID NO: 75 or SEQ ID NO: 147 or an equivalent thereof;
a T2A self-cleaving peptide coding sequence comprising SEQ ID NO: 148 or SEQ ID NO: 149 or an equivalent of each thereof;
a signal peptide coding sequence comprising SEQ ID NO: 56 or SEQ ID NO: 150 or SEQ ID NO: 200, SEQ ID NO: 127, or SEQ ID NO: 151 or an equivalent of each thereof;
a cytokine coding sequence comprising at least one of SEQ ID NO: 152, SEQ ID NO: 153, SEQ ID NO: 154, SEQ ID NO: 155, SEQ ID NO: 156, SEQ ID NO: 157, SEQ ID NO: 158 or an equivalent of each thereof, optionally wherein the cytokine coding sequence further comprises a transmembrane domain coding sequence comprising SEQ ID NO: 164 or an equivalent thereof, and optionally wherein the cytokine coding sequence comprise SEQ ID NO: 152, or SEQ ID NO: 153 or an equivalent of each thereof, an optional peptide linker coding sequence comprising SEQ ID NO: 165 or SEQ ID NO: 166 or SEQ ID NO: 57 or an equivalent of each thereof, and SEQ ID NO: 167 or an equivalent thereof;
a P2A self-cleaving peptide coding sequence comprising SEQ ID NO: 159 or SEQ ID NO: 160 or an equivalent thereof;
an optional signal peptide coding sequence comprising SEQ ID NO: 161 or SEQ ID NO: 127 or an equivalent thereof; and
a detectable marker and suicide gene product coding sequence or a suicide gene comprising SEQ ID NO: 162 or SEQ ID NO: 163 or an equivalent of each thereof.

75. The polynucleotide of claim 73 or 74, comprising
a signal peptide coding sequence comprising SEQ ID NO: 55 or SEQ ID NO: 127;
an anti-BCMA antigen binding sequence coding sequence comprising a light chain variable region coding sequence that comprises SEQ ID NO: 58 or SEQ ID NO: 59, a peptide linker coding sequence that comprises SEQ ID NO: 57), and a heavy chain variable region coding sequence that comprises SEQ ID NO: 62 or SEQ ID NO: 63;
a hinge domain coding sequence comprising SEQ ID NO: 66;
an CD28 transmembrane domain coding sequence comprising SEQ ID NO: 90;
an CD28 costimulatory signaling region coding sequence comprising SEQ ID NO: 96;
an CD3 zeta signaling domain coding sequence comprising SEQ ID NO: 68;
a T2A self-cleaving peptide coding sequence comprising SEQ ID NO: 69;
a signal peptide coding sequence comprising SEQ ID NO: 56 or SEQ ID NO: 127;
an anti-GPRC5D antigen binding sequence coding sequence comprising a light chain variable region coding sequence that comprises SEQ ID NO: 71, a peptide linker coding sequence that comprises SEQ ID NO: 57, and a heavy chain variable region coding sequence that comprises SEQ ID NO: 72;
a human muscle aldolase (HMA) linker coding sequence comprising SEQ ID NO: 70;
an anti-NKG2D antigen binding sequence coding sequence comprising a light chain variable region coding sequence that comprises SEQ ID NO: 73, a peptide linker coding sequence that comprises SEQ ID NO: 57, and a heavy chain variable region coding sequence that comprises SEQ ID NO: 74;
an Fc region coding sequence comprising SEQ ID NO: 75;
a T2A self-cleaving peptide coding sequence comprising SEQ ID NO: 148;
a signal peptide coding sequence comprising SEQ ID NO: 56 or SEQ ID NO: 150 or SEQ ID NO: 200 or SEQ ID NO: 127;
a cytokine coding sequence comprising at least one of SEQ ID NO: 152, SEQ ID NO: 153, SEQ ID NO: 154, SEQ ID NO: 155, SEQ ID NO: 156, SEQ ID NO: 157, optionally wherein the cytokine coding sequence further comprises a transmembrane domain coding sequence comprising SEQ ID NO: 164, and optionally wherein the cytokine coding sequence comprise SEQ ID NO: 152, or SEQ ID NO: 153, an optional peptide linker coding sequence comprising SEQ ID NO: 165 or SEQ ID NO: 166 or SEQ ID NO: 57, and SEQ ID NO: 167;
a P2A self-cleaving peptide coding sequence comprising SEQ ID NO: 159;
a signal peptide coding sequence comprising SEQ ID NO: 161 or SEQ ID NO: 127; and
a suicide gene product coding sequence or a suicide gene comprising SEQ ID NO: 162.

76. The polynucleotide of claim 74 or 75, components of which are listed in an order from the 5′ to the 3′ in the polynucleotide.

77. The polynucleotide of claim 74 or 75, components of which are listed in an order from the 3′ to the 5′ in the polynucleotide.

78. The polynucleotide of claim 74 or 75, components of which are listed in an order from the 5′ to the 3′ or from the 3′ to the 5′ in the polynucleotide except that one or more of the light chain variable region coding sequence is switched with the heavy chain variable region coding sequence of the same antigen binding sequence.

79. The polynucleotide of claim 74 or 75, components of which are listed in an order from the 5′ to the 3′ or from the 3′ to the 5′ in the polynucleotide except that at least two of the antigen binding sequence coding sequences are switched.

80. The polynucleotide of claim 74 or 75, components of which are listed in an order from the 5′ to the 3′ or from the 3′ to the 5′ in the polynucleotide except that at least two of the antigen binding sequence coding sequences are switched and one or more of the light chain variable region coding sequence is switched with the heavy chain variable region coding sequence of the same antigen binding sequence.

81. The polynucleotide of any one of claims 73 to 80, comprising one or more of the nucleotide sequence as disclosed in any one of Tables 1a, 1b, 1c, 1f, 1g, 2a, 2d, 2e, 2h, 2i, 21, 2m, 2p, 2q, 2t, 2u, 3a, 3b, 3c, or 3d, optionally free a detectable marker.

82. The polynucleotide of any one of claims 73 to 81, comprising one or more of the following sequences or a fragment thereof: nucleotide sequences of P4 to P9, P11E, P12E, P19, P20, P61, P62, or P72, as disclosed herein, optionally free a detectable marker.

83. The polynucleotide of claim 73, comprising any one of: SEQ ID NO: 168 or 169.

84. The polynucleotide of any one of claims 73 to 83, further comprising one or more regulatory sequence directing the expression of the polypeptide or the expression of a fragment of the polypeptide.

85. The polynucleotide of claim 84, wherein the regulatory sequences comprise one or more of the following: a promoter, an intron, an enhancer, or a polyadenylation signal.

86. The polynucleotide of any one of claims 73 to 85, further comprising one or more regulatory sequence directing the replication of the polynucleotide.

87. The polynucleotide of any one of claims 73 to 86, further comprising one or more of internal ribosome entry sites between any two of the following: a Chimeric Antigen Receptor (CAR) coding sequence, an antibody coding sequence, a cytokine coding sequence, or an suicide gene product coding sequence.

88. The polynucleotide of any one of claims 73 to 87, further comprising one or both of: an additional detectable marker and an additional suicide gene optionally an inducible caspase (iCasp) suicide gene.

89. A vector comprising a polynucleotide of any of claims 73 to 88.

90. The vector of claim 89, wherein the vector is a non-viral vector optionally a plasmid, or wherein the vector is a viral vector optionally selected from the group of a retroviral vector, a lentiviral vector, an adenoviral vector, an adeno-associated viral vector, or Herpes viral vector.

91. The vector of any one of claims 89 to 90, further comprising a regulatory sequence directing the expression or the replication of the polynucleotide.

92. A vector system comprising more than one vectors, wherein the vectors encodes
(i) an amino acid sequence of a Chimeric Antigen Receptor (CAR) comprising:
(1) an antigen binding amino acid sequence that recognizes and binds B-cell maturation antigen (BCMA) (anti-BCMA antigen binding sequence),
(2) a hinge domain,
(3) a transmembrane domain, and
(4) an intracellular domain;
(ii) an amino acid sequence of a bispecific antibody comprising
(1) an antigen binding sequence that recognizes and binds NKG2D (anti-NKG2D antigen binding sequence), and
(2) an antigen binding sequence that recognizes and binds G Protein-Coupled Receptor Class C Group 5 Member D (GPRC5D) (anti-GPRC5D antigen binding sequence);
(iii) an optional amino acid sequence of a cytokine selected from IL15 or IL-21; and
(iv) an optional amino acid sequence of a suicide gene product, optionally a truncated epidermal growth factor receptor (tEGFR) or RQR8.

93. The vector system of claim 92, comprising at least two vectors, wherein the first vector encodes
(i) an amino acid sequence of a Chimeric Antigen Receptor (CAR) comprising:
(1) an antigen binding amino acid sequence that recognizes and binds B-cell maturation antigen (BCMA) (anti-BCMA antigen binding sequence),
(2) a hinge domain,
(3) a transmembrane domain, and
(4) an intracellular domain;
(iii) an optional amino acid sequence of a cytokine selected from IL15 or IL-21; and
(iv) an optional amino acid sequence of a suicide gene product, optionally a truncated epidermal growth factor receptor (tEGFR) or RQR8; and
wherein the second vector encodes
(ii) an amino acid sequence of a bispecific antibody comprising
(1) an antigen binding sequence that recognizes and binds NKG2D (anti-NKG2D antigen binding sequence), and
(2) an antigen binding sequence that recognizes and binds G Protein-Coupled Receptor Class C Group 5 Member D (GPRC5D) (anti-GPRC5D antigen binding sequence);
(iii) an optional amino acid sequence of a cytokine selected from IL15 or IL-21; and
(iv) an optional amino acid sequence of a suicide gene product, optionally a truncated epidermal growth factor receptor (tEGFR) or RQR8,
And optionally wherein the suicide gene product of the first vector is different from the suicide gene product of the second vector.

94. An isolated cell comprising one or more of the following: a polypeptide of any one of claims 1 to 72, a polynucleotide of any one of claims 73 to 88, a vector of any one of claims 89 to 91, or a vector system of claim 92 or 93.

95. The isolated cell of claim 94, wherein the cell is a prokaryotic cell or a eukaryotic cell.

96. The isolated cell of claim 94 or 95, wherein the cell is a eukaryotic cell.

97. The isolated cell of claim 95 or 96, wherein the eukaryotic cell is selected from an animal cell, a mammalian cell, a bovine cell, a feline cell, a canine cell, a murine cell, an equine cell, or a human cell.

98. The isolated cell of any one of claims 95 to 97, wherein the eukaryotic cell is an immune cell, optionally a T-cell, a B cell, a NK cell, a NKT cell, a dendritic cell, a myeloid cell, a monocyte, or a macrophage, optionally derived from Hematopoietic stem cells (HSCs) or induced pluripotent stem cells (iPSCs).

99. The isolated cell of any one of claims 95 to 97, wherein the eukaryotic cell is a stem cell, optionally Hematopoietic stem cells (HSCs) or induced pluripotent stem cells (iPSCs).

100. The isolated cell of any one of claims 94 to 99, wherein the isolated cell secretes the bispecific antibody.

101. The isolated cell of any one of claims 94 to 100, wherein the isolated cell secretes or expresses the cytokine.

102. The isolated cell of any one of claims 94 to 101, wherein the isolated cell expresses the CAR.

103. The isolated cell of any one of claims 94 to 102, wherein the isolated cell expresses the detectable marker and suicide gene product.

104. A cell population comprising one or more isolated cell of any one of claims 94-103.

105. The cell population of claim 104, wherein the cell population is homogenous or heterogeneous.

106. The cell population, of claim 104 or 105, wherein the cell population is substantively homogenous.

107. A composition comprising a carrier and at least of the following: a polypeptide of any one of claims 1 to 72, one or more fragment(s) of the polypeptide, a polynucleotide of any one of claims 73 to 88, a vector of any one of claims 89 to 91, or a vector system of claim 92 or 93, an isolated cell of any one of claims 94 to 103, or a cell population of any one of claims 104 to 106.

108. The composition of claim 107, wherein the carrier is a pharmaceutically acceptable carrier.

109. An isolated complex comprising one or more of the following:
a polypeptide of any one of claims 1 to 72 bound to a cancer cell,
an isolated cell of any one of claims 94 to 103 bound to a cancer cell,
a cancer cell bound to an isolated cell of any one of claims 94-103 and a bispecific antibody,
an isolated cell of any one of claims 94-103 bound to a cytokine and a cancer cell; or
a cancer cell bound to a bispecific antibody and an isolated cell of any one of claims 94-103 which is further bound to a cytokine.

110. A method of producing a cell expressing a CAR and a detectable marker and suicide gene product optionally selected from tEGFR or RQR8 and secreting a bispecific antibody and a cytokine, comprising transducing a cell or a population thereof with a polynucleotide of any one of claims 73 to 88 or a vector of any one of claims 89 to 91, or a vector system of claim 92 or 93, or culturing the cell of any one of claims 94-103 or the cell population of any one of claims 104-106.

111. The method of claim 110, wherein the cell is selected from a Hematopoietic stem cell (HSC), an induced pluripotent stem cell (iPSCs), or an immune cell.

112. The method of claim 110 or 111, wherein the cell population comprises one or more of the following: an HSC, an iPSC, or an immune cell.

113. The method of claim 111 or 112, wherein the immune cell is selected from T-cells, B cells, NK cells, dendritic cells, myeloid cells, monocytes, or macrophages.

114. The method of any one of claims 111 to 113, wherein the immune cell is derived from an HSC or an iPSC.

115. A method of inhibiting the growth of a cancer cell expressing one or both of BCMA and GPRCSD or a tissue comprising the cancer cell, comprising contacting the cancer cell or the tissue with an isolated cell of any one of claims 94 to 103 or the cell population of any one of claims 104 to 106.

116. The method of claim 115, wherein the contacting is in vitro, or ex vivo, or in vivo.

117. The method of claim 115 or 116, wherein the contacting is in vivo and the isolated cells are autologous or allogeneic to a subject being treated.

118. The method of claim 115 or 116, wherein the contacting is in vivo and the isolated cells are allogenic to a subject being treated.

119. The method of any one of claims 115 to 118, further comprising contacting the cancer cell or the tissue with an effective amount of a cytoreductive therapy or a therapy that upregulates the expression of one or both of BCMA and GPRCSD on the cancer cell.

120. A method for treating a cancer in a subject in need thereof, comprising administering the isolated cell of any one of claims 94 to 103 or the cell population of any one of claims 104 to 106 to the subject.

121. The method of claim 120, wherein the subject is selected for the therapy by determining expression of one or both of BCMA and GPRCSD in a sample isolated from the subject and administering the cell that expresses a CAR that recognizes and bind the one or both of BCMA and GPRCSD or a population thereof.

122. The method of claim 121, wherein the expression is determined by contacting the sample with one or both of an anti-BCMA antibody or an antigen binding fragment thereof or an anti-GPRCSD antibody or an antigen binding fragment thereof in vitro, or ex vivo, or in vivo and detecting binding between the sample and the antibody or antigen binding fragment thereof.

123. The method of claim 122, wherein the antibody comprises a detectable marker.

124. A method for treating a cancer in a subject selected for the treatment, comprising administering the isolated cell of any one of claims 94 to 103 or the cell population of any one of claims 104 to 106 to the subject.

125. The method of claim 124, wherein the subject is selected via if a cancer cell of the subject expresses one or both of BCMA and GPRCSD.

126. The method of claim 125, wherein the expression is determined by contacting a cancer cell of the subject with one or both of an anti-BCMA antibody or an antigen binding fragment or an anti-GPRCSD antibody or an antigen binding fragment thereof in vitro, or ex vivo, or in vivo.

127. The method of claim 126, wherein the antibody comprises a detectable marker.

128. The method of any one of claims 120 to 127, wherein the isolated cell is autologous or allogeneic to the subject in need.

129. The method of any one of claims 120 to 127 wherein the isolated cell is allogenic to the subject in need.

130. The method of any one of claims 120 to 129, further comprising administering to the subject a cytoreductive therapy or a therapy that upregulates the expression of one or both of BCMA and GPRC5D.

131. The method of any one of claims 120 to 130, wherein the administration is applied to the subject as a first line therapy, or a second line therapy, or third line therapy, or a fourth line therapy.

132. The method of any one of claims 120 to 131, further comprising administering an antibody that recognizes and binds the suicide gene product optionally tEGFR (an anti-tEGFR antibody) or RQR8 (an anti-RQR8 antibody) after the administration of the cells, thereby eliminating suicide gene product expressing cells.

133. The method of claim 132, wherein the administration of the antibody recognizing and binding the suicide gene product is about 4 weeks, or about 1.5 months, or about 2 months, or about 3 months, or about 4 months, or about 5 months, or about 6 months, or about 7 months, or about 8 months, or about 9 months, or about 10 months, or about 11 months, or about 12 months, or about 1.5 years after the administration of the cells.

134. The method of claim 119 or 130, wherein the cytoreductive therapy comprises one or more of the following: a chemotherapy, a cryotherapy, a hyperthermia, a targeted therapy, or a radiation therapy.

135. The method of any one of claims 117 to 134, wherein the subject is a mammal, a canine, a feline, an equine, a murine, or a human patient.

136. The method of any one of claims 115 to 135, wherein the cancer is multiple myeloma (MM).

137. A kit comprising optional instructions for use and one or more of the following: a polypeptide of any one of claims 1 to 72, a polynucleotide of any one of claims 73 to 88, a vector of any one of claims 89 to 91, a vector system of claim 92 or 93, an isolated cell of any one of claims 94 to 103, a cell population of any one of claims 104-106, a composition of claim 107 or 108, or an isolated complex of claim 109.
